(19) United States
(12) Patent Application Publication
(10) Pub. No.: US 2023/0374034 A1
(43) Pub. Date: Nov. 23, 2023

(54) NOVEL BICYCLIC COMPOUNDS

(71) Applicant: PRISM BIOLAB CO., LTD., Kanagawa (JP)

(72) Inventors: Hiroyuki KOUJI, Kanagawa (JP); Atsushi YOSHIMORI, Kanagawa (JP); Takenao ODAGAMI, Osaka (JP); Yoichiro HIROSE, Kanagawa (JP); Masaji KASHAI, Kanagawa (JP); Hajime TAKASHIMA, Kanagawa (JP); Eiji HONDA, Kanagawa (JP)

(73) Assignee: PRISM BIOLAB CO., LTD., Kanagawa (JP)

(21) Appl. No.: 18/030,328

(22) PCT Filed: Oct. 8, 2021
(86) PCT No.: PCT/JP2021/039188

§ 371 Date: Apr. 5, 2023

Related U.S. Application Data
(60) Provisional application No. 63/090,185, filed on Oct. 10, 2020.

(51) Int. Cl. C07D 498/04; C07D 498/20; C07D 519/00

(52) U.S. Cl. CPC C07D 498/04; C07D 498/20; C07D 519/00

(57) ABSTRACT

A compound of the formula (I): wherein each symbol is as defined in the DESCRIPTION, or a pharmaceutically acceptable salt thereof has a superior Notch signal transduction inhibitory action, and is useful for preventing or treating various diseases involving Notch signal transduction.

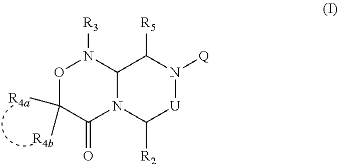

### TECHNICAL FIELD

The present invention relates to a novel bicyclic compound. More specifically, the present invention relates to a novel bicyclic compound having Notch inhibitory action.

### BACKGROUND ART

Notch signaling is an evolutionary conserved pathway that plays an integral role in development and tissue homeostasis in mammals. The Notch receptors and ligands contain single-pass transmembrane domains, are expressed on the cell surface and, for that reason, Notch signaling is particularly important in mediating communication between adjacent cells expressing the receptors and ligands. There are four known Notch receptors found in rodents and humans, termed Notch 1 to Notch 4. The Notch receptors are heterodimeric proteins composed of extracellular and intracellular domains that are initially synthesized as a single polypeptide. Receptor-ligand interaction triggers a series of proteolytic cleavages of the Notch receptor polypeptide in which γ-secretase activity is involved. γ-Secretase activity cleaves Notch intracellular domain from the internal side of the plasma membrane which translocates to the nucleus to form a transcription factor complex. Notch intracellular domain (NICD) is the active form of the protein. Various Notch signaling functions include proliferation, differentiation, apoptosis, angiogenesis, migration and self-renewal (Non-patent documents 1-3).

In addition, NICD activates transcription of the target genes Hes1 and Hes5 by translocation into the nucleus and forming a stable complex with RBP-J and MAML, which are DNA binding proteins.

Therefore, a compound that can inhibit various Notch signal function can be a medicament useful for various diseases involving the function.

### DOCUMENT LIST

### Non-Patent Documents

non-patent document 1: Bray, Nature Reviews Molecular Cell Biology, 7:678-689 (2006).
non-patent document 2: Fortini, Developmental Cell 16:633-647 (2009).
non-patent document 3: Ables, J. L. et al., Neurosci., 12: 269-283 (2011).

### SUMMARY OF THE INVENTION

### Problems to be Solved by the Invention

The present invention aims to provide a compound having a Notch inhibitory action and a medicament containing the compound and useful for various diseases.

### Means of Solving the Problems

The present inventors have conducted intensive studies in an attempt to solve the aforementioned problems and found that a compound having a particular structure shows a superior Notch signal transduction inhibitory action (hereinafter to be also referred to as Notch inhibitor), and completed the present invention.

That is, the present invention relates to the following.
[1] A compound represented by the following formula (I):

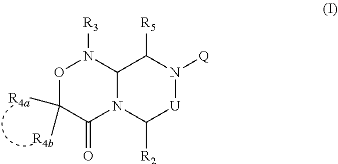

wherein
Q is represented by any of the following formulas (I-1) to (I-2):

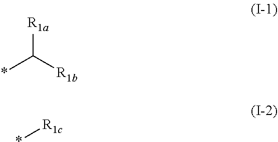

* is a binding site with N (nitrogen atom);
R₁ₐ is hydrogen, optionally substituted alkyl, optionally substituted alkenyl, optionally substituted alkynyl, optionally substituted cycloalkyl, optionally substituted arylalkyl, optionally substituted heteroarylalkyl, optionally substituted cycloalkylalkyl, or optionally substituted heterocycloalkylalkyl;
R_{1b} is hydrogen, optionally substituted alkyl or —W₁₁-W₁₂—R₁₃
wherein
W₁₁ is —(CO)— or —(SO₂)—,
W₁₂ is a bond, —O— or —N(R₁₄)—,
R₁₃ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl,
R₁₄ is hydrogen or optionally substituted alkyl,
R₁₃ and R₁₄ may combine to form a saturated or unsaturated 4 to 7-membered ring, which may contain carbon atom, nitrogen atom, or oxygen atom, and an aryl ring or a heteroaryl ring may be fused to the ring,
a substituent —X₁₅—R₁₅ may be substituted on the formed saturated or unsaturated 4-7 membered ring or on the fused aryl ring or heteroaryl ring,
X₁₅ is —O—, —NH— or single bond,
R₁₅ is hydrogen, optionally substituted alkyl, optionally substituted aryl or optionally substituted heteroaryl; R_{1c} is optionally substituted cycloalkyl, optionally substituted heterocycloalkyl, optionally substituted aryl or optionally substituted heteroaryl;
U is —(CO)— or —CH₂—,
R₂ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ is —W₃₁-W₃₂—R₃₃
wherein
   W₃₁ is —(CO)—, —(SO₂)—, or —CH₂—
   W₃₂ is —O—, —NH—, or single bond, and
   R₃₃ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4b} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐ and R_{4b} optionally form a spiro ring together with a carbon bonded thereto, which optionally has oxygen atom or nitrogen atom in the ring; and
R₅ is hydrogen or optionally substituted alkyl,
or a pharmaceutically acceptable salt thereof;
provided that (3R,6S)isopropyl-3-ethyl-8-isopentyl-6-(2-(methylthio)ethyl)-4,7-dioxohexahydropyrazino[2,1-c][1,2,4]oxadiazine-1(6H)-carboxylate is excluded.
[2] The compound of [1], wherein
R₁ₐ is hydrogen, optionally substituted alkyl, optionally substituted aryl, optionally substituted heteroaryl, optionally substituted cycloalkyl, or optionally substituted arylalkyl;
R_{1b} is hydrogen, optionally substituted alkyl or —W_{11′}-W_{12′}—R_{13′}
wherein
W_{11′} is —(CO)—,
W_{12′} is —NH—, and
R_{13′} is optionally substituted alkyl, or optionally substituted arylalkyl;
R₂ is optionally substituted alkyl, optionally substituted arylalkyl, optionally substituted heteroarylalkyl, or optionally substituted cycloalkylalkyl;
R₃ is —W_{31′}-W_{32′}—R_{33′}
wherein
   W_{31′} is —(CO)—, —(SO₂)—, or —CH₂—,
   W_{32′} is —O—, —NH—, or single bond, and
   R_{33′} is optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted arylalkyl, or optionally substituted cycloalkylalkyl;
R_{4b} is hydrogen or optionally substituted alkyl;
R₄ₐ and R_{4b} optionally form a spiro ring together with a carbon bonded thereto, which optionally has oxygen atom or nitrogen atom in the ring;
R₅ is hydrogen or optionally substituted alkyl;
or a pharmaceutically acceptable salt thereof.
[3] The compound of [1], which is represented by the following formula (Ia):

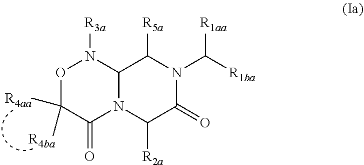

wherein
R₁ₐₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl;
R_{1ba} is hydrogen, optionally substituted alkyl or W₁₁ₐ-W₁₂ₐ—R₁₃ₐ
wherein
W₁₁ₐ is —(CO)— or —(SO₂)—,
W₁₂ₐ is a bond, —O— or —NH—, and
R₁₃ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl;
R₂ₐ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ₐ is —W₃₁ₐ-W₃₂ₐ—R₃₃ₐ
wherein
   W₃₁ₐ is —(CO)— or —(SO₂)—,
   W₃₂ₐ is —O— or —NH—, and
   R₃₃ₐ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4ba} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐₐ and R_{4ba} optionally form a spiro ring together with a carbon bonded thereto; and
R₅ₐ is hydrogen or optionally substituted alkyl,
or a pharmaceutically acceptable salt thereof;
provided that (3R,6S)isopropyl-3-ethyl-8-isopentyl-6-(2-(methylthio)ethyl)-4,7-dioxohexahydropyrazino[2,1-c][1,2,4]oxadiazine-1(6H)-carboxylate is excluded.
[4] The compound of [3], wherein
R₁ₐₐ is hydrogen, optionally substituted alkyl, optionally substituted aryl, optionally substituted heteroaryl, optionally substituted cycloalkyl, or optionally substituted arylalkyl;
R_{1ba} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl, or optionally substituted arylalkyl;
R₂ₐ is optionally substituted alkyl, optionally substituted arylalkyl, or optionally substituted cycloalkylalkyl;
R₁ₐ is —W_{31a′}-W_{32a′}—R_{33a′}
wherein
   W_{31a′} is —(CO)—,
   W_{32a′} is —O— or —NH—, and
   R_{33a′} is optionally substituted alkyl, optionally substituted arylalkyl, optionally substituted heteroarylalkyl, or optionally substituted cycloalkylalkyl;
R₄ₐₐ is optionally substituted alkyl, optionally substituted arylalkyl, or optionally substituted cycloalkylalkyl;
R_{4ba} is hydrogen;
R₅ₐ is hydrogen,
or a pharmaceutically acceptable salt thereof.
[5] The compound of [1] or [2], wherein
R₁ₐ is optionally substituted alkyl, optionally substituted aryl, optionally substituted cycloalkyl, or optionally substituted arylalkyl;
R_{1b} is hydrogen, optionally substituted alkyl or —W_{11′}-W_{12′}—R_{13′}
wherein
W_{11′} is —(CO)—,
W_{12′} is —NH—, and
R_{13′} is optionally substituted alkyl, or optionally substituted arylalkyl,
or a pharmaceutically acceptable salt thereof.
[6] The compound of [3] or [4], wherein
R₁ₐₐ is optionally substituted alkyl, optionally substituted aryl, optionally substituted cycloalkyl, or optionally substituted arylalkyl;
R_{1ba} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl, or optionally substituted arylalkyl,
or a pharmaceutically acceptable salt thereof.
[7] The compound of [1] or [2], wherein
R_{1c} is optionally substituted heterocycloalkyl,
or a pharmaceutically acceptable salt thereof.
[8] The compound of [1] or [2], wherein
U is —(CO)—,
or a pharmaceutically acceptable salt thereof.
[9] The compound of [1] or [2], wherein
R₂ is optionally substituted alkyl, optionally substituted arylalkyl, or optionally substituted cycloalkylalkyl,
or a pharmaceutically acceptable salt thereof.
[10] The compound of [3] or [4], wherein
R₂ₐ is optionally substituted alkyl, optionally substituted arylalkyl, or optionally substituted cycloalkylalkyl,
or a pharmaceutically acceptable salt thereof.
[11] The compound of [1] or [2], wherein
R₃ is —W_{31′}-W_{32′}—R_{33′}
wherein
   W_{31′} is —(CO)— or —CH₂—,
   W_{32′} is —O—, —NH—, or single bond, and
   R_{33′} is optionally substituted alkyl, optionally substituted arylalkyl, optionally substituted cycloalkylalkyl or optionally substituted heterocycloalkylalkyl,
or a pharmaceutically acceptable salt thereof.
[12] The compound of [1] or [2], wherein
R₄ₐ is optionally substituted alkyl, optionally substituted arylalkyl, or optionally substituted cycloalkylalkyl,
R_{4b} is hydrogen, and
R₅ is hydrogen,
or a pharmaceutically acceptable salt thereof.
[13] The compound of [3] or [4], wherein
R₄ₐₐ is optionally substituted alkyl, optionally substituted arylalkyl, or optionally substituted cycloalkylalkyl,
R_{4ba} is hydrogen, and
R₅ₐ is hydrogen,
or a pharmaceutically acceptable salt thereof.
[14] A pharmaceutical composition comprising a compound of any one of [1] to [13] or a pharmaceutically acceptable salt thereof, and optionally a pharmaceutically acceptable carrier or diluent.
[15] A pharmaceutical composition of [14], wherein the composition comprises an effective amount of the compound.
[16] A method of treating or preventing a disease involving Notch signal transduction comprising administering to a subject in need thereof a compound of any one of [1] to [13] or a pharmaceutically acceptable salt thereof, or a composition of
[14] or [15], in an amount effective to treat or prevent the disease.
[17] An agent for treating or preventing a disease involving Notch signal transduction comprising a compound of any one of
[1] to [13] or a pharmaceutically acceptable salt thereof.
[18] A compound according of any one of [1] to [13] or a pharmaceutically acceptable salt thereof, or a composition of
[14] or [15] for the use as a medicament for treating or preventing a disease involving Notch signal transduction.

### Effect of the Invention

The compound of the formula (I) of the present invention inhibits Notch signal transduction and thus can be used for treating various diseases involving Notch signal transduction.

### BRIEF DESCRIPTION OF THE DRAWINGS

FIG. 1 shows a ¹H NMR (400 MHz, CDCl₃) data of D-10.

FIG. 2 shows a ¹H NMR (400 MHz, CDCl₃) data of D-13-Int1.

FIG. 3 shows a ¹H NMR (400 MHz, CDCl₃) data of D-13.

FIG. 4 shows a ¹H NMR (400 MHz, CDCl₃) data of D-8.

FIG. 5 shows a ¹H NMR (400 MHz, CDCl₃) data of D-12.

FIG. 6 shows a ¹H NMR (400 MHz, CDCl₃) data of D-82.

FIG. 7 shows a ¹H NMR (400 MHz, CDCl₃) data of B-128-Int1.

FIG. 8 shows a ¹H NMR (400 MHz, CDCl₃) data of B-128.

FIG. 9 shows a ¹H NMR (400 MHz, CDCl₃) data of B-72.

FIG. 10 shows a ¹H NMR (400 MHz, CDCl₃) data of A-22-Int2.

FIG. 11 shows a ¹H NMR (400 MHz, CDCl₃) data of A-22-Int3.

FIG. 12 shows a 1H NMR (400 MHz, CDCl₃) data of A-22-Int4.

FIG. 13 shows a ¹H NMR (400 MHz, CDCl₃) data of A-22-Int5.

FIG. 14 shows a ¹H NMR (400 MHz, CDCl₃) data of A-22-Int6.

FIG. 15 shows a ¹H NMR (400 MHz, CDCl₃) data of A-22.

FIG. 16 shows a ¹H NMR (400 MHz, CDCl₃) data of A-43-Int3.

FIG. 17 shows a ¹H NMR (400 MHz, DMSO) data of A-43.

FIG. 18 shows a ¹H NMR (400 MHz, CDCl₃) data of A-60-Int2.

FIG. 19 shows a ¹H NMR (400 MHz, DMSO) data of A-60-Int3.

FIG. 20 shows a ¹H NMR (400 MHz, CDCl₃) data of A-60.

FIG. 21 shows a ¹H NMR (400 MHz, DMSO) data of A-52-Int2.

FIG. 22 shows a ¹H NMR (400 MHz, CDCl₃) data of A-52-Int3.

FIG. 23 shows a ¹H NMR (400 MHz, DMSO) data of A-52-Int4.

FIG. 24 shows a ¹H NMR (400 MHz, DMSO) data of A-52-Int5.

FIG. 25 shows a ¹H NMR (400 MHz, DMSO) data of A-52-Int6.

FIG. 26 shows a ¹H NMR (400 MHz, CDCl₃) data of A-52.

FIG. 27 shows a ¹H NMR (400 MHz, CDCl₃) data of I-128.

FIG. 28 shows a ¹H NMR (300 MHz, CDCl₃) data of I-182.

### DESCRIPTION OF EMBODIMENTS

### Definition

Unless otherwise stated, the following terms used in the specification and claims shall have the following meanings for the purposes of this application.

“Lower”, unless indicated otherwise, means that the number of the carbon atoms constituting the given radicals is between one and six.

“Optionally substituted”, unless otherwise stated, means that a given radical may consist of only hydrogen substituents through available valencies or may further comprise one or more non-hydrogen substituents through available valencies. In general, a non-hydrogen substituent may be any substituent that may be bound to an atom of the given radical that is specified to be substituted. Examples of substituents include, but are not limited to, —R₆, —OR₆, —COR₆, —COOR₆, —OCOR₆, —CONR₆R₇, —NR₆R₇, —NR₇COR₆, —NR₇COOR₆, —SR₆, —SO₂R₆, —SO₂NR₆R₇, —SO₂OR₆, —OSO₂R₆, —NHC(NHR₆)NR₇, —NHC(NH₂)NH, —CN, —NO₂, halogen and methylenedioxy, wherein R₆ and R₇ is independently selected from hydrogen, linear or branched chain, cyclic or noncyclic, substituted or unsubstituted, alkyl chain, aryl, heteroaryl, arylalkyl and heteroarylalkyl moieties.

“Halogen” means fluorine, chlorine, bromine or iodine. “Halo” means fluoro, chloro, bromo or iodo.

“Alkyl” means a linear or branched, saturated, aliphatic radical having a chain of carbon atoms. C_{X-Y} alkyl is typically used where X and Y indicate the number of carbon atoms in the chain. The number of carbon atoms in the chain is preferably 1 to 10, more preferably 1 to 6, further preferably 1 to 4. Non-exclusive examples of alkyl include methyl, ethyl, propyl, isopropyl, butyl, sec-butyl, isobutyl, tert-butyl, pentyl, isopentyl, neopentyl, tert-pentyl, hexyl, isohexyl, and the like.

“Alkenyl” means a linear or branched, carbon chain that contains at least one carbon-carbon double bond. C_{X-Y} alkenyl is typically used where X and Y indicate the number of carbon atoms in the chain. The number of carbon atoms in the chain is preferably 2 to 10, more preferably 2 to 6. Non-exclusive examples of alkenyl include ethenyl (vinyl), allyl, isopropenyl, 2-methylallyl, 1-pentenyl, hexenyl, heptenyl, 1-propenyl, 2-butenyl, 2-methyl-2-butenyl, and the like.

“Alkynyl” means a linear or branched, carbon chain that contains at least one carbon-carbon triple bond. C_{X-Y} alkynyl is typically used where X and Y indicate the number of carbon atoms in the chain. The number of carbon atoms in the chain is preferably 2 to 10, more preferably 2 to 6. Non-exclusive examples of alkynyl include ethynyl, propargyl, 3-methyl-1-pentynyl, 2-heptynyl and the like.

“Alkylene”, unless indicated otherwise, means a linear or branched, saturated, aliphatic, polyvalent carbon chain. C_{X-Y} alkylene is typically used where X and Y indicate the number of carbon atoms in the chain. The number of carbon atoms in the chain is preferably 1 to 10, more preferably 1 to 6. Non-exclusive examples of alkylene include methylene (—CH₂—), ethylene (—CH₂CH₂—), methylmethylene (—CH(CH₃)—), 1,2-propylene (—CH₂CH(CH₃)—), 1,3-propylene (—CH₂CH₂CH₂—), 1,2-butylene (—CH₂CH(CH₂CH₃)—), 1,3-butylene (—CH₂CH₂CH(CH₃)—), 1,4-butylene (—CH₂CH₂CH₂CH₂—), 2-methyltetramethylene (—CH₂CH(CH₃)CH₂CH₂—), pentamethylene (—CH₂CH₂CH₂CH₂CH₂—), 1,2,3-propanetriyl, 1,3,3-propanetriyl and the like.

“Heteroatom” refers to an atom that is not a carbon atom and hydrogen atom. Particular examples of heteroatoms include, but are not limited to nitrogen, oxygen, and sulfur.

“Aryl” means a monocyclic or polycyclic radical wherein each ring is aromatic or when fused with one or more rings forms an aromatic ring. C_{X-Y} aryl is typically used where X and Y indicate the number of carbon atoms in the ring assembly. The number of carbon atoms in the ring is preferably 6 to 14, more preferably 6 to 10. Non-exclusive examples of aryl include phenyl, naphthyl, indenyl, azulenyl, biphenyl, fluorenyl, anthracenyl, phenalenyl and the like. “Aryl” may partially be hydrogenated. Non-exclusive examples of partially hydrogenated aryl include tetrahydronaphthyl, indanyl and the like.

“Heteroaryl” means a monocyclic or polycyclic aromatic radical wherein at least one ring atom is a heteroatom and the remaining ring atoms are carbon. “X-Y membered heteroaryl” is typically used where X and Y indicate the number of carbon atoms and heteroatoms in the ring assembly. The number of carbon atoms and heteroatoms in the ring is preferably 5 to 14, more preferably 5 to 10. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternerized and the sulfur atoms can be optionally oxidized. Non-exclusive examples of monocyclic heteroaryl group of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrrole, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, and another monocyclic heteroaryl or heterocycloalkyl ring. Non-exclusive examples of bicyclic or tricyclic heteroaryl include, but are not limited to, those derived from benzofuran (ex. benzo[b]furan), benzothiophene (ex. benzo[b]thiophene), benzimidazole, benzotriazine (ex. benzo[e][1,2,4]triazine, benzo[d][1,2,3]triazine), pyridopyrimidine (ex. pyrido[4,3-d]pyrimidine, pyrido[3,4-d]pyrimidine, pyrido[3,2-d]pyrimidine, pyrido[2,3-d]pyrimidine), pyridopyrazine (ex. pyrido[3,4-b]pyrazine, pyrido[2,3-b]pyrazine), pyridopyridazine (ex. pyrido[2,3-c]pyridazine, pyrido[3,4-c]pyridazine, pyrido[4,3-c]pyridazine, pyrido[3,2-c]pyridazine), pyridotriazine (ex. pyrido[2,3-d][1,2,3]triazine, pyrido[3,4-d][1,2,3]triazine, pyrido[4,3-d][1,2,3]triazine, pyrido[3,2-d][1,2,3]triazine, pyrido[3,4-e][1,2,4]triazine, pyrido[3,2-e][1,2,4]triazine), benzothiadiazole (ex. benzo[c][1,2,5]thiadiazole), furopyridine (ex. furo[3,2-b]pyridine, furo[3,2-c]pyridine, furo[2,3-c]pyridine, furo[2,3-b]pyridine), oxazolopyridine (ex. oxazolo[4,5-b]pyridine, oxazolo[4,5-c]pyridine, oxazolo[5,4-c]pyridine, oxazolo[5,4-b]pyridine), thiazolopyridine (ex. thiazolo[4,5-b]pyridine, thiazolo[4,5-c]pyridine, thiazolo[5,4-c]pyridine, thiazolo[5,4-b]pyridine), imidazopyridine (ex. imidazo[1,2-a]pyridine, imidazo[4,5-c]pyridine, imidazo[1,5-a]pyridine), quinazoline, thienopyridine (ex. thieno[2,3-c]pyridine, thieno[3,2-b]pyridine, thieno[2,3-b]pyridine), indolizine, quinoline, isoquinoline, phthalazine, quinoxaline, cinnoline, naphthyridine, quinolizine, indole, isoindole, indazole, indoline, benzoxazole, benzopyrazole, benzothiazole, pyrazolopyridine (ex. pyrazolo[1,5-a]pyridine), imidazopyrimidine (ex. imidazo[1,2-a]pyrimidine, imidazo[1,2-c]pyrimidine, imidazo[1,5-a]pyrimidine, imidazo[1,5-c]pyrimidine), pyrrolopyridine (ex. pyrrolo[2,3-b]pyridine, pyrrolo[2,3-c]pyridine, pyrrolo[3,2-c]pyridine, pyrrolo[3,2-b]pyridine), pyrrolopyrimidine (ex. pyrrolo[2,3-d]pyrimidine, pyrrolo[3,2-d]pyrimidine, pyrrolo[1,2-c]pyrimidine, pyrrolo[1,2-a]pyrimidine), pyrrolopyrazine (ex. pyrrolo[2,3-b]pyrazine, pyrrolo[1,2-a]pyrazine), pyrrolopyridazine (ex. pyrrolo[1,2-b]pyridazine), triazopyridine (ex. triazo[1,5-a]pyridine), pteridine, purine, carbazole, acridine, perimidine, 1,10-phenenthroline, phenoxathiin, phenoxazine, phenothiazine, phenazine and the like. The bicyclic or tricyclic heteroaryl rings can be attached to the parent molecule through either the heteroaryl group itself or the aryl, cycloalkyl, or heterocycloalkyl group to which it is fused.

“Cycloalkyl” means a non-aromatic, saturated or partially unsaturated, monocyclic, fused bicyclic or bridged polycyclic ring radical. C_{X-Y} cycloalkyl is typically used where X and Y indicate the number of carbon atoms in the ring assembly. The number of carbon atoms in the ring is preferably 3 to 10, more preferably 3 to 8. Non-exclusive examples of cycloalkyl include cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, bicyclo[2.2.1]hept-1-yl, and the like.

“Heterocycloalkyl” means cycloalkyl, as defined in this application, provided that one or more of the atoms forming the ring is a heteroatom selected, independently from N, O, or S. C_{X-Y} heterocycloalkyl is typically used where X and Y indicate the number of carbon atoms and heteroatoms in the ring assembly. The number of carbon atoms and heteroatoms in the ring is preferably 3 to 10, more preferably 3 to 8. Non-exclusive examples of heterocycloalkyl include piperidyl, 4-morpholyl, 4-piperazinyl, pyrrolidinyl, perhydropyrrolidinyl, 1,4-diazaperhydroepinyl, 1,3-dioxanyl, 1,4-dioxanyl, and the like.

Moreover, the above-mentioned definitions can apply to groups wherein the above-mentioned substituents are connected. For example, “arylalkyl” means linear or branched alkyl group which is substituted by one or more aryl groups, such as benzyl, 1-phenylethyl, 2-phenylethyl, 3-phenylpropyl, 1-naphthylmethyl, 2-naphthylmethyl and the like. “Heteroarylalkyl” means linear or branched alkyl group which is substituted by one or more heteroaryl groups.

“Cycloalkylalkyl” means linear or branched alkyl group which is substituted by one or more cycloalkyl group (e.g., cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, bicyclo[2.2.1]hept-1-yl). “Heterocycloalkylalkyl” means linear or branched alkyl group which is substituted by one or more heterocycloalkyl groups.

“Monocyclic ring” as used herein refers to a monocyclic, saturated or unsaturated carbocyclic ring or a monocyclic, saturated or unsaturated heterocyclic ring. “X-membered monocyclic ring” is typically used where X indicate the number of carbon atoms and heteroatoms in the ring assembly. The number of carbon atoms and heteroatoms in the ring is preferably 4 to 7, more preferably 5 or 6. “Monocyclic heterocyclic ring” means a monocyclic, aromatic or nonaromatic ring wherein at least one ring atom is a heteroatom (preferably S, N or O) and the remaining ring atoms are carbon. The nitrogen atoms can be optionally quaternerized and the sulfur atoms can be optionally oxidized.

Non-exclusive examples of monocyclic saturated carbocyclic ring include cyclopropane, cyclobutane, cyclopentane, cyclohexane, cycloheptane and the like.

Non-exclusive examples of monocyclic unsaturated carbocyclic ring include cyclopropene, cyclobutene, cyclopentene, cyclohexene, cycloheptene, cyclopentadiene, benzene, and the like.

Non-exclusive examples of monocyclic saturated heterocyclic ring include pyrrolidine, piperidine, morpholine, piperazine, 1,3-dioxane, 1,4-dioxane and the like.

Non-exclusive examples of monocyclic unsaturated heterocyclic ring include pyrazole, dihydro-pyrrole, pyrrole, dihydro-pyrazole, imidazole, thiophene, thiazole, isothiazole, thiadiazole, furan, oxazole, isoxazole, oxadiazole, pyridine, pyridazine, pyrimidine, pyrazine, triazine and the like.

“Spiro ring” as used herein refers to saturated or unsaturated cycloalkane or saturated or unsaturated heterocycloalkane.

“Cycloalkane” means a non-aromatic, saturated or partially unsaturated, monocyclic, fused bicyclic or bridged polycyclic ring. C_{X-Y} cycloalkane is typically used where X and Y indicate the number of carbon atoms in the ring assembly. The number of carbon atoms in the ring is preferably 3 to 10, more preferably 3 to 8. Non-exclusive examples of cycloalkane include cyclopropane, cyclobutane, cyclopentane, cyclohexane, cycloheptane, cyclooctane and the like.

“Heterocycloalkane” means cycloalkane, as defined in this application, provided that one or more of the atoms forming the ring is a heteroatom selected, independently from N, O, and S. C_{X-Y} heterocycloalkane is typically used where X and Y indicate the number of carbon atoms and heteroatoms in the ring assembly. The number of carbon atoms and heteroatoms in the ring is preferably 3 to 10, more preferably 3 to 8. Non-exclusive examples of heterocycloalkane include piperidine, morpholine, piperazine, pyrrolidine, perhydropyrrolizine, tetrahydrofuran, tetrahydropyran, 1,3-dioxane, 1,4-dioxane and the like.

“A saturated or unsaturated 4 to 7-membered ring” means 4 to 7-membered one among the above-mentioned monocyclic ring.

“Protected derivatives” means derivatives of compound in which a reactive site or sites are blocked with protecting groups. A comprehensive list of suitable protecting groups can be found in T. W. Greene, Protecting Groups in Organic Synthesis, 5th edition, John Wiley&Sons, Inc. 2014.

“Isomers” mean any compound having identical molecular formulas but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992). The compounds of the present invention may include these isomers.

“Animal” includes humans, non-human mammals (e.g., mice, rats, dogs, cats, rabbits, cattle, horses, sheep, goats, swine, deer, and the like) and non-mammals (e.g., birds, and the like).

“Disease” specifically includes any unhealthy condition of an animal or part thereof and includes an unhealthy condition that may be caused by, or incident to, medical or veterinary therapy applied to that animal, i.e., the “side effects” of such therapy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salt” or “salt” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, trifluoroacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Amount effective to treat” means that amount which, when administered to an animal for treating a disease, is sufficient to effect such treatment for the disease.

“Amount effective to prevent” means that amount which, when administered to an animal for preventing a disease, is sufficient to effect such prophylaxis for the disease.

“Effective amount” equals to “amount effective to treat” and “amount effective to prevent”.

“Treatment” or “treat” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included.

In the present invention, a compound represented by the following formula (I):

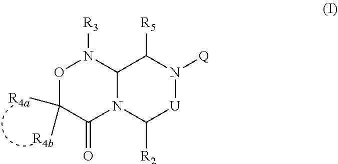

wherein
Q is represented by any of the following formulas (I-1) to (I-2):

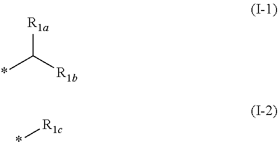

* is a binding site with N (nitrogen atom);
R₁ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl;
R_{1b} is hydrogen, optionally substituted alkyl or —W₁₁-W₁₂—R₁₃
wherein
W₁₁ is —(CO)— or —(SO₂)—,
W₁₂ is a bond, —O— or —N(R₁₄)—,
R₁₃ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl,
R₁₄ is hydrogen or optionally substituted alkyl,
R₁₃ and R₁₄ may combine to form a saturated or unsaturated 4 to 7-membered ring, which may contain carbon atom, nitrogen atom, or oxygen atom, and an aryl ring or a heteroaryl ring may be fused to the ring,
a substituent —X₁₅—R₁₅ may be substituted on the formed saturated or unsaturated 4-7 membered ring or on the fused aryl ring or heteroaryl ring,
X₁₅ is —O—, —NH— or single bond,
R₁₅ is hydrogen, optionally substituted alkyl, optionally substituted aryl or optionally substituted heteroaryl;
R_{1c} is optionally substituted cycloalkyl, optionally substituted heterocycloalkyl, optionally substituted aryl or optionally substituted heteroaryl;
U is —(CO)— or —CH₂—,
R₂ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ is —W₃₁-W₃₂—R₃₃
wherein
   W₃₁ is —(CO)—, —(SO₂)—, or —CH₂—
   W₃₂ is —O—, —NH—, or single bond, and
   R₃₃ is hydrogen, optionally substituted alkyl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4b} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐ and R_{4b} optionally form a spiro ring together with a carbon bonded thereto, which optionally has oxygen atom or nitrogen atom in the ring; and
R₅ is hydrogen or optionally substituted alkyl,
or a pharmaceutically acceptable salt thereof
is disclosed.

In another embodiment of the formula (I), a compound having the following formula (Ia):

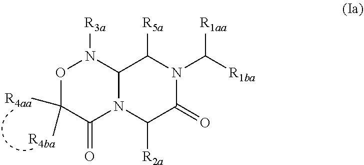

wherein
R₁ₐₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl;
R_{1ba} is hydrogen, optionally substituted alkyl or —W₁₁ₐ-W₁₂ₐ—R₁₃ₐ
wherein
W₁₁ₐ is —(CO)— or —(SO₂)—,
W₁₂ₐ is a bond, —O— or —NH—, and
R₁₃ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl;
R₂ₐ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ₐ is —W₃₁ₐ-W₃₂ₐ—R₃₃ₐ
wherein
   W₃₁ₐ is —(CO)— or —(SO₂)—,
   W₃₂ₐ is —O— or —NH—, and
   R₃₃ₐ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4ba} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐₐ and R_{4ba} optionally form a spiro ring together with a carbon bonded thereto; and
R₅ₐ is hydrogen or optionally substituted alkyl,
or a pharmaceutically acceptable salt thereof
is disclosed.

In one embodiment of the formula (I) or (Ia), R₁ₐ (R₁ₐₐ in the case of formula (Ia)) is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

In another embodiment of the formula (I) or (Ia), R₁ₐ (R₁ₐₐ in the case of formula (Ia)) is hydrogen, optionally substituted alkyl (e.g., butyl, propyl, methyl, CH₂CONH₂, CH₂OH, 2,2-diphenylethyl, naphthylmethyl, CH₂COOH, phenylethyl), optionally substituted aryl (e.g., naphthyl, phenyl), optionally substituted heteroaryl (e.g., pyridyl), optionally substituted cycloalkyl (e.g., cyclohexyl), optionally substituted arylalkyl (e.g., benzyl, hydroxybenzyl), optionally substituted heteroarylalkyl, or optionally substituted cycloalkylalkyl.

In one embodiment of the formula (I), R_{1b} is hydrogen, optionally substituted alkyl or —W₁₁-W₁₂—R₁₃
wherein
W₁₁ is —(CO)— or —(SO₂)—,
W₁₂ is a bond, —O— or —N(R₁₄)—,
R₁₃ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl,
R₁₄ is hydrogen or optionally substituted alkyl,
R₁₃ and R₁₄ may combine to form a saturated or unsaturated 4 to 7-membered ring, which may contain carbon atom, nitrogen atom, or oxygen atom, and an aryl ring or a heteroaryl ring may be fused to the ring,
a substituent —X₁₅—R₁₅ may be substituted on the formed saturated or unsaturated 4-7 membered ring or on the fused aryl ring or heteroaryl ring,
X₁₅ is —O—, —NH— or single bond,
R₁₅ is hydrogen, optionally substituted alkyl, optionally substituted aryl or optionally substituted heteroaryl.

In one embodiment of the formula (Ia), R_{1ba} is hydrogen, optionally substituted alkyl or W₁₁ₐ-W₁₂ₐ—R₁₃ₐ
wherein
W₁₁ₐ is —(CO)— or —(SO₂)—,
W₁₂ₐ is a bond, —O— or —NH—, and
R₁₃ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

Examples of the saturated or unsaturated 4 to 7-membered ring, which may contain carbon atom, nitrogen atom, or oxygen atom include a hydrocarbon ring such as benzene, tropilidene, cyclopentane, cyclohexane, cycloheptane, 1-cyclopentene, 2-cyclopentene, 3-cyclopentene, 1-cyclohexene, 2-cyclohexene, 3-cyclohexene, 1-cycloheptene, 2-cycloheptene, 3-cycloheptene, 2.4-cycloheptadiene, etc. and a heterocyclic ring such as pyridine, pyrazine, pyrimidine, imidazole, furan, thiophene, dihydropyridine, diazepine, oxazepine, pyrrolidine, piperidine, hexamethyleneimine, heptamethyleneimine, tetrahydrofuran, piperazine, homopiperazine, tetrahydroxazepine, morpholine, thiomorpholine, pyrrole, pyrazole, 1,2,3-triazole, oxazole, oxazolidine, thiazole, thiazolidine, isoxazole, imidazoline, triazole, thiadiazole, oxadiazole, oxathiadiazole, triazine, etc. and the like.

In another embodiment of the formula (I), R_{1b} is hydrogen, optionally substituted alkyl or —W_{11′}-W_{12′}—R_{13′}
wherein
W_{11′} is —(CO)—,
W_{12′} is —NH—, and
R_{13′} is optionally substituted alkyl (e.g., —(CH₂)₂OH, —(CH₂)₃OH, pentyl, hexyl, —(CH₂)₂CONH₂, —(CH₂)₃CONH₂), optionally substituted cycloalkyl, optionally substituted arylalkyl (e.g., —(CH₂)₂Ph, —(CH₂)₃Ph, —(CH₂)₃Ph(OH)), optionally substituted heteroarylalkyl, optionally substituted cycloalkylalkyl or optionally substituted heterocycloalkylalkyl. In another embodiment of the formula (Ia), R_{1ba} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl (e.g., —(CH₂)₂OH, —(CH₂)₃OH, pentyl, hexyl, —(CH₂)₂CONH₂, —(CH₂)₃CONH₂), optionally substituted cycloalkyl, optionally substituted arylalkyl (e.g., —(CH₂)₂Ph, —(CH₂)₃Ph, —(CH₂)₃Ph(OH)), optionally substituted heteroarylalkyl, optionally substituted cycloalkylalkyl or optionally substituted heterocycloalkylalkyl. In another embodiment of the formula (I) or (Ia), R₂ (R₂ₐ in the case of formula (Ia)) is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

In another embodiment of the formula (I) or (Ia), R₂ (R₂ₐ in the case of formula (Ia)) is hydrogen, optionally substituted alkyl (e.g., methyl, propyl, butyl, —(CH₂)₄NH₂, —CH₂OH, —CH₂CONH₂, —CH₂CH₂CONH₂, —CH₂COOH, guanidinopropyl), optionally substituted arylalkyl (e.g., benzyl, hydroxybenzyl), optionally substituted heteroarylalkyl, optionally substituted cycloalkylalkyl (e.g., cyclohexylmethyl).

In another embodiment of the formula (I), R₃ is —W₃₁-W₃₂—R₃₃
wherein
   W₃₁ is —(CO)—, —(SO₂)—, or —CH₂—
   W₃₂ is —O—, —NH—, or single bond, and
   R₃₃ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl.

In another embodiment of the formula (Ia), R₃ₐ is —W₃₁ₐ-W₃₂ₐ—R₃₃ₐ
wherein
   W₃₁ₐ is —(CO)— or —(SO₂)—,
   W₃₂ₐ is —O— or —NH—, and
   R₃₃ₐ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted aryl and optionally substituted heteroaryl include biphenyl, phenyl, pyridyl, pyrimidyl, pyridazinyl, pyrazinyl, triazinyl, pyrrolyl, thienyl, furyl, thiazolyl, oxazolyl, imidazolyl, tetrahydronaphthyl, naphthyl, quinolinyl, isoquinolinyl, quinazolinyl, quinoxalinyl, cinnolinyl, naphthyridinyl, benzotriazinyl, indenyl, pyridopyrimidinyl, pyridopyrazinyl, pyridopyridazinyl, pyridotriazinyl, benzofuryl, benzothienyl, indolyl, indazolyl, benzoxazolyl, benzimidazolyl, benzothiazolyl, benzothiadiazolyl, furopyridinyl, thienopyridinyl, pyrropyridinyl, oxazolopyridinyl, thiazolopyridinyl, imidazopyridinyl and the like.

Examples of optionally substituted cycloalkyl include cyclopropyl, cyclobutyl, cyclopentyl, cyclohexyl, cycloheptyl, adamantyl and the like.

Examples of optionally substituted heterocycloalkyl include piperidyl, 4-morpholyl, 4-piperazinyl, pyrrolidinyl, perhydropyrrolidinyl, 1,3-dioxanyl, 1,4-dioxanyl, tetrazolyl, and the like.

Examples of the optionally substituted arylalkyl group include unsubstituted arylalkyl or arylalkyl having an alkyl group such as benzyl, α-methylbenzyl, phenethyl, α-methyiphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

In another embodiment of the formula (I), R₃ is —W_{31′}-W_{32′}—R_{33′}
wherein
   W_{31′} is —(CO)—, —(SO₂)—, or —CH₂—,
   W_{32′} is —O—, —NH—, or single bond, and
   R_{33′} is optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl.

In another embodiment of the formula (Ia), R₃ₐ is —W_{31′}-W_{32′}—R_{33′}
wherein
   W_{31′} is —(CO)—,
   W_{32′} is —O— or —NH—, and
   R_{33′} is optionally substituted alkyl (e.g., methyl, propyl, butyl, pentyl, hexyl, diphenylpropyl), optionally substituted arylalkyl (e.g., benzyl, naphthylmethyl, hydroxyphenylethyl, phenylethyl), optionally substituted heteroarylalkyl (e.g., pyridinylmethyl), optionally substituted cycloalkylalkyl (e.g., cyclohexylmethyl) or optionally substituted heterocycloalkylalkyl.

In one embodiment of the formula (I) or (Ia), R₄ₐ (R₄ₐₐ in the case of formula (Ia)) is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-benzothienylmethyl, 5-benzothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

In another embodiment of the formula (I) or (Ia), R₄ₐ (R₄ₐ in the case of formula (Ia)) is optionally substituted alkyl (e.g., methyl, ethyl, propyl, butyl, guanidinopropyl, —CH₂OH, —CH₂CONH₂, —CH₂CH₂CONH₂), optionally substituted arylalkyl (e.g., benzyl, hydroxybenzyl), optionally substituted cycloalkylalkyl (e.g., cyclohexylmethyl).

In one embodiment of the formula (I) or (Ia), R_{4b} (R_{4ba} in the case of formula (Ia)) is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

Examples of optionally substituted alkenyl group including ethenyl, allyl, 1-propenyl, 2-methylallyl and the like.

Examples of optionally substituted alkynyl group include ethynyl, 1-propynyl, and the like.

Examples of the optionally substituted arylalkyl group include unsubstituted arylalkyl or arylalkyl having an alkyl group such as benzyl, α-methylbenzyl, phenethyl, α-methylphenethyl, α,α-dimethylbenzyl, α,α-dimethylphenethyl, 4-methylphenethyl, 4-methylbenzyl, 4-isopropylbenzyl and the like; arylalkyl having an aryl group or an arylalkyl group such as 4-benzylbenzyl, 4-phenethylbenzyl, 4-phenylbenzyl and the like; arylalkyl having a substituted oxy group such as 4-methoxybenzyl, 4-n-tetradecyloxybenzyl, 4-n-heptadecyloxybenzyl, 3,4-dimethoxybenzyl, 4-methoxymethylbenzyl, 4-vinyloxymethylbenzyl, 4-benzyloxybenzyl, 4-phenethyloxybenzyl and the like; arylalkyl having a hydroxyl group such as 4-hydroxybenzyl, 4-hydroxy-3-methoxybenzyl and the like; arylalkyl having a halogen atom such as 4-fluorobenzyl, 3-chlorobenzyl, 3,4-dichlorobenzyl and the like; 2-furfuryl, diphenylmethyl, 1-naphthylmethyl, 2-naphthylmethyl and the like.

Examples of the optionally substituted heteroarylalkyl group include 2-pyridylmethyl, 3-pyridylmethyl, 2-pyrimidinylmethyl, 5-pyrimidinylmethyl, 3-pyridazinylmethyl, 2-indolylmethyl, 5-indolylmethyl, 2-benzofuranylmethyl, 5-indolylmethyl, 2-ben zothienylmethyl, 5-ben zothienylmethyl, 6-fluoro-2-benzofuranylmethyl, 6-chloro-2-benzofuranylmethyl, 6-methoxy-2-benzofuranylmethyl, 6-fluoro-2-benzothienylmethyl, 6-chloro-2-benzothienylmethyl, 6-methoxy-2-benzothienylmethyl and 6-phenyl-3-pyridazinylmethyl and the like.

Examples of the optionally substituted cycloalkylalkyl group include cyclopropylmethyl, fluorocyclopropylmethyl, chlorocyclopropylmethyl, bromocyclopropylmethyl, iodocyclopropylmethyl, methylcyclopropylmethyl, 1,1-dimethylcyclopropylmethyl, 1,2-dimethylcyclopropylmethyl, hydroxycyclopropylmethyl, methoxycyclopropylmethyl, ethoxycyclopropylmethyl, methoxycarbonylcyclopropylmethyl, methylcarbamoylcyclopropylmethyl, cyclopropylethyl, cyclohexylmethyl, cyclopropylhexyl and the like.

Examples of the optionally substituted heterocycloalkylalkyl group include (2-tetrahydrofuryl)methyl, (2-tetrahydrothiofuranyl)methyl and the like.

In another embodiment of the formula (I), R_{4b} is hydrogen or optionally substituted alkyl (e.g., methyl).

In another embodiment of the formula (Ia), R_{4ba} is hydrogen.

In another embodiment of the formula (I), R₄ₐ and R_{4b} may form a spiro ring together with a carbon bonded thereto. The Spiro ring that may be formed by R₄ₐ and R_{4b} together with a carbon bonded thereto is, for example, cyclopropane or the like.

The Spiro ring may contain oxygen atom or nitrogen atom in the ring.

In one embodiment of the formula (I) or (Ia), R₅ (R₅ₐ in the case of formula (Ia)) is hydrogen or optionally substituted alkyl.

Examples of optionally substituted alkyl group include methyl, ethyl, propyl, isopropyl, butyl, isobutyl, sec-butyl, tert-butyl, pentyl, isopentyl, neopentyl, tert-pentyl, aminomethyl, aminoethyl, aminopropyl, aminobutyl, carboxymethyl, carboxyethyl, carboxypropyl, carboxybutyl, carbamoylmethyl, carbamoylethyl, carbamoylpropyl, carbamoylbutyl, methoxymethyl, methoxyethyl, methoxypropyl, methoxybutyl, methylthiomethyl, methylthioethyl, methylthiopropyl, methylthiobutyl, hydroxymethyl, hydroxyethyl, hydroxypropyl, hydroxybutyl, ethoxycarbonylmethyl, ethoxycarbonylethyl, benzyloxymethyl, benzyloxyethyl, benzyloxypropyl, benzyloxybutyl, guanidinomethyl, guanidinoethyl, guanidinopropyl and the like.

In another embodiment of the formula (I) or (Ia), R₅ (R₅ₐ in the case of formula (Ia)) is hydrogen.

A preferred embodiment of the formula (I) is a compound having the following formula (II) or a pharmaceutically acceptable salt thereof:

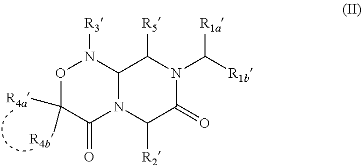

wherein R_{1a′} is hydrogen, optionally substituted alkyl, optionally substituted aryl, optionally substituted heteroaryl, optionally substituted cycloalkyl, or optionally substituted arylalkyl;
R_{1b′} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl, or optionally substituted arylalkyl;
R_{2′} is optionally substituted alkyl, optionally substituted arylalkyl, or optionally substituted cycloalkylalkyl;
R_{3′} is —W_{31a′}-W_{32a′}—R_{33a′}
wherein
   W_{31a′} is —(CO)—,
   W_{32a′} is —O— or —NH—, and
   R_{33a′} is optionally substituted alkyl, optionally substituted arylalkyl, optionally substituted heteroarylalkyl, or optionally substituted cycloalkylalkyl;
R_{4a′} is optionally substituted alkyl, optionally substituted arylalkyl, or optionally substituted cycloalkylalkyl;
R_{4b′} is hydrogen; and
R_{5′} is hydrogen.

In the following, a compound having the formula (I), (Ia) or the formula (II) is to be also referred to as “the compound of the present invention”.

The general synthesis of the compound of the present invention is described in the following “Production Method”. Abbreviations used in the Production Method and Examples are as follows.
AcOH: acetic acid
Boc: tert-butoxycarbonyl
t-BuOH: tert-butanol
Cbz: benzyloxycarbonyl
CDCl₃: deuterated chloroform
CIP: 2-chloro-1,3-dimethylimidazolinium hexafluorophosphate
DBU: 1,8-diazabicyclo[5.4.0]undec-7-ene
DCE: 1 2-dichloroethane
DCM: dichloromethane
DIAD: diisopropyl azodicarboxylate
DIBAL: diisobutylaluminium
DIC: N,N′-methanediylidenebis[1-methylethanamine]
DIEA: N,N-diisopropylethylamine
DIPEA: N-ethyl-N-isopropyl-propan-2-amine
DMF: N,N-dimethylformamide
DMSO: dimethyl sulfoxide
DMT-MM: 4-(4,6-dimethoxy-1,3,5-triazin-2-yl)-4-methylmorpholinium chloride
EtO(OEt): ethoxy
EtOAc: ethyl acetate
EtOH: ethanol
Fmoc: 9-fluorenylmethyloxycarbonyl
HATU: 1-[bis(dimethylamino)methylene]-1H-1,2,3-triazolo[4,5-b]pyridinium 3-oxide hexafluorophosphate
NeON: acetonitrile
MeO(OMe): methoxy
NeOH: methanol
NHPI: N-Hydroxyphthalimide
NMM: N-methylmorpholine
OAc(AcO): acetoxy
Pbf: 2,2,4,6,7-Pentamethyldihydrobenzofuran-5-sulfonyl
PE:EA: Petroleum ether:Ethyl acetate
PG: amino-protecting group
Ph: phenyl
PhthN: N-Phthalimidyl
PPh3: triphenylphosphine
rt: room temperature
TBS: tert-butyldimethylsilyl
TBSCl: tert-Butyl(chloro)dimethylsilane
tBu: tert-butyl
THF: tetrahydrofuran
TLC: Thin-layer chromatography
Trt: trityl

### Production Method

### 1) Production Method 1

In the case of wherein U is —(CO)—;

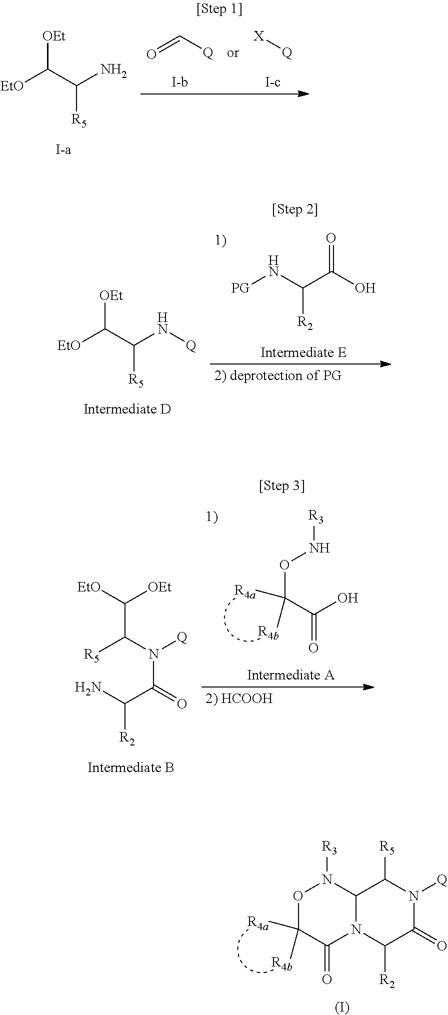

### [Step 1] Synthesis of Intermediate D

Intermediate D wherein Q and R₅ are as defined above can be synthesized according to a known method such as reductive alkylation using a compound represented by the formula I-a wherein R₅ is as defined above, and an aldehyde compound represented by the formula I-b wherein Q are as defined above, or a substitution reaction using a compound represented by the formula I-c having Q and leaving group X, wherein Q are as defined above. The “leaving group” means an atom or an atomic group that is released from an organic compound that undergoes a reaction in an elimination reaction or substitution reaction. Examples of the leaving group include, but are not limited to, halogen group, methanesulfonyloxy group, trifluoromethanesulfonyloxy group and toluenesulfonyloxy group.

In addition, Intermediate D can also be synthesized by a known method such as reductive alkylation shown below or substitution reaction using leaving group X.

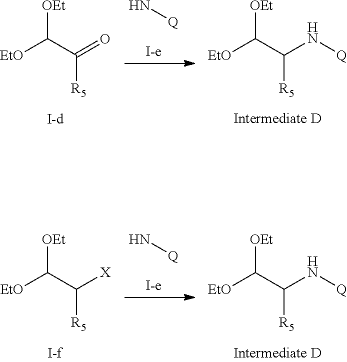

In each of the formulas of the compounds represented by the formulas I-d, I-e and I-f, Q and R₅ are as defined above, and X is a leaving group.

### [Step 2] Synthesis of Intermediate B

Intermediate B can be synthesized by an amidation condensation reaction of Intermediate D and a carboxylic acid derivative of Intermediate E wherein R₂ is as defined above, and PG is an amino-protecting group, followed by a deprotection reaction. In the amidation condensation reaction, generally known amidation reagents and conditions can be applied. HATU and DMT-MM are preferable as the condensing agent, DMF, MeOH, THF and the like are preferable as the solvent, and the reaction temperature is preferably from 0° C. to the boiling point of the solvent. Examples of the amino-protecting group include benzyloxycarbonyl (Cbz), tert-butoxycarbonyl (Boc), tert-pentyloxycarbonyl, isobornyloxycarbonyl, 4-methoxybenzyloxycarbonyl, benzyl chloroformate (Cl-Z), benzyl bromoformate (Br-Z), adamantyloxycarbonyl, trifluoroacetyl, phthaloyl, formyl, 2-nitrophenylsulphenyl, diphenylphosphinothioyl, 9-fluorenylmethyloxycarbonyl (Fmoc), trityl (Trt) and the like. In the deprotection reaction, a generally known reaction can be applied. When PG in the formula is an Fmoc group, a deprotection reaction using piperidine or DBU as a deprotecting agent in ethyl acetate, THF, MeOH or dichloromethane as a solvent is preferable. When PG is a Cbz group, a deprotection reaction using a palladium catalyst such as Pd(OH)₂, Pd/C or the like in methanol, ethanol or THF as a solvent under H₂ atmosphere is preferable. The reaction temperature is preferably 0° C. to the boiling point of the solvent.

### [Step 3] Synthesis of Compound (I)

Compound (I) wherein symbols in the formula are as defined above can be synthesized by an amidation condensation reaction of Intermediate B and Intermediate A (R₃, R₄ₐ and R_{4b}, are as defined above), followed by a ring closure reaction in the presence of an acid. In the amidation condensation reaction, generally known amidation reagents and conditions can be applied. HATU or DMT-MM is preferable as the condensing agent, DMF, MeOH, THF or the like is preferable as the solvent, and the reaction temperature is preferably 0° C. to the boiling point of the solvent. As the acid to be used for the ring closure reaction, formic acid is preferable and formic acid can also be used as the solvent. The reaction temperature is preferably 0° C. to the boiling point of the used solvent.

Intermediate A can be prepared by the following method.

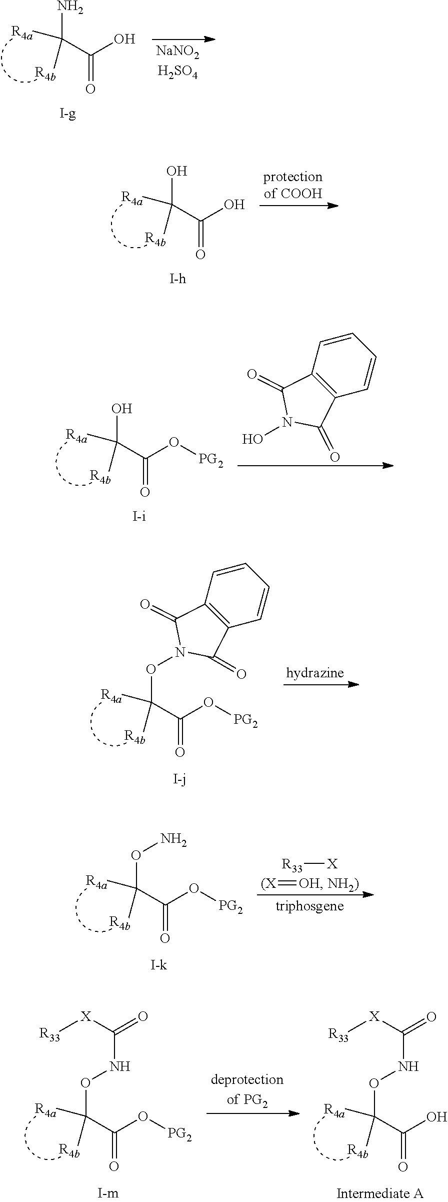

The amino group of an amino acid derivative I-g (R₄ₐ and R_{4b} are as defined above) can be converted to a hydroxy group under NaNO₃/H₂SO₄ condition. The carboxylic acid group of the obtained intermediate I-h (R₄ₐ and R_{4b} are as defined above) can be converted to intermediate I-i (PG₂ is a protecting group of carboxylic acid and other symbols in the formula are as defined above) by generally known methods such as those described in “Greene's Protective Groups in Organic Synthesis, 5th Edition” by John Wiley & Sons, Inc. Examples of the protecting group of carboxylic acid include methyl, ethyl, t-butyl, benzyl, methoxymethyl, triphenylmethyl and like. The hydroxy group of I-i can be converted to N-hydroxyphthalimide group under Mitsunobu reaction condition. Deprotection of the phthalimide group of the intermediate I-j (symbols in the formula are as defined above) using hydrazine leads to intermediate I-k (symbols in the formula are as defined above). Intermediate I-m (symbols in the formula are as defined above) can be synthesized by a condensation reaction with I-k and R₃₃—X (R₃₃ is as defined above, and X is an amino or alcohol group) using triphosgene. Intermediate A can be obtained by deprotection of the protecting group PG₂.

### 2) Production Method 2

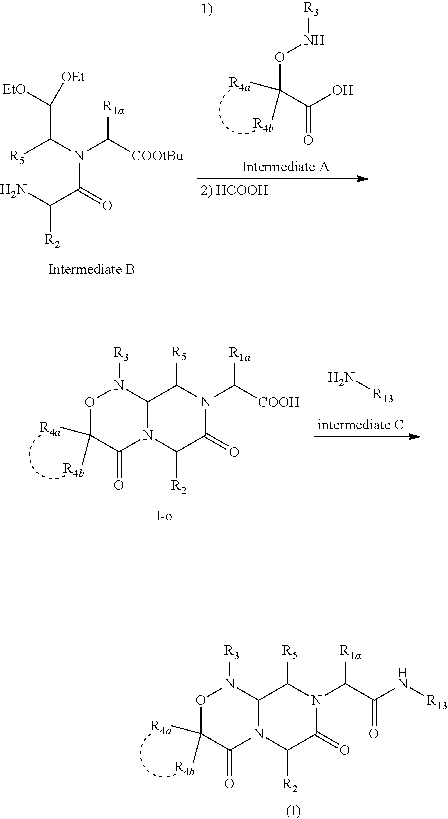

Compound (I) wherein Q is —CH(—R₁ₐ)(—R_{1b}), R_{1b} is —(CO)—NH—R₁₃ and other symbols in the formula are as defined above can be synthesized by the following scheme using intermediate B wherein Q is —CH(—R₁ₐ)(—COOtBu) and other symbols in the formula are as defined. In the ring closure reaction, the t-Butyl ester group is deprotected and converted to carboxylic acid group in the presence of HCOOH. In the amidation condensation reaction of the I-o (symbols in the formula are as defined above) and Intermediate C (R₁₃ is as defined above), generally known amidation reagents and conditions can be applied. HATU, CIP or DMT-MM is preferable as the condensing agent, DMF, MeOH, THF or the like is preferable as the solvent, and the reaction temperature is preferably 0° C. to the boiling point of the solvent.

### 3) Production Method 3

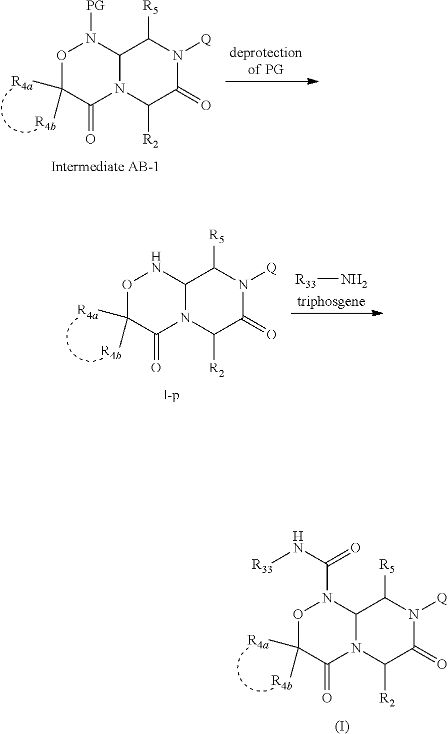

Compound (I) wherein R₃ is —(CO)—NH—R₃₃ can be synthesized by deprotection reaction of Intermediate AB-1 (PG is an amino-protecting group other symbols in the formula are as defined above) followed by the condensation reaction with an amine derivative R₃₃—NH₂ using triphosgene. AB-1 can be prepared by generally known methods, or the step-1, step-2 and step 3 described above.

Representative Examples of the amino-protecting group PG include benzyloxycarbonyl (Cbz), 9-fluorenylmethyloxycarbonyl (Fmoc) and the like. In the deprotection reaction, a generally known reaction can be applied. When PG in the formula is an Fmoc group, a deprotection reaction using piperidine or DBU as a deprotecting agent in ethyl acetate or dichloromethane as a solvent is preferable. When PG is a Cbz group, a deprotection reaction using a palladium catalyst such as Pd(OH)₂, Pd/C or the like in methanol, ethanol or THF as a solvent under H₂ atmosphere is preferable. The reaction temperature is preferably 0° C. to the boiling point of the used solvent. In the condensation reaction, DCM, DCE, THF or the like is a preferable solvent and the reaction temperature is preferably 0° C. to the boiling point of the used solvent.

### 4) Production Method 4

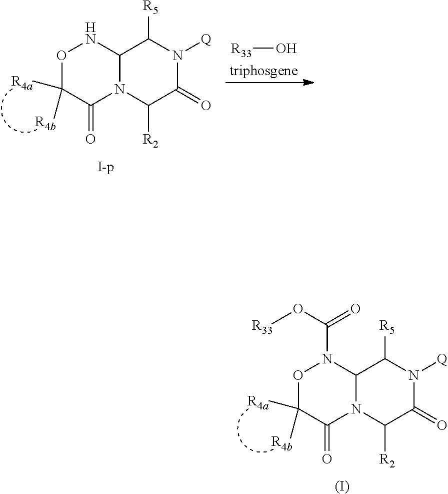

Compound (I) wherein R₃ is —(CO)—O—R₃₃ can be synthesized by a reaction using the intermediate I-p, an alcohol derivative R₃₃—OH and triphosgene. DCM, DCE, THF or the like is a preferable solvent, and the reaction temperature is preferably 0° C. to the boiling point of the used solvent.

### 5) Production Method 5

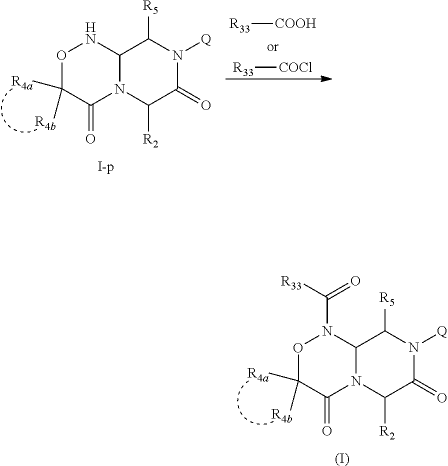

Compound (I) wherein R₃ is —(CO)—R₃₃ can be synthesized by an amidation condensation reaction using the intermediate I-p and R₃₃—COOH (or R₃₃—COCl). DMF, DCM, DCE, THF, MeOH or the like is preferable as the solvent, and the reaction temperature is preferably 0° C. to the boiling point of the solvent. When R₃₃—COOH is used, generally known amidation reagents such as HATU, CIP or DMT-MM are used as the condensing agent.

### 6) Production Method 6

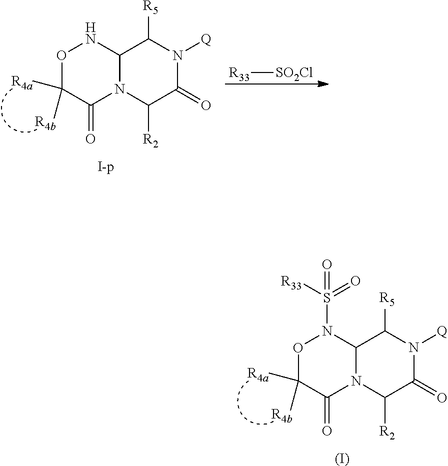

Compound (I) wherein R₃ is —(SO₂)—R₃₃ can be synthesized by n reaction using the intermediate I-p and R₃₃—SO₂Cl. DCM, DCE, THF or the like is a preferable solvent. The reaction temperature is preferably 0° C. to the boiling point of the used solvent.

### 7) Production Method 7

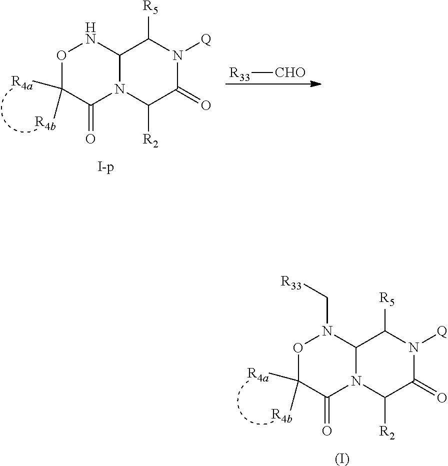

Compound (I) wherein R₃ is —(CH₂)—R₃₃ can be synthesized by a reaction using the intermediate I-p and an aldehyde reagent R₃₃—CHO under the presence of a reducing agent. Generally known reducing reagents such as sodium triacetoxyborohydride, sodium cyanoborohydride, sodium tetrahydroborate, lithium tetrahydroborate, THF-borane complex, pyridine-borane complex, picoline-borane complex, diisobutylaluminium hydride (DIBAL), lithium aluminium hydride and the like are preferable as the reducing reagent. MeOH, THF, chloroform, DCM, DCE, diethylether, diisopropylether and the like are preferable as solvent. The reaction temperature is preferably from −78° C. to the boiling point of the solvent.

### 8) Production Method 8

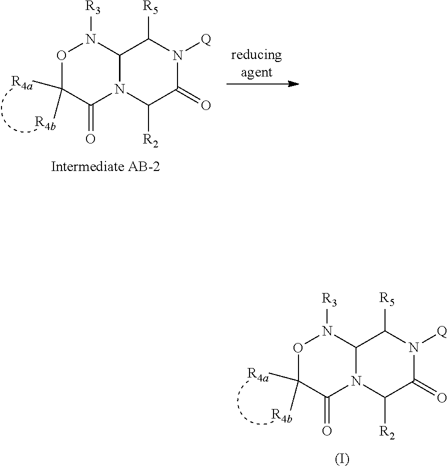

Compound (I) wherein U is —(CH₂)— can be synthesized by the following scheme using Intermediate AB-2 and a reducing agent. AB-2 can be prepared by generally known methods, or step-1, step-2 and step 3 described above. Generally known reducing reagents such as sodium triacetoxyborohydride, sodium cyanoborohydride, sodium tetrahydroborate, lithium tetrahydroborate, THF-borane complex, pyridine-borane complex, picoline-borane complex, diisobutylaluminium hydride (DIBAL), lithium aluminium hydride and the like are preferable as the reducing reagent. MeOH, THF, chloroform, DCM, DCE, diethylether, diisopropylether and the like are preferable as solvent, and the reaction temperature is preferably from −78° C. to the boiling point of the solvent.

The protecting group in each step is not limited to the protecting group (e.g., a diethyl acetal group) specifically indicated in the scheme, and a generally-known protecting group such as a dimethylacetal group may be used. Deprotection in Step 2 or Step 3 can be performed by a general method corresponding to the protecting group. When R₁ₐ, R_{1b}, R₁₃, R₂, R₃, R₄ₐ, R_{4b}, or R₅ has a protected functional group, deprotection can be performed in any step. The compound synthesized in each step of the reaction may be directly used in the next reaction without isolation. Under the conditions of Step 3, the ring closure reaction and the deprotection reaction may proceed simultaneously.

The compound to be obtained in the cyclization reaction can be isolated and purified by a conventional method such as extraction, water-washing, acid washing, alkali washing, crystallization, recrystallization, silica gel column chromatography.

Explaining further, the compounds of the present invention, salts thereof and derivatives thereof are excellent in selectivity pharmacological action selectivity, safety (various toxicities and safety pharmacology), pharmacokinetic performance, physicochemical property and the like, and therefore the usefulness as active ingredients of medicaments can be confirmed.

Examples of tests concerning pharmacological action selectivity include, but not be limited to, inhibition or activation assays on various pharmacological target receptors, inhibition assays on various pharmacological target enzymes, ion channels or transporters, cellular tests to be used for the evaluation for various pharmacological action, and the like.

Examples of tests concerning safety include, but not be limited to, the following list including cytotoxic tests (e.g., tests using HL60 cells, hepatocytes, etc., and the like), genotoxicity tests (e.g., Ames test, mouse lymphoma TK test, chromosomal aberration test, micronucleus test and the like), skin sensitization tests (e.g., Buehler method, GPMT method, APT method, LLNA test and the like), skin photosensitization tests (e.g., Adjuvant and Strip method and the like), eye irritation tests (e.g., single instillation, short-term continuation instillation, repetitive instillation and the like), safety pharmacology tests for the cardiovascular system (e.g., telemetry method, APD method, hERG inhibition assay and the like), safety pharmacology tests for the central nervous system (e.g., FOB method, modified version of Irwin method and the like), safety pharmacology tests for the respiratory system (e.g., measurement method using a respiratory function measuring apparatus, measurement method using a blood gas analyzer and the like), general toxicity tests, and the like.

Examples of tests concerning pharmacokinetic performance include, but not be limited to, the following list including cytochrome P450 enzyme inhibition or induction tests, cell permeability tests (e.g., tests using CaCO-2 cells, MDCK cells etc., and the like), drug transporter ATPase assay, oral absorption tests, blood concentration transition measurement tests, metabolism tests (e.g., stability test, metabolite molecular species test, reactivity test and the like), solubility tests (e.g., solubility test based on turbidity method and the like), and the like.

Examples of tests concerning physicochemical property include, but not be limited to, the following list including chemical stability test (e.g., stability test using HPLC etc., and the like), partition coefficient (e.g., partition test using octanol phase/water phase and the like), ionization constant test, crystallization test, and the like.

In another embodiment, a method for treating various diseases by administering the compound of the present invention is provided. The compound of the present invention may be used for preventing or treating diseases controlled by Notch signal transduction pathway.

In one embodiment, screening relating to the inhibitory action of the Notch signal transduction pathway is performed using a doxycycline-inducing lentiviral vector (see Examples for specific procedures).

The test compound here is a compound described in the present specification, that is, the compound of the present invention. Typically, test compounds are tested at several different concentrations, and the concentrations are partly selected according to the assay conditions.

The compound of the present invention may inhibit Notch signal transduction by interacting with the Notch intracellular domain.

The present invention is also related to prodrugs using the libraries containing one or more compounds of the present invention. A prodrug is typically designed to release the active drug in the body during or after absorption by enzymatic and/or chemical hydrolysis. The prodrug approach is an effective means of improving the oral bioavailability or i.v. administration of poorly water-soluble drugs by chemical derivatization to more water-soluble compounds. The most commonly used prodrug approach for increasing aqueous solubility of drugs containing a hydroxyl group is to produce esters containing an ionizable group; e.g., phosphate group, carboxylate group, alkylamino group (Fleisher et al., Advanced Drug Delivery Reviews, 115-130, 1996; Davis et al., Cancer Res., 7247-7253).

In other aspects, the present invention provides pharmaceutical compositions containing a compound of the present invention. These compositions may be used in various methods of the present invention as described in detail below.

The pharmaceutical composition of the present invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions (e.g., injection) used for parenteral (particularly, intravenous), intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. In addition, pH may be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound, e.g., a compound having general formula (I) in the required amount, in an appropriate solvent with one or a combination of ingredients enumerated above, as required, followed by filtered sterilization. Generally, dispersions are prepared by incorporating the active compound into a sterile vehicle that contains a dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules.

Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent I such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It can be advantageous to formulate oral or parenteral compositions in dosage unit form for ease of administration and uniformity of dosage. Dosage unit form as used herein refers to physically discrete units suited as unitary dosages for the subject to be treated; each unit containing a 5 predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For instance, in certain embodiments, a pharmaceutical composition of the present invention is one suitable for oral administration in unit dosage form such as a tablet or capsule that contains from about 1 mg to about 1 g of the compound of this invention. In some other embodiments, a pharmaceutical composition of the present invention is one suitable for intravenous, subcutaneous or intramuscular injection. A patient may receive, for example, an intravenous, subcutaneous or intramuscular dose of about 1 μg/kg to about 1 g/kg of the compound of the present invention. The intravenous, subcutaneous and intramuscular dose may be given by means of a bolus injection or by continuous infusion over a period of time. Alternatively a patient will receive a daily oral dose approximately equivalent to the daily parenteral dose, the composition being administered 1 to 4 times per day.

Preferably, the compound of the formula (I) of the present invention can be administered intravenously (particularly preferably, by continuous drip infusion or rapid intravenous administration) to mammals inclusive of human.

In the case, the dose is selected appropriately depending on various factors such as the body weight and/or age of patients, and/or the degree of the symptom and an administration route. For example, the dose of the compound of the formula (I) for intravenous administration is generally in the range of 1 to 10000 mg/day/m² human body surface area, preferably in the range of 1 to 5000 mg/day/m² human body surface area, and more preferably 10 to 5000 mg/day/m² human body surface area by continuous drip infusion administration.

A pharmaceutical composition containing the compound of the present invention can be used for diseases regulated by Notch signal transduction pathway. More specifically, a compound that inhibits Notch signal provides a method for suppressing expression of Hes1 and Hes5 and promoting differentiation of neural stem cells, and is expected to be a candidate for anew nerve regeneration drug.

The present invention also provides methods for promoting differentiation of a neural stem cell comprising contacting a neural stem cell with a compound according to formula (I) in an amount effective to promote differentiation of a neural stem cell. Such methods are also useful in treating neurodegenerative diseases (e.g., glaucoma, macular degeneration, Parkinson's Disease, and Alzheimer's disease) and injuries to nervous system. “Neural stem cell” refers to a clonogenic, undifferentiated, multipotent cell capable of differentiating into a neuron, an astrocyte or an oligodendrocyte under appropriate conditions. A compound promotes differentiation of neural stem cells if neural stem cells exhibit a statistically significantly higher degree of differentiation in the presence of the compound than in the absence of the compound. Such a compound may be identified using assays involving in vitro cultured stem cells or animal models (Albranches et al., Biotechnol. Lett. 25: 725-30, 2003; Deng et al., Exp. Neurol. 182: 373-82, 2003; Munoz-Elias et al., Stem Cells 21: 437-48, 2003; Kudo et al, Biochem. Pharmacol. 66: 289-95, 2003; Wan et al., Chin. Med. J. 116: 428-31, 2003; Kawamorita et al., Hum. Cell 15: 178-82, 2002; Stavridis and Smith, Biochem. Soc. Trans. 31: 45-9, 2003; Pachemik et al., Reprod. Nutr. Dev. 42: 317-26, 2002; Fukunaga et al., supra). The neural stem cell may be a cultured stem cell, a stem cell freshly isolated from its source tissue, or a stem cell within its source organism. Thus, contacting the neural stem cell with a compound according to the present invention may be carried out either in vitro (for a cultured or freshly isolated stem cell) or in vivo (for a stem cell within its source organism). The resulting differentiated neural cell, if generated in vitro, may be transplanted into a tissue in need thereof (Lacza et al., supra; Chu et al., supra; Fukunaga et al., supra). Such a tissue includes a brain tissue or other nervous tissue that suffers from a trauma or a neurodegenerative disease.

The following non-limiting examples illustrate the compounds, compositions, and methods of use of this invention.

### EXAMPLES

The present invention is explained in more detail in the following by referring to Production Examples, Examples, Reference Examples and Experimental Examples; however, the scope of the present invention is not limited thereto.

In the Examples, ¹H NMR was measured using Bruker AVANCE III 400; Bruker AVANCE III 400 HD and Bruker AVANCE NEO 40, or Bruker AVANCE III 300.

Preparative HPLC (prep-HPLC) was performed using GILSON-GX-28 or Waters FractionLynx system. Preparation conditions used are as follows.

### General Prep-HPLC Condition (AcOH):

Column: C30-UG 25 mmID*150 mmL, 5 μm
Mobile phase A: water with 0.10% v/v acetic acid
Mobile phase B: acetonitrile
UV detection wavelength: 220 nm
Flow rate: 25 ml/min
Temperature: room temperature

Gradient time table:
0 min B = x %, A = 100 − x %
0.01 − 10.99 min linear gradient
11.00 min B = y %, A = 100 − y %
11.01 − 11.20 min B = y %, A = 100 − y %
11.21 − 13.00 min B = 100 %
13.01 − 15.00 min B = z %, A = 100 − z %

x, y and z values depend on the kind of compounds.

### General Prep-HPLC Condition (TFA):

Column: L-Column2 ODS 20 mmID*150 mmL, 5 μm
Mobile phase A: water with 0.10% v/v TFA
Mobile phase B: acetonitrile with 0.10% v/v TFA
UV detection wavelength: 220 nm
Flow rate: 20 ml/min

### Temperature: Room Temperature

Gradient time table:
0 min B = x %, A = 100 − x %
0.01 − 6.99 min linear gradient
7.00 min B = y %, A = 100 − y %
7.01 − 10.99 min B = 100 %
11.00 − 12.00 min B = z %, A = 100 − z %

x, y and z values depend on the kind of compounds.

LCMS analysis was performed using the following Methods A to D.

### (Method A)

LCMS was measured using Agilent 1100 LC & Agilent G1956A, (ELSD: 1260 Infinity). Analysis was performed under the conditions described in Table 1 or Table 2.

TABLE 1

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 1 | 0.8 |
| | | 0.40 | 1 | 0.8 |
| | | 3.40 | 90 | 0.8 |
| | | 3.90 | 100 | 0.8 |
| | | 3.91 | 1 | 0.8 |
| | | 4.00 | 1 | 1.0 |
| | | 4.50 | 1 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer Pressure | 2070(Torr) |
| | Drying Gas | 350° C. |
| | Temperature | |
| | Capillary Voltage | 2500(V) |
| | MS Polarity | Positive |
| | MS Mode | Scan |
| | Mass Range | 100-1500 |

TABLE 2

| Instrument | | Agilent 1100 LC & Agilent G1956A | | |
|---|---|---|---|---|
| Software | | Agilent Chemstation Rev. B. 04.03[16] | | |
| HPLC | Column | Agilent ZORBAX 5 μm SB-Aq, 2.1*50 mm | | |
| | Mobile Phase | A: 0.0375% TFA in water (v/v) | | |
| | | B: 0.01875% TFA in Acetonitrile (v/v) | | |
| | Gradient | Time(min) | B(%) | Flow(mL/min) |
| | | 0.00 | 10 | 0.8 |
| | | 0.40 | 10 | 0.8 |
| | | 3.40 | 100 | 0.8 |
| | | 3.90 | 100 | 0.8 |
| | | 3.91 | 10 | 0.8 |
| | | 4.00 | 10 | 1.0 |
| | | 4.50 | 10 | 1.0 |

| | | |
|---|---|---|
| | Post time(min) | 0 |
| | Column Temp | 50° C. |
| | Detector | DAD |
| MS | Ionization source | ESI |
| | Drying Gas | N2 |
| | Drying Gas Flow | 10(L/min) |
| | Nebulizer Pressure | 2070(Torr) |
| | Drying Gas | 350° C. |
| | Temperature | |
| | Capillary Voltage | 2500(V) |
| | MS Polarity | Positive |
| | MS Mode | Scan |
| | Mass Range | 50-1500 |

### (Method B)

System: Shimadzu UFLC/MS System (Shimadzu-2020 mass spectrometer)
Column: ODS column for the chromatography column
Eluents: A (water with 0.04% TFA) and B (acetonitrile with 0.04% TFA)

### (Method C)

System: Shimadzu UFLC/MS System (Shimadzu-2020 mass spectrometer)
Column: ODS column for the chromatography column
Eluents: A (5 mM AcONH₄ in water) and B (5 mM AcONH₄ in acetonitrile)

### (Method D)

System: Water 2795 System
Column: Develosil C30-UG-5, 50×4.6 mm, Nomura Chemical Co., Ltd.
Eluents: A(water with 0.1% HCOOH) and B(acetonitrile with 0.1% HCOOH).
Flow rate: 1.0 mL/min

### Production Example 1: Synthesis of Intermediate D-10

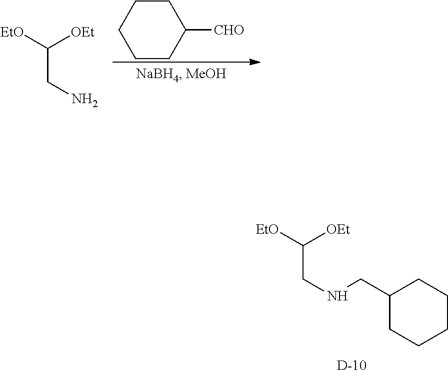

To a solution of 2,2-diethoxyethan-1-amine 75 g (0.67 mol) in MeOH (1.0 L) was added cyclohexanecarbaldehyde 89 g (0.67 mol). After stirring overnight, the mixture was added sodium borohydride 38.2 g (1.0 mol) at 0° C. with stirring at room temperature for 1.5 hrs. The mixture was concentrated and the residue was dissolved in ethyl acetate (1.0 L), washed with water (1.0 L) and brine (1.0 L), dried over sodium sulfate, filtered and evaporated. The target material D-10 (130 g, colorless oil, yield 85%) was obtained by the reduced-pressure distillation (140° C., 5-10 mmHg).

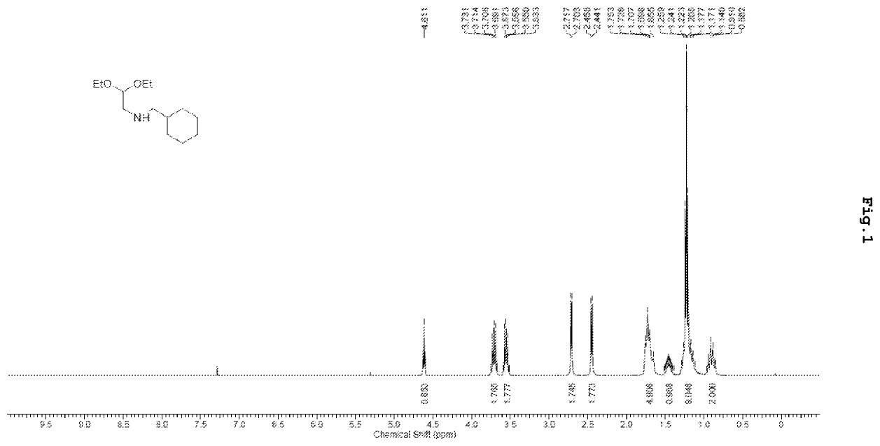

¹H NMR (400 MHz, CDCl₃) data of D-10 is shown in FIG. 1.

Intermediates D-1, D-2, D-3, D-55, D-67, D-135, D-137, D-157, D-158, D-178, D-179 and D-182 were synthesized according to the same method as described above or a known method.

### Production Example 2: Synthesis of Intermediate D-13

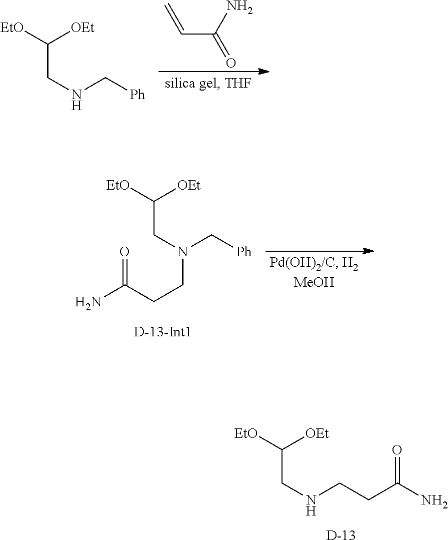

### 2-1) Synthesis of D-13-Int1

To a solution of N-benzyl-2,2-diethoxyethan-1-amine (200 g, 0.90 mol) and acrylamide (77 g, 1.08 mol) in THF (4.0 L) was added SiO₂ (40 g) at room temperature. After refluxing for 7 days, the mixture was filtered and evaporated. The target material D-13-Int1 (194 g, yellow liquid, yield 82%) was obtained by flash chromatography.

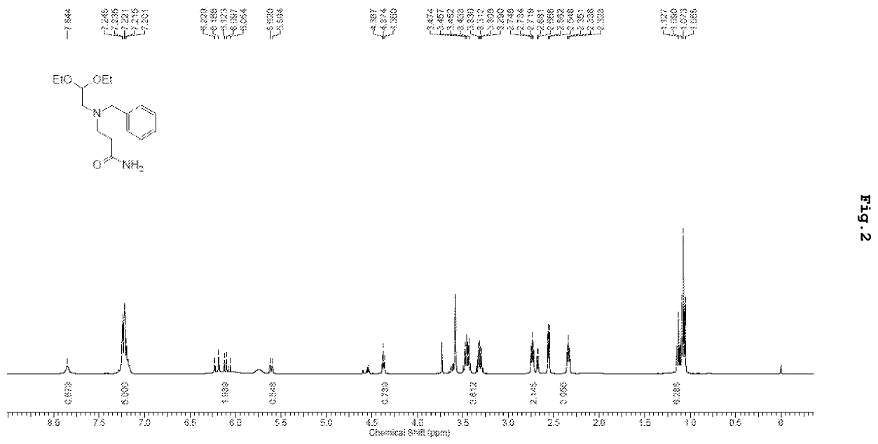

¹H NMR (400 MHz, CDCl₃) data of the obtained compound is shown in FIG. 2.

### 2-2) Synthesis of D-13

A mixture of D-13-Int1 (90 g, 0.3 mol) and Pd(OH)₂/C (9 g) in MeOH was stirred at room temperature under H₂ (50 psi) for 3 hrs. TLC showed that the reaction was completed. The reaction mixture was filtered and the filtrate was concentrated to afford crude product D-13 as yellow oil which was used directly.

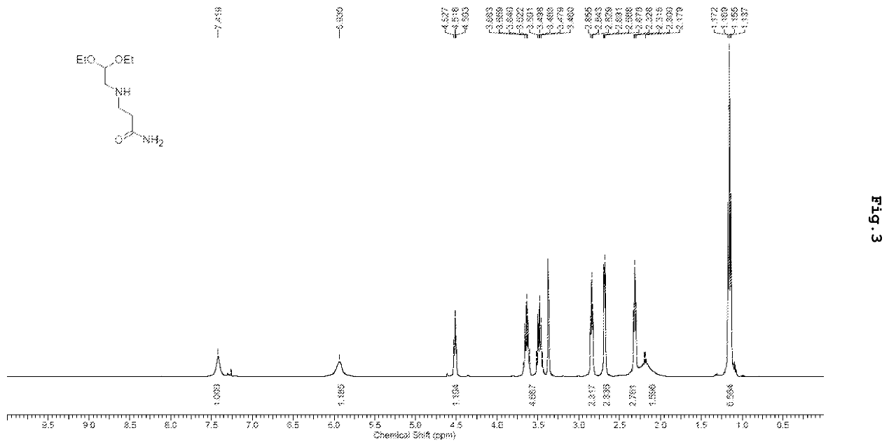

1H NMR (400 MHz, CDCl₃) data of D-13 is shown in FIG. 3.

Intermediate D-58 was synthesized according to the same method as described above or a known method.

### Production Example 3: Synthesis of Intermediate D-8

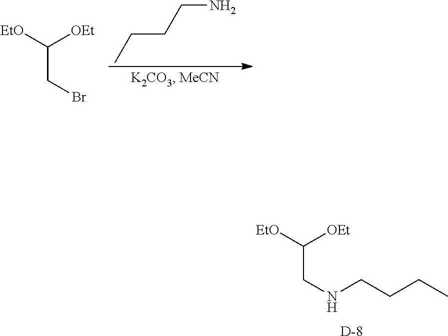

To a solution of 2-bromo-1,1-diethoxyethane (50 g, 0.69 mol) in MeCN (0.7 L) was added potassium carbonate (94.5 g, 0.69 mol) and n-butylamine (50 g, 0.69 mol). After refluxing overnight, the mixture was filtered and evaporated. The residue was dissolved in ethyl acetate (1.0 L), washed with water (0.5 L) and brine (0.5 L), dried over sodium sulfate, filtered and evaporated. The target material D-8 (71 g, colorless oil, 55% yield) was obtained by the reduced-pressure distillation (70° C., 5-10 mmHg).

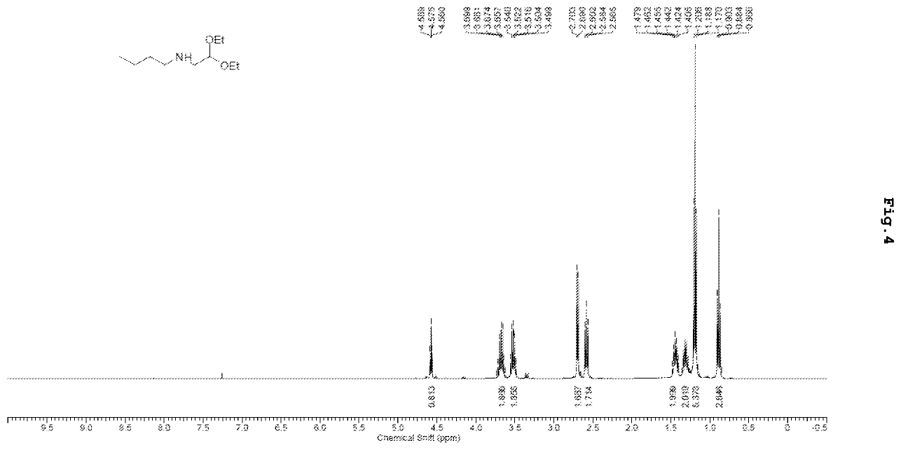

¹H NMR (400 MHz, CDCl₃) data of D-8 is shown in FIG. 4.

Intermediates D-32, D-50, D-53, D-73, D-128 and D-161 were synthesized according to the same method as described above or a known method.

### Production Example 4: Synthesis of Intermediate D-12

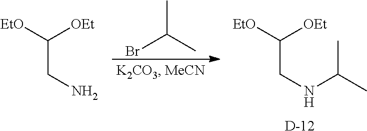

A solution of 2-bromopropane (300 g, 2.44 mol), 2,2-diethoxyethanamine (341 g, 2.56 mol) and potassium carbonate (674 g, 4.88 mol) in MeCN (3.0 L) was stirred at 80° C. for 16 hrs. The solution was filtered and the filtrate was concentrated. The crude product was dissolved in ethyl acetate (2.0 L), washed with water (1.0 L) and brine (1.0 L); dried over anhydrous sodium sulfate, and then filtered and evaporated to give the desired product of D-12 (300 g, 1.71 mol, 70% yield) as yellow oil, which was used for next step without purification.

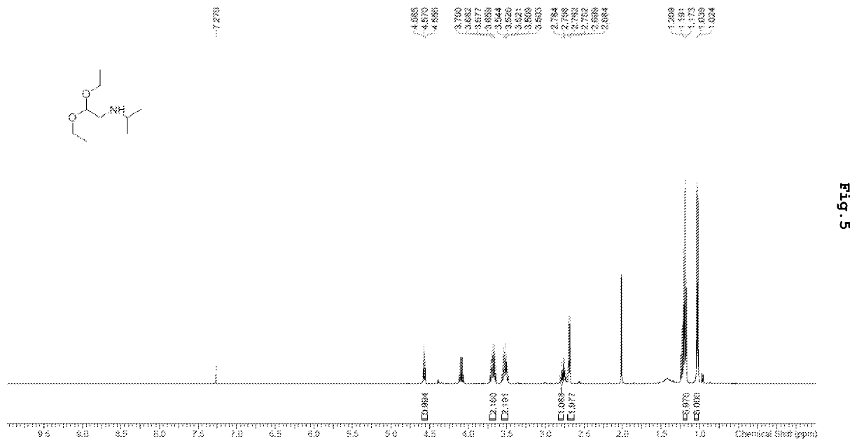

¹H NMR (400 MHz, CDCl₃) data of D-12 is shown in FIG. 5.

Intermediates D-28, D-30, D-31, D-33 and D-70 were synthesized according to the same method as described above or a known method.

### Production Example 5: Synthesis of Intermediate D-82

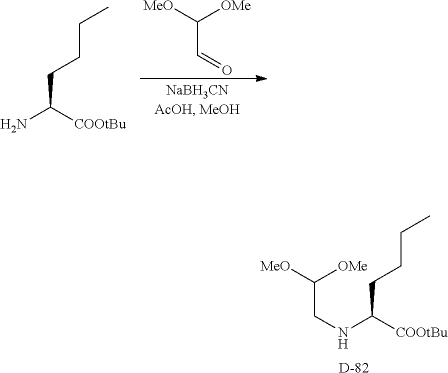

To a solution of tert-butyl (2S)-2-aminohexanoate (310 g, 1.66 mol) in methanol (2.50 L) was added 2,2-dimethoxyacetaldehyde (315.92 g, 1.82 mol, 274.72 mL, 60% purity), then CH₃COOH (2.10 g, 34.97 mmol, 2 mL) was added, the mixture was stirred for 16 hrs. Then NaBH₃CN (124.82 g, 1.99 mol) was added at 0° C., the mixture was allowed to warm to 20° C. and was stirred for 2 hrs. TLC (Petroleum ether:Ethyl acetate=2:1) showed most of the starting material (tert-butyl (2S)-2-aminohexanoate) was consumed. The reaction mixture was concentrated under vacuo to give yellow oil. The yellow oil was purified by column chromatography (SiO₂, Petroleum ether:Ethyl acetate=20:1 to 5:1) to afford D-82 (530 g, 1.92 mol, 58% yield) as a light yellow oil.

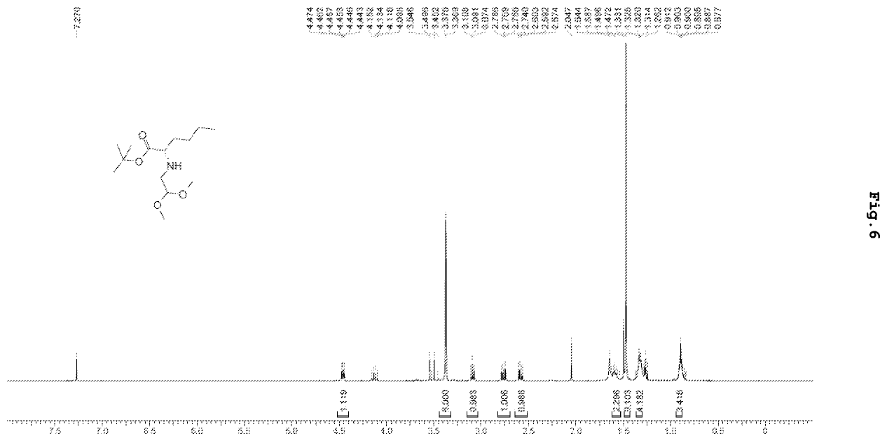

¹H NMR (400 MHz, CDCl₃) data of D-82 is shown in FIG. 6.

Intermediates D-80, D-91, D-94, D-180 and D-183 were synthesized according to the same method as described above or a known method.

A list of Intermediate D is shown in the Table 3 (Table 3-1 and Table 3-2).

TABLE 3-1

| Int. D | Structure |
|---|---|
| D-1 | 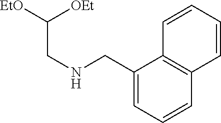 [SMILES: CCOC(CNCc1cccc2ccccc12)OCC] |
| D-2 | 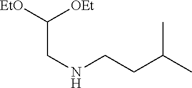 |
| D-3 | 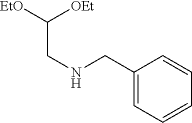 [SMILES: CCOC(CNCc1ccccc1)OCC] |
| D-8 | 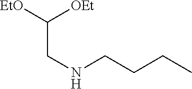 |
| D-10 | 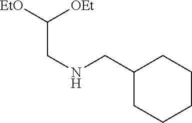 [SMILES: CCOC(CNCC1CCCCC1)OCC] |
| D-12 | 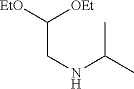 |
| D-13 | 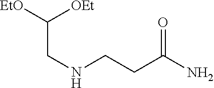 [SMILES: CCOC(CNCCC(N)=O)OCC] |
| D-28 | 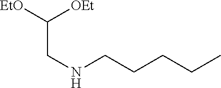 |
| D-30 | 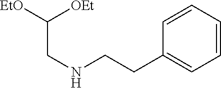 [SMILES: CCOC(CNCCc1ccccc1)OCC] |
| D-31 | 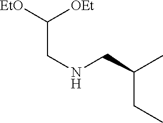 |
| D-32 | 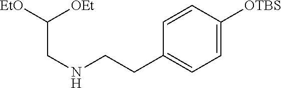 [SMILES: CCOC(CNCCc1ccc(O[Si](C)(C)C(C)(C)C)cc1)OCC] |
| D-33 | 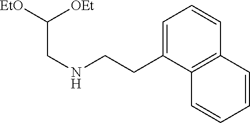 [SMILES: CCOC(CNCCc1cccc2ccccc12)OCC] |
| D-50 | 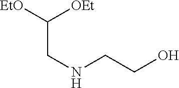 [SMILES: CCOC(CNCCO)OCC] |
| D-53 | 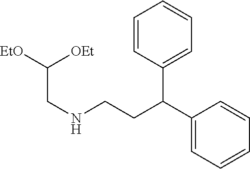 [SMILES: CCOC(CNCCC(c1ccccc1)c1ccccc1)OCC] |
| D-55 | 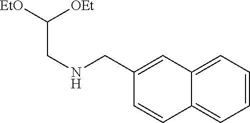 [SMILES: CCOC(CNCc1ccc2ccccc2c1)OCC] |

TABLE 3-2

| Int. D | Structure |
|---|---|
| D-58 | 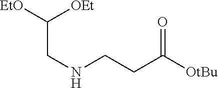 [SMILES: CCOC(CNCCC(=O)OC(C)(C)C)OCC] |
| D-67 | 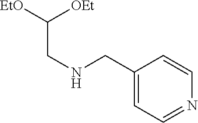 [SMILES: CCOC(CNCc1ccncc1)OCC] |
| D-70 | 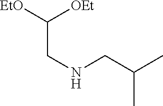 |
| D-73 | 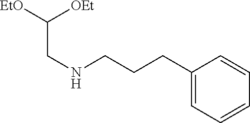 [SMILES: CCOC(CNCCCc1ccccc1)OCC] |
| D-80 | 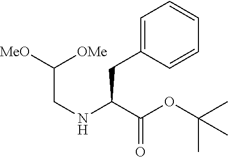 [SMILES: COC(CN[C@@H](Cc1ccccc1)C(=O)OC(C)(C)C)OC] |
| D-82 | 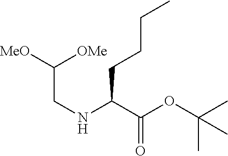 [SMILES: CCCC[C@H](NCC(OC)OC)C(=O)OC(C)(C)C] |
| D-91 | 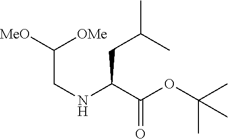 [SMILES: COC(CN[C@@H](CC(C)C)C(=O)OC(C)(C)C)OC] |
| D-94 | 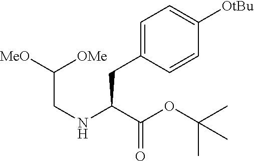 [SMILES: COC(CN[C@@H](Cc1ccc(OC(C)(C)C)cc1)C(=O)OC(C)(C)C)OC] |
| D-128 | 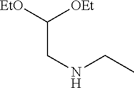 |
| D-135 | 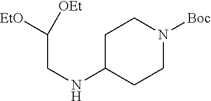 [SMILES: CCOC(CNC1CCN(C(=O)OC(C)(C)C)CC1)OCC] |
| D-137 | 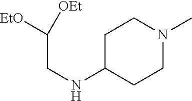 [SMILES: CCOC(CNC1CCN(C)CC1)OCC] |
| D-157 | 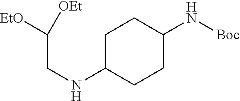 [SMILES: CCOC(CNC1CCC(NC(=O)OC(C)(C)C)CC1)OCC] |
| D-158 | 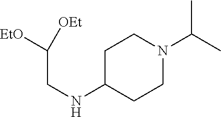 [SMILES: CCOC(CNC1CCN(C(C)C)CC1)OCC] |
| D-161 | 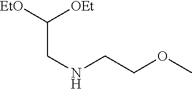 |
| D-178 | 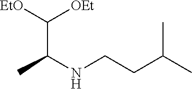 |
| D-179 | 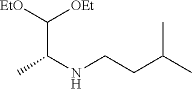 |
| D-180 | 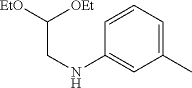 [SMILES: CCOC(CNc1cccc(C)c1)OCC] |
| D-182 | 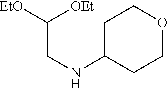 [SMILES: CCOC(CNC1CCOCC1)OCC] |
| D-183 | 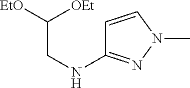 |

All the Intermediates E shown in Table 4 (Table 4-1 and Table 4-2) are commercially available or were synthesized according to a known method.

TABLE 4-1

| Int. E | Structure |
|---|---|
| E-1 | 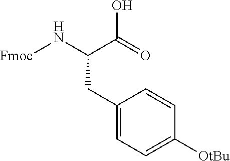 |
| E-13 | 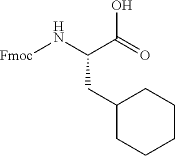 |
| E-15 | 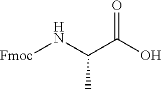 |
| E-18 | 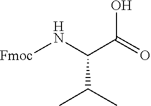 |
| E-20 | 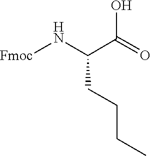 |
| E-21 | 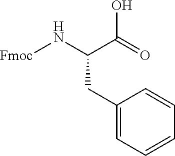 |
| E-28 | 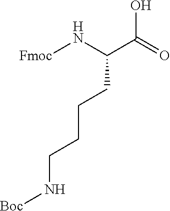 |
| E-32 | 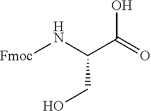 |
| E-39 | 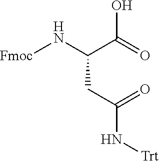 |
| E-41 | 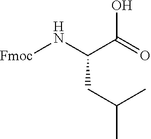 |
| E-46 | 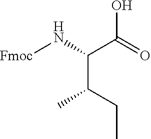 |
| E-55 | 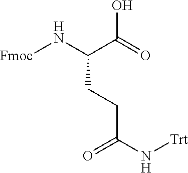 |

TABLE 4-2

| Int. E | Structure |
|---|---|
| E-60 | 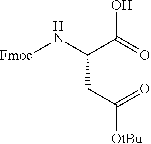 |
| E-69 | 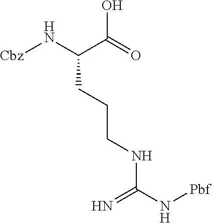 |
| E-142 | 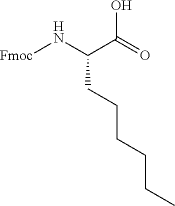 |
| E-158 | 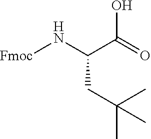 |
| E-176 | 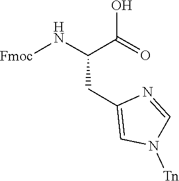 |
| E-178 | 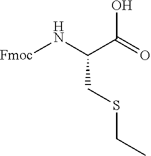 |

### Production Example 6: Synthesis of Intermediate B-128

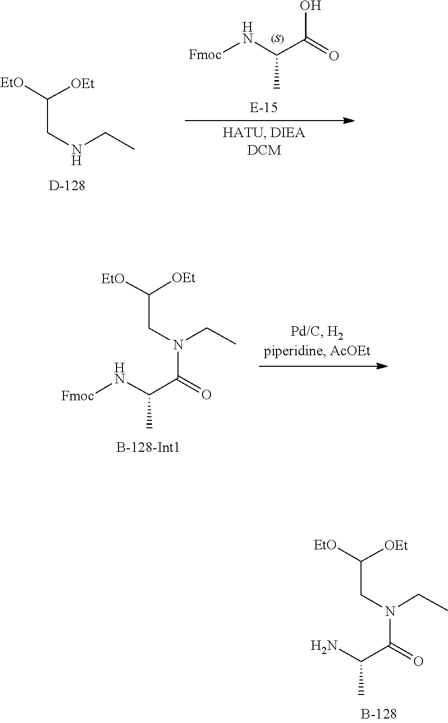

### 6-1) Synthesis of B-128-Int1

To a stirring mixture of 2,2-diethoxy-N-ethyl-ethanamine (D-128, 11.0 g, 68.2 mmol), (2S)-2-(9H-fluoren-9-ylmethoxycarbonylamino)propanoic acid (E-15, 21.2 g, 68.2 mmol) and N,N-diisopropylethylamine (17.6 g, 136 mmol, 24.0 mL) in DCM (200 mL) was added HATU (25.9 g, 68.2 mmol) in portions in an ice-bath. After stirred for 30 mins at 0° C., the reaction mixture was stirred for 16 hrs at 10° C. TLC (Petroleum ether/Ethyl acetate=3:1) showed the reaction completed. The reaction mixture was washed with water, brine, dried over anhydrous sodium sulfate, filtered and concentrated to give the crude product which was purified by column chromatography (SiO₂, Petroleum ether/Ethyl acetate=10:1 to 2:1) to afford the product B-128-Int1 (23.0 g, 50.6 mmol, 74% yield).

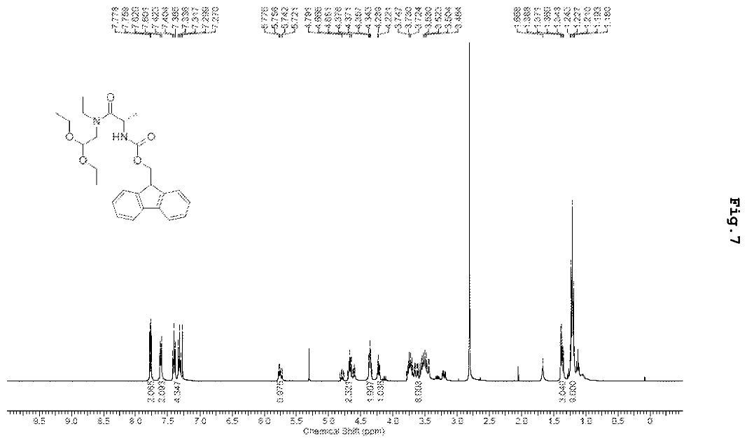

¹H NMR (400 MHz, CDCl₃) data of B-128-Int1 is shown in FIG. 7.

### 6-2) Synthesis of Intermediate B-128

A mixture of B-128-Int1 (23.0 g, 50.6 mmol), Pd/C (5.00 g, 10% w/w) in EtOAc (200 mL, contained 1% v/v piperidine) was stirred under H₂ at 50 psi for 48 hrs. The reaction mixture was filtered and the filtrate was concentrated under reduced pressure to give the crude product which was purified by column chromatography (SiO₂, CH₂Cl₂:MeOH=1:0 to 20:1) to give B-128 (10.5 g, 45.3 mmol, 89% yield).

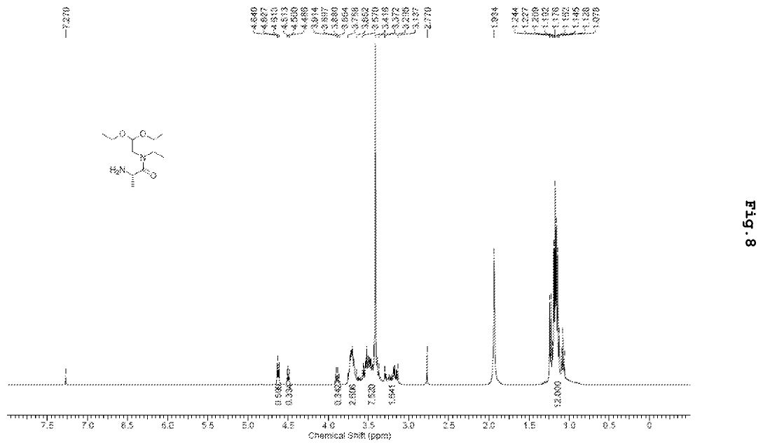

¹H NMR (400 MHz, CDCl₃) data of B-128 is shown in FIG. 8.

### Production Example 7: Synthesis of Intermediate B-72

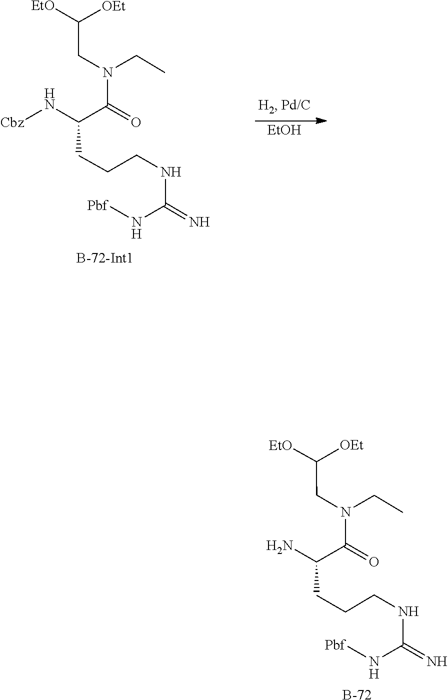

B-72-Int1 was synthesized by using D-128 and E-69 in the same manner as in Production Example 6:6-1).

To a solution of B-72-Int1 (56.0 g, 79.6 mmol) in EtOH (300 mL) was added Pd/C (6 g, 10% purity) under nitrogen. The suspension was degassed under vacuum and purged with hydrogen several times. The mixture was stirred under hydrogen (15 psi) at 25° C. for 72 hrs. The reaction mixture was filtered and the filtrate was concentrated under reduced pressure to give B-72 (35.0 g, 45.3 mmol, 57% yield).

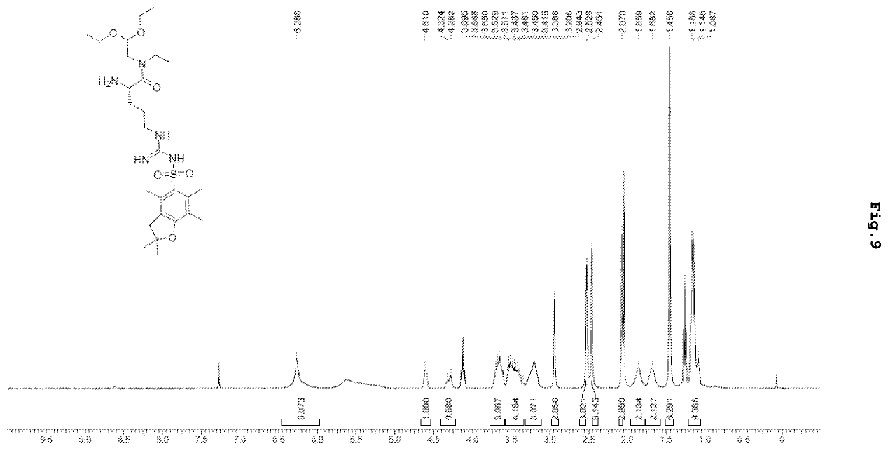

¹H NMR (400 MHz, CDCl₃) data of B-72 is shown in FIG. 9.

A list of Intermediate B is shown in the Table 5 (Table 5-1 to Table 5-15). Intermediate B was synthesized using corresponding Intermediate D and Intermediate E according to Production Example 6, Production Example 7 or a known method.

TABLE 5-1

| Int. B | Structure | Int. D | Int. E |
|---|---|---|---|
| B-1 | 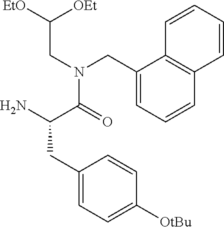 [SMILES: CCOC(CN(Cc1cccc2ccccc12)C(=O)[C@@H](N)Cc1ccc(OC(C)(C)C)cc1)OCC] | D-1 | E-1 |
| B-2 | 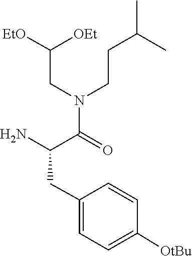 [SMILES: CCOC(CN(CCC(C)C)C(=O)[C@@H](N)Cc1ccc(OC(C)(C)C)cc1)OCC] | D-2 | E-1 |
| B-3 | 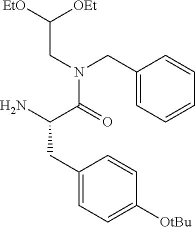 [SMILES: CCOC(CN(Cc1ccccc1)C(=O)[C@@H](N)Cc1ccc(OC(C)(C)C)cc1)OCC] | D-3 | E-1 |
| B-8 | 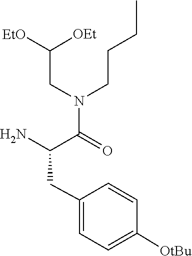 [SMILES: CCCCN(CC(OCC)OCC)C(=O)[C@@H](N)Cc1ccc(OC(C)(C)C)cc1] | D-8 | E-1 |
| B-10 | 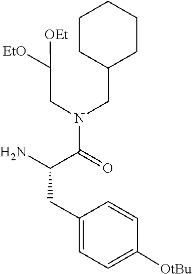 [SMILES: CCOC(CN(CC1CCCCC1)C(=O)[C@@H](N)Cc1ccc(OC(C)(C)C)cc1)OCC] | D-10 | E-1 |

TABLE 5-2

| Int. B | Structure | Int. D | Int. E |
|---|---|---|---|
| B-12 | 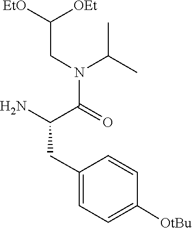 [SMILES: CCOC(CN(C(=O)[C@@H](N)Cc1ccc(OC(C)(C)C)cc1)C(C)C)OCC] | D-12 | E-1 |
| B-13 | 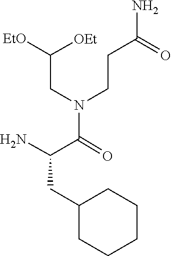 [SMILES: CCOC(CN(CCC(N)=O)C(=O)[C@@H](N)CC1CCCCC1)OCC] | D-13 | E-13 |
| B-15 | 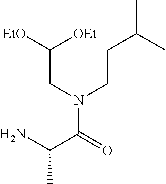 [SMILES: CCOC(CN(CCC(C)C)C(=O)[C@H](C)N)OCC] | D-2 | E-15 |
| B-18 | 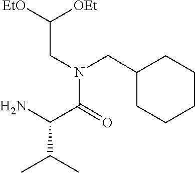 [SMILES: CCOC(CN(CC1CCCCC1)C(=O)[C@@H](N)C(C)C)OCC] | D-10 | E-18 |
| B-19 | 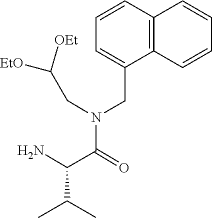 [SMILES: CCOC(CN(Cc1cccc2ccccc12)C(=O)[C@@H](N)C(C)C)OCC] | D-1 | E-18 |
| B-20 | 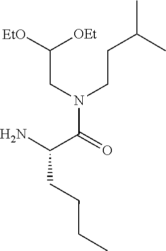 [SMILES: CCCC[C@H](N)C(=O)N(CCC(C)C)CC(OCC)OCC] | D-2 | E-20 |

TABLE 5-3

| Int. B | Structure | Int. D | Int. E |
|---|---|---|---|
| B-21 | 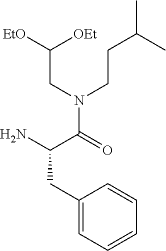 [SMILES: CCOC(CN(CCC(C)C)C(=O)[C@@H](N)Cc1ccccc1)OCC] | D-2 | E-21 |
| B-25 | 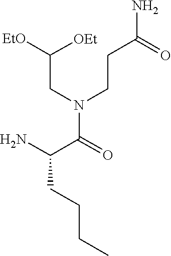 [SMILES: CCCC[C@H](N)C(=O)N(CCC(N)=O)CC(OCC)OCC] | D-13 | E-20 |
| B-26 | 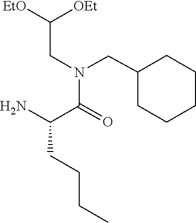 [SMILES: CCCC[C@H](N)C(=O)N(CC1CCCCC1)CC(OCC)OCC] | D-10 | E-20 |
| B-27 | 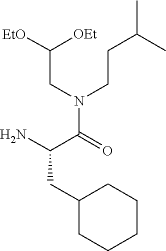 [SMILES: CCOC(CN(CCC(C)C)C(=O)[C@@H](N)CC1CCCCC1)OCC] | D-2 | E-13 |
| B-28 | 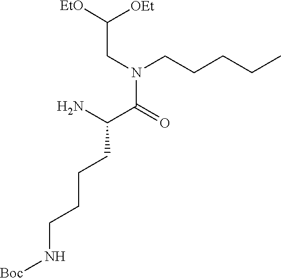 [SMILES: CCCCCN(CC(OCC)OCC)C(=O)[C@@H](N)CCCCNC(=O)OC(C)(C)C] | D-28 | E-28 |

TABLE 5-4

| Int. B | Structure | Int. D | Int. E |
|---|---|---|---|
| B-29 | 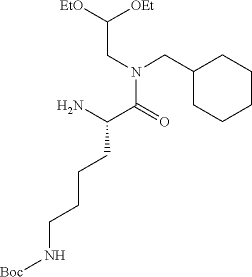 [SMILES: CCOC(CN(CC1CCCCC1)C(=O)[C@@H](N)CCCCNC(=O)OC(C)(C)C)OCC] | D-10 | E-28 |
| B-30 | 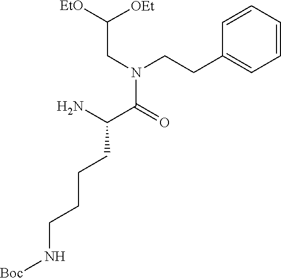 [SMILES: CCOC(CN(CCc1ccccc1)C(=O)[C@@H](N)CCCCNC(=O)OC(C)(C)C)OCC] | D-30 | E-28 |
| B-31 | 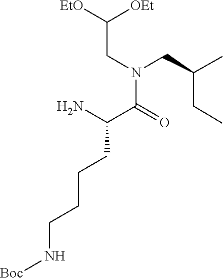 [SMILES: CCOC(CN(C[C@@H](C)CC)C(=O)[C@@H](N)CCCCNC(=O)OC(C)(C)C)OCC] | D-31 | E-28 |
| B-32 | 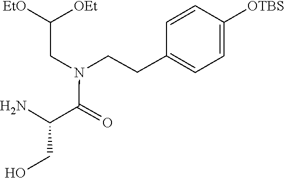 [SMILES: CCOC(CN(CCc1ccc(O[Si](C)(C)C(C)(C)C)cc1)C(=O)[C@@H](N)CO)OCC] | D-32 | E-32 |
| B-33 | 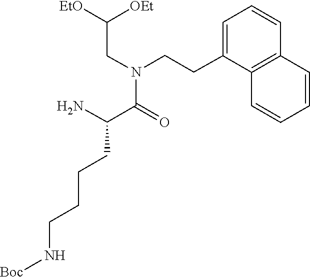 [SMILES: CCOC(CN(CCc1cccc2ccccc12)C(=O)[C@@H](N)CCCCNC(=O)OC(C)(C)C)OCC] | D-33 | E-28 |

TABLE 5-5

| Int. B | Structure | Int. D | Int. E |
|---|---|---|---|
| B-35 | 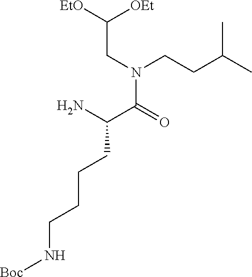 [SMILES: CCOC(CN(CCC(C)C)C(=O)[C@@H](N)CCCCNC(=O)OC(C)(C)C)OCC] | D-2 | E-28 |
| B-39 | 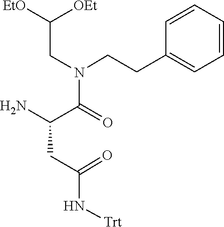 | D-30 | E-39 |
| B-41 | 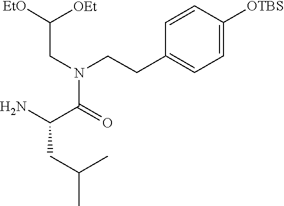 [SMILES: CCOC(CN(CCc1ccc(O[Si](C)(C)C(C)(C)C)cc1)C(=O)[C@@H](N)CC(C)C)OCC] | D-32 | E-41 |
| B-43 | 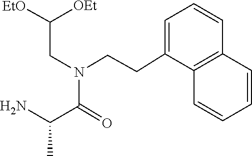 [SMILES: CCOC(CN(CCc1cccc2ccccc12)C(=O)[C@H](C)N)OCC] | D-33 | E-15 |
| B-44 | 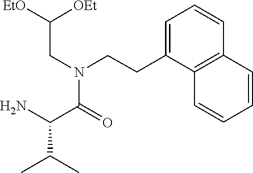 [SMILES: CCOC(CN(CCc1cccc2ccccc12)C(=O)[C@@H](N)C(C)C)OCC] | D-33 | E-18 |
| B-45 | 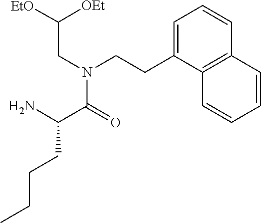 [SMILES: CCCC[C@H](N)C(=O)N(CCc1cccc2ccccc12)CC(OCC)OCC] | D-33 | E-20 |

TABLE 5-6

| Int. B | Structure | Int. D | Int. E |
|---|---|---|---|
| B-46 | 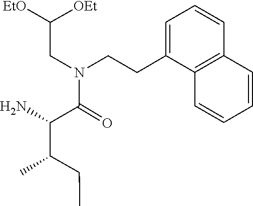 [SMILES: CCOC(CN(CCc1cccc2ccccc12)C(=O)[C@@H](N)[C@@H](C)CC)OCC] | D-33 | E-46 |
| B-47 | 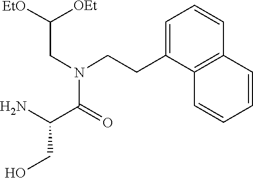 [SMILES: CCOC(CN(CCc1cccc2ccccc12)C(=O)[C@@H](N)CO)OCC] | D-33 | E-32 |
| B-49 | 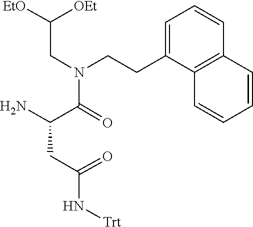 | D-33 | E-39 |
| B-50 | 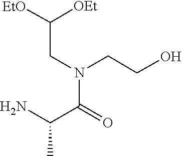 [SMILES: CCOC(CN(CCO)C(=O)[C@H](C)N)OCC] | D-50 | E-15 |
| B-51 | 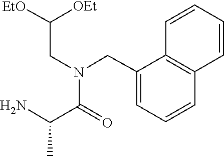 [SMILES: CCOC(CN(Cc1cccc2ccccc12)C(=O)[C@H](C)N)OCC] | D-1 | E-15 |
| B-53 | 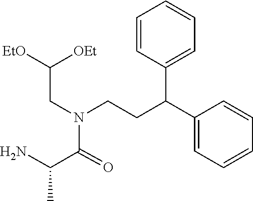 [SMILES: CCOC(CN(CCC(c1ccccc1)c1ccccc1)C(=O)[C@H](C)N)OCC] | D-53 | E-15 |
| B-54 | 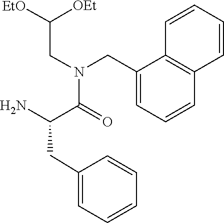 [SMILES: CCOC(CN(Cc1cccc2ccccc12)C(=O)[C@@H](N)Cc1ccccc1)OCC] | D-1 | E-21 |

TABLE 5-7

| Int. B | Structure | Int. D | Int. E |
|---|---|---|---|
| B-55 | 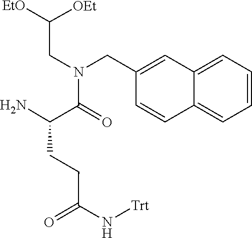 | D-55 | E-55 |
| B-56 | 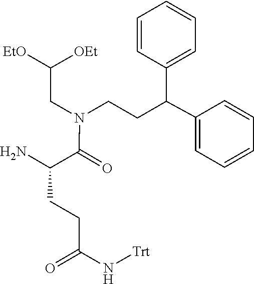 | D-53 | E-55 |
| B-58 | 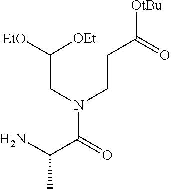 [SMILES: CCOC(CN(CCC(=O)OC(C)(C)C)C(=O)[C@H](C)N)OCC] | D-58 | E-15 |
| B-59 | 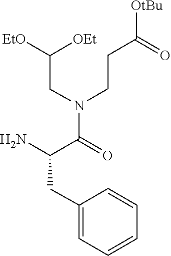 [SMILES: CCOC(CN(CCC(=O)OC(C)(C)C)C(=O)[C@@H](N)Cc1ccccc1)OCC] | D-58 | E-21 |
| B-60 | 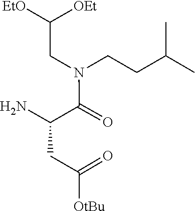 [SMILES: CCOC(CN(CCC(C)C)C(=O)[C@@H](N)CC(=O)OC(C)(C)C)OCC] | D-2 | E-60 |
| B-61 | 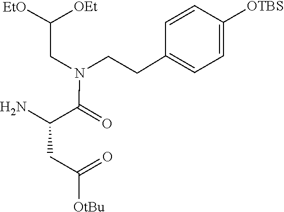 [SMILES: CCOC(CN(CCc1ccc(O[Si](C)(C)C(C)(C)C)cc1)C(=O)[C@@H](N)CC(=O)OC(C)(C)C)OCC] | D-32 | E-60 |

TABLE 5-8

| Int. B | Structure | Int. D | Int. E |
|---|---|---|---|
| B-62 | 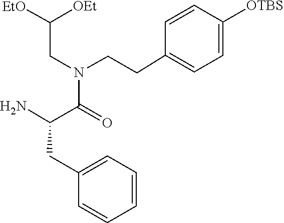 [SMILES: CCOC(CN(CCc1ccc(O[Si](C)(C)C(C)(C)C)cc1)C(=O)[C@@H](N)Cc1ccccc1)OCC] | D-32 | E-21 |
| B-63 | 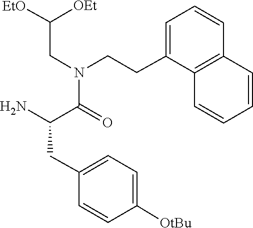 [SMILES: CCOC(CN(CCc1cccc2ccccc12)C(=O)[C@@H](N)Cc1ccc(OC(C)(C)C)cc1)OCC] | D-33 | E-1 |
| B-64 | 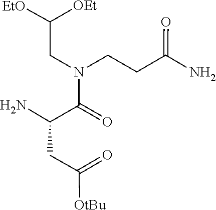 [SMILES: CCOC(CN(CCC(N)=O)C(=O)[C@@H](N)CC(=O)OC(C)(C)C)OCC] | D-13 | E-60 |
| B-67 | 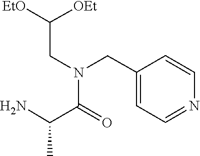 [SMILES: CCOC(CN(Cc1ccncc1)C(=O)[C@H](C)N)OCC] | D-67 | E-15 |
| B-68 | 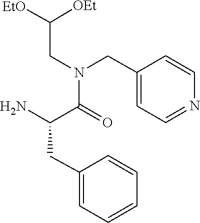 [SMILES: CCOC(CN(Cc1ccncc1)C(=O)[C@@H](N)Cc1ccccc1)OCC] | D-67 | E-21 |
| B-69 | 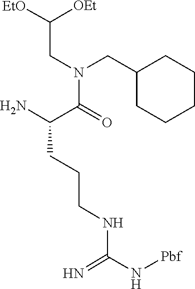 | D-10 | E-69 |

TABLE 5-9

| Int. B | Structure | Int. D | Int. E |
|---|---|---|---|
| B-70 | 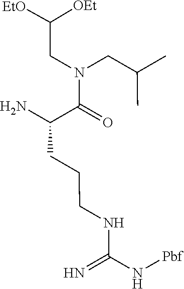 | D-70 | E-69 |
| B-72 | 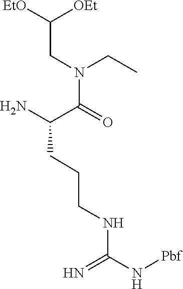 | D-128 | E-69 |
| B-73 | 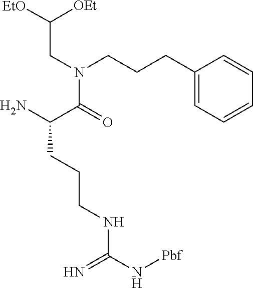 | D-73 | E-69 |
| B-74 | 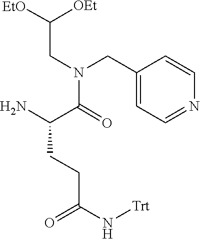 | D-67 | E-55 |
| B-78 | 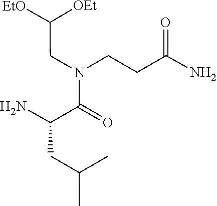 [SMILES: CCOC(CN(CCC(N)=O)C(=O)[C@@H](N)CC(C)C)OCC] | D-13 | E-41 |

TABLE 5-10

| Int. B | Structure | Int. D | Int. E |
|---|---|---|---|
| B-79 | 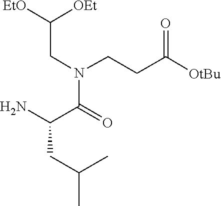 [SMILES: CCOC(CN(CCC(=O)OC(C)(C)C)C(=O)[C@@H](N)CC(C)C)OCC] | D-58 | E-41 |
| B-80 | 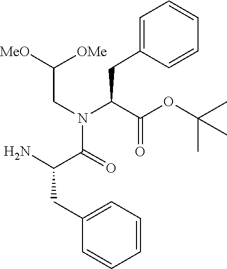 [SMILES: COC(CN(C(=O)[C@@H](N)Cc1ccccc1)[C@@H](Cc1ccccc1)C(=O)OC(C)(C)C)OC] | D-80 | E-21 |
| B-82 | 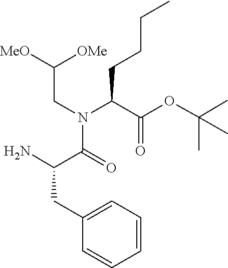 [SMILES: CCCC[C@@H](C(=O)OC(C)(C)C)N(CC(OC)OC)C(=O)[C@@H](N)Cc1ccccc1] | D-82 | E-21 |
| B-91 | 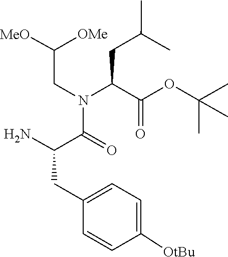 [SMILES: COC(CN(C(=O)[C@@H](N)Cc1ccc(OC(C)(C)C)cc1)[C@@H](CC(C)C)C(=O)OC(C)(C)C)OC] | D-91 | E-1 |
| B-92 | 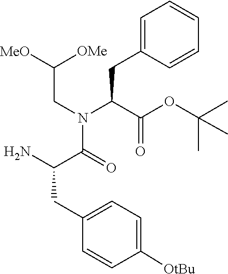 [SMILES: COC(CN(C(=O)[C@@H](N)Cc1ccc(OC(C)(C)C)cc1)[C@@H](Cc1ccccc1)C(=O)OC(C)(C)C)OC] | D-80 | E-1 |

TABLE 5-11

| Int. B | Structure | Int. D | Int. E |
|---|---|---|---|
| B-94 | 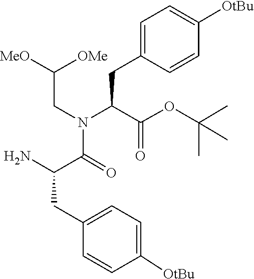 [SMILES: COC(CN(C(=O)[C@@H](N)Cc1ccc(OC(C)(C)C)cc1)[C@@H](Cc1ccc(OC(C)(C)C)cc1)C(=O)OC(C)(C)C)OC] | D-94 | E-1 |
| B-97 | 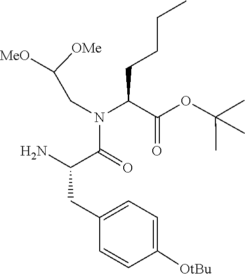 [SMILES: CCCC[C@@H](C(=O)OC(C)(C)C)N(CC(OC)OC)C(=O)[C@@H](N)Cc1ccc(OC(C)(C)C)cc1] | D-82 | E-1 |
| B-120 | 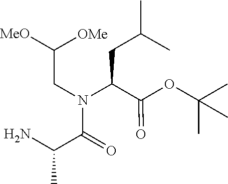 [SMILES: COC(CN(C(=O)[C@H](C)N)[C@@H](CC(C)C)C(=O)OC(C)(C)C)OC] | D-91 | E-15 |
| B-122 | 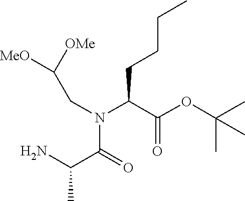 [SMILES: CCCC[C@@H](C(=O)OC(C)(C)C)N(CC(OC)OC)C(=O)[C@H](C)N] | D-82 | E-15 |
| B-123 | 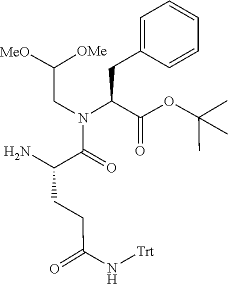 | D-80 | E-55 |

TABLE 5-12

| Int. B | Structure | Int. D | Int. E |
|---|---|---|---|
| B-124 | 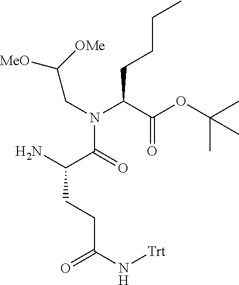 | D-82 | E-55 |
| B-127 | 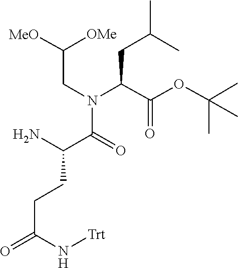 | D-91 | E-55 |
| B-128 | 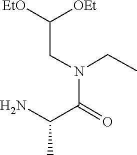 [SMILES: CCOC(CN(CC)C(=O)[C@H](C)N)OCC] | D-128 | E-15 |
| B-129 | 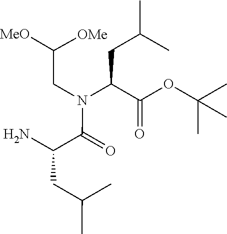 [SMILES: COC(CN(C(=O)[C@@H](N)CC(C)C)[C@@H](CC(C)C)C(=O)OC(C)(C)C)OC] | D-91 | E-41 |
| B-130 | 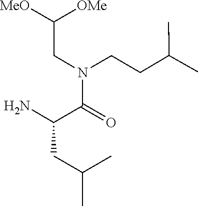 [SMILES: COC(CN(CCC(C)C)C(=O)[C@@H](N)CC(C)C)OC] | D-2 | E-41 |
| B-131 | 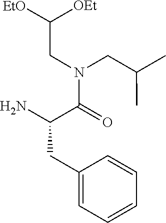 [SMILES: CCOC(CN(CC(C)C)C(=O)[C@@H](N)Cc1ccccc1)OCC] | D-70 | E-21 |

TABLE 5-13

| Int. B | Structure | Int. D | Int. E |
|---|---|---|---|
| B-132 | 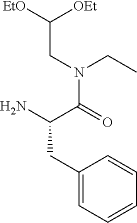 [SMILES: CCOC(CN(CC)C(=O)[C@@H](N)Cc1ccccc1)OCC] | D-128 | E-21 |
| B-135 | 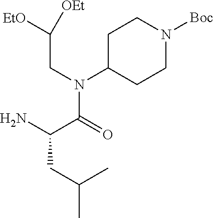 [SMILES: CCOC(CN(C(=O)[C@@H](N)CC(C)C)C1CCN(C(=O)OC(C)(C)C)CC1)OCC] | D-135 | E-41 |
| B-137 | 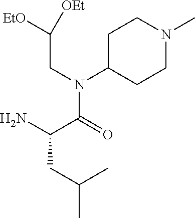 [SMILES: CCOC(CN(C(=O)[C@@H](N)CC(C)C)C1CCN(C)CC1)OCC] | D-137 | E-41 |
| B-139 | 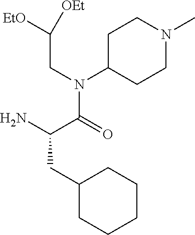 [SMILES: CCOC(CN(C(=O)[C@@H](N)CC1CCCCC1)C1CCN(C)CC1)OCC] | D-137 | E-13 |
| B-142 | 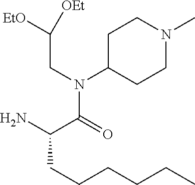 [SMILES: CCCCCC[C@H](N)C(=O)N(CC(OCC)OCC)C1CCN(C)CC1] | D-137 | E-142 |
| B-144 | 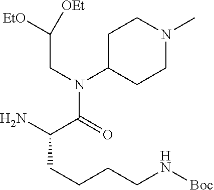 [SMILES: CCOC(CN(C(=O)[C@@H](N)CCCCNC(=O)OC(C)(C)C)C1CCN(C)CC1)OCC] | D-137 | E-28 |

TABLE 5-14

| Int. B | Structure | Int. D | Int. E |
|---|---|---|---|
| B-154 | 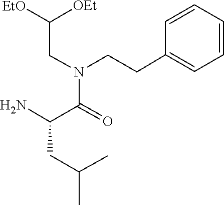 [SMILES: CCOC(CN(CCc1ccccc1)C(=O)[C@@H](N)CC(C)C)OCC] | D-30 | E-41 |
| B-157 | 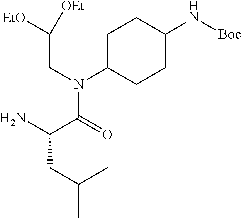 [SMILES: CCOC(CN(C(=O)[C@@H](N)CC(C)C)C1CCC(NC(=O)OC(C)(C)C)CC1)OCC] | D-157 | E-41 |
| B-158 | 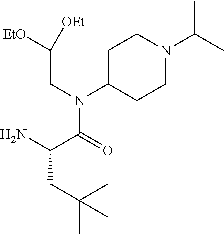 [SMILES: CCOC(CN(C(=O)[C@@H](N)CC(C)(C)C)C1CCN(C(C)C)CC1)OCC] | D-158 | E-158 |
| B-161 | 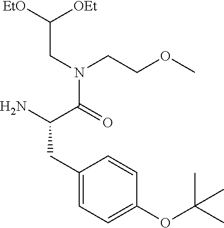 [SMILES: CCOC(CN(CCOC)C(=O)[C@@H](N)Cc1ccc(OC(C)(C)C)cc1)OCC] | D-161 | E-1 |
| B-176 | 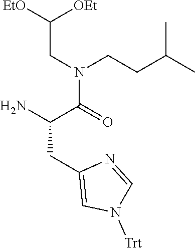 | D-2 | E-176 |

TABLE 5-15

| Int. B | Structure | Int. D | Int. E |
|---|---|---|---|
| B-177 | 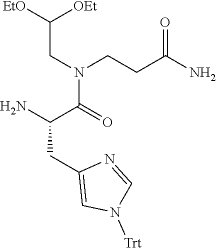 | D-13 | E-176 |
| B-178 | 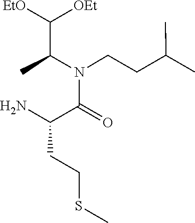 [SMILES: CCOC(OCC)[C@H](C)N(CCC(C)C)C(=O)[C@@H](N)CCSC] | D-178 | E-178 |
| B-179 | 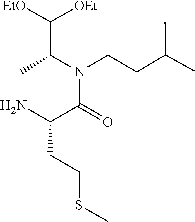 [SMILES: CCOC(OCC)[C@@H](C)N(CCC(C)C)C(=O)[C@@H](N)CCSC] | D-179 | E-178 |
| B-180 | 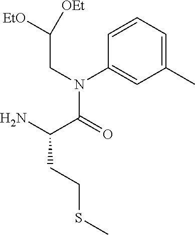 [SMILES: CCOC(CN(C(=O)[C@@H](N)CCSC)c1cccc(C)c1)OCC] | D-180 | E-178 |
| B-182 | 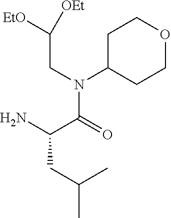 [SMILES: CCOC(CN(C(=O)[C@@H](N)CC(C)C)C1CCOCC1)OCC] | D-182 | E-41 |

### Production Example 8: Synthesis of Intermediate A-22

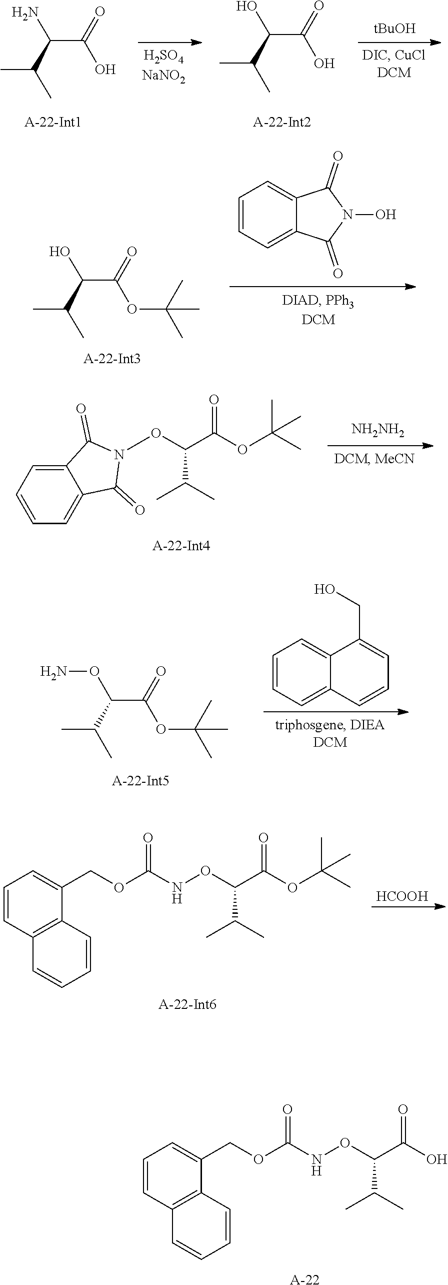

### 8-1) Synthesis of A-22-Int2

A-22-Int1 (400 g, 3.42 mol) was placed into a 10-L, three-necked flask, and 0.5M H₂SO₄ (5.2 L) was added. The reaction was cooled to 0° C. and then 2 mol/L NaNO₂ (2.6 L) was added drop wise, after the addition was completed; the reaction was stirred at room temperature overnight. After this time, the reaction mixture was extracted with ethyl acetate (EA) (3×3 L). The combined EA extracts were dried over Na₂SO₄, filtered and concentrated. The resulting crude solid was recrystallized from petroleum ether to afford compound A-22-Int2 (230 g).

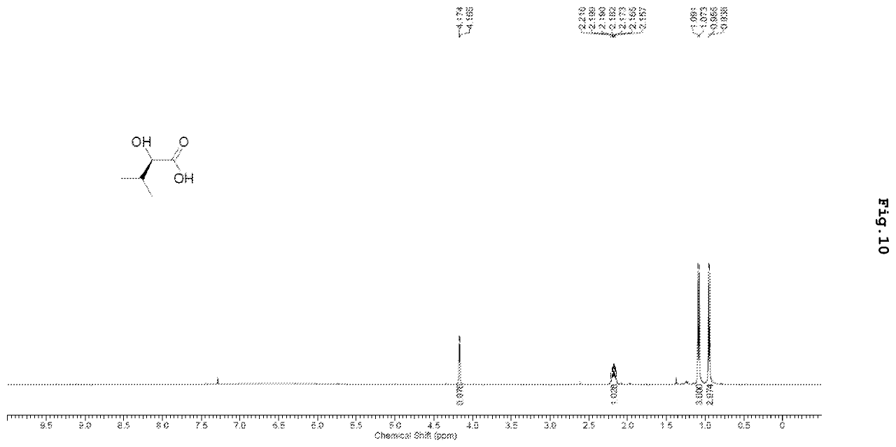

¹H NMR (400 MHz, CDCl₃) data of A-22-Int2 is shown in FIG. 10.

### 8-2) Synthesis of A-22-Int3

A solution of DIC (533 g, 4.23 mol) and CuCl (8 g, 0.08 mol) in t-BuOH (325.6 g, 4.4 mol) was stirred under nitrogen at room temperature for 5 days. The reaction was diluted with DCM (1 L). A-22-Int2 (200 g, 1.69 mol) was added to the solution in portion wise at 0° C. The reaction was stirred at room temperature for 5 hrs before filtering over a pad of celite. The filtrate was washed with saturated aqueous Na₂CO₃ and brine, then dried over with Na₂SO₄, concentrated and the residue was purified by distillation to afford A-22-Int3 (200 g).

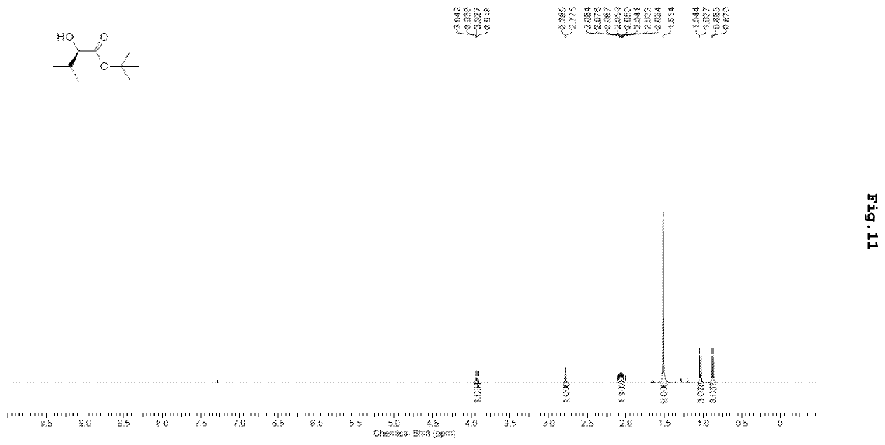

¹H NMR (400 MHz, CDCl₃) data of A-22-Int3 is shown in FIG. 11.

### 8-3) Synthesis of A-22-Int4

To a stirred solution of A-22-Int3 (100 g, 0.57 mol), PPh₃ (194.1 g, 0.74 mol) and compound N-hydroxyphthalimide (110.8 g, 0.68 mol) in DCM (1 L) was added DIAD (157.6 g, 0.74 mol) at −20° C. to −40° C. After 40 min at the same temperature, the reaction mixture was concentrated and directly purified by flash chromatography (PE:EA=100:1-10:1) to afford A-22-Int4 (73.8 g).

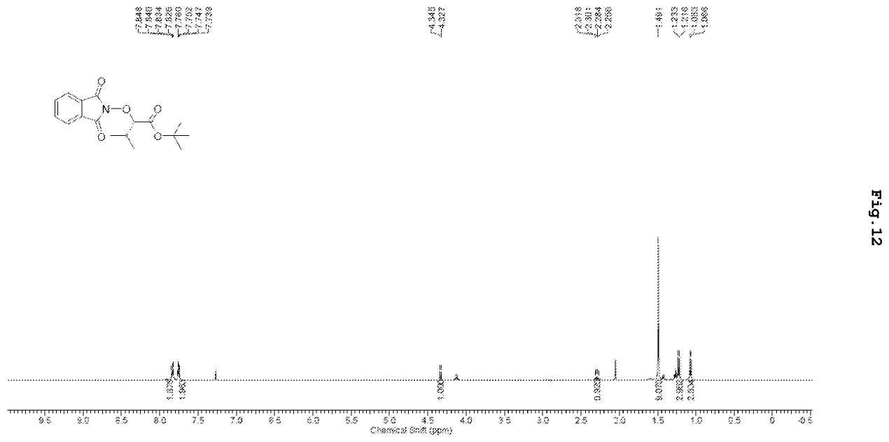

¹H NMR (400 MHz, CDCl₃) data of A-22-Int4 is shown FIG. 12.

### 8-4) Synthesis of A-22-Int5

To a stirring solution of A-22-Int4 (100 g, 0.31 mol) in DCM (500 mL) and MeCN (100 mL) was added NH₂NH₂·H₂O (39.2 g, 0.78 mol) at 0° C. After additional, the mixture was warmed to room temperature and stirred at the same temperature for 3 hrs before filtering. The filtrate was washed with saturated aqueous Na₂CO₃ and brine, then dried over Na₂SO₄, filtered and concentrated to afford A-22-Int5 (54 g). The crude product was used directly.

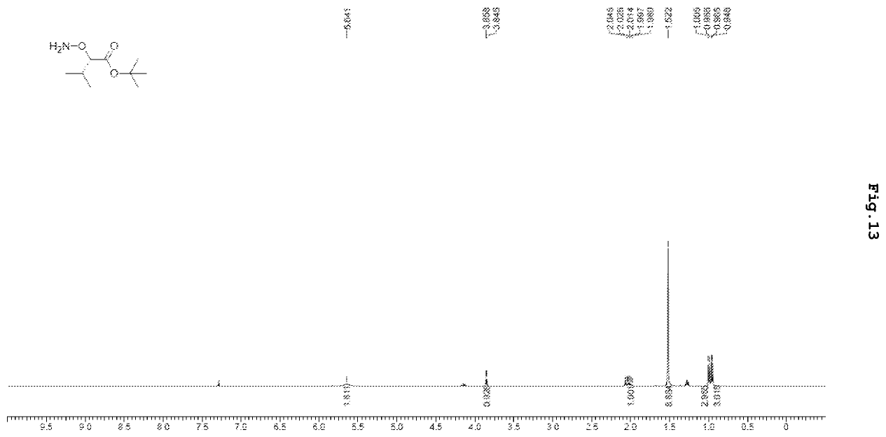

¹H NMR (400 MHz, CDCl₃) data of A-22-Int5 is shown in FIG. 13.

### 8-5) Synthesis of A-22-Int6

A 1000 mL of three-neck flask was charged with 1-naphthalene methanol (31.6 g, 200 mmol) dissolved in DCM (200 mL) at −10° C. Then DIEA (35 mL) was added dropwise slowly and the solution turned to be light red. After stirring for 5 min, triphosgene (solid, 29.6 g, 0.1 mol) was added in portions slowly to keep the temperature below 0° C. After addition, the mixture was stirred at the temperature for 20 min, TLC was used for monitoring the reaction. We can find the starting material was consumed. A-22-Int5 (32 g, 0.17 mol) in DCM (100 mL) was added dropwise slowly to keep the temperature below 0° C. and the mixture was stirred at the temperature for 20 mins. DIEA was added until no smoke was formed. Then the mixture was stirred at room temperature for 30 min. TLC identified the reaction was finished. Then the mixture was diluted with 300 mL of DCM and the organic layer was washed with water and brine, dried over Na₂SO₄, filtered and concentrated to give the residue, which was purified by flash chromatography (PE:EA 100:1-10:1) to give A-22-Int6 (36 g).

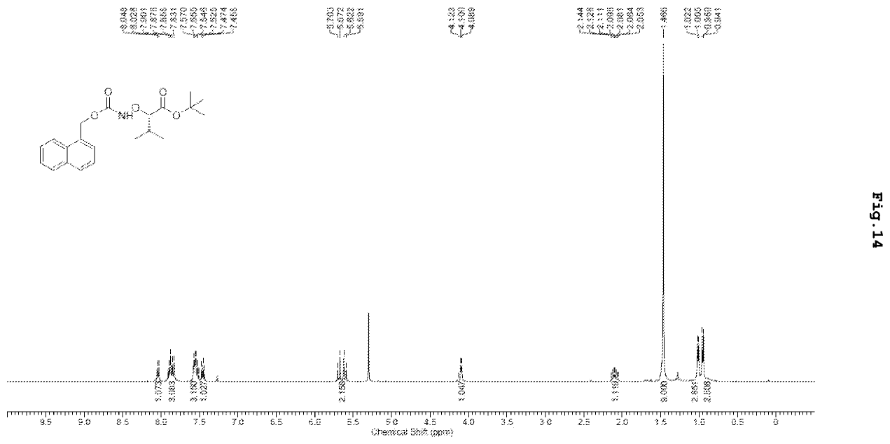

¹H NMR (400 MHz, CDCl₃) data of A-22-Int6 is shown in FIG. 14.

### 8-6) Synthesis of Intermediate A-22

A-22-Int6 (25 g, 67 mmol) was dissolved in formic acid (250 mL). After being stirred at room temperature for 6 hrs, the mixture was concentrated. The residue was purified by pre-HPLC to afford A-22 (13 g).

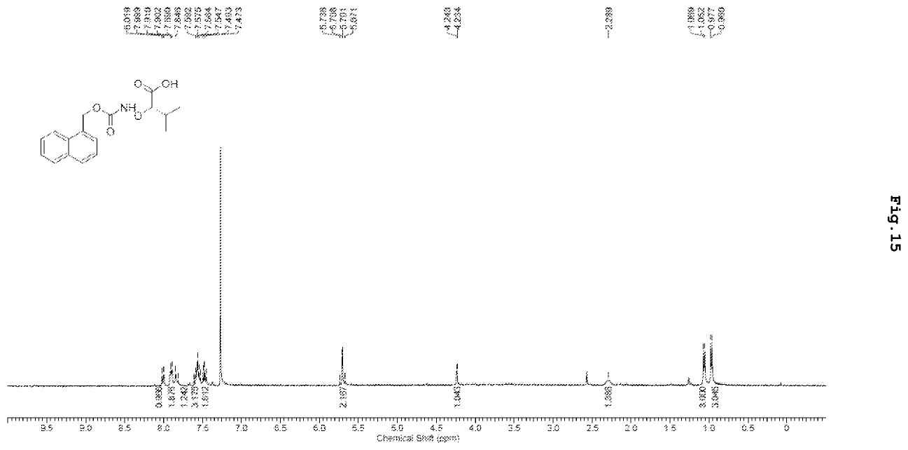

¹H NMR (400 MHz, CDCl₃) data of A-22 is shown in FIG. 15.

### Production Example 9: Synthesis of Intermediate A-43

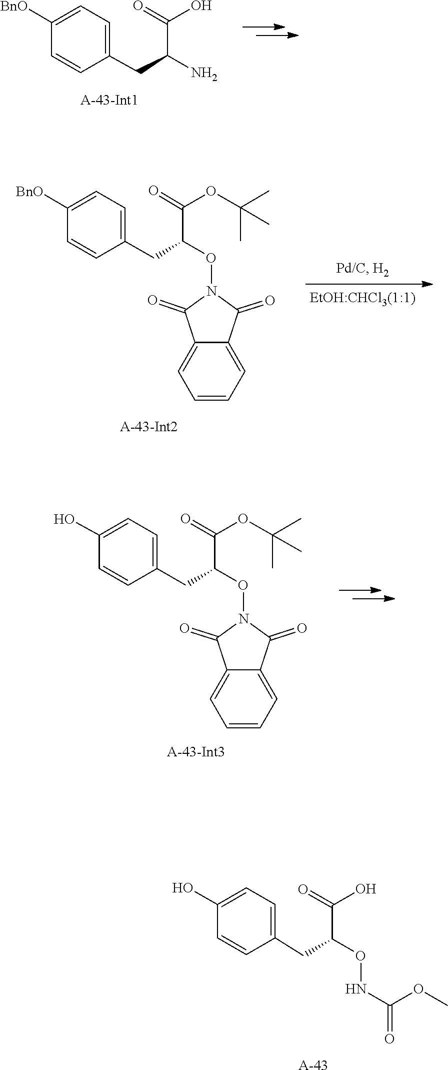 [SMILES: N[C@@H](Cc1ccc(OCc2ccccc2)cc1)C(=O)O ; CC(C)(C)OC(=O)[C@@H](Cc1ccc(OCc2ccccc2)cc1)ON1C(=O)c2ccccc2C1=O ; CC(C)(C)OC(=O)[C@@H](Cc1ccc(O)cc1)ON1C(=O)c2ccccc2C1=O ; COC(=O)NO[C@H](Cc1ccc(O)cc1)C(=O)O ; "A-43"]

A-43-Int2 was synthesized in the same manner as in Production Examples 8: 8-1)-8-3).

To a solution of A-43-Int2 (10.0 g, 21.1 mmol) in CHCl₃ (100 mL) and ethyl alcohol (100 mL) was added Pd/C (1.20 g, 10% purity) at 25° C. under nitrogen protection. The suspension was degassed under vacuum and purged with H₂ for 3 times and stirred for 8 hrs under hydrogen at 15 psi. The mixture was filtered and the filtrate was concentrated to get the A-43-Int3 (43.0 g, 96.5 mmol, 91% yield) as a yellow oil.

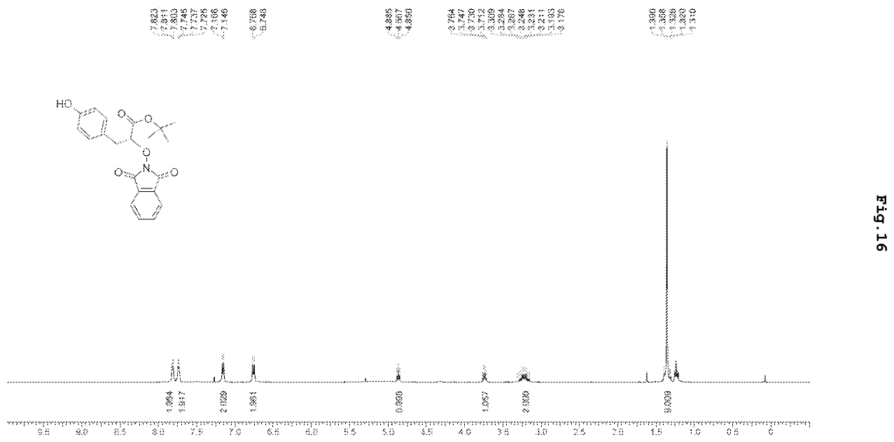

¹H NMR (400 MHz, CDCl₃) data of A-43-Int3 is shown in FIG. 16.

A-43 was synthesized by using A-43-Int3 in the same manner as in Production Examples 8: 8-4)-8-6).

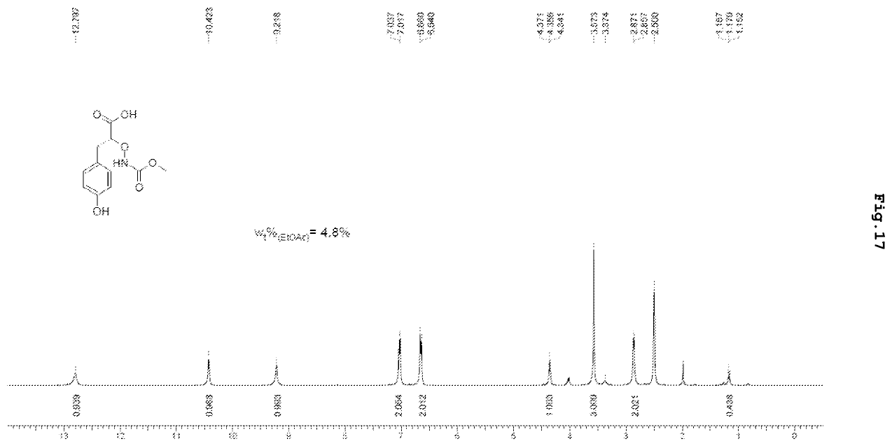

¹H NMR (400 MHz, DMSO) data of A-43 is shown in FIG. 17.

### Production Example 10: Synthesis of Intermediate A-60

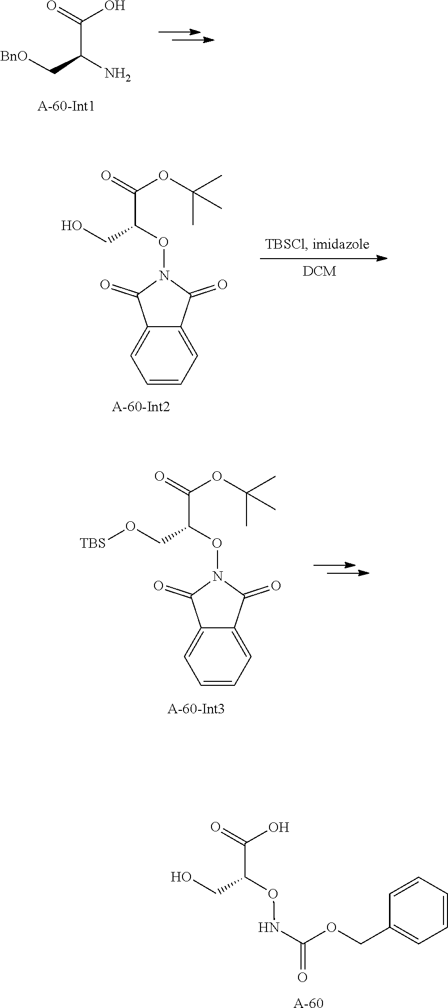

By a method similar to that for synthesizing A-43-Int2 from A-43-Int1 in Production Example 9, A-60-Int2 was synthesized.

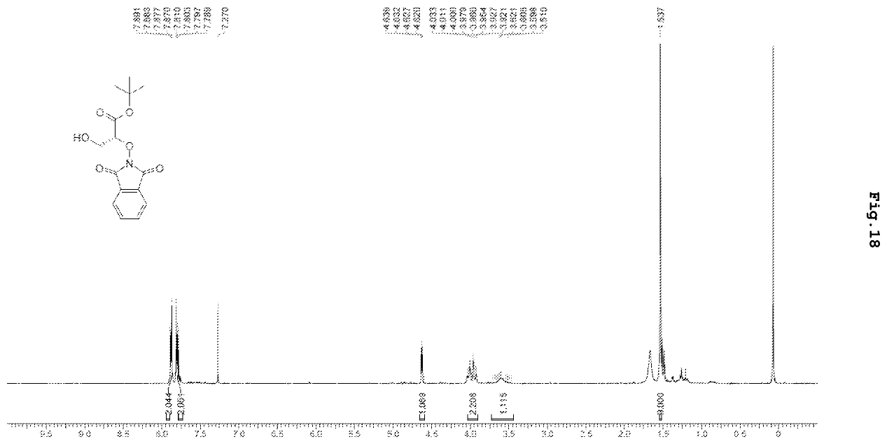

¹H NMR (400 MHz, CDCl₃) data of A-60-Int1 is shown in FIG. 18.

To a solution of A-60-Int2 (500 mg, 1.63 mmol) and imidazole (222 mg, 3.26 mmol) in dichloromethane (5 mL) was added TBSCl (369 mg, 2.49 mmol) at 0° C., and the mixture was stirred at 20° C. for 2 hrs. The reaction mixture was diluted with dichloromethane (10 mL) and washed with water (10 mL×2), the organic phase was concentrated and purified by column chromatography (petroleum ether/ethyl acetate=100/1 to 20/1) to get the product A-60-Int3 (500 mg, 1.19 mmol, 73%) as a colorless oil.

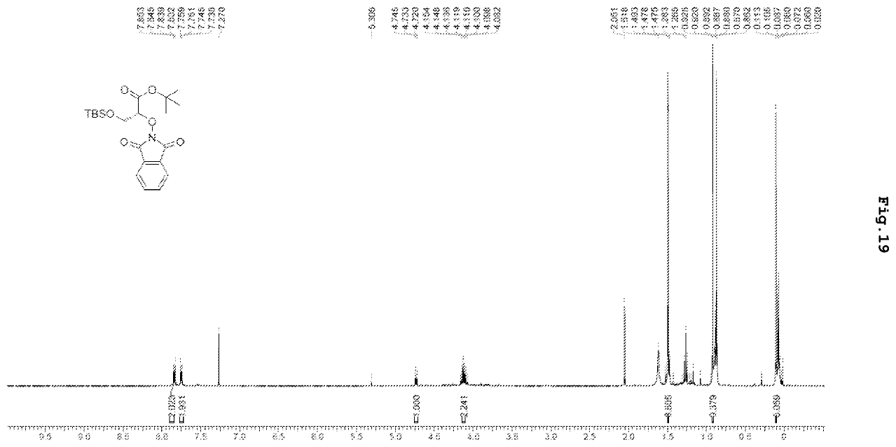

¹H NMR (400 MHz, DMSO) data of A-60-Int3 is shown in FIG. 19.

A-60 was synthesized by using A-60-Int3 in the same manner as in Production Example 8:8-4)-8-6).

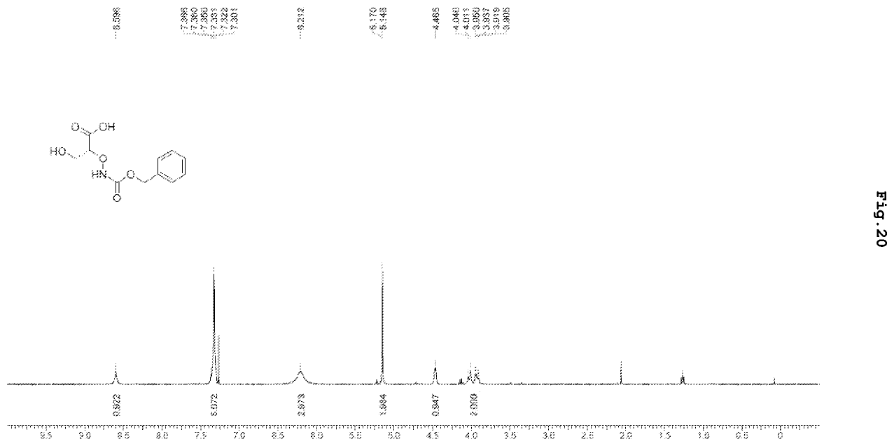

¹H NMR (400 MHz, CDCl₃) data of A-60 is shown in FIG. 20.

### Production Example 11: Synthesis of Intermediate A-52

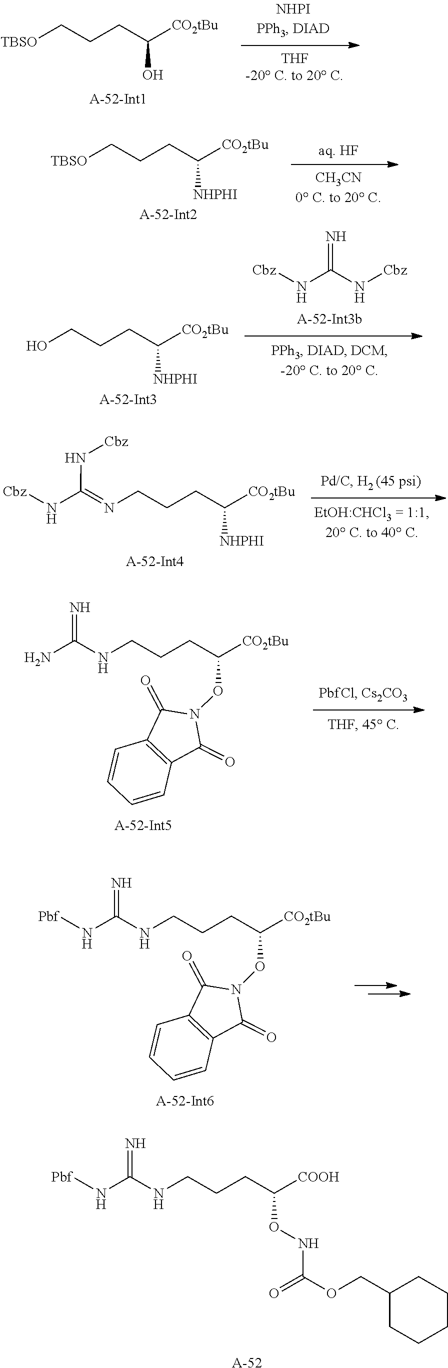

### 11-1) Synthesis of A-52-Int2

### (For Two Batches)

To a solution of A-52-Int1 (665 g, 2.18 mol), 2-hydroxyisoindoline-1,3-dione (391 g, 2.4 mol) and triphenylphosphane (745 g, 2.84 mol) in THF (7.0 L) was added diisopropyl azodicarboxylate (574 g, 2.84 mol, 552 mL) under nitrogen at −20° C. Then the mixture was warmed to 20° C. and stirred for 16 hrs. TLC (Petroleum ether:Ethyl acetate=5:1) showed starting material (A-52-Int1) was consumed completely. The reaction mixture was concentrated under reduced pressure to give a residue then poured into a mixture of ethyl acetate (1.0 L) and petroleum ether (10.0 L), amount of white solid was separate out, filtered and the organic layer was concentrated under reduced pressure to give a residue and purified by column chromatography (SiO₂, Petroleum ether:Ethyl acetate=50:1 to 15:1) to give A-52-Int2 (1.80 kg, 83% yield) as a white solid.

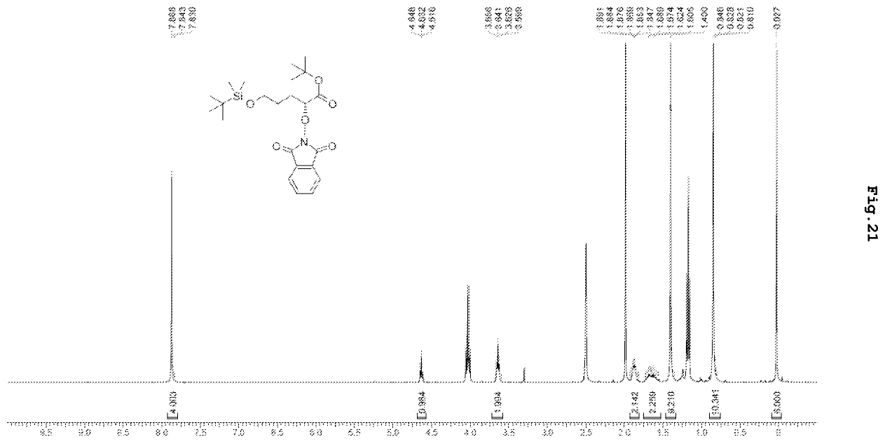

¹H NMR (400 MHz, DMSO) data of A-52-Int2 is shown in FIG. 21.

### 11-2) Synthesis of A-52-Int3

### (For Fourteen Batches)

To a solution of A-52-Int2 (120 g, 267 mmol) in MeCN (720 mL) was added a solution of hydrofluoric acid (33.4 g, 801 mmol, 30.3 mL, 48% purity) in H₂O at 0° C. Then the mixture was stirred at 20° C. for 15 mins. TLC (Petroleum ether:Ethyl acetate=5:1) showed starting material (A-52-Int2) was consumed completely. The reaction mixture was quenched by addition sat. NaHCO₃ aqueous (1.0 L) and then extracted with ethyl acetate (1.0 L). The combined organic layers were washed with brine (750 mL), dried over anhydrous sodium sulfate, filtered and concentrated under reduced pressure to give A-52-Int3 (1.20 kg, 3.58 mol, 96% yield) was obtained as yellow oil.

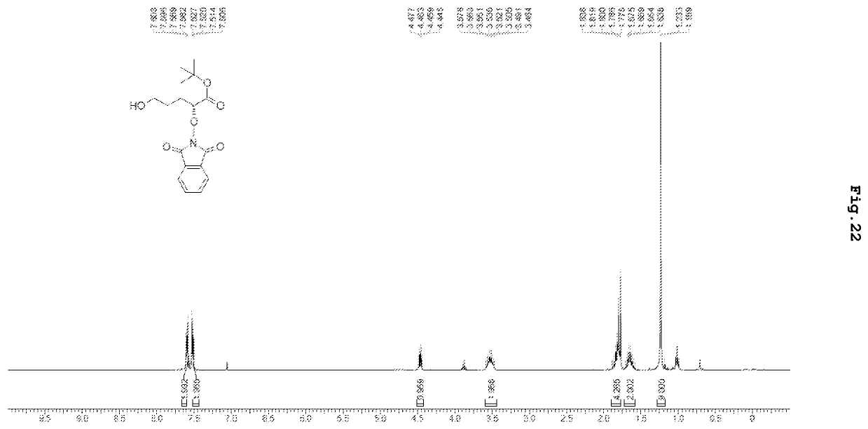

¹H NMR (400 MHz, CDCl₃) data of A-52-Int3 is shown in FIG. 22.

### 11-3) Synthesis of A-52-Int4

### (For Two Batches)

To a solution of A-52-Int3 (600 g, 1.79 mol) and 4-4-3b (586 g, 1.79 mol), PPh₃ (563 g, 2.15 mol) in DCM (8.0 L) was added diisopropyl azodicarboxylate (434 g, 2.15 mol, 417 mL) at −20° C. slowly over 2 hrs. The mixture was stirred at 20° C. for 16 hrs. TLC (Petroleum ether:Ethyl acetate=3:1) showed a major spot was desired compound. The reaction mixture was concentrated under reduced pressure to give a residue. The residue was triturated with petroleum ether (4.0 L) and ethyl acetate (4.0 L) and a lot of white solid was separated out, filtered and the organic layer was concentrated under reduced pressure to give a residue. The residue was purified by column chromatography (SiO₂, Petroleum ether:Ethyl acetate=50:1 to 5:1) to give a crude product, then the crude product was dissolved in EtOH (8.0 L) and stirred for about 2 hrs and a lot of white solid was precipitated, filtered to afford A-52-Int4 (2.0 kg, 3.10 mol, 87% yield) as a white solid.

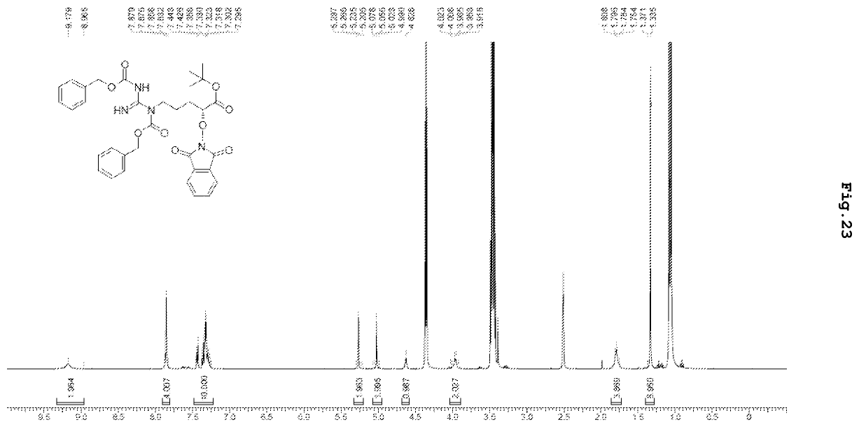

¹H NMR (400 MHz, DMSO) data of A-52-Int4 is shown in FIG. 23.

### 11-4) Synthesis of A-52-Int5

### (For Three Batches)

To a solution of A-52-Int4 (100 g, 155 mmol) in EtOH (400 mL) and CHCl₃ (400 mL) was added Pd/C (5.0 g, 155 mmol, 10% purity) under nitrogen at 20° C. The suspension was degassed under vacuum and purged with hydrogen several times. The mixture was stirred under hydrogen (45 psi) at 40° C. for 48 hrs. TLC (Petroleum ether:Ethyl acetate=2:1) showed starting material (A-52-Int4) was consumed completely. The reaction mixture was filtered and concentrated under reduced pressure to give a residue. The residue was dissolved in THF (1.0 L) and concentrated under reduced pressure to give A-52-Int5 (110 g, 292 mmol, 63% yield) as a white solid.

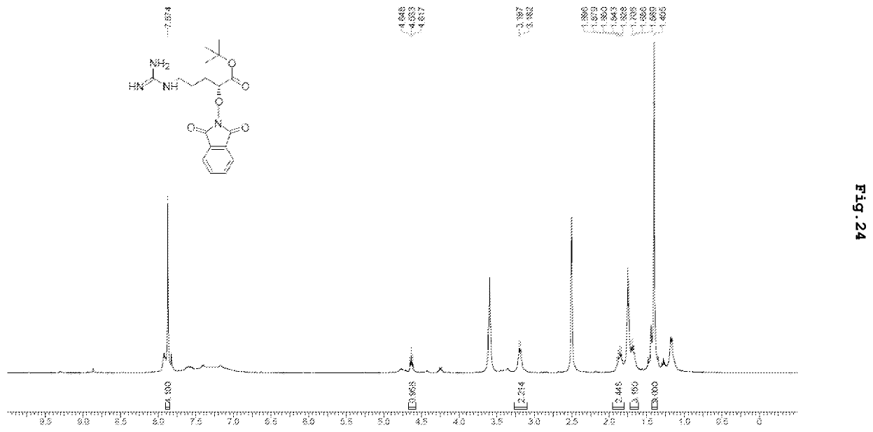

¹H NMR (400 MHz, DMSO) data of A-52-Int5 is shown in FIG. 24.

### 11-5) Synthesis of A-52-Int6 and A-52

### (For Five Batches)

To a solution of A-52-Int5 (136 g, 292 mmol) in THF (1.8 L) was added cesium carbonate (353 g, 1.08 mol) and 2,2,4,6,7-pentamethyl-3H-benzofuran-5-sulfonyl chloride (209 g, 723 mmol). The mixture was stirred at 45° C. for 16 hrs. TLC (Petroleum ether:Ethyl acetate=1:1) showed anew major spot was detected. The reaction mixture was quenched by addition water (2.0 L) at 23° C., and then extracted with ethyl acetate (2.0 L×2). The combined organic layers were dried over anhydrous sodium sulfate, filtered and concentrated under reduced pressure to give a residue. The residue was purified by column chromatography (Petroleum ether:Ethyl acetate=20:1 to 1:1) to afford A-52-Int6 (400 g, 636 mmol, 44% yield) as a white solid.

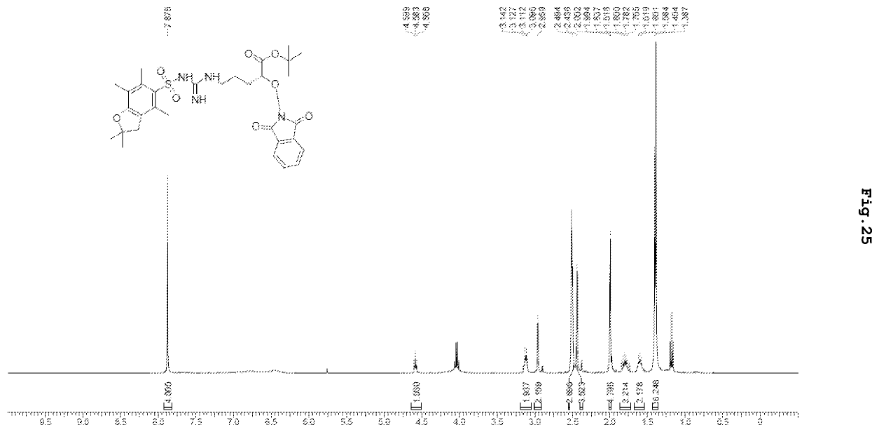

¹H NMR (400 MHz, DMSO) data of A-52-Int6 is shown in FIG. 25.

A-52 was synthesized by using A-52-Int6 in the same manner as in Production Example 8.

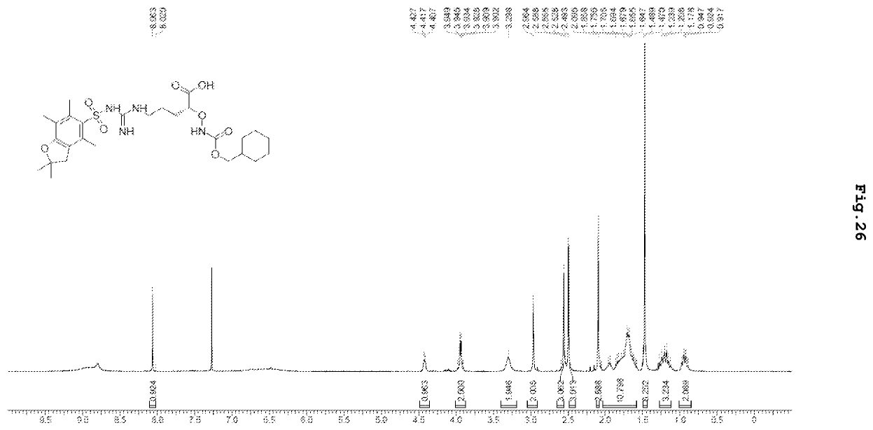

¹H NMR (400 MHz, CDCl₃) data of A-52 is shown in FIG. 26.

Intermediate A shown in Table 6 (Table 6-1 to Table 6-7) was synthesized by using the corresponding starting materials according to the same method as described above or a known method.

TABLE 6-1

| Int. A | Structure |
|---|---|
| A-1 | 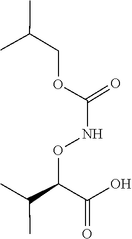 [SMILES: CC(C)COC(=O)NO[C@@H](C(=O)O)C(C)C] |
| A-2 | 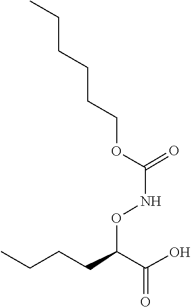 [SMILES: CCCCCCOC(=O)NO[C@H](CCCC)C(=O)O] |
| A-3 | 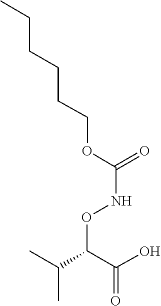 [SMILES: CCCCCCOC(=O)NO[C@H](C(=O)O)C(C)C] |
| A-4 | 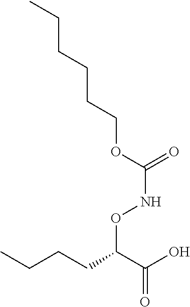 [SMILES: CCCCCCOC(=O)NO[C@@H](CCCC)C(=O)O] |
| A-5 | 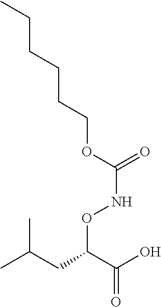 [SMILES: CCCCCCOC(=O)NO[C@@H](CC(C)C)C(=O)O] |
| A-6 | 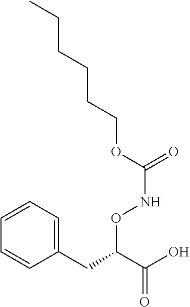 [SMILES: CCCCCCOC(=O)NO[C@@H](Cc1ccccc1)C(=O)O] |
| A-7 | 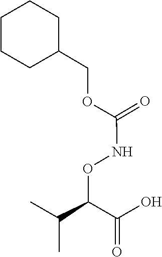 [SMILES: CC(C)[C@@H](ONC(=O)OCC1CCCCC1)C(=O)O] |
| A-8 | 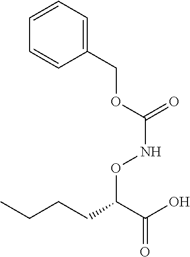 [SMILES: CCCC[C@H](ONC(=O)OCc1ccccc1)C(=O)O] |
| A-9 | 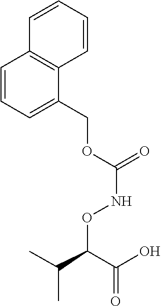 [SMILES: CC(C)[C@@H](ONC(=O)OCc1cccc2ccccc12)C(=O)O] |
| A-10 | 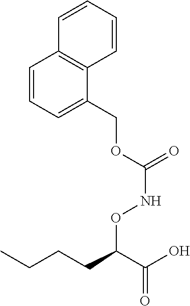 [SMILES: CCCC[C@@H](ONC(=O)OCc1cccc2ccccc12)C(=O)O] |
| A-13 | 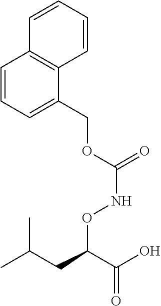 [SMILES: CC(C)C[C@@H](ONC(=O)OCc1cccc2ccccc12)C(=O)O] |
| A-15 | 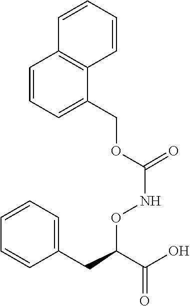 [SMILES: O=C(NO[C@H](Cc1ccccc1)C(=O)O)OCc1cccc2ccccc12] |

TABLE 6-2

| Int. A | Structure |
|---|---|
| A-18 | 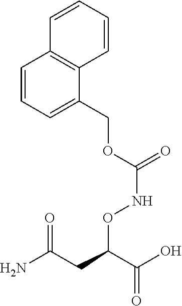 [SMILES: NC(=O)C[C@@H](ONC(=O)OCc1cccc2ccccc12)C(=O)O] |
| A-22 | 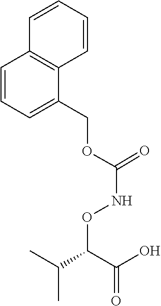 [SMILES: CC(C)[C@H](ONC(=O)OCc1cccc2ccccc12)C(=O)O] |
| A-23 | 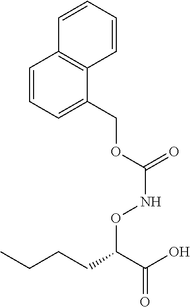 [SMILES: CCCC[C@H](ONC(=O)OCc1cccc2ccccc12)C(=O)O] |
| A-24 | 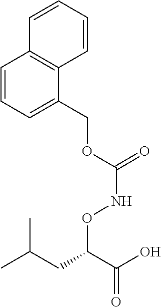 [SMILES: CC(C)C[C@H](ONC(=O)OCc1cccc2ccccc12)C(=O)O] |
| A-25 | 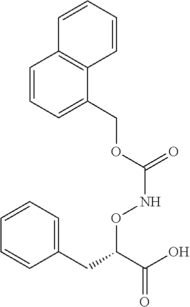 [SMILES: O=C(NO[C@@H](Cc1ccccc1)C(=O)O)OCc1cccc2ccccc12] |
| A-26 | 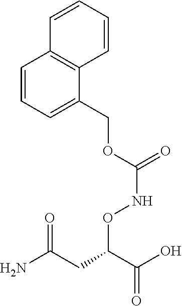 [SMILES: NC(=O)C[C@H](ONC(=O)OCc1cccc2ccccc12)C(=O)O] |
| A-28 | 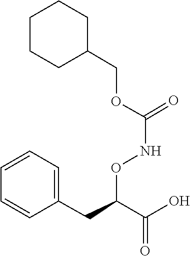 [SMILES: O=C(NO[C@H](Cc1ccccc1)C(=O)O)OCC1CCCCC1] |
| A-30 | 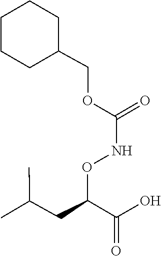 [SMILES: CC(C)C[C@@H](ONC(=O)OCC1CCCCC1)C(=O)O] |
| A-33 | 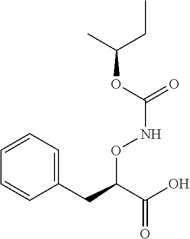 [SMILES: CC[C@H](C)OC(=O)NO[C@H](Cc1ccccc1)C(=O)O] |
| A-34 | 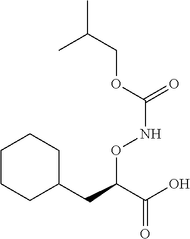 [SMILES: CC(C)COC(=O)NO[C@H](CC1CCCCC1)C(=O)O] |
| A-35 | 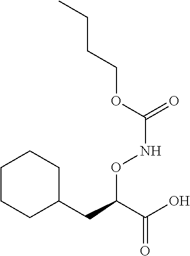 [SMILES: CCCCOC(=O)NO[C@H](CC1CCCCC1)C(=O)O] |
| A-36 | 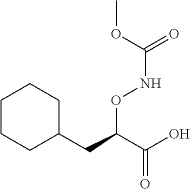 [SMILES: COC(=O)NO[C@H](CC1CCCCC1)C(=O)O] |

TABLE 6-3

| Int. A | Structure |
|---|---|
| A-37 | 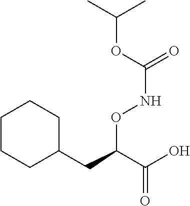 [SMILES: CC(C)OC(=O)NO[C@H](CC1CCCCC1)C(=O)O] |
| A-38 | 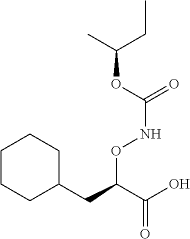 [SMILES: CC[C@H](C)OC(=O)NO[C@H](CC1CCCCC1)C(=O)O] |
| A-39 | 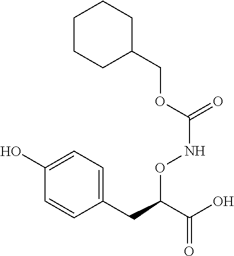 [SMILES: O=C(NO[C@H](Cc1ccc(O)cc1)C(=O)O)OCC1CCCCC1] |
| A-41 | 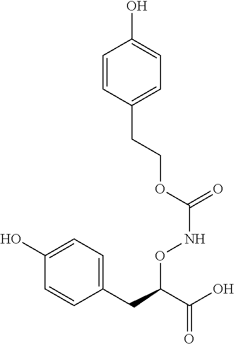 [SMILES: O=C(NO[C@H](Cc1ccc(O)cc1)C(=O)O)OCCc1ccc(O)cc1] |
| A-43 | 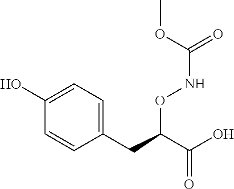 [SMILES: COC(=O)NO[C@H](Cc1ccc(O)cc1)C(=O)O] |
| A-44 | 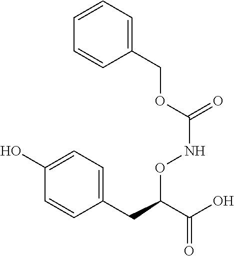 [SMILES: O=C(NO[C@H](Cc1ccc(O)cc1)C(=O)O)OCc1ccccc1] |
| A-50 | 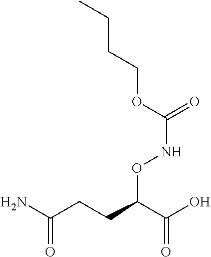 [SMILES: CCCCOC(=O)NO[C@H](CCC(N)=O)C(=O)O] |
| A-51 | 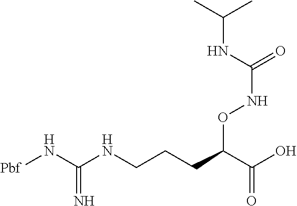 |
| A-52 | 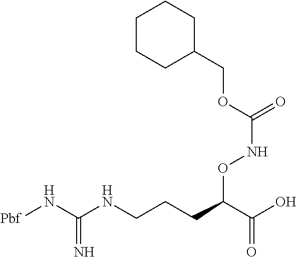 |
| A-54 | 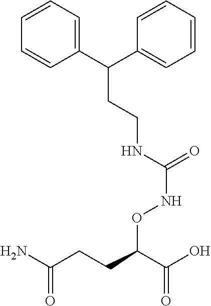 [SMILES: NC(=O)CC[C@@H](ONC(=O)NCCC(c1ccccc1)c1ccccc1)C(=O)O] |
| A-55 | 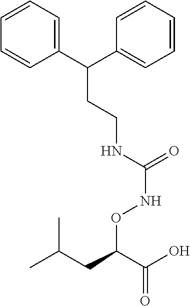 [SMILES: CC(C)C[C@@H](ONC(=O)NCCC(c1ccccc1)c1ccccc1)C(=O)O] |
| A-57 | 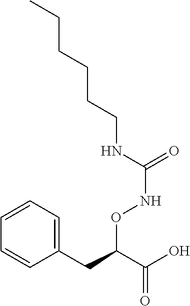 [SMILES: CCCCCCNC(=O)NO[C@H](Cc1ccccc1)C(=O)O] |

TABLE 6-4

| Int. A | Structure |
|---|---|
| A-58 | 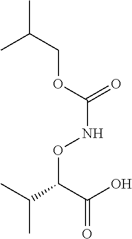 [SMILES: CC(C)COC(=O)NO[C@H](C(=O)O)C(C)C] |
| A-59 | 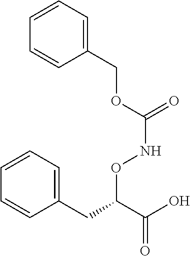 [SMILES: O=C(NO[C@@H](Cc1ccccc1)C(=O)O)OCc1ccccc1] |
| A-60 | 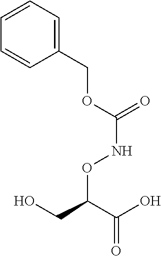 [SMILES: O=C(NO[C@H](CO)C(=O)O)OCc1ccccc1] |
| A-62 | 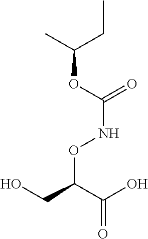 |
| A-66 | 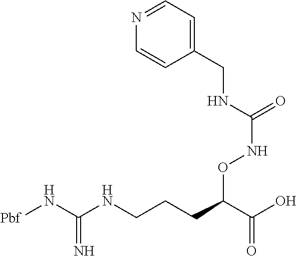 |
| A-68 | 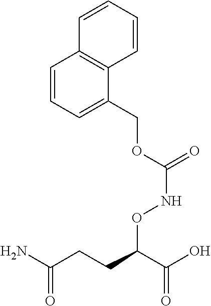 [SMILES: NC(=O)CC[C@@H](ONC(=O)OCc1cccc2ccccc12)C(=O)O] |
| A-69 | 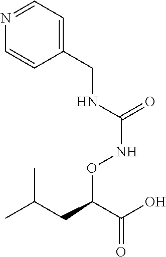 [SMILES: CC(C)C[C@@H](ONC(=O)NCc1ccncc1)C(=O)O] |
| A-70 | 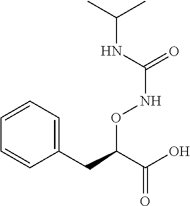 [SMILES: CC(C)NC(=O)NO[C@H](Cc1ccccc1)C(=O)O] |
| A-71 | 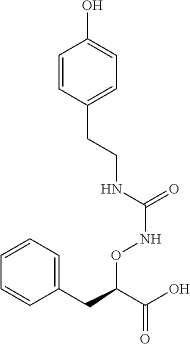 [SMILES: O=C(NCCc1ccc(O)cc1)NO[C@H](Cc1ccccc1)C(=O)O] |
| A-72 | 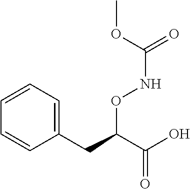 [SMILES: COC(=O)NO[C@H](Cc1ccccc1)C(=O)O] |
| A-74 | 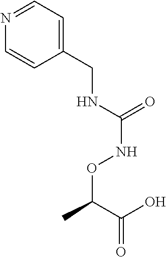 [SMILES: C[C@@H](ONC(=O)NCc1ccncc1)C(=O)O] |
| A-75 | 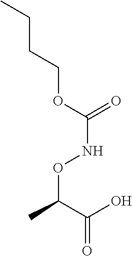 |

TABLE 6-5

| Int. A | Structure |
|---|---|
| A-76 | 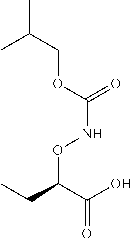 |
| A-77 | 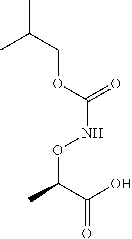 |
| A-78 | 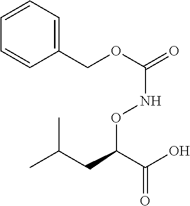 [SMILES: CC(C)C[C@@H](ONC(=O)OCc1ccccc1)C(=O)O] |
| A-80 | 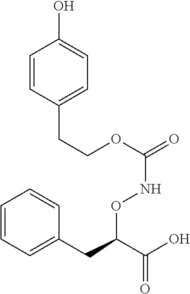 [SMILES: O=C(NO[C@H](Cc1ccccc1)C(=O)O)OCCc1ccc(O)cc1] |
| A-82 | 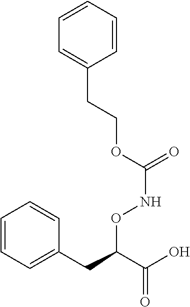 [SMILES: O=C(NO[C@H](Cc1ccccc1)C(=O)O)OCCc1ccccc1] |
| A-84 | 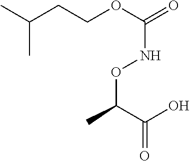 |
| A-85 | 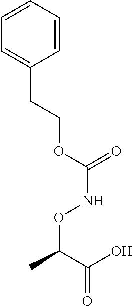 [SMILES: C[C@@H](ONC(=O)OCCc1ccccc1)C(=O)O] |
| A-86 | 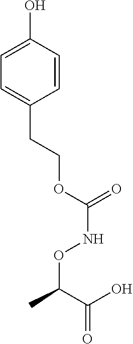 [SMILES: C[C@@H](ONC(=O)OCCc1ccc(O)cc1)C(=O)O] |
| A-90 | 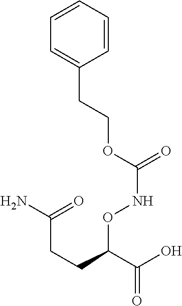 [SMILES: NC(=O)CC[C@@H](ONC(=O)OCCc1ccccc1)C(=O)O] |
| A-91 | 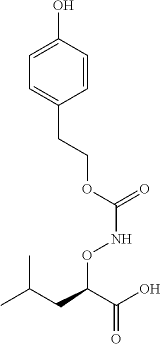 [SMILES: CC(C)C[C@@H](ONC(=O)OCCc1ccc(O)cc1)C(=O)O] |
| A-92 | 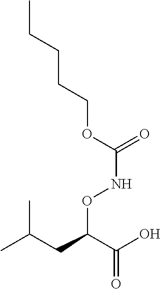 [SMILES: CCCCCOC(=O)NO[C@H](CC(C)C)C(=O)O] |
| A-94 | 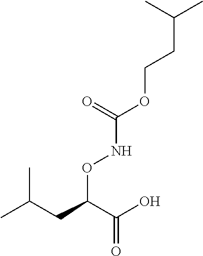 [SMILES: CC(C)CCOC(=O)NO[C@H](CC(C)C)C(=O)O] |

TABLE 6-6

| Int. A | Structure |
|---|---|
| A-99 | 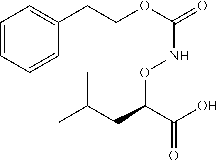 [SMILES: CC(C)C[C@@H](ONC(=O)OCCc1ccccc1)C(=O)O] |
| A-105 | 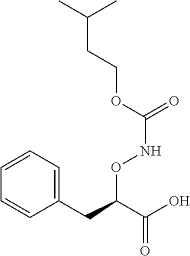 [SMILES: CC(C)CCOC(=O)NO[C@H](Cc1ccccc1)C(=O)O] |
| A-111 | 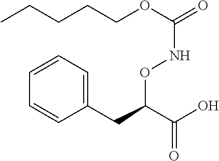 [SMILES: CCCCCOC(=O)NO[C@H](Cc1ccccc1)C(=O)O] |
| A-114 | 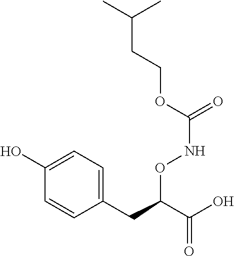 [SMILES: CC(C)CCOC(=O)NO[C@H](Cc1ccc(O)cc1)C(=O)O] |
| A-115 | 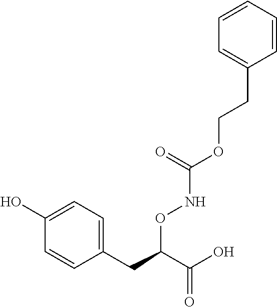 [SMILES: O=C(NO[C@H](Cc1ccc(O)cc1)C(=O)O)OCCc1ccccc1] |
| A-118 | 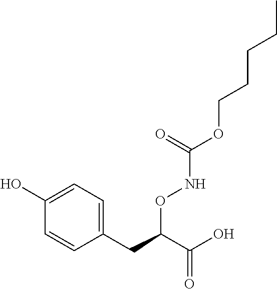 [SMILES: CCCCCOC(=O)NO[C@H](Cc1ccc(O)cc1)C(=O)O] |
| A-129 | 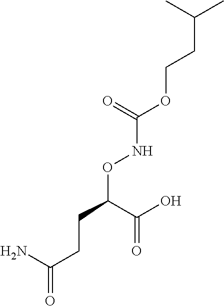 [SMILES: CC(C)CCOC(=O)NO[C@H](CCC(N)=O)C(=O)O] |
| A-130 | 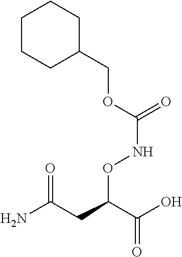 [SMILES: NC(=O)C[C@@H](ONC(=O)OCC1CCCCC1)C(=O)O] |
| A-132 | 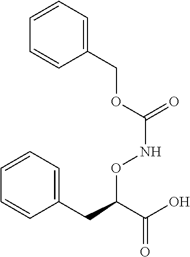 [SMILES: O=C(NO[C@H](Cc1ccccc1)C(=O)O)OCc1ccccc1] |
| A-133 | 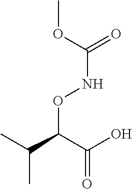 |
| A-134 | 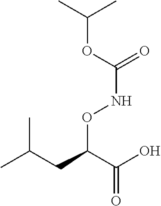 [SMILES: CC(C)C[C@@H](ONC(=O)OC(C)C)C(=O)O] |
| A-138 | 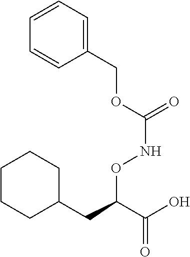 [SMILES: O=C(NO[C@H](CC1CCCCC1)C(=O)O)OCc1ccccc1] |

TABLE 6-7

| Int. A | Structure |
|---|---|
| A-153 | 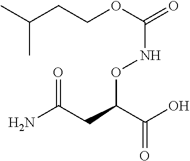 [SMILES: CC(C)CCOC(=O)NO[C@H](CC(N)=O)C(=O)O] |
| A-155 | 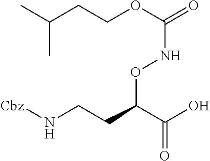 [SMILES: CC(C)CCOC(=O)NO[C@H](CCNC(=O)OCc1ccccc1)C(=O)O] |
| A-161 | 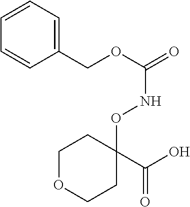 [SMILES: O=C(NOC1(C(=O)O)CCOCC1)OCc1ccccc1] |
| A-162 | 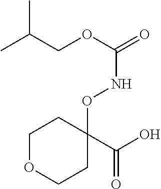 [SMILES: CC(C)COC(=O)NOC1(C(=O)O)CCOCC1] |
| A-163 | 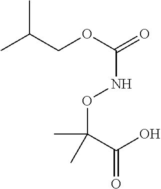 |
| A-164 | 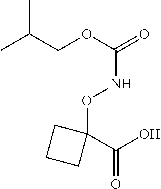 [SMILES: CC(C)COC(=O)NOC1(C(=O)O)CCC1] |
| A-165 | 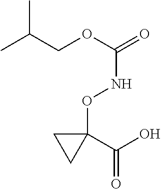 |
| A-176 | 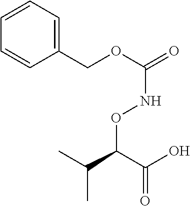 [SMILES: CC(C)[C@@H](ONC(=O)OCc1ccccc1)C(=O)O] |
| A-178 | 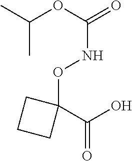 |
| A-180 | 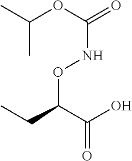 |

All the Intermediates C shown in Table 7 are commercially available or were synthesized according to a known method.

TABLE 7

| Int. C | Structure |
|---|---|
| C-80 | 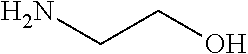 |
| C-81 | 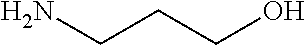 |
| C-82 | 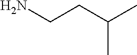 |
| C-83 | 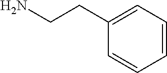 |
| C-84 | 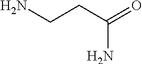 |
| C-87 | 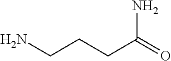 |
| C-98 | 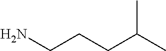 |
| C-123 | 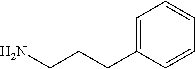 |
| C-127 | 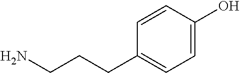 [SMILES: NCCCc1ccc(O)cc1] |

### Example 1: Synthesis of I-128

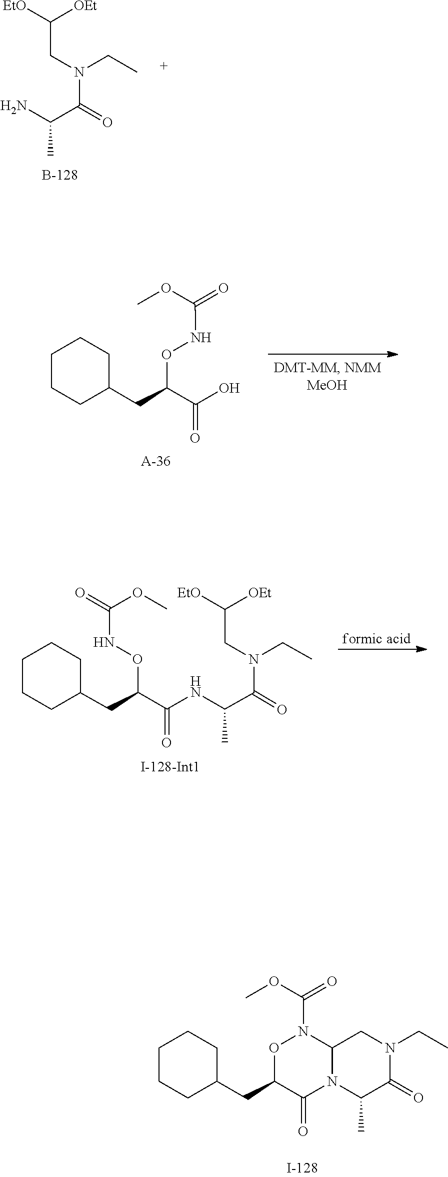 [SMILES: CCOC(CN(CC)C(=O)[C@H](C)N)OCC ; COC(=O)NO[C@H](CC1CCCCC1)C(=O)O ; "A-36" ; CCOC(CN(CC)C(=O)[C@H](C)NC(=O)[C@@H](CC1CCCCC1)ONC(=O)OC)OCC ; CCN1CC2N(C(=O)OC)O[C@H](CC3CCCCC3)C(=O)N2[C@@H](C)C1=O]

To a solution of A-36 (123 mg, 0.50 mmol, 0.50 mmol/mL in MeOH), B-128 (0.40 mmol, 0.40 mmol/mL in MeOH, 1.0 eq.) in methanol (2 mL) was added 4-(4,6-dimethoxy-1,3,5-triazin-2-yl)-4-methylmorpholinium chloride n-hydrate (DMT-MM) (0.45 mmol, 0.45 mmol/mL in MeOH, 1.1 eq.). The mixture stood 2 hours at 10° C. and then was concentrated under reduced pressure. The residue was diluted with dichloromethane (10 mL) and washed with saturated sodium hydrogen carbonate solution (5 mL), brine (5 mL), dried over anhydrous sodium sulfate, filtered and concentrated. The crude product was dissolved in formic acid (3 mL) and stood for 2 days at 10° C. The mixture was concentrated under reduced pressure and the residue was purified by prep-HPLC (FA) to give the product I-128.

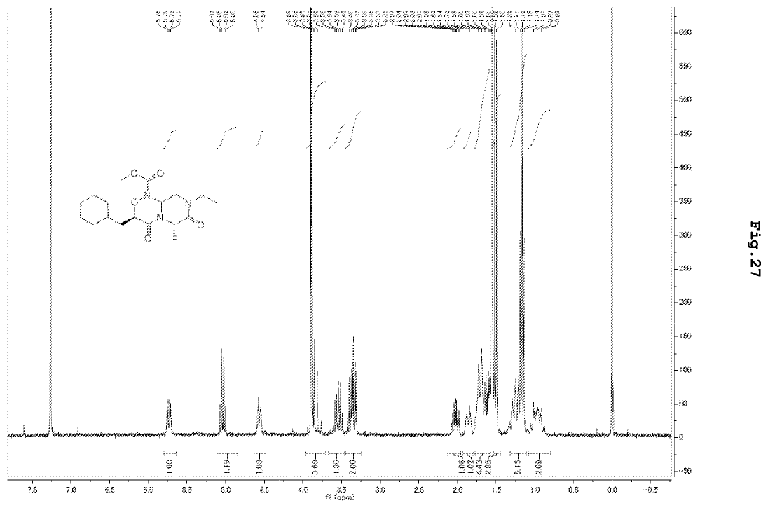

¹H NMR (400 MHz, CDCl₃) data of I-128 is shown in FIG. 27.

### Reference Example 1

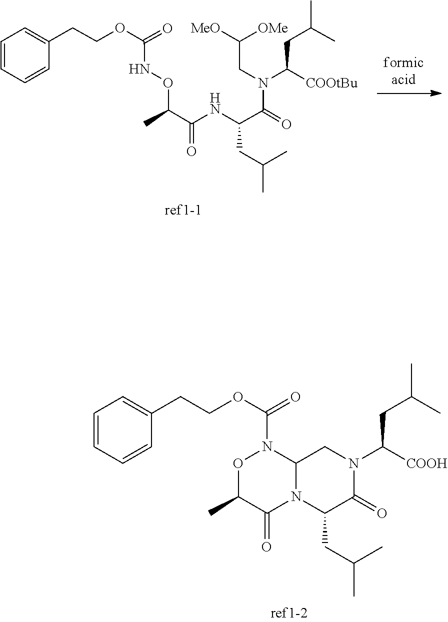

Compound ref1-1 (4.05 g, 6.5 mmol) was dissolved in formic acid (30 mL) and the mixture was stirred for 1 day at 25° C. The mixture was concentrated under reduced pressure and the residue was purified by prep-HPLC to give the compound ref1-2.

### Example 2: Synthesis of I-129

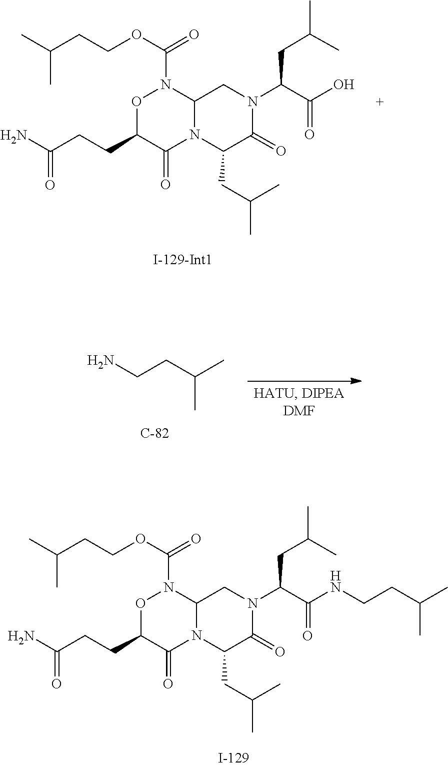

I-129-Int1 was synthesized using A-129 and B-129 in the same manner as in Example 1 and Reference Example 1.

To a solution of I-129-Int1 (0.09 mmol, 0.045 mmol/mL in N,N-DMF, 1.0 eq.), C-82 (0.18 mmol, 0.09 mmol/mL in DMF, 2.0 eq.) in DMF (2.0 mL) was added 1-[bis(dimethylamino)methylene]-1H-1,2,3-triazolo[4,5-b]pyridinium 3-oxide hexafluorophosphate (HATU) (0.14 mmol, 0.28 mmol/mL in DMF, 1.5 eq.) in DMF (0.5 mL) and N-ethyl-N-isopropyl-propan-2-amine (DIPEA) (0.28 mmol, 0.56 mmol/mL in DMF, 3.0 eq.) in DMF (0.5 mL) at 25° C. The mixture was stirred at 25° C. for 2 hours. The reaction mixture was filtered to give a filtrate and the filtrate was purified by prep-HPLC (FA condition) to give the target compound I-129.

Compounds I-1 to I-140, I-153 to I-157, I-161 to I-165 and I-176 to I-180 were synthesized according to the above method or a known method using the Intermediates shown in Table 8 (Table 8-1 to Table 8-3).

TABLE 8-1

| Compound | | | |
|---|---|---|---|
| No. | Int. A | Int. B | Int. C |
| I-1 | A-1 | B-1 | none |
| I-2 | A-2 | B-2 | none |
| I-3 | A-3 | B-3 | none |
| I-4 | A-4 | B-3 | none |
| I-5 | A-5 | B-3 | none |
| I-6 | A-6 | B-3 | none |
| I-7 | A-7 | B-3 | none |
| I-8 | A-8 | B-8 | none |
| I-9 | A-9 | B-2 | none |
| I-10 | A-10 | B-10 | none |
| I-11 | A-10 | B-3 | none |
| I-12 | A-10 | B-12 | none |
| I-13 | A-13 | B-13 | none |
| I-14 | A-13 | B-12 | none |
| I-15 | A-15 | B-15 | none |
| I-16 | A-15 | B-2 | none |
| I-17 | A-15 | B-12 | none |
| I-18 | A-18 | B-18 | none |
| I-19 | A-18 | B-19 | none |
| I-20 | A-18 | B-20 | none |
| I-21 | A-18 | B-21 | none |
| I-22 | A-22 | B-12 | none |
| I-23 | A-23 | B-12 | none |
| I-24 | A-24 | B-12 | none |
| I-25 | A-25 | B-25 | none |
| I-26 | A-26 | B-26 | none |
| I-27 | A-26 | B-27 | none |
| I-28 | A-28 | B-28 | none |
| I-29 | A-28 | B-29 | none |
| I-30 | A-30 | B-30 | none |
| I-31 | A-30 | B-31 | none |
| I-32 | A-28 | B-32 | none |
| I-33 | A-33 | B-33 | none |
| I-34 | A-34 | B-28 | none |
| I-35 | A-35 | B-35 | none |
| I-36 | A-36 | B-29 | none |
| I-37 | A-37 | B-30 | none |
| I-38 | A-38 | B-30 | none |
| I-39 | A-39 | B-39 | none |
| I-40 | A-34 | B-31 | none |
| I-41 | A-41 | B-41 | none |
| I-42 | A-36 | B-33 | none |
| I-43 | A-43 | B-43 | none |
| I-44 | A-44 | B-44 | none |
| I-45 | A-41 | B-45 | none |
| I-46 | A-41 | B-46 | none |
| I-47 | A-39 | B-47 | none |
| I-48 | A-44 | B-47 | none |
| I-49 | A-39 | B-49 | none |
| I-50 | A-50 | B-50 | none |
| I-51 | A-51 | B-51 | none |
| I-52 | A-52 | B-51 | none |
| I-53 | A-52 | B-53 | none |
| I-54 | A-54 | B-54 | none |
| I-55 | A-55 | B-55 | none |
| I-56 | A-55 | B-56 | none |
| I-57 | A-57 | B-56 | none |
| I-58 | A-58 | B-58 | none |
| I-59 | A-59 | B-59 | none |
| I-60 | A-60 | B-60 | none |

TABLE 8-2

| Compound | | | |
|---|---|---|---|
| No. | Int. A | Int. B | Int. C |
| I-61 | A-41 | B-61 | none |
| I-62 | A-62 | B-62 | none |
| I-63 | A-60 | B-63 | none |
| I-64 | A-41 | B-64 | none |
| I-65 | A-62 | B-25 | none |
| I-66 | A-66 | B-15 | none |
| I-67 | A-52 | B-67 | none |
| I-68 | A-68 | B-68 | none |
| I-69 | A-69 | B-69 | none |
| I-70 | A-70 | B-70 | none |
| I-71 | A-71 | B-70 | none |
| I-72 | A-72 | B-72 | none |
| I-73 | A-71 | B-73 | none |
| I-74 | A-74 | B-74 | none |
| I-75 | A-75 | B-74 | none |
| I-76 | A-76 | B-20 | none |
| I-77 | A-77 | B-74 | none |
| I-78 | A-78 | B-78 | none |
| I-79 | A-78 | B-79 | none |
| I-80 | A-80 | B-80 | C-80 |
| I-81 | A-80 | B-80 | C-81 |
| I-82 | A-82 | B-82 | C-82 |
| I-83 | A-41 | B-80 | C-83 |
| I-84 | A-84 | B-80 | C-84 |
| I-85 | A-85 | B-80 | C-84 |
| I-86 | A-86 | B-80 | C-84 |
| I-87 | A-84 | B-80 | C-87 |
| I-88 | A-84 | B-80 | C-80 |
| I-89 | A-84 | B-80 | C-81 |
| I-90 | A-90 | B-80 | C-82 |
| I-91 | A-91 | B-91 | C-82 |
| I-92 | A-92 | B-92 | C-82 |
| I-93 | A-91 | B-92 | C-83 |
| I-94 | A-94 | B-94 | C-82 |
| I-95 | A-91 | B-94 | C-82 |
| I-96 | A-92 | B-94 | C-82 |
| I-97 | A-91 | B-97 | C-82 |
| I-98 | A-94 | B-94 | C-98 |
| I-99 | A-99 | B-94 | C-98 |
| I-100 | A-92 | B-94 | C-98 |
| I-101 | A-91 | B-97 | C-98 |
| I-102 | A-99 | B-94 | C-83 |
| I-103 | A-91 | B-97 | C-83 |
| I-104 | A-80 | B-91 | C-98 |
| I-105 | A-105 | B-92 | C-83 |
| I-106 | A-105 | B-92 | C-81 |
| I-107 | A-105 | B-94 | C-82 |
| I-108 | A-80 | B-94 | C-82 |
| I-109 | A-80 | B-97 | C-82 |
| I-110 | A-80 | B-94 | C-98 |
| I-111 | A-111 | B-94 | C-98 |
| I-112 | A-80 | B-97 | C-98 |
| I-113 | A-111 | B-94 | C-83 |
| I-114 | A-114 | B-91 | C-83 |
| I-115 | A-115 | B-91 | C-83 |
| I-116 | A-41 | B-92 | C-83 |
| I-117 | A-114 | B-94 | C-83 |
| I-118 | A-118 | B-94 | C-83 |
| I-119 | A-41 | B-97 | C-83 |
| I-120 | A-80 | B-120 | C-82 |

TABLE 8-3

| Compound | | | |
|---|---|---|---|
| No. | Int. A | Int. B | Int. C |
| I-121 | A-80 | B-120 | C-83 |
| I-122 | A-80 | B-122 | C-98 |
| I-123 | A-99 | B-123 | C-123 |
| I-124 | A-99 | B-124 | C-123 |
| I-125 | A-82 | B-123 | C-123 |
| I-126 | A-80 | B-123 | C-123 |
| I-127 | A-111 | B-127 | C-127 |
| I-128 | A-36 | B-128 | none |
| I-129 | A-129 | B-129 | C-82 |
| I-130 | A-130 | B-130 | none |
| I-131 | A-33 | B-131 | none |
| I-132 | A-132 | B-132 | none |
| I-133 | A-133 | B-131 | none |
| I-134 | A-134 | B-130 | none |
| I-135 | A-30 | B-135 | none |
| I-136 | A-13 | B-135 | none |
| I-137 | A-13 | B-137 | none |
| I-138 | A-138 | B-137 | none |
| I-139 | A-138 | B-139 | none |
| I-140 | A-78 | B-139 | none |
| I-153 | A-153 | B-130 | none |
| I-154 | A-153 | B-154 | none |
| I-155 | A-155 | B-130 | none |
| I-156 | A-155 | B-154 | none |
| I-157 | A-78 | B-157 | none |
| I-161 | A-161 | B-161 | none |
| I-162 | A-162 | B-161 | none |
| I-163 | A-163 | B-161 | none |
| I-164 | A-164 | B-161 | none |
| I-165 | A-165 | B-161 | none |
| I-176 | A-176 | B-176 | none |
| I-177 | A-176 | B-177 | none |
| I-178 | A-178 | B-178 | none |
| I-179 | A-178 | B-179 | none |
| I-180 | A-180 | B-180 | none |
| I-183 | A-78 | B-183 | none |

### Example 3: Synthesis of I-141

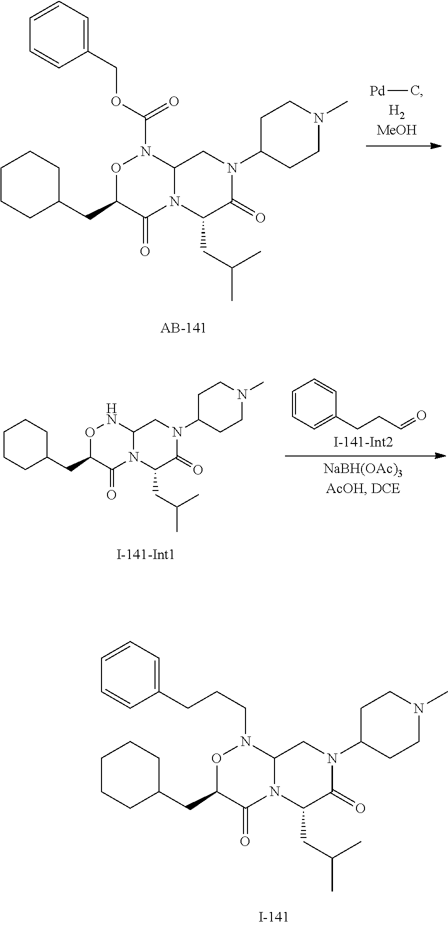

### Ex3-1) Synthesis of I-141-Int1

A stirred mixture of AB-141 (86 mg) and Pd/C (5 wt %, 33 mg) in THF (3 mL) was exposed to H₂ (balloon pressure) at ambient temperature. The reaction mixture was stirred for two hours and then H₂ was removed. Filtration over Celite pad and evaporation of the filtrate afforded I-141-Int1 (70 mg) as dark brown solid. The crude solid was used in the next reaction without further purification.

LCMS(method B): m/z=421.4 [M+H]⁺.

### Ex3-2) Synthesis of I-141

To a solution of I-141-Int1 (70 mg), AcOH (60 μL), 3-phenylpropanal (I-141-Int2, 88 μL) in DCE (4 mL) was added NaBH(OAc)₃ (140 mg) at ambient temperature. After stirring for one day, 1 mol/L aqueous sodium hydroxide solution (30 mL) was added. The organic layer was separated, washed with water (10 mL), dried over Na₂SO₄, concentrated in vacuo, and purified by preparative HPLC (Column: C30-UG-5, MeCN/0.1% solution of AcOH=20/80-70/30) to give I-141 (21 mg) as white solid.

LCMS(method B): m/z=539.6 [M+H]⁺.

### Example 4: Synthesis of I-145

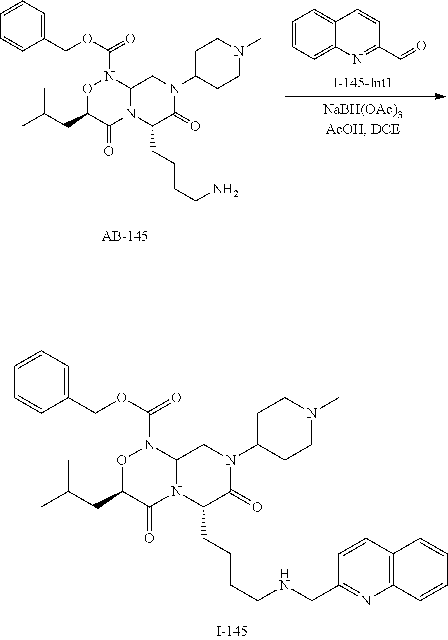

To a solution of AB-145 (25 mg), AcOH (10 μL), quinoline-2-carbaldehyde (7.8 mg) in DCE (1 mL) was added NaBH(OAc)₃ (71 mg) at ambient temperature. After stirring for one day, 1 mol/L aqueous sodium hydroxide solution (1.0 mL) was added. The organic layer was separated, dried over Na₂SO₄, concentrated in vacuo, and purified by preparative HPLC (Column: C30-UG-5, MeCN/0.1% solution of AcOH=10/90-60/40) to give I-145 (5.6 mg) as light yellow solid.

LCMS(method B): m/z=671.4 [M+H]⁺.

### Example 5: Synthesis of I-147

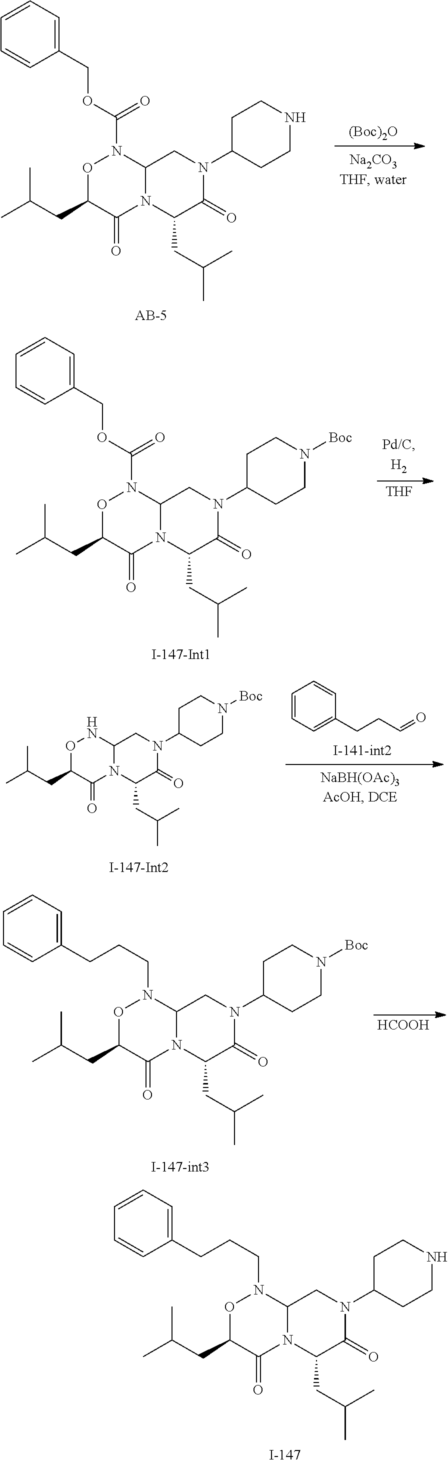

### Ex5-1) Synthesis of I-147-Int1

To a stirred two-phase mixture of AB-5 (0.26 g) in THF (5 mL) and Na₂CO₃ (0.11 g) in water (5 mL) was added Boc₂O (0.13 g) at ambient temperature. After stirring for one hour, the mixture was diluted with EtOAc (50 mL) and sat. aqueous NaHCO₃ solution (20 mL). The organic layer was separated, dried over Na₂SO₄, concentrated in vacuo, and purified by chromatography (SiO₂, n-hexane:AcOEt=90:10-20:80, gradient) to give I-147-Int1 (0.32 g) as white amorphous.

LCMS(method B): m/z=545.3 [M-tBu+H]⁺, 623.3 [M+Na]⁺.

### Ex5-2) Synthesis of I-147-Int2

A stirred mixture of I-147-Int1 (0.32 g) and Pd/C (5 wt %, 57 mg) in THF (5 mL) was exposed to H₂ (balloon pressure) at ambient temperature. The reaction mixture was stirred for one hour and then H₂ was removed. Filtration over Celite pad and evaporation of the filtrate afforded I-147-Int2 (0.22 g) as white solid. The crude solid was used in the next reaction without further purification.

LCMS(method B): m/z=367.2 [M-Boc+H]⁺, 411.2 [M-tBu+H]⁺.

### Ex5-3) Synthesis of I-147-Int3

To a solution of I-147-Int2 (80 mg), AcOH (10 μL), 3-phenylpropanal (I-141-Int2, 53 μL) in DCE (2 mL) was added NaBH(OAc)₃ (0.17 g) at ambient temperature. After stirring for one day, 2 mol/L aqueous sodium hydroxide solution (10 mL) was added. The organic layer was separated, washed with water (10 mL), dried over Na₂SO₄, concentrated in vacuo, and purified by preparative HPLC (Column: C30-UG-5, MeCN/0.1% solution of AcOH=50/50-100/0) to give I-147-Int3 (42 mg).

LCMS(method D): m/z=529.1 [M-tBu+H]⁺, 607.2 [M+Na]⁺.

### Ex5-4) Synthesis of I-147

To a flask charged with I-147-Int3 (20 mg) was added formic acid (1 mL), and the mixture was allowed to stand still at ambient temperature. After 4 days, the mixture was concentrated in vacuo. The residue was purified by preparative HPLC (Column: C30-UG-5, MeCN/0.1% solution of AcOH=30/70-80/20) to give I-147 (13 mg) as white solid.

LCMS(method D): m/z=485.1 [M+H]⁺.

### Example 6: Synthesis of I-148

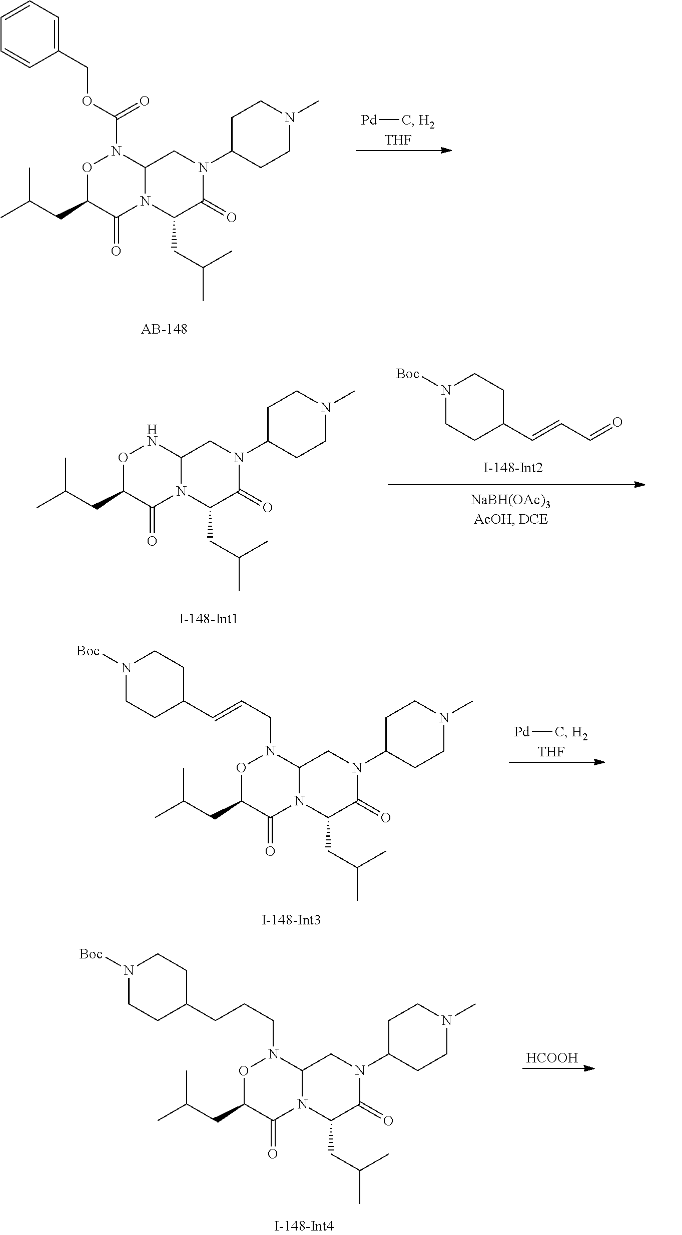

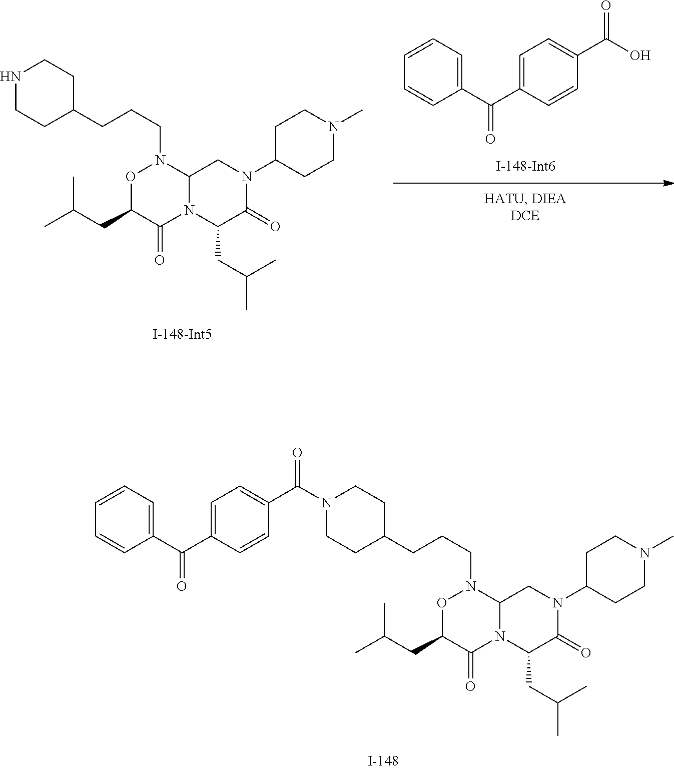 [SMILES: CC(C)C[C@H]1ON(CCCC2CCNCC2)C2CN(C3CCN(C)CC3)C(=O)[C@H](CC(C)C)N2C1=O ; O=C(O)c1ccc(C(=O)c2ccccc2)cc1 ; CC(C)C[C@H]1ON(CCCC2CCN(C(=O)c3ccc(C(=O)c4ccccc4)cc3)CC2)C2CN(C3CCN(C)CC3)C(=O)[C@H](CC(C)C)N2C1=O]

### Ex6-1) Synthesis of I-148-Int1

A mixture of AB-148 (0.99 g) and palladium carbon (10% weight) (0.31 g) in THF (20 mL) was stirred at room temperature for 1 hour under hydrogen atmosphere. The mixture was filtered by Celite. The filtrate was concentrated in vacuo to give I-148-Int1 (0.74 g) as dark brown solid.

LCMS(method B): m/z=381.4 [M+H]⁺

### Ex6-2) Synthesis of I-148-Int3

To a solution of I-148-Int1 (0.15 g), I-148-Int2 (0.22 g) and acetic acid (99 μL) in 1,2-dichloroethane (7 mL) was added sodium triacetoxyborohydride (0.25 g). The mixture was stirred at room temperature for 16 hours. To the mixture were added 1 mol/L NaOH aqueous solution (20 mL) and chloroform (40 mL). The organic layer was separated, then washed with water (20 mL). The organic layer was dried over Na₂SO₄ and concentrated in vacuo. The residue was purified by column chromatography (SiO₂, AcOEt:MeOH=80:20-20:80, gradient) to give I-148-Int3 (0.16 g) as colorless oil.

LCMS(method B): m/z=604.6 [M+H]⁺

### Ex6-3) Synthesis of I-148-Int4

A mixture of I-148-Int4 (0.16 g) and palladium carbon (10% weight) (56 mg) in THF (3 mL) was stirred at room temperature for 1 hour under hydrogen atmosphere. The mixture was filtered by Celite. The filtrate was concentrated in vacuo to give I-148-Int4 (0.16 g) as dark brown oil.

LCMS(method B): m/z=606.6 [M+H]⁺

### Ex6-4) Synthesis of I-148-Int5

A mixture of I-148-Int4 (0.14 g) and formic acid (2.0 mL) was stood at room temperature for 1 hour. The mixture was concentrated in vacuo. To the residue were added chloroform (30 mL) and Na₂CO₃ (3.0 g). The mixture was stirred at room temperature for 30 minutes. The mixture was filtered, and the filtrate was concentrated in vacuo to give I-148-Int5 (0.17 g) as pale yellow oil.

LCMS(method B): m/z=506.5 [M+H]⁺

### Ex6-5) Synthesis of I-148

To a mixture of I-148-Int5 (22 mg), I-148-Int6 (11 mg), and N-ethyl-N-isopropylpropan-2-amine (11 μL) in 1,2-dichloroethane (2 mL) was added HATU (25 mg). After stirring at room temperature for 2.5 hours, to the mixture were added I-148-Int6 (11 mg), N-ethyl-N-isopropylpropan-2-amine (11 μL), and HATU (25 mg). After stirring at room temperature for 1 hour, the mixture was added 1 mol/L HCl aqueous solution (10 mL), then extracted with chloroform (20 mL). The organic layer was washed with NaHCO₃ aqueous solution (10 mL) then water (10 mL). The organic layer was dried over Na₂SO₄ and concentrated in vacuo. The residue was purified by preparative HPLC (Column: C30-UG-5, MeCN/0.1% solution of AcOH=20/80-70/30) to give I-148 (0.6 mg) as a white solid.

LCMS(method B): m/z=714.6 [M+H]⁺

### Example 7: Synthesis of I-149

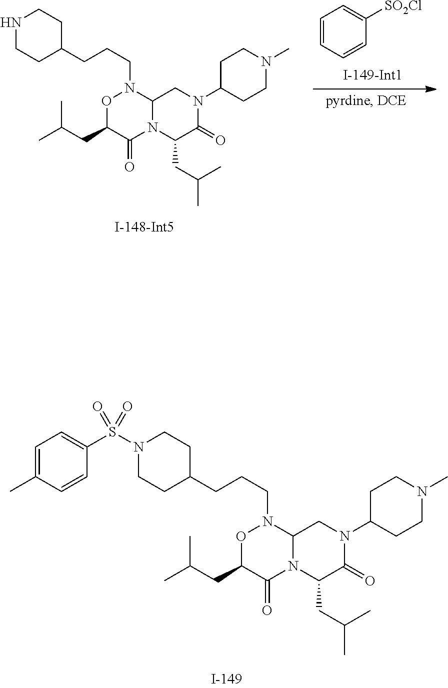

To a solution of I-148-Int5 (31 mg) in 1,2-dichloroethane (2 mL), were added p-toluenesulfonyl chloride (12 mg) and pyridine (49 mg) at room temperature. After stirring for 17 hours at same temperature, chloroform (10 mL) and water (20 mL) were added. The organic layer was separated, washed with brine (10 mL), dried over Na₂SO₄, and concentrated in vacuo. The residue was purified by prep-HPLC (condition (AcOH): B=20 to 70%). The collected fractions were combined and concentrated in vacuo. The aqueous solution was freeze-dried to give I-149 (0.5 mg) as a white solid.

LCMS(method B): m/z=660.5 [M+H]⁺.

### Example 8: Synthesis of I-159 and I-160

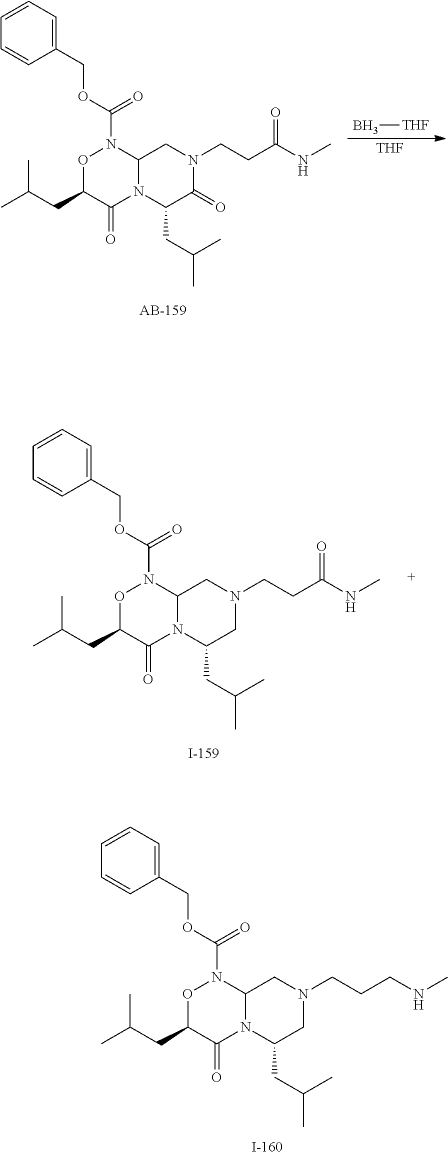

To a solution of AB-159 (0.96 g) in THF (20 mL), cooled by ice/water bath, was added BH3·THF (0.9 mol/L in THF, 7.5 mL). After stirring for 1 hour at 0° C. and for 2 hours at room temperature, MeOH (4 mL) was added dropwise. The mixture was concentrated in vacuo and purified by column chromatography (NH SiO₂, n-hexane:AcOEt=80:20-0:100, gradient) to give I-159 (0.17 g) and I-160 (0.27 g) as colorless oil.

LCMS(I-159, method D): m/z=489.1 [M+H]⁺

LCMS(I-160, method D): m/z=475.2 [M+H]⁺

### Example 9: Synthesis of I-173

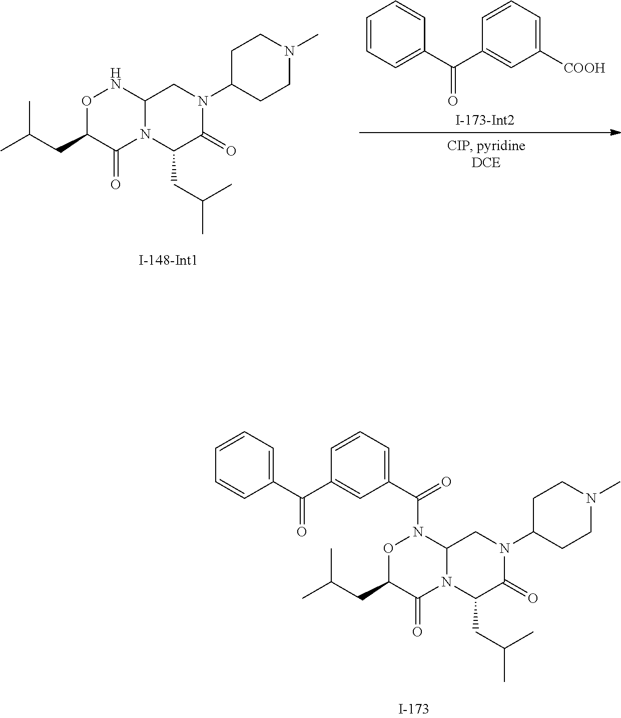 [SMILES: CC(C)C[C@H]1ONC2CN(C3CCN(C)CC3)C(=O)[C@H](CC(C)C)N2C1=O ; O=C(O)c1cccc(C(=O)c2ccccc2)c1 ; CC(C)C[C@H]1ON(C(=O)c2cccc(C(=O)c3ccccc3)c2)C2CN(C3CCN(C)CC3)C(=O)[C@H](CC(C)C)N2C1=O]

To a solution of I-148-Int1 (20 mg) in 1,2-dichloroethane (4 mL), were added 3-benzoylbenzoic acid (21 mg), pyridine (67 mg) and 2-chloro-1,3-dimethyl-4,5-dihydro-1H-imidazol-3-ium hexafluorophosphate (CIP, 100 mg) at room temperature. After stirring for 4 hours at same temperature, saturated aqueous sodium bicarbonate solution (50 mL) and chloroform (20 mL) were added. The organic layer was separated, then washed with 1 mol/L HCl (15 mL), saturated aqueous sodium bicarbonate solution (20 mL) and brine (10 mL), dried over Na₂SO₄, and concentrated in vacuo. The residue was purified by prep-HPLC (condition (AcOH): B=10 to 60%). The collected fractions were combined and concentrated in vacuo. The aqueous solution was freeze-dried to give I-173 (10 mg) as a white solid.

LCMS(method B): m/z=589.4 [M+H]⁺.

### Example 10: Synthesis of I-182

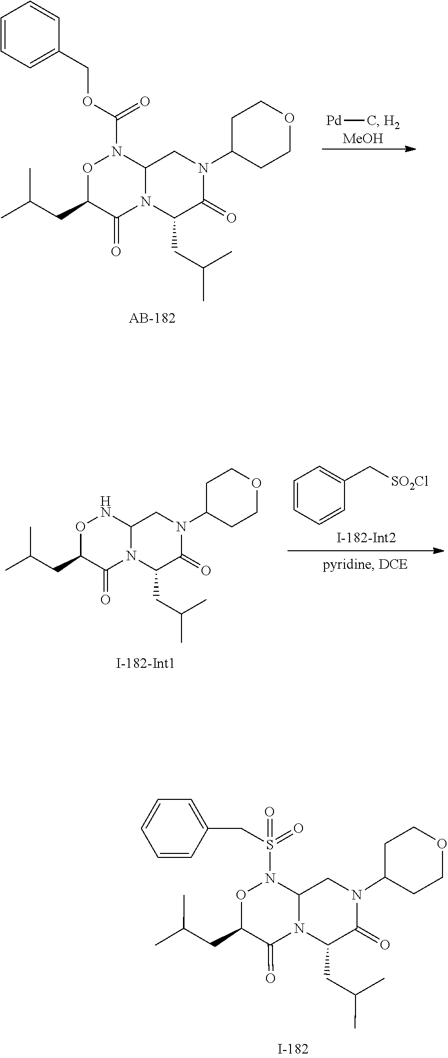

### Ex10-1) Synthesis of I-182-Int1

A mixture of AB-182 (2.0 g) and palladium carbon (10% weight) (0.51 g) in THF (20 mL) was stirred at room temperature for 100 minutes under hydrogen atmosphere. The mixture was filtered by Celite. The filtrate was concentrated in vacuo to give I-182-Int1 (1.5 g) as pale yellow green solid.

LCMS(method B): m/z=368.2 [M+H]⁺

### Ex10-2) Synthesis of I-182

To a mixture of I-182-Int1 (0.10 g) and pyridine (88 μL) in 1,2-dichloroethane (3 mL) was added I-148-Int2 (0.16 g). After stirring at room temperature for 2 hours and at 60° C. for 3 days, the mixture was poured into saturated aqueous sodium bicarbonate solution and then extracted with chloroform. The organic layer was washed with NaHCO₃ aqueous solution (10 mL) then water (10 mL). The organic phase was dried over MgSO₄ and concentrated in vacuo to give a crude product (0.12 g), which was purified by preparative HPLC to give I-182 (20 mg) as a white solid.

LCMS(method B): m/z=522.2 [M+H]⁺

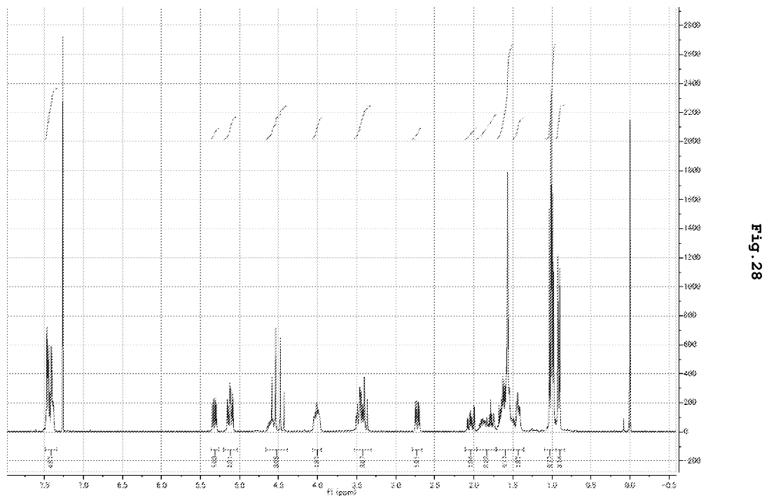

¹H NMR (300 MHz, CDCl₃) data of I-182 is shown in FIG. 28. Compounds I-141 to I-143 were synthesized according to a method similar to the Example 3 (Production Method 7) or a known method using the Intermediates AB and reagents shown in Table 9.

Compounds I-144 to I-146 were synthesized according to a method similar to the Example 4 or a known method using the Intermediates AB and reagents shown in Table 9.

Compounds I-149 to I-152 were synthesized according to a method similar to the Example 7 or a known method using the Intermediates AB and reagents shown in Table 9.

Compounds I-158 to I-160 were synthesized according to a method similar to the Example 8 (Production Method 8) or a known method using the Intermediates AB shown in Table 9.

Compounds I-166 to I-175 were synthesized according to a method similar to the Example 9 (Production Method 5) or a known method using the Intermediates AB and reagents shown in Table 9.

Compounds I-181 to I-182 were synthesized according to a method similar to the Example 10 (Production Method 6) or, a known method using the Intermediates AB and reagents shown in Table 9.

TABLE 9

| Comound | | | |
|---|---|---|---|
| No. | Int. AB | Reagents | Procedures |
| I-141 | AB-141 | I-141-Int2 | Example 3 |
| I-142 | AB-142 | I-141-Int2 | Example 3 |
| I-143 | AB-143 | I-141-Int2 | Example 3 |
| I-144 | AB-145 | I-144-Int1 | Example 4 |
| I-145 | AB-145 | I-145-Int1 | Example 4 |
| I-146 | AB-145 | I-146-Int1 | Example 4 |
| I-147 | AB-147 | I-141-Int2 | Example 5 |
| I-148 | AB-148 | I-148-Int6 | Example 6 |
| I-149 | AB-148 | I-149-Int1 | Example 7 |
| I-150 | AB-148 | I-150-Int1 | Example 7 |
| I-151 | AB-148 | I-151-Int1 | Example 7 |
| I-152 | AB-148 | I-152-Int1 | Example 7 |
| I-158 | AB-158 | | Example 8 |
| I-159 | AB-159 | | Example 8 |
| I-160 | AB-159 | | Example 8 |
| I-166 | AB-159 | I-166-Int1 | Example 9 |
| I-167 | AB-159 | I-167-Int1 | Example 9 |
| I-168 | AB-159 | I-168-Int1 | Example 9 |
| I-169 | AB-159 | I-169-Int1 | Example 9 |
| I-170 | AB-159 | I-170-Int1 | Example 9 |
| I-171 | AB-148 | I-171-Int1 | Example 9 |
| I-172 | AB-148 | I-172-Int1 | Example 9 |
| I-173 | AB-148 | I-173-Int2 | Example 9 |
| I-174 | AB-148 | I-174-Int1 | Example 9 |
| I-175 | AB-148 | I-175-Int1 | Example 9 |
| I-181 | AB-182 | I-151-Int1 | Example 10 |
| I-182 | AB-182 | I-182-Int2 | Example 10 |

Chemical structures of Intermediates AB are shown in Table 10 (Table 10-1 to Table 10-2). Intermediates AB were synthesized by using the corresponding Intermediates A and B listed in Table 10 according to the method in the Example 1 or a known method.

TABLE 10-1

| Int. AB | Structure | Int. A | Int. B |
|---|---|---|---|
| AB-141 | 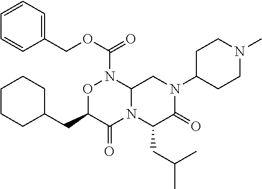 [SMILES: CC(C)C[C@H]1C(=O)N(C2CCN(C)CC2)CC2N(C(=O)OCc3ccccc3)O[C@H](CC3CCCCC3)C(=O)N21] | A-138 | B-137 |
| AB-142 | 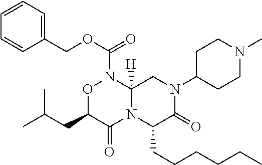 [SMILES: CCCCCC[C@H]1C(=O)N(C2CCN(C)CC2)C[C@@H]2N(C(=O)OCc3ccccc3)O[C@H](CC(C)C)C(=O)N21] | A-78 | B-142 |
| AB-143 | 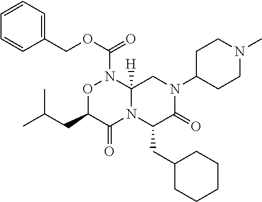 [SMILES: CC(C)C[C@H]1ON(C(=O)OCc2ccccc2)[C@H]2CN(C3CCN(C)CC3)C(=O)[C@H](CC3CCCCC3)N2C1=O] | A-78 | B-139 |
| AB-145 | 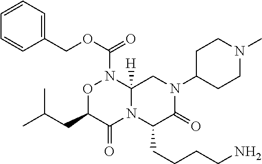 [SMILES: CC(C)C[C@H]1ON(C(=O)OCc2ccccc2)[C@H]2CN(C3CCN(C)CC3)C(=O)[C@H](CCCCN)N2C1=O] | A-78 | B-144 |
| AB-147 | 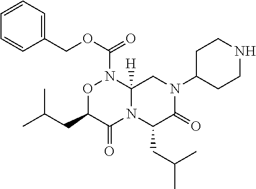 [SMILES: CC(C)C[C@H]1ON(C(=O)OCc2ccccc2)[C@H]2CN(C3CCNCC3)C(=O)[C@H](CC(C)C)N2C1=O] | A-78 | B-135 |

TABLE 10-2

| Int. AB | Structure | Int. A | Int. B |
|---|---|---|---|
| AB-148 | 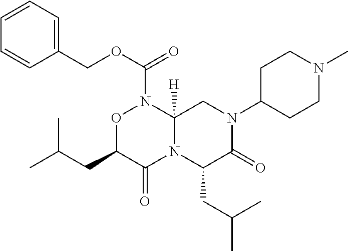 [SMILES: CC(C)C[C@H]1ON(C(=O)OCc2ccccc2)[C@H]2CN(C3CCN(C)CC3)C(=O)[C@H](CC(C)C)N2C1=O] | A-78 | B-137 |
| AB-158 | 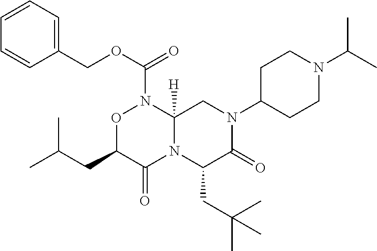 [SMILES: CC(C)C[C@H]1ON(C(=O)OCc2ccccc2)[C@H]2CN(C3CCN(C(C)C)CC3)C(=O)[C@H](CC(C)(C)C)N2C1=O] | A-78 | B-158 |
| AB-159 | 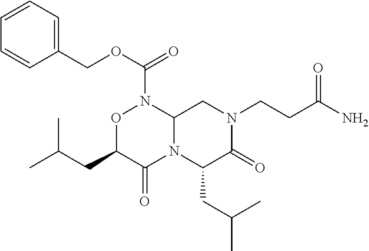 [SMILES: CC(C)C[C@H]1ON(C(=O)OCc2ccccc2)C2CN(CCC(N)=O)C(=O)[C@H](CC(C)C)N2C1=O] | A-78 | B-78 |
| AB-182 | 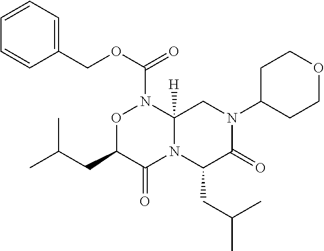 | A-78 | B-182 |

Chemical structures of Intermediates A and B are shown in Table 6 and 5, respectively.

Chemical structures of Reagents listed in Table 9 are shown in Table 11.

TABLE 11

| Reagents | Structure |
|---|---|
| I-141- Int2 | 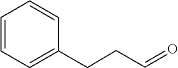 |
| I-144- Int1 | 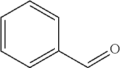 |
| I-145- Int1 | 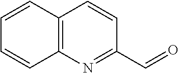 [SMILES: O=Cc1ccc2ccccc2n1] |
| I-146- Int1 | 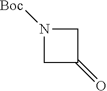 [SMILES: CC(C)(C)OC(=O)N1CC(=O)C1] |
| I-148- Int6 | 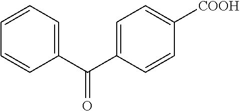 [SMILES: O=C(O)c1ccc(C(=O)c2ccccc2)cc1] |
| I-149- Int1 | 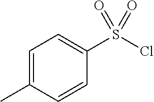 |
| I-150- Int1 | 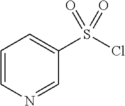 |
| I-151- Int1 | 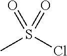 |
| I-152- Int1 | 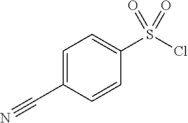 [SMILES: N#Cc1ccc(S(=O)(=O)Cl)cc1] |
| I-166- Int1 | 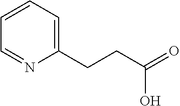 [SMILES: O=C(O)CCc1ccccn1] |
| I-167- Int1 | 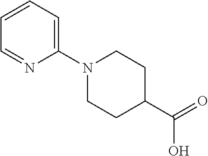 [SMILES: O=C(O)C1CCN(c2ccccn2)CC1] |
| I-168- Int1 | 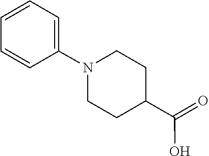 [SMILES: O=C(O)C1CCN(c2ccccc2)CC1] |
| I-169- Int1 | 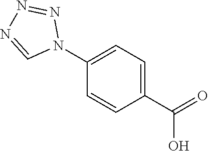 [SMILES: O=C(O)c1ccc(-n2cnnn2)cc1] |
| I-170- Int1 | 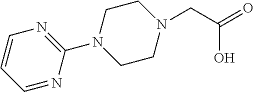 [SMILES: O=C(O)CN1CCN(c2ncccn2)CC1] |
| I-171- Int1 | 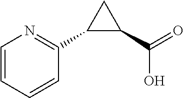 [SMILES: O=C(O)[C@@H]1C[C@H]1c1ccccn1] |
| I-172- Int1 | 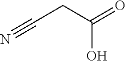 |
| I-173- Int2 | 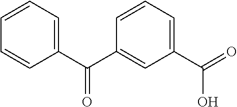 [SMILES: O=C(O)c1cccc(C(=O)c2ccccc2)c1] |
| I-174- Int1 | 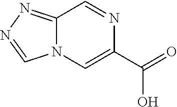 [SMILES: O=C(O)c1cn2cnnc2cn1] |
| I-175- Int1 | 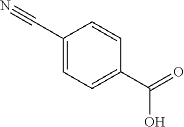 [SMILES: N#Cc1ccc(C(=O)O)cc1] |
| I-182- Int1 | 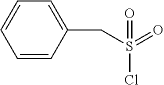 [SMILES: O=S(=O)(Cl)Cc1ccccc1] |

The compound names and the measurement results of the molecular weight of compounds I-1 to I-183 are shown in Table 12 (Table 12-1 to Table 12-28).

TABLE 12-1

| | | | | Exact | LCMS | | LCMS |
|---|---|---|---|---|---|---|---|
| Compound No | Structure | Name | MW | Mass | Observed Mass | | Method |
| I-1 | 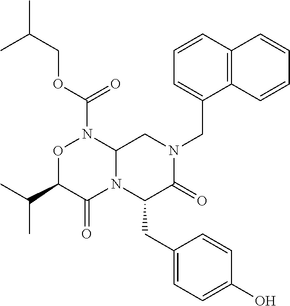 [SMILES: CC(C)COC(=O)N1O[C@H](C(C)C)C(=O)N2C1CN(Cc1cccc3ccccc13)C(=O)[C@@H]2Cc1ccc(O)cc1] | (3R,6S)-isobutyl 6-(4- hydroxybenzyl)-3-isopropyl- 8-(naphthalen-1-ylmethyl)- 4,7- dioxohexahydropyrazino[2,1- c][1,2,4]oxadiazine-1(6H)- carboxylate | 559.7 | 559.3 | 560.2 | [M + H]⁺ | A |
| I-2 | 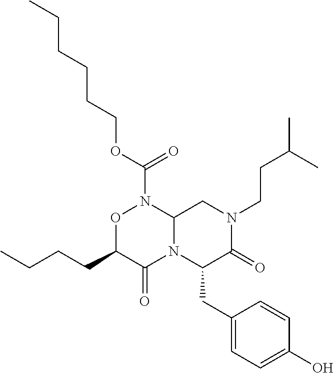 [SMILES: CCCCCCOC(=O)N1O[C@H](CCCC)C(=O)N2C1CN(CCC(C)C)C(=O)[C@@H]2Cc1ccc(O)cc1] | (3R,6S)-hexyl 3-butyl-6-(4- hydroxybenzyl)-8-isopentyl- 4,7- dioxohexahydropyrazino[2,1- c][1,2,4]oxadiazine-1(6H)- carboxylate | 531.7 | 531.3 | 532.4 | [M + H]⁺ | A |
| I-3 | 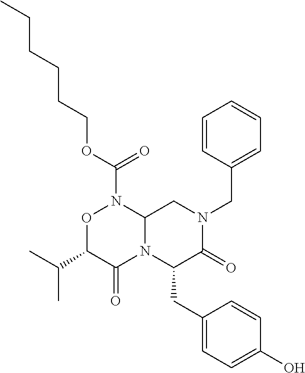 [SMILES: CCCCCCOC(=O)N1O[C@@H](C(C)C)C(=O)N2C1CN(Cc1ccccc1)C(=O)[C@@H]2Cc1ccc(O)cc1] | (3S,6S)-hexyl 8-benzyl-6-(4- hydroxybenzyl)-3-isopropyl- 4,7- dioxohexahydropyrazino[2,1- c][1,2,4]oxadiazine-1(6H)- carboxylate | 537.7 | 537.3 | 538.2 | [M + H]⁺ | A |
| I-4 | 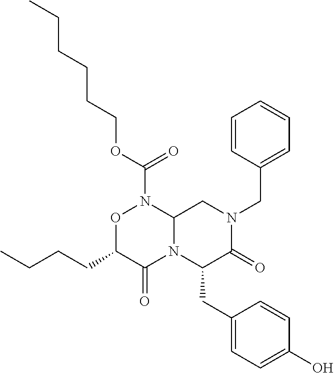 [SMILES: CCCCCCOC(=O)N1O[C@@H](CCCC)C(=O)N2C1CN(Cc1ccccc1)C(=O)[C@@H]2Cc1ccc(O)cc1] | (3S,6S)-hexyl 8-benzyl-3- butyl-6-(4-hydroxybenzyl)- 4,7- dioxohexahydropyrazino[2,1- c][1,2,4]oxadiazine-1(6H)- carboxylate | 551.7 | 551.3 | 552.3 | [M + H]⁺ | A |
| I-5 | 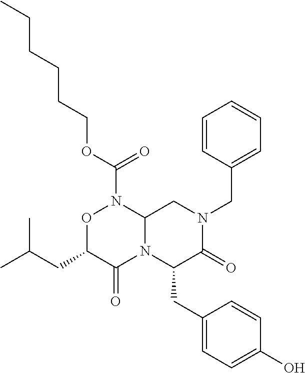 [SMILES: CCCCCCOC(=O)N1O[C@@H](CC(C)C)C(=O)N2C1CN(Cc1ccccc1)C(=O)[C@@H]2Cc1ccc(O)cc1] | (3S,6S)-hexyl 8-benzyl-6-(4- hydroxybenzyl)-3-isobutyl- 4,7- dioxohexahydropyrazino[2,1- c][1,2,4]oxadiazine-1(6H)- carboxylate | 551.7 | 551.3 | 552.3 | [M + H]⁺ | A |
| I-6 | 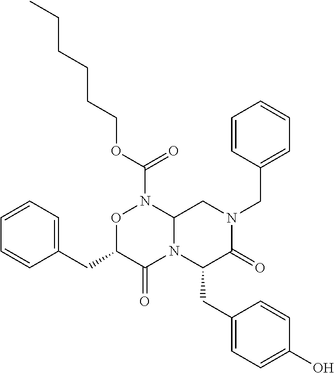 [SMILES: CCCCCCOC(=O)N1O[C@@H](Cc2ccccc2)C(=O)N2C1CN(Cc1ccccc1)C(=O)[C@@H]2Cc1ccc(O)cc1] | (35,6S)-hexyl 3,8-dibenzyl-6- (4-hydroxybenzyl)-4,7- dioxohexahydropyrazino[2,1- c][1,2,4]oxadiazine-1(6H)- carboxylate | 585.7 | 585.3 | 586.4 | [M + H]⁺ | A |

TABLE 12-2

| | | | | | | | |
|---|---|---|---|---|---|---|---|
| I-7 | 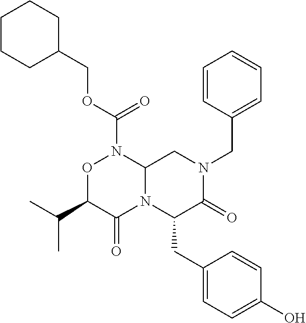 [SMILES: CC(C)[C@H]1ON(C(=O)OCC2CCCCC2)C2CN(Cc3ccccc3)C(=O)[C@H](Cc3ccc(O)cc3)N2C1=O] | (3R,6S)-cyclohexylmethyl 8- benzyl-6-(4-hydroxybenzyl)- 3-isopropyl-4,7- dioxohexahydropyrazino[2,1- c][1,2,4]oxadiazine-1(6H)- carboxylate | 549.7 | 549.3 | 550.3 | [M + H]⁺ | A |
| I-8 | 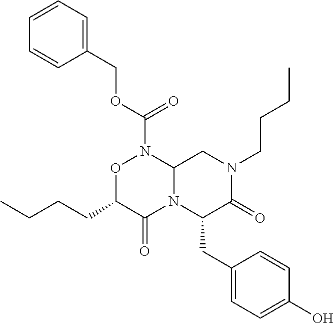 [SMILES: CCCC[C@@H]1ON(C(=O)OCc2ccccc2)C2CN(CCCC)C(=O)[C@H](Cc3ccc(O)cc3)N2C1=O] | (3S,6S)-benzyl 3,8-dibutyl-6- (4-hydroxybenzyl)-4,7- dioxohexahydropyrazino[2,1- c][1,2,4]oxadiazine-1(6H)- carboxylate | 523.6 | 523.3 | 524.3 | [M + H]⁺ | A |
| I-9 | 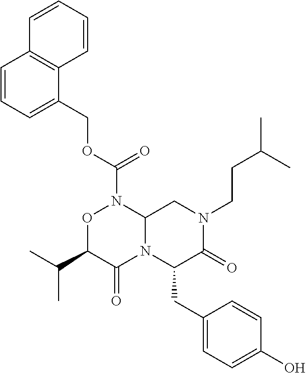 [SMILES: CC(C)CCN1CC2N(C(=O)OCc3cccc4ccccc34)O[C@H](C(C)C)C(=O)N2[C@@H](Cc2ccc(O)cc2)C1=O] | (3R,6S)-naphthalen-1- ylmethyl 6-(4- hydroxybenzyl)-8-isopentyl- 3-isopropyl-4,7- dioxohexahydropyrazino[2,1- c][1,2,4]oxadiazine-1(6H)- carboxylate | 573.7 | 573.3 | 574.2 | [M + H]⁺ | A |
| I-10 | 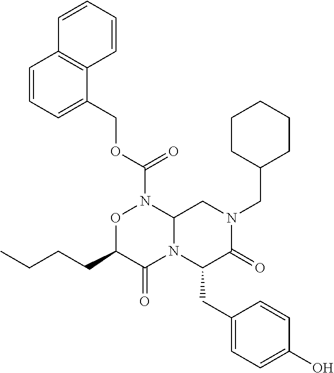 [SMILES: CCCC[C@H]1ON(C(=O)OCc2cccc3ccccc23)C2CN(CC3CCCCC3)C(=O)[C@H](Cc3ccc(O)cc3)N2C1=O] | (3R,6S)-naphthalen-1- ylmethyl 3-butyl-8- (cyclohexylmethyl)-6-(4- hydroxybenzyl)-4,7- dioxohexahydropyrazino[2,1- c][1,2,4]oxadiazine-1(6H)- carboxylate | 613.7 | 613.3 | 614.4 | [M + H]⁺ | A |
| I-11 | 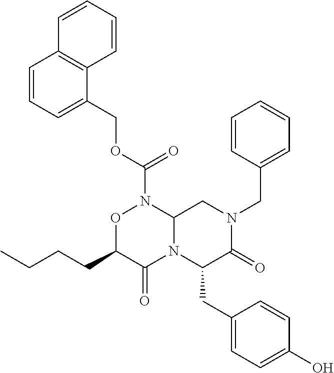 [SMILES: CCCC[C@H]1ON(C(=O)OCc2cccc3ccccc23)C2CN(Cc3ccccc3)C(=O)[C@H](Cc3ccc(O)cc3)N2C1=O] | (3R,6S)-naphthalen-1- ylmethyl 8-benzyl-3-butyl-6- (4-hydroxybenzyl)-4,7- dioxohexahydropyrazino[2,1- c][1,2,4]oxadiazine-1(6H)- carboxylate | 607.7 | 607.3 | 608.3 | [M + H]⁺ | A |
| I-12 | 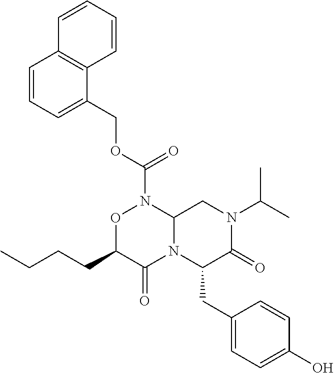 [SMILES: CCCC[C@H]1ON(C(=O)OCc2cccc3ccccc23)C2CN(C(C)C)C(=O)[C@H](Cc3ccc(O)cc3)N2C1=O] | (3R,6S)-naphthalen-1- ylmethyl 3-butyl-6-(4- hydroxybenzyl)-8-isopropyl- 4,7- dioxohexahydropyrazino[2,1- c][1,2,4]oxadiazine-1(6H)- carboxylate | 559.7 | 559.3 | 560.2 | [M + H]⁺ | A |
| I-13 | 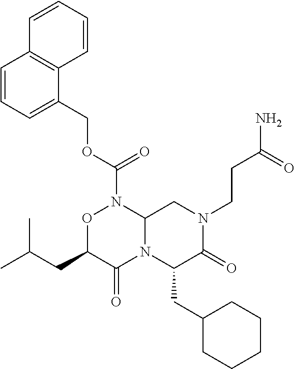 [SMILES: CC(C)C[C@H]1ON(C(=O)OCc2cccc3ccccc23)C2CN(CCC(N)=O)C(=O)[C@H](CC3CCCCC3)N2C1=O] | (3R,6S)-naphthalen-1- ylmethyl 8-(3-amino-3- oxopropyl)-6- (cyclohexylmethyl)-3- isobutyl-4,7- dioxohexahydropyrazino[2,1- c][1,2,4]oxadiazine-1(6H)- carboxylate | 578.7 | 578.3 | 579.4 | [M + H]⁺ | A |

TABLE 12-3

| | | | | | | | |
|---|---|---|---|---|---|---|---|
| I-14 | 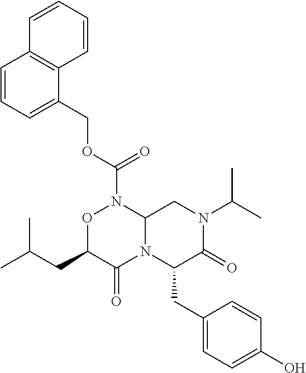 [SMILES: CC(C)C[C@H]1ON(C(=O)OCc2cccc3ccccc23)C2CN(C(C)C)C(=O)[C@H](Cc3ccc(O)cc3)N2C1=O] | (3R,6S)-naphthalen-1- ylmethyl 6-(4- hydroxybenzyl)-3-isobutyl-8- isopropyl-4,7- dioxohexahydropyrazino[2,1- c][1,2,4]oxadiazine-1(6H)- carboxylate | 559.7 | 559.3 | 560.2 | [M + H]⁺ | A |
| I-15 | 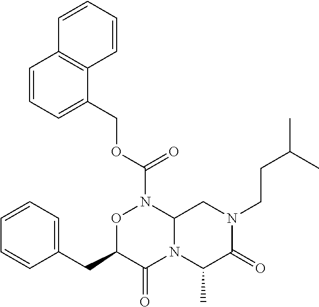 [SMILES: CC(C)CCN1CC2N(C(=O)OCc3cccc4ccccc34)O[C@H](Cc3ccccc3)C(=O)N2[C@@H](C)C1=O] | (3R,6S)-naphthalen-1- ylmethyl 3-benzyl-8- isopentyl-6-methyl-4,7- dioxohexahydropyrazino[2,1- c][1,2,4]oxadiazine-1(6H)- carboxylate | 529.6 | 529.3 | 530.3 | [M + H]⁺ | A |
| I-16 | 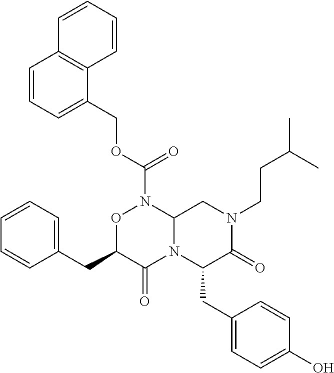 [SMILES: CC(C)CCN1CC2N(C(=O)OCc3cccc4ccccc34)O[C@H](Cc3ccccc3)C(=O)N2[C@@H](Cc2ccc(O)cc2)C1=O] | (3R,6S)-naphthalen-1- ylmethyl 3-benzyl-6-(4- hydroxybenzyl)-8-isopentyl- 4,7- dioxohexahydropyrazino[2,1- c][1,2,4]oxadiazine-1(6H)- carboxylate | 621.7 | 621.3 | 622.3 | [M + H]⁺ | A |
| I-17 | 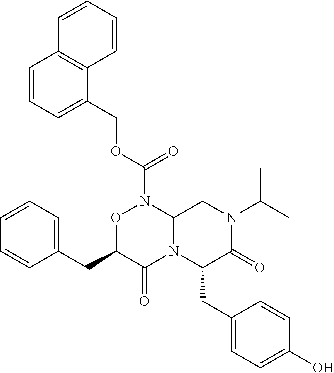 [SMILES: CC(C)N1CC2N(C(=O)OCc3cccc4ccccc34)O[C@H](Cc3ccccc3)C(=O)N2[C@@H](Cc2ccc(O)cc2)C1=O] | (3R,6S)-naphthalen-1- ylmethyl 3-benzyl-6-(4- hydroxybenzyl)-8-isopropyl- 4,7- dioxohexahydropyrazino[2,1- c][1,2,4]oxadiazine-1(6H)- carboxylate | 593.7 | 593.3 | 594.2 | [M + H]⁺ | A |
| I-18 | 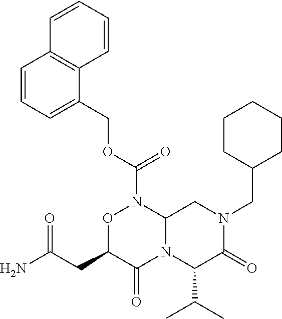 [SMILES: CC(C)[C@H]1C(=O)N(CC2CCCCC2)CC2N(C(=O)OCc3cccc4ccccc34)O[C@H](CC(N)=O)C(=O)N21] | (3R,6S)-naphthalen-1- ylmethyl 3-(2-amino-2- oxoethyl)-8- (cyclohexylmethyl)-6- isopropyl-4,7- dioxohexahydropyrazino[2,1- c][1,2,4]oxadiazine-1(6H)- carboxylate | 550.7 | 550.3 | 551.3 | [M + H]⁺ | A |
| I-19 | 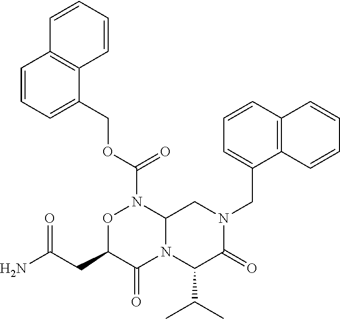 [SMILES: CC(C)[C@H]1C(=O)N(Cc2cccc3ccccc23)CC2N(C(=O)OCc3cccc4ccccc34)O[C@H](CC(N)=O)C(=O)N21] | (3R,6S)-naphthalen-1- ylmethyl 3-(2-amino-2- oxoethyl)-6-isopropyl-8- (naphthalen-1-ylmethyl)-4,7- dioxohexahydropyrazino[2,1- c][1,2,4]oxadiazine-1(6H)- carboxylate | 594.7 | 594.2 | 595.2 | [M + H]⁺ | A |
| I-20 | 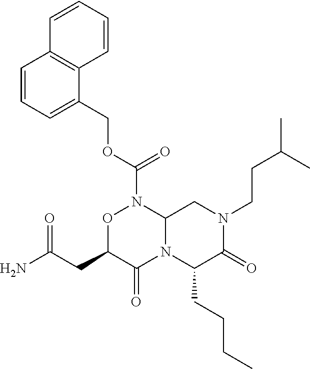 [SMILES: CCCC[C@H]1C(=O)N(CCC(C)C)CC2N(C(=O)OCc3cccc4ccccc34)O[C@H](CC(N)=O)C(=O)N21] | (3R,6S)-naphthalen-1- ylmethyl 3-(2-amino-2- oxoethyl)-6-butyl-8- isopentyl-4,7- dioxohexahydropyrazino[2,1- c][1,2,4]oxadiazine-1(6H)- carboxylate | 538.6 | 538.3 | 539.3 | [M + H]⁺ | A |

TABLE 12-4

| | | | | | | | |
|---|---|---|---|---|---|---|---|
| I-21 | 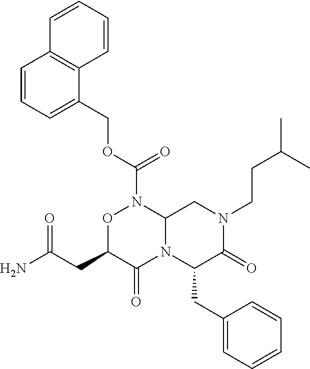 [SMILES: CC(C)CCN1CC2N(C(=O)OCc3cccc4ccccc34)O[C@H](CC(N)=O)C(=O)N2[C@@H](Cc2ccccc2)C1=O] | (3R,6S)-naphthalen-1- ylmethyl 3-(2-amino-2- oxoethyl)-6-benzyl-8- isopentyl-4,7- dioxohexahydropyrazino[2,1- c][1,2,4]oxadiazine-1(6H)- carboxylate | 572.7 | 572.3 | 573.2 | [M + H]⁺ | A |
| I-22 | 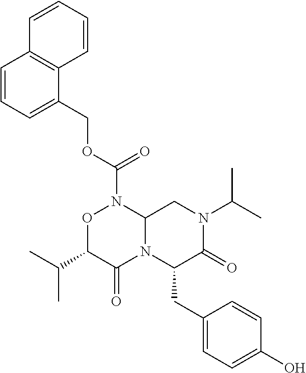 [SMILES: CC(C)[C@@H]1ON(C(=O)OCc2cccc3ccccc23)C2CN(C(C)C)C(=O)[C@H](Cc3ccc(O)cc3)N2C1=O] | (3S,6S)-naphthalen-1- ylmethyl 6-(4- hydroxybenzyl)-3,8- diisopropyl-4,7- dioxohexahydropyrazino[2,1- c][1,2,4]oxadiazine-1(6H)- carboxylate | 545.6 | 545.3 | 546.2 | [M + H]⁺ | A |
| I-23 | 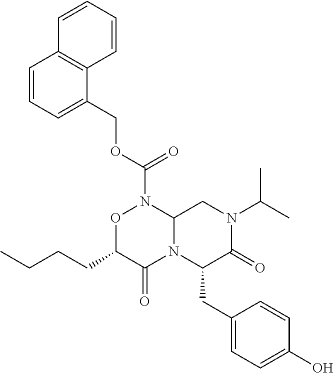 [SMILES: CCCC[C@@H]1ON(C(=O)OCc2cccc3ccccc23)C2CN(C(C)C)C(=O)[C@H](Cc3ccc(O)cc3)N2C1=O] | (3S,6S)-naphthalen-1- ylmethyl 3-butyl-6-(4- hydroxybenzyl)-8-isopropyl- 4,7- dioxohexahydropyrazino[2,1- c][1,2,4]oxadiazine-1(6H)- carboxylate | 559.7 | 559.3 | 560.2 | [M + H]⁺ | A |
| I-24 | 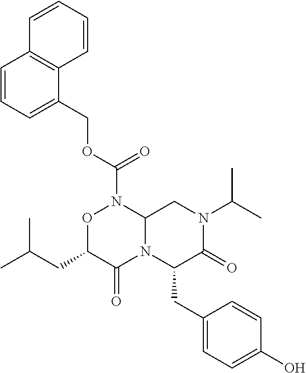 [SMILES: CC(C)C[C@@H]1ON(C(=O)OCc2cccc3ccccc23)C2CN(C(C)C)C(=O)[C@H](Cc3ccc(O)cc3)N2C1=O] | (3S,6S)-naphthalen-1- ylmethyl 6-(4- hydroxybenzyl)-3-isobutyl-8- isopropyl-4,7- dioxohexahydropyrazino[2,1- c][1,2,4]oxadiazine-1(6H)- carboxylate | 559.7 | 559.3 | 560.3 | [M + H]⁺ | A |
| I-25 | 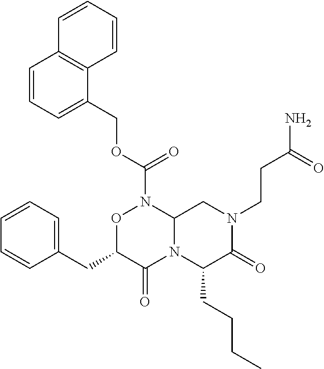 [SMILES: CCCC[C@H]1C(=O)N(CCC(N)=O)CC2N(C(=O)OCc3cccc4ccccc34)O[C@@H](Cc3ccccc3)C(=O)N21] | (3S,6S)-naphthalen-1- ylmethyl 8-(3-amino-3- oxopropyl)-3-benzyl-6-butyl- 4,7- dioxohexahydropyrazino[2,1- c][1,2,4]oxadiazine-1(6H)- carboxylate | 572.7 | 572.3 | 573.3 | [M + H]⁺ | A |
| I-26 | 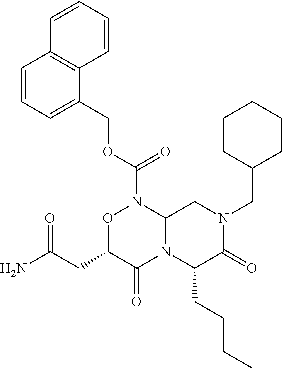 [SMILES: CCCC[C@H]1C(=O)N(CC2CCCCC2)CC2N(C(=O)OCc3cccc4ccccc34)O[C@@H](CC(N)=O)C(=O)N21] | (3S,6S)-naphthalen-1- ylmethyl 3-(2-amino-2- oxoethyl)-6-butyl-8- (cyclohexylmethyl)-4,7- dioxohexahydropyrazino[2,1- c][1,2,4]oxadiazine-1(6H)- carboxylate | 564.7 | 564.3 | 565.3 | [M + H]⁺ | A |

TABLE 12-5

| | | | | | | | |
|---|---|---|---|---|---|---|---|
| I-27 | 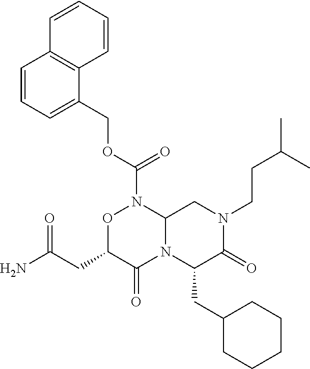 [SMILES: CC(C)CCN1CC2N(C(=O)OCc3cccc4ccccc34)O[C@@H](CC(N)=O)C(=O)N2[C@@H](CC2CCCCC2)C1=O] | (3S,6S)-naphthalen-1- ylmethyl 3-(2-amino-2- oxoethyl)-6- (cyclohexylmethyl)-8- isopentyl-4,7- dioxohexahydropyrazino[2,1- c][1,2,4]oxadiazine-1(6H)- carboxylate | 578.7 | 578.3 | 579.2 | [M + H]⁺ | A |
| I-28 | 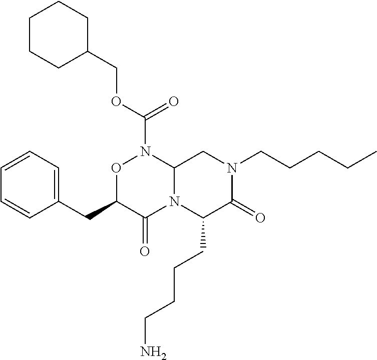 [SMILES: CCCCCN1CC2N(C(=O)OCC3CCCCC3)O[C@H](Cc3ccccc3)C(=O)N2[C@@H](CCCCN)C1=O] | (3R,6S)-cyclohexylmethyl 6- (4-aminobutyl)-3-benzyl-4,7- dioxo-8- pentylhexahydropyrazino [2,1-c][1,2,4]oxadiazine- 1(6H)-carboxylate | 542.7 | 542.3 | 543.5 | [M + H]⁺ | A |
| I-29 | 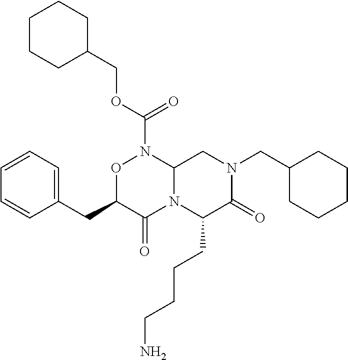 [SMILES: NCCCC[C@H]1C(=O)N(CC2CCCCC2)CC2N(C(=O)OCC3CCCCC3)O[C@H](Cc3ccccc3)C(=O)N21] | (3R,6S)-cyclohexylmethyl 6- (4-aminobutyl)-3-benzyl-8- (cyclohexylmethyl)-4,7- dioxohexahydropyrazino[2,1- c][1,2,4]oxadiazine-1(6H)- carboxylate | 568.8 | 568.4 | 569.4 | [M + H]⁺ | A |
| I-30 | 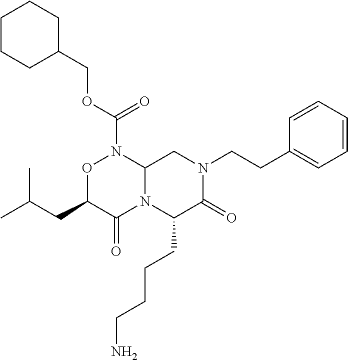 [SMILES: CC(C)C[C@H]1ON(C(=O)OCC2CCCCC2)C2CN(CCc3ccccc3)C(=O)[C@H](CCCCN)N2C1=O] | (3R,6S)-cyclohexylmethyl 6- (4-aminobutyl)-3-isobutyl- 4,7-dioxo-8- phenethylhexahydropyrazino [2,1-c][1,2,4]oxadiazine- 1(6H)-carboxylate | 542.7 | 542.3 | 543.4 | [M + H]⁺ | A |
| I-31 | 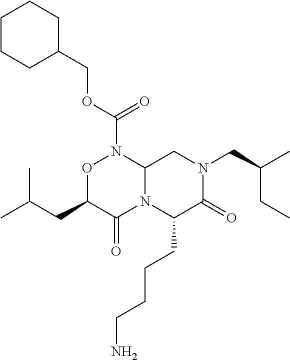 [SMILES: CC[C@H](C)CN1CC2N(C(=O)OCC3CCCCC3)O[C@H](CC(C)C)C(=O)N2[C@@H](CCCCN)C1=O] | (3R,6S)-cyclohexylmethyl 6- (4-aminobutyl)-3-isobutyl-8- ((S)-2-methylbutyl)-4,7- dioxohexahydropyrazino[2,1- c][1,2,4]oxadiazine-1(6H)- carboxylate | 508.7 | 508.4 | 509.4 | [M + H]⁺ | A |
| I-32 | 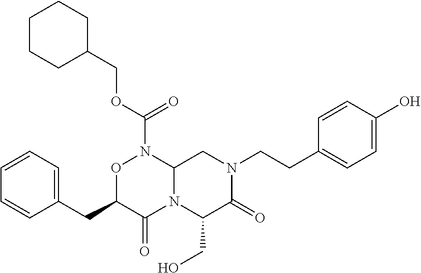 [SMILES: O=C1[C@H](CO)N2C(=O)[C@@H](Cc3ccccc3)ON(C(=O)OCC3CCCCC3)C2CN1CCc1ccc(O)cc1] | (3R,6S)-cyclohexylmethyl 3- benzyl-6-(hydroxymethyl)-8- (4-hydroxyphenethyl)-4,7- dioxohexahydropyrazino[2,1- c][1,2,4]oxadiazine-1(6H)- carboxylate | 551.6 | 551.3 | 552.3 | [M + H]⁺ | A |

TABLE 12-6

| | | | | | | | |
|---|---|---|---|---|---|---|---|
| I-33 | 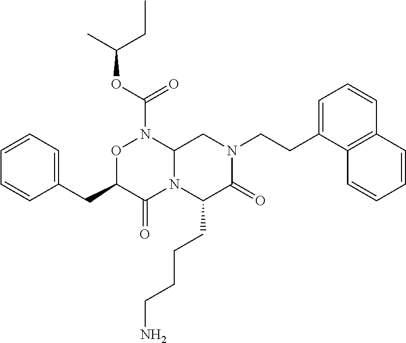 [SMILES: CC[C@H](C)OC(=O)N1O[C@H](Cc2ccccc2)C(=O)N2C1CN(CCc1cccc3ccccc13)C(=O)[C@@H]2CCCCN] | (3R,6S)-(S)-sec-butyl 6-(4- aminobutyl)-3-benzyl-8-(2- (naphthalen-1-yl)ethyl)-4,7- dioxohexahydropyrazino[2,1- c][1,2,4]oxadiazine-1(6H)- carboxylate | 586.7 | 586.3 | 587.3 | [M + H]⁺ | A |
| I-34 | 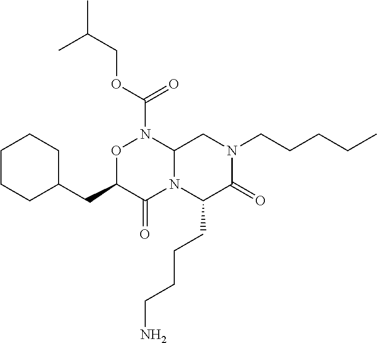 [SMILES: CCCCCN1CC2N(C(=O)OCC(C)C)O[C@H](CC3CCCCC3)C(=O)N2[C@@H](CCCCN)C1=O] | (3R,6S)-isobutyl 6-(4- aminobutyl)-3- (cyclohexylmethyl)-4,7- dioxo-8- pentylhexahydropyrazino [2,1-c][1,2,4]oxadiazine- 1(6H)-carboxylate | 508.7 | 508.4 | 509.4 | [M + H]⁺ | A |
| I-35 | 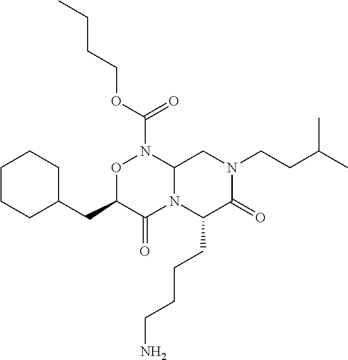 [SMILES: CCCCOC(=O)N1O[C@H](CC2CCCCC2)C(=O)N2C1CN(CCC(C)C)C(=O)[C@@H]2CCCCN] | (3R,6S)-butyl 6-(4- aminobutyl)-3- (cyclohexylmethyl)-8- isopentyl-4,7- dioxohexahydropyrazino[2,1- c][1,2,4]oxadiazine-1(6H)- carboxylate | 508.7 | 508.4 | 509.4 | [M + H]⁺ | A |
| I-36 | 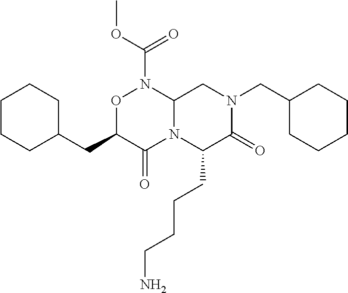 [SMILES: COC(=O)N1O[C@H](CC2CCCCC2)C(=O)N2C1CN(CC1CCCCC1)C(=O)[C@@H]2CCCCN] | (3R,6S)-methyl 6-(4- aminobutyl)-3,8- bis(cyclohexylmethyl)-4,7- dioxohexahydropyrazino[2,1- c][1,2,4]oxadiazine-1(6H)- carboxylate | 492.7 | 492.3 | 493.3 | [M + H]⁺ | A |
| I-37 | 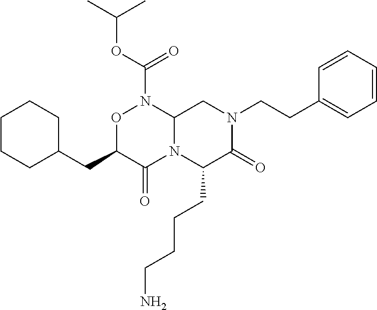 [SMILES: CC(C)OC(=O)N1O[C@H](CC2CCCCC2)C(=O)N2C1CN(CCc1ccccc1)C(=O)[C@@H]2CCCCN] | (3R,6S)-isopropyl 6-(4- aminobutyl)-3- (cyclohexylmethyl)-4,7- dioxo-8- phenethylhexahydropyrazino [2,1-c][1,2,4]oxadiazine- 1(6H)-carboxylate | 528.7 | 528.3 | 529.3 | [M + H]⁺ | A |
| I-38 | 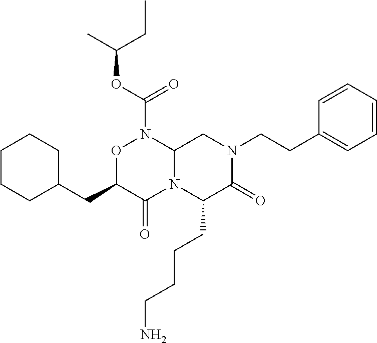 [SMILES: CC[C@H](C)OC(=O)N1O[C@H](CC2CCCCC2)C(=O)N2C1CN(CCc1ccccc1)C(=O)[C@@H]2CCCCN] | (3R,6S)-(S)-sec-butyl 6-(4- aminobutyl)-3- (cyclohexylmethyl)-4,7- dioxo-8- phenethylhexahydropyrazino [2,1-c][1,2,4]oxadiazine- 1(6H)-carboxylate | 542.7 | 542.3 | 543.3 | [M + H]⁺ | A |

TABLE 12-7

| | | | | | | | |
|---|---|---|---|---|---|---|---|
| I- 39 | 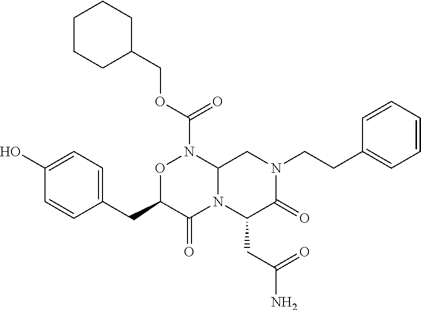 [SMILES: NC(=O)C[C@H]1C(=O)N(CCc2ccccc2)CC2N(C(=O)OCC3CCCCC3)O[C@H](Cc3ccc(O)cc3)C(=O)N21] | (3R,6S)-cyclohexylmethyl 6- (2-amino-2-oxoethyl)-3-(4- hydroxybenzyl)-4,7-dioxo-8- phenethylhexahydropyrazino [2,1-c][1,2,4]oxadiazine- 1(6H)-carboxylate | 578.7 | 578.3 | 579.3 | [M + H]⁺ | A |
| I- 40 | 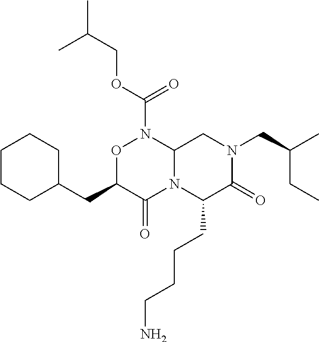 [SMILES: CC[C@H](C)CN1CC2N(C(=O)OCC(C)C)O[C@H](CC3CCCCC3)C(=O)N2[C@@H](CCCCN)C1=O] | (3R,6S)-isobutyl 6-(4- aminobutyl)-3- (cyclohexylmethyl)-8-((S)-2- methylbutyl)-4,7- dioxohexahydropyrazino[2,1- c][1,2,4]oxadiazine-1(6H)- carboxylate | 508.7 | 508.4 | 509.3 | [M + H]⁺ | A |
| I- 41 | 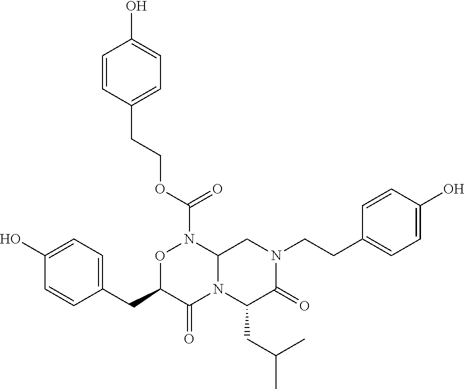 [SMILES: CC(C)C[C@H]1C(=O)N(CCc2ccc(O)cc2)CC2N(C(=O)OCCc3ccc(O)cc3)O[C@H](Cc3ccc(O)cc3)C(=O)N21] | (3R,6S)-4-hydroxyphenethyl 3-(4-hydroxybenzyl)-8-(4- hydroxyphenethyl)-6- isobutyl-4,7- dioxohexahydropyrazino[2,1- c][1,2,4]oxadiazine-1(6H)- carboxylate | 617.7 | 617.3 | 618.3 | [M + H]⁺ | A |
| I- 42 | 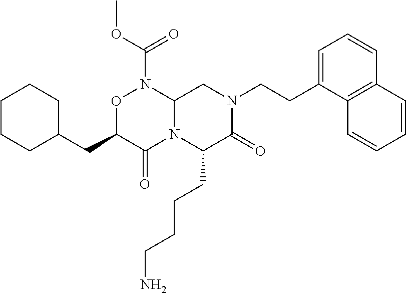 [SMILES: COC(=O)N1O[C@H](CC2CCCCC2)C(=O)N2C1CN(CCc1cccc3ccccc13)C(=O)[C@@H]2CCCCN] | (3R,6S)-methyl 6-(4- aminobutyl)-3- (cyclohexylmethyl)-8-(2- (naphthalen-1-yl)ethyl)-4,7- dioxohexahydropyrazino[2,1- c][1,2,4]oxadiazine-1(6H)- carboxylate | 550.7 | 550.3 | 551.3 | [M + H]⁺ | A |
| I- 43 | 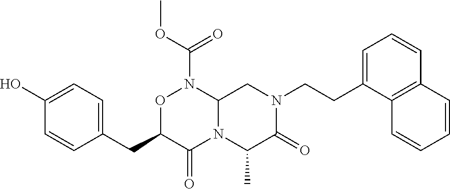 [SMILES: COC(=O)N1O[C@H](Cc2ccc(O)cc2)C(=O)N2C1CN(CCc1cccc3ccccc13)C(=O)[C@@H]2C] | (3R,6S)-methyl 3-(4- hydroxybenzyl)-6-methyl-8- (2-(naphthalen-1-yl)ethyl)- 4,7- dioxohexahydropyrazino[2,1- c][1,2,4]oxadiazine-1(6H)- carboxylate | 503.6 | 503.2 | 504.3 | [M + H]⁺ | A |
| I- 44 | 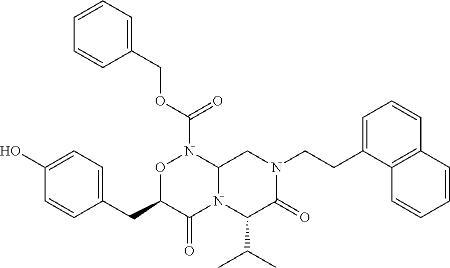 [SMILES: CC(C)[C@H]1C(=O)N(CCc2cccc3ccccc23)CC2N(C(=O)OCc3ccccc3)O[C@H](Cc3ccc(O)cc3)C(=O)N21] | (3R,6S)-benzyl 3-(4- hydroxybenzyl)-6-isopropyl- 8-(2-(naphthalen-1-yl)ethyl)- 4,7- dioxohexahydropyrazino[2,1- c][1,2,4]oxadiazine-1(6H)- carboxylate | 607.7 | 607.3 | 608.3 | [M + H]⁺ | A |
| I- 45 | 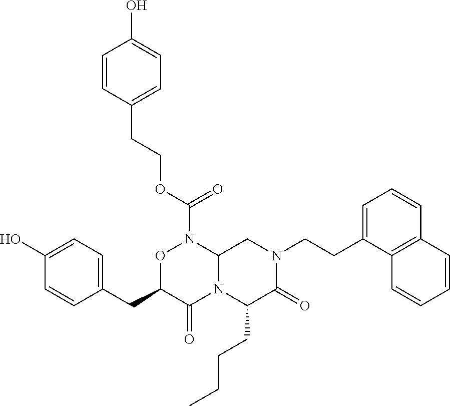 [SMILES: CCCC[C@H]1C(=O)N(CCc2cccc3ccccc23)CC2N(C(=O)OCCc3ccc(O)cc3)O[C@H](Cc3ccc(O)cc3)C(=O)N21] | (3R,6S)-4-hydroxyphenethyl 6-butyl-3-(4-hydroxybenzyl)- 8-(2-(naphthalen-1-yl)ethyl)- 4,7- dioxohexahydropyrazino[2,1- c][1,2,4]oxadiazine-1(6H)- carboxylate | 651.8 | 651.3 | 652.3 | [M + H]⁺ | A |

TABLE 12-8

| | | | | | | | |
|---|---|---|---|---|---|---|---|
| I- 46 | 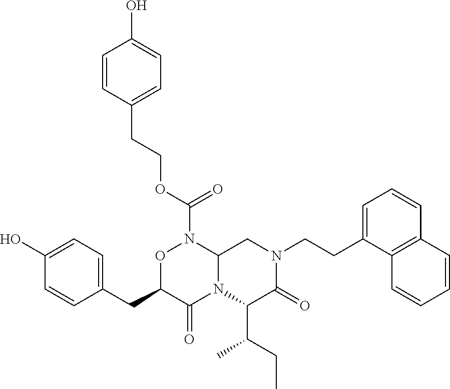 | (3R,6S)-4-hydroxyphenethyl 6-((S)-sec-butyl)-3-(4- hydroxybenzyl)-8-(2- (naphthalen-1-yl)ethyl)-4,7- dioxohexahydropyrazino[2,1- c][1,2,4]oxadiazine-1(6H)- carboxylate | 651.8 | 651.3 | 652.3 | [M + H]⁺ | A |
| I- 47 | 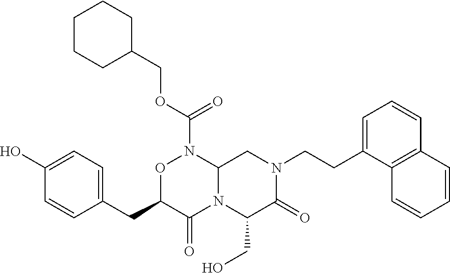 [SMILES: O=C1[C@H](CO)N2C(=O)[C@@H](Cc3ccc(O)cc3)ON(C(=O)OCC3CCCCC3)C2CN1CCc1cccc2ccccc12] | (3R,6S)-cyclohexylmethyl 3- (4-hydroxybenzyl)-6- (hydroxymethyl)-8-(2- (naphthalen-1-yl)ethyl)-4,7- dioxohexahydropyrazino[2,1- c][1,2,4]oxadiazine-1(6H)- carboxylate | 601.7 | 601.3 | 602.3 | [M + H]⁺ | A |
| I- 48 | 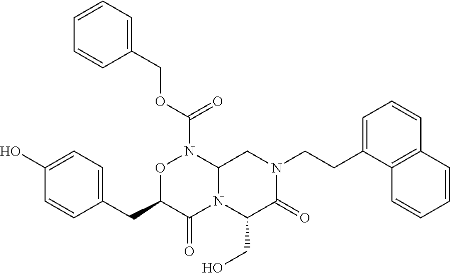 [SMILES: O=C1[C@H](CO)N2C(=O)[C@@H](Cc3ccc(O)cc3)ON(C(=O)OCc3ccccc3)C2CN1CCc1cccc2ccccc12] | (3R,6S)-benzyl 3-(4- hydroxybenzyl)-6- (hydroxymethyl)-8-(2- (naphthalen-1-yl)ethyl)-4,7- dioxohexahydropyrazino[2,1- c][1,2,4]oxadiazine-1(6H)- carboxylate | 595.6 | 595.2 | 596.3 | [M + H]⁺ | A |
| I- 49 | 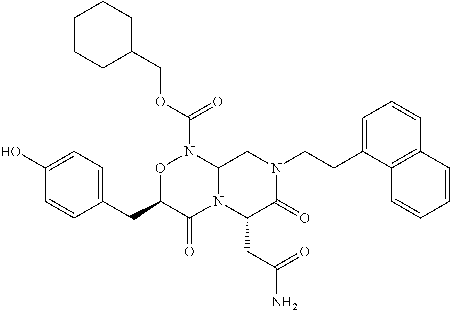 [SMILES: NC(=O)C[C@H]1C(=O)N(CCc2cccc3ccccc23)CC2N(C(=O)OCC3CCCCC3)O[C@H](Cc3ccc(O)cc3)C(=O)N21] | (3R,6S)-cyclohexylmethyl 6- (2-amino-2-oxoethyl)-3-(4- hydroxybenzyl)-8-(2- (naphthalen-1-yl)ethyl)-4,7- dioxohexahydropyrazino[2,1- c][1,2,4]oxadiazine-1(6H)- carboxylate | 628.7 | 628.3 | 629.3 | [M + H]⁺ | A |
| I- 50 | 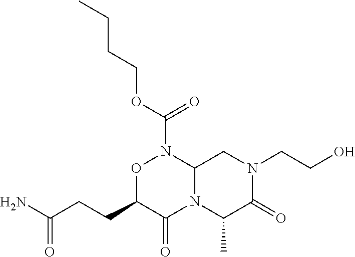 [SMILES: CCCCOC(=O)N1O[C@H](CCC(N)=O)C(=O)N2C1CN(CCO)C(=O)[C@@H]2C] | (3R,6S)-butyl 3-(3-amino-3- oxopropyl)-8-(2- hydroxyethyl)-6-methyl-4,7- dioxohexahydropyrazino[2,1- c][1,2,4]oxadiazine-1(6H)- carboxylate | 400.4 | 400.2 | 401.2 | [M + H]⁺ | A |
| I- 51 | 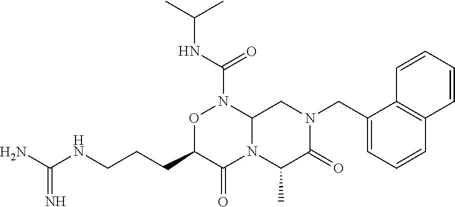 [SMILES: CC(C)NC(=O)N1O[C@H](CCCNC(=N)N)C(=O)N2C1CN(Cc1cccc3ccccc13)C(=O)[C@@H]2C] | (3R,6S)-3-(3- guanidinopropyl)-N- isopropyl-6-methyl-8- (naphthalen-1-ylmethyl)-4,7- dioxohexahydropyrazino[2,1- c][1,2,4]oxadiazine-1(6H)- carboxamide | 509.6 | 509.3 | 510.3 | [M + H]⁺ | A |
| I- 52 | 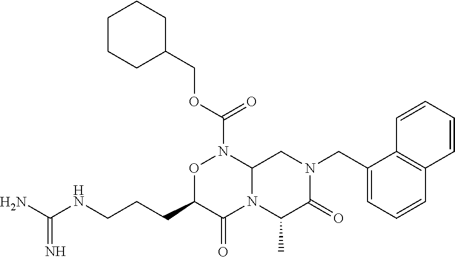 [SMILES: C[C@H]1C(=O)N(Cc2cccc3ccccc23)CC2N(C(=O)OCC3CCCCC3)O[C@H](CCCNC(=N)N)C(=O)N21] | (3R,6S)-cyclohexylmethyl 3- (3-guanidinopropyl)-6- methyl-8-(naphthalen-1- ylmethyl)-4,7- dioxohexahydropyrazino[2,1- c][1,2,4]oxadiazine-1(6H)- carboxylate | 564.7 | 564.3 | 565.3 | [M + H]⁺ | A |

TABLE 12-9

| | | | | | | | |
|---|---|---|---|---|---|---|---|
| I- 53 | 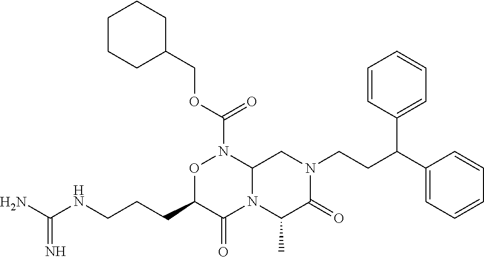 [SMILES: C[C@H]1C(=O)N(CCC(c2ccccc2)c2ccccc2)CC2N(C(=O)OCC3CCCCC3)O[C@H](CCCNC(=N)N)C(=O)N21] | (3R,6S)-cyclohexylmethyl 8- (3,3-diphenylpropyl)-3-(3- guanidinopropyl)-6-methyl- 4,7- dioxohexahydropyrazino[2,1- c][1,2,4]oxadiazine-1(6H)- carboxylate | 618.8 | 618.4 | 619.3 | [M + H]⁺ | A |
| I- 54 | 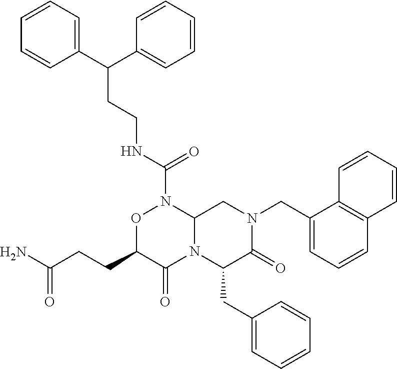 [SMILES: NC(=O)CC[C@H]1ON(C(=O)NCCC(c2ccccc2)c2ccccc2)C2CN(Cc3cccc4ccccc34)C(=O)[C@H](Cc3ccccc3)N2C1=O] | (3R,6S)-3-(3-amino-3- oxopropyl)-6-benzyl-N-(3,3- diphenylpropyl)-8- (naphthalen-1-ylmethyl)-4,7- dioxohexahydropyrazino[2,1- c][1,2,4]oxadiazine-1(6H)- carboxamide | 709.8 | 709.3 | 710.4 | [M + H]⁺ | A |
| I- 55 | 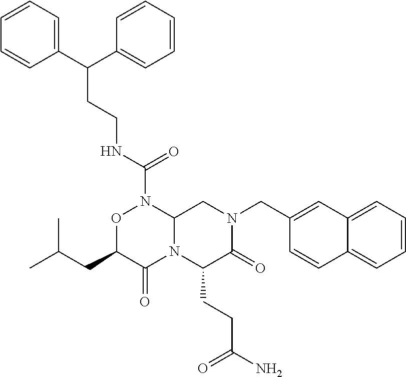 [SMILES: CC(C)C[C@H]1ON(C(=O)NCCC(c2ccccc2)c2ccccc2)C2CN(Cc3ccc4ccccc4c3)C(=O)[C@H](CCC(N)=O)N2C1=O] | (3R,6S)-6-(3-amino-3- oxopropyl)-N-(3,3- diphenylpropyl)-3-isobutyl-8- (naphthalen-2-ylmethyl)-4,7- dioxohexahydropyrazino[2,1- c][1,2,4]oxadiazine-1(6H)- carboxamide | 675.8 | 675.3 | 676.4 | [M + H]⁺ | A |
| I- 56 | 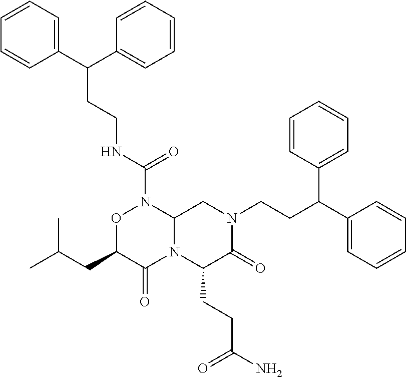 [SMILES: CC(C)C[C@H]1ON(C(=O)NCCC(c2ccccc2)c2ccccc2)C2CN(CCC(c3ccccc3)c3ccccc3)C(=O)[C@H](CCC(N)=O)N2C1=O] | (3R,6S)-6-(3-amino-3- oxopropyl)-N,8-bis(3,3- diphenylpropyl)-3-isobutyl- 4,7- dioxohexahydropyrazino[2,1- c][1,2,4]oxadiazine-1(6H)- carboxamide | 729.9 | 729.4 | 730.3 | [M + H]⁺ | A |
| I- 57 | 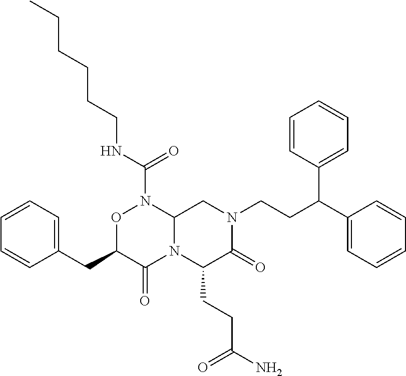 [SMILES: CCCCCCNC(=O)N1O[C@H](Cc2ccccc2)C(=O)N2C1CN(CCC(c1ccccc1)c1ccccc1)C(=O)[C@@H]2CCC(N)=O] | (3R,6S)-6-(3-amino-3- oxopropyl)-3-benzyl-8-(3,3- diphenylpropyl)-N-hexyl-4,7- dioxohexahydropyrazino[2,1- c][1,2,4]oxadiazine-1(6H)- carboxamide | 653.8 | 653.4 | 654.4 | [M + H]⁺ | A |
| I- 58 | 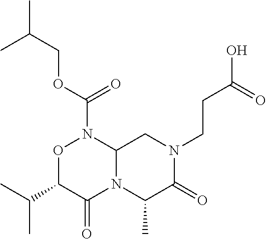 [SMILES: CC(C)COC(=O)N1O[C@@H](C(C)C)C(=O)N2C1CN(CCC(=O)O)C(=O)[C@@H]2C] | 3-((3S,6S)-1- (isobutoxycarbonyl)-3- isopropyl-6-methyl-4,7- dioxohexahydropyrazino[2,1- c][1,2,4]oxadiazin-8(1H)- yl)propanoic acid | 399.4 | 399.2 | 400.2 | [M + H]⁺ | A |
| I- 59 | 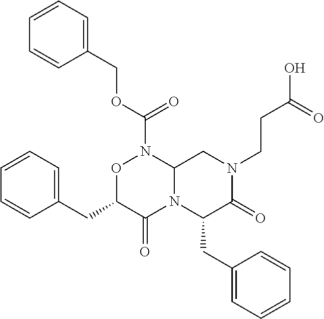 [SMILES: O=C(O)CCN1CC2N(C(=O)OCc3ccccc3)O[C@@H](Cc3ccccc3)C(=O)N2[C@@H](Cc2ccccc2)C1=O] | 3-((3S,6S)-3,6-dibenzyl-1- ((benzyloxy)carbonyl)-4,7- dioxohexahydropyrazino[2,1- c][1,2,4]oxadiazin-8(1H)- yl)propanoic acid | 557.6 | 557.2 | 558.2 | [M + H]⁺ | A |

TABLE 12-10

| | | | | | | | |
|---|---|---|---|---|---|---|---|
| I- 60 | 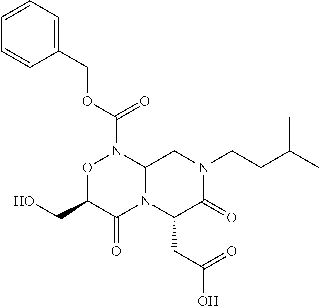 [SMILES: CC(C)CCN1CC2N(C(=O)OCc3ccccc3)O[C@H](CO)C(=O)N2[C@@H](CC(=O)O)C1=O] | 2-((3R,6S)-1- ((benzyloxy)carbonyl)-3- (hydroxymethyl)-8-isopentyl- 4,7- dioxooctahydropyrazino[2,1- c][1,2,4]oxadiazin-6- yl)acetic acid | 463.5 | 463.2 | 464.2 | [M + H]⁺ | A |
| I- 61 | 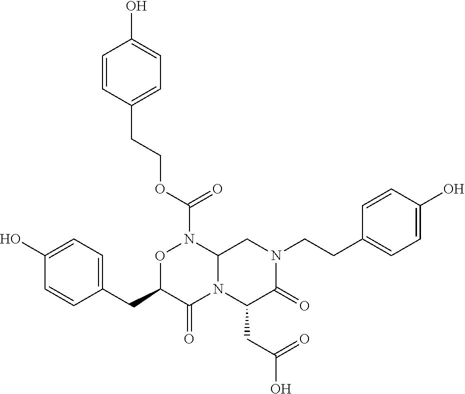 [SMILES: O=C(O)C[C@H]1C(=O)N(CCc2ccc(O)cc2)CC2N(C(=O)OCCc3ccc(O)cc3)O[C@H](Cc3ccc(O)cc3)C(=O)N21] | 2-((3R,6S)-3-(4- hydroxybenzyl)-1-((4- hydroxyphenethoxy) carbonyl)-8-(4- hydroxyphenethyl)-4,7- dioxooctahydropyrazino[2,1- c][1,2,4]oxadiazin-6- yl)acetic acid | 619.6 | 619.2 | 620.2 | [M + H]⁺ | A |
| I- 62 | 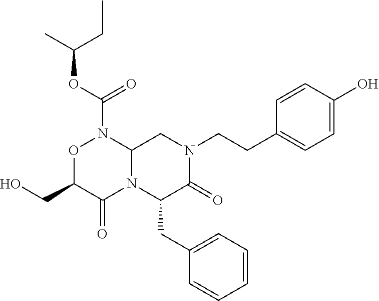 [SMILES: CC[C@H](C)OC(=O)N1O[C@H](CO)C(=O)N2C1CN(CCc1ccc(O)cc1)C(=O)[C@@H]2Cc1ccccc1] | (3R,6S)-(S)-sec-butyl 6- benzyl-3-(hydroxymethyl)-8- (4-hydroxyphenethyl)-4,7- dioxohexahydropyrazino[2,1- c][1,2,4]oxadiazine-1(6H)- carboxylate | 511.6 | 511.2 | 512.2 | [M + H]⁺ | A |
| I- 63 | 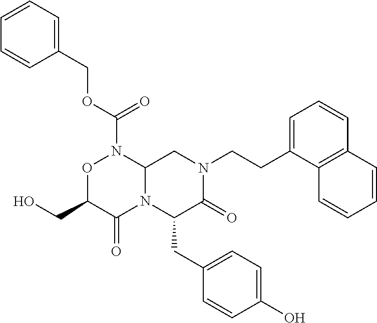 [SMILES: O=C1[C@H](Cc2ccc(O)cc2)N2C(=O)[C@@H](CO)ON(C(=O)OCc3ccccc3)C2CN1CCc1cccc2ccccc12] | (3R,6S)-benzyl 6-(4- hydroxybenzyl)-3- (hydroxymethyl)-8-(2- (naphthalen-1-yl)ethyl)-4,7- dioxohexahydropyrazino[2,1- c][1,2,4]oxadiazine-1(6H)- carboxylate | 595.6 | 595.2 | 596.2 | [M + H]⁺ | A |
| I- 64 | 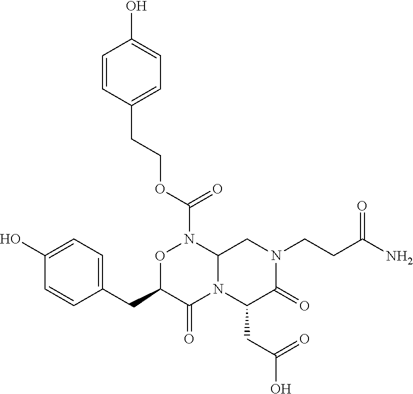 [SMILES: NC(=O)CCN1CC2N(C(=O)OCCc3ccc(O)cc3)O[C@H](Cc3ccc(O)cc3)C(=O)N2[C@@H](CC(=O)O)C1=O] | 2-((3R,6S)-8-(3-amino-3- oxopropyl)-3-(4- hydroxybenzyl)-1-((4- hydroxyphenethoxy) carbonyl)-4,7- dioxooctahydropyrazino[2,1- c][1,2,4]oxadiazin-6- yl)acetic acid | 570.6 | 570.2 | 571.2 | [M + H]⁺ | A |
| I- 65 | 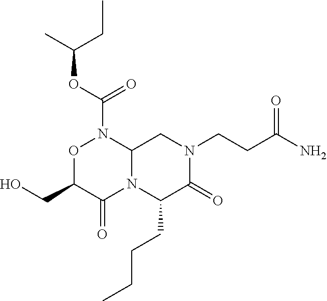 [SMILES: CCCC[C@H]1C(=O)N(CCC(N)=O)CC2N(C(=O)O[C@@H](C)CC)O[C@H](CO)C(=O)N21] | (3R,6S)-(S)-sec-butyl 8-(3- amino-3-oxopropyl)-6-butyl- 3-(hydroxymethyl)-4,7- dioxohexahydropyrazino[2,1- c][1,2,4]oxadiazine-1(6H)- carboxylate | 428.5 | 428.2 | 429.2 | [M + H]⁺ | A |
| I- 66 | 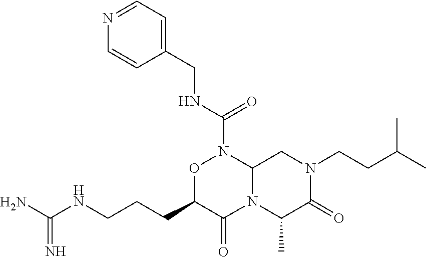 [SMILES: CC(C)CCN1CC2N(C(=O)NCc3ccncc3)O[C@H](CCCNC(=N)N)C(=O)N2[C@@H](C)C1=O] | (3R,6S)-3-(3- guanidinopropyl)-8- isopentyl-6-methyl-4,7- dioxo-N-(pyridin-4- ylmethyl)hexahydropyrazino [2,1-c][1,2,4]oxadiazine- 1(6H)-carboxamide | 488.6 | 488.3 | 489.3 | [M + H]⁺ | A |

TABLE 12-11

| | | | | | | | |
|---|---|---|---|---|---|---|---|
| I-67 | 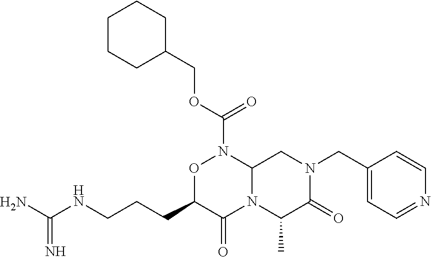 [SMILES: C[C@H]1C(=O)N(Cc2ccncc2)CC2N(C(=O)OCC3CCCCC3)O[C@H](CCCNC(=N)N)C(=O)N21] | (3R,6S)-cyclohexylmethyl 3- (3-guanidinopropyl)-6- methyl-4,7-dioxo-8-(pyridin- 4- ylmethyl)hexahydropyrazino [2,1-c][1,2,4]oxadiazine- 1(6H)-carboxylate | 515.6 | 515.3 | 516.3 | [M + H]⁺ | A |
| I-68 | 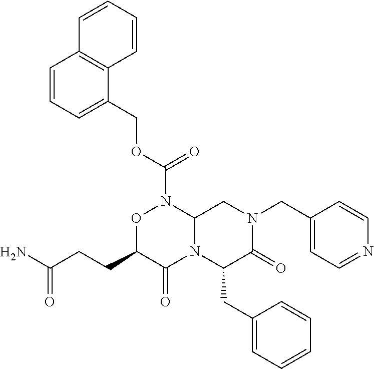 [SMILES: NC(=O)CC[C@H]1ON(C(=O)OCc2cccc3ccccc23)C2CN(Cc3ccncc3)C(=O)[C@H](Cc3ccccc3)N2C1=O] | (3R,6S)-naphthalen-1- ylmethyl 3-(3-amino-3- oxopropyl)-6-benzyl-4,7- dioxo-8-(pyridin-4- ylmethyl)hexahydropyrazino [2,1-c][1,2,4]oxadiazine- 1(6H)-carboxylate | 607.7 | 607.2 | 608.2 | [M + H]⁺ | A |
| I-69 | 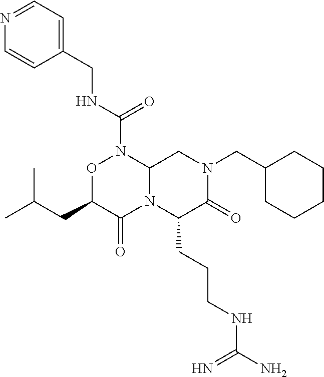 [SMILES: CC(C)C[C@H]1ON(C(=O)NCc2ccncc2)C2CN(CC3CCCCC3)C(=O)[C@H](CCCNC(=N)N)N2C1=O] | (3R,6S)-8- (cyclohexylmethyl)-6-(3- guanidinopropyl)-3-isobutyl- 4,7-dioxo-N-(pyridin-4- ylmethyl)hexahydropyrazino [2,1-c][1,2,4]oxadiazine- 1(6H)-carboxamide | 556.7 | 556.3 | 557.3 | [M + H]⁺ | A |
| I-70 | 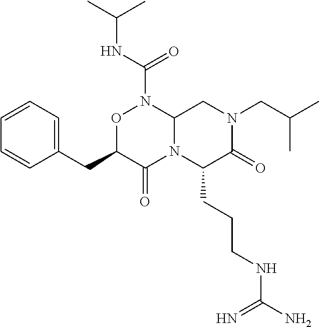 [SMILES: CC(C)CN1CC2N(C(=O)NC(C)C)O[C@H](Cc3ccccc3)C(=O)N2[C@@H](CCCNC(=N)N)C1=O] | (3R,6S)-3-benzyl-6-(3- guanidinopropyl)-8-isobutyl- N-isopropyl-4,7- dioxohexahydropyrazino[2,1- c][1,2,4]oxadiazine-1(6H)- carboxamide | 501.6 | 501.3 | 502.3 | [M + H]⁺ | A |
| I-71 | 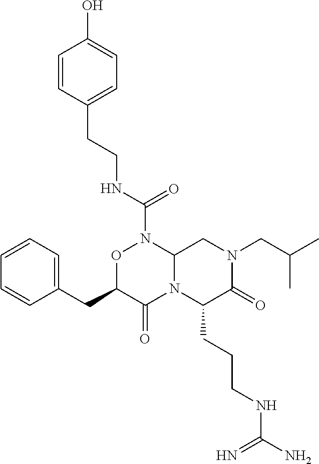 [SMILES: CC(C)CN1CC2N(C(=O)NCCc3ccc(O)cc3)O[C@H](Cc3ccccc3)C(=O)N2[C@@H](CCCNC(=N)N)C1=O] | (3R,6S)-3-benzyl-6-(3- guanidinopropyl)-N-(4- hydroxyphenethyl)-8- isobutyl-4,7- dioxohexahydropyrazino[2,1- c][1,2,4]oxadiazine-1(6H)- carboxamide | 579.7 | 579.3 | 580.3 | [M + H]⁺ | A |
| I-72 | 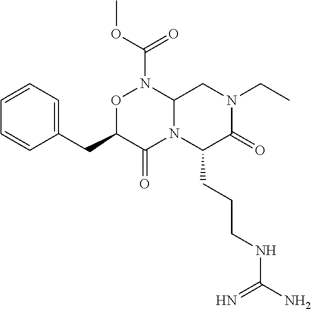 [SMILES: CCN1CC2N(C(=O)OC)O[C@H](Cc3ccccc3)C(=O)N2[C@@H](CCCNC(=N)N)C1=O] | (3R,6S)-methyl 3-benzyl-8- ethyl-6-(3-guanidinopropyl)- 4,7- dioxohexahydropyrazino[2,1- c][1,2,4]oxadiazine-1(6H)- carboxylate | 446.5 | 446.2 | 447.2 | [M + H]⁺ | A |
| I-73 | 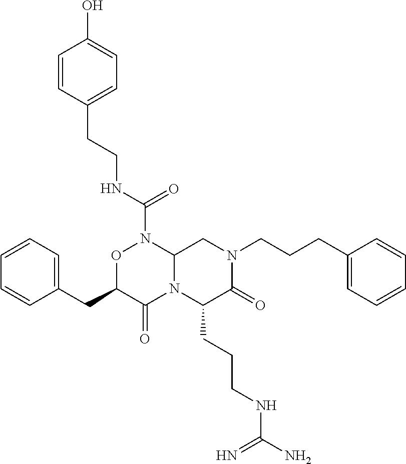 [SMILES: N=C(N)NCCC[C@H]1C(=O)N(CCCc2ccccc2)CC2N(C(=O)NCCc3ccc(O)cc3)O[C@H](Cc3ccccc3)C(=O)N21] | (3R,6S)-3-benzyl-6-(3- guanidinopropyl)-N-(4- hydroxyphenethyl)-4,7- dioxo-8-(3- phenylpropyl) hexahydropyra zino[2,1-c][1,2,4]oxadiazine- 1(6H)-carboxamide | 641.8 | 641.3 | 642.3 | [M + H]⁺ | A |

TABLE 12-12

| | | | | | | | |
|---|---|---|---|---|---|---|---|
| I-74 | 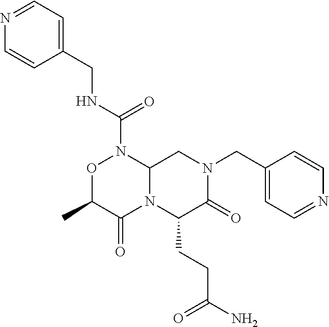 [SMILES: C[C@H]1ON(C(=O)NCc2ccncc2)C2CN(Cc3ccncc3)C(=O)[C@H](CCC(N)=O)N2C1=O] | (3R,6S)-6-(3-amino-3- oxopropyl)-3-methyl-4,7- dioxo-N,8-bis(pyridin-4- ylmethyl)hexahydropyrazino [2,1-c][1,2,4]oxadiazine- 1(6H)-carboxamide | 481.5 | 481.2 | 482.2 | [M + H]⁺ | A |
| I-75 | 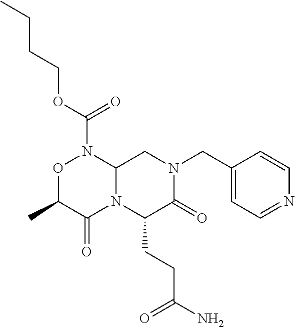 [SMILES: CCCCOC(=O)N1O[C@H](C)C(=O)N2C1CN(Cc1ccncc1)C(=O)[C@@H]2CCC(N)=O] | (3R,6S)-butyl 6-(3-amino-3- oxopropyl)-3-methyl-4,7- dioxo-8-(pyridin-4- ylmethyl)hexahydropyrazino [2,1-c][1,2,4]oxadiazine- 1(6H)-carboxylate | 447.5 | 447.2 | 448.2 | [M + H]⁺ | A |
| I-76 | 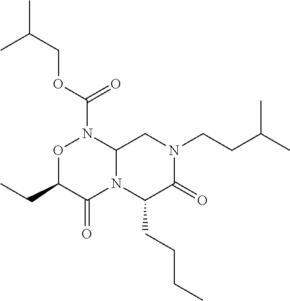 [SMILES: CCCC[C@H]1C(=O)N(CCC(C)C)CC2N(C(=O)OCC(C)C)O[C@H](CC)C(=O)N21] | isobutyl (3R,6S)-6-butyl-3- ethyl-8-isopentyl-4,7- dioxohexahydropyrazino[2,1- c][1,2,4]oxadiazine-1(6H)- carboxylate | 425.6 | 425.3 | 426.3 | [M + H]⁺ | A |
| I-77 | 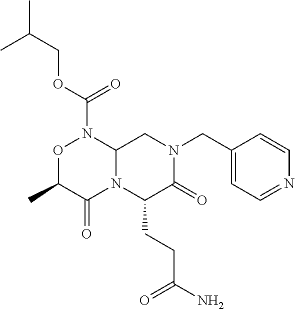 [SMILES: CC(C)COC(=O)N1O[C@H](C)C(=O)N2C1CN(Cc1ccncc1)C(=O)[C@@H]2CCC(N)=O] | isobutyl (3R,6S)-6-(3-amino- 3-oxopropyl)-3-methyl-4,7- dioxo-8-(pyridin-4- ylmethyl)hexahydropyrazino [2,1-c][1,2,4]oxadiazine- 1(6H)-carboxylate | 447.5 | 447.2 | 448.2 | [M + H]⁺ | A |
| I-78 | 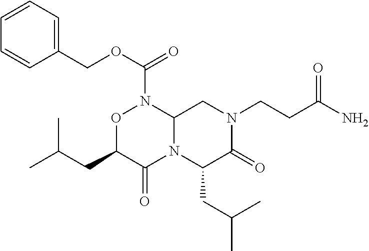 [SMILES: CC(C)C[C@H]1ON(C(=O)OCc2ccccc2)C2CN(CCC(N)=O)C(=O)[C@H](CC(C)C)N2C1=O] | benzyl (3R,6S)-8-(3-amino-3- oxopropyl)-3,6-diisobutyl- 4,7- dioxohexahydropyrazino[2,1- c][1,2,4]oxadiazine-1(6H)- carboxylate | 488.6 | 488.3 | 489.2 | [M + H]⁺ | A |
| I-79 | 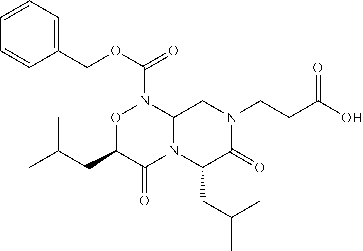 [SMILES: CC(C)C[C@H]1ON(C(=O)OCc2ccccc2)C2CN(CCC(=O)O)C(=O)[C@H](CC(C)C)N2C1=O] | 3-((3R,6S)-1- ((benzyloxy)carbonyl)-3,6- diisobutyl-4,7- dioxohexahydropyrazino[2,1- c][1,2,4]oxadiazin-8(1H)- yl)propanoic acid | 489.6 | 489.2 | 490.3 | [M + H]⁺ | A |
| I-80 | 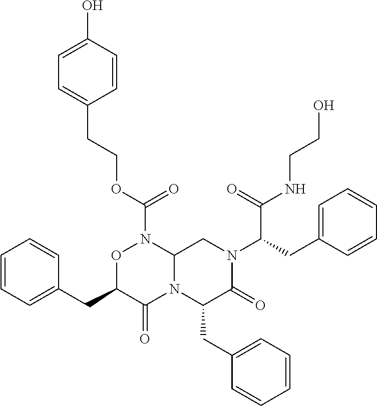 [SMILES: O=C(NCCO)[C@H](Cc1ccccc1)N1CC2N(C(=O)OCCc3ccc(O)cc3)O[C@H](Cc3ccccc3)C(=O)N2[C@@H](Cc2ccccc2)C1=O] | 4-hydroxyphenethyl (3R,6S)- 3,6-dibenzyl-8-(S)-1-(2- hydroxyethyl)amino)-1-oxo- 3-phenylpropan-2-yl)-4,7- dioxohexahydropyrazino[2,1- c][1,2,4]oxadiazine-1(6H)- carboxylate | 706.8 | 706.3 | 707.3 | [M + H]⁺ | A |

TABLE 12-13

| | | | | | | | |
|---|---|---|---|---|---|---|---|
| I- 81 | 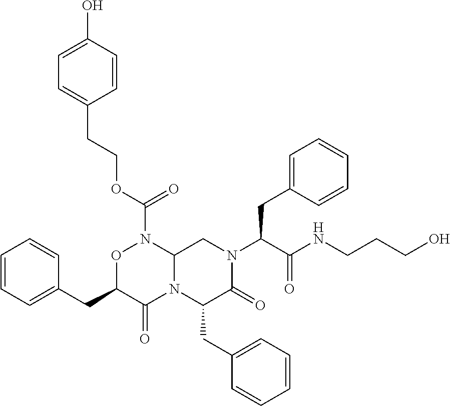 [SMILES: O=C(NCCCO)[C@H](Cc1ccccc1)N1CC2N(C(=O)OCCc3ccc(O)cc3)O[C@H](Cc3ccccc3)C(=O)N2[C@@H](Cc2ccccc2)C1=O] | 4-hydroxyphenethyl (3R,6S)- 3,6-dibenzyl-8-((S)-1-((3- hydroxypropyl) amino)-1-oxo- 3-phenylpropan-2-yl)-4,7- dioxohexahydro- pyrazino[2,1-c] [1,2,4]oxadiazine-1(6H)- carboxylate | 720.8 | 720.3 | 721.3 | [M + H]⁺ | A |
| I- 82 | 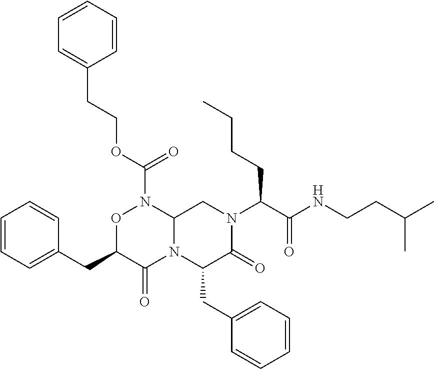 [SMILES: CCCC[C@@H](C(=O)NCCC(C)C)N1CC2N(C(=O)OCCc3ccccc3)O[C@H](Cc3ccccc3)C(=O)N2[C@@H](Cc2ccccc2)C1=O] | phenethyl (3R,6S)-3,6- dibenzyl-8-((S)-1- (isopentylamino)-1- oxohexan-2-yl)-4,7- dioxohexahydro- pyrazino[2,1-c] [1,2,4]oxadiazine-1(6H)- carboxylate | 682.9 | 682.4 | 683.4 | [M H]⁺ | A |
| I- 83 | 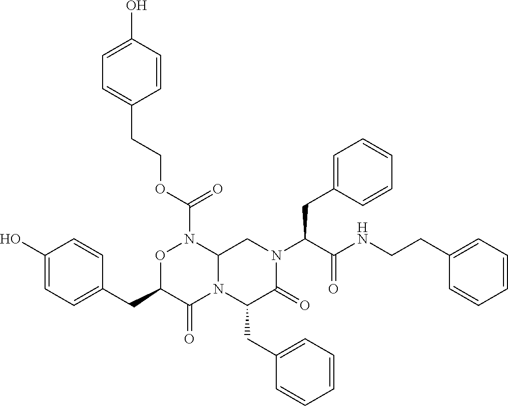 [SMILES: O=C(NCCc1ccccc1)[C@H](Cc1ccccc1)N1CC2N(C(=O)OCCc3ccc(O)cc3)O[C@H](Cc3ccc(O)cc3)C(=O)N2[C@@H](Cc2ccccc2)C1=O] | 4-hydroxyphenethyl (3R,6S)- 6-benzyl-3-(4- hydroxybenzyl)- 4,7-dioxo-8- ((S)-1-oxo-1- (phenethylamino)-3- phenylpropan-2- yl)hexahydropyrazino [2,1-c][1,2,4]oxadiazine- 1(6H)- carboxylate | 782.9 | 782.3 | 783.2 | [M + H]⁺ | A |
| I- 84 | 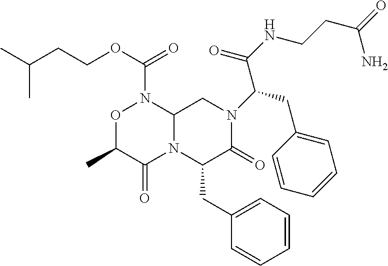 [SMILES: CC(C)CCOC(=O)N1O[C@H](C)C(=O)N2C1CN([C@@H](Cc1ccccc1)C(=O)NCCC(N)=O)C(=O)[C@@H]2Cc1ccccc1] | isopentyl (3R,6S)-8-((S)-1- ((3-amino-3- oxopropyl)amino)-1-oxo- 3-phenylpropan- 2-yl)-6-benzyl- 3-methyl-4,7- dioxohexahydro- pyrazino[2,1-c] [1,2,4]oxadiazine-1(6H)- carboxylate | 607.7 | 607.3 | 608.2 | [M + H]⁺ | A |
| I- 85 | 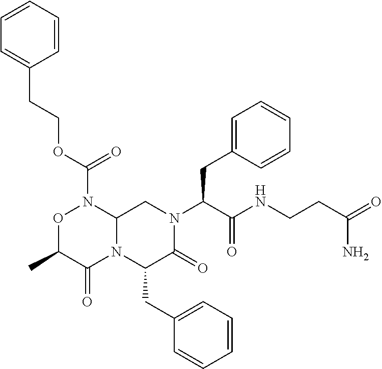 [SMILES: C[C@H]1ON(C(=O)OCCc2ccccc2)C2CN([C@@H](Cc3ccccc3)C(=O)NCCC(N)=O)C(=O)[C@H](Cc3ccccc3)N2C1=O] | phenethyl (3R,6S)-8-((S)-1- ((3-amino-3- oxopropyl)amino)-1-oxo- 3-phenylpropan- 2-yl)-6-benzyl- 3-methyl-4,7- dioxohexahydro- pyrazino[2,1-c] [1,2,4]oxadiazine-1(6H)- carboxylate | 641.7 | 641.3 | 642.2 | [M + H]⁺ | A |
| I- 86 | 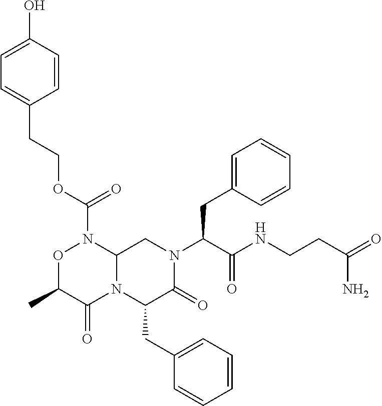 [SMILES: C[C@H]1ON(C(=O)OCCc2ccc(O)cc2)C2CN([C@@H](Cc3ccccc3)C(=O)NCCC(N)=O)C(=O)[C@H](Cc3ccccc3)N2C1=O] | 4-hydroxyphenethyl (3R,6S)- 8-((S)-1-((3-amino-3- oxopropyl)amino)-1-oxo- 3-phenylpropan-2- yl)-6-benzyl- 3-methyl-4,7- dioxohexahydro- pyrazino[2,1-c] [1,2,4]oxadiazine-1(6H)- carboxylate | 657.7 | 657.3 | 658.2 | [M + H]⁺ | A |
| I- 87 | 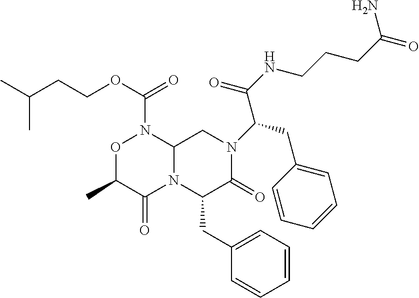 [SMILES: CC(C)CCOC(=O)N1O[C@H](C)C(=O)N2C1CN([C@@H](Cc1ccccc1)C(=O)NCCCC(N)=O)C(=O)[C@@H]2Cc1ccccc1] | isopentyl (3R,6S)-8-((S)-1- ((4-amino-4- oxobutyl)amino)-1-oxo- 3-phenylpropan-2- yl)-6-benzyl- 3-methyl-4,7- dioxohexahydro- pyrazino[2,1-c] [1,2,4]oxadiazine-1(6H)- carboxylate | 621.7 | 621.3 | 622.2 | [M + H]⁺ | A |

TABLE 12-14

| | | | | | | | |
|---|---|---|---|---|---|---|---|
| I-88 | 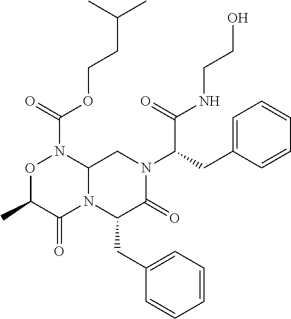 [SMILES: CC(C)CCOC(=O)N1O[C@H](C)C(=O)N2C1CN([C@@H](Cc1ccccc1)C(=O)NCCO)C(=O)[C@@H]2Cc1ccccc1] | isopentyl (3R,6S)-6-benzyl-8- ((S)-1-((2- hydroxyethyl)amino)-1-oxo- 3-phenylpropan-2-yl)-3- methyl-4,7- dioxohexahydropyrazino[2,1- c][1,2,4]oxadiazine-1(6H)- carboxylate | 580.7 | 580.3 | 581.3 | [M + H]⁺ | A |
| I-89 | 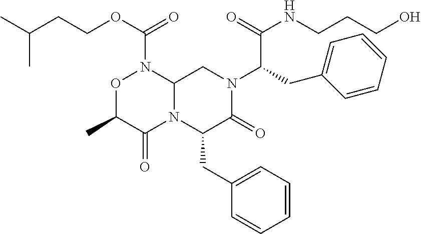 [SMILES: CC(C)CCOC(=O)N1O[C@H](C)C(=O)N2C1CN([C@@H](Cc1ccccc1)C(=O)NCCCO)C(=O)[C@@H]2Cc1ccccc1] | isopentyl (3R,6S)-6-benzyl-8- ((S)-1-((3- hydroxypropyl)amino)-1-oxo- 3-phenylpropan-2-yl)-3- methyl-4,7- dioxohexahydropyrazino[2,1- c][1,2,4]oxadiazine-1(6H)- carboxylate | 594.7 | 594.3 | 595.2 | [M + H]⁺ | A |
| I-90 | 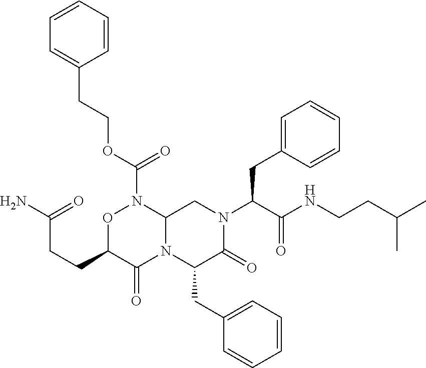 [SMILES: CC(C)CCNC(=O)[C@H](Cc1ccccc1)N1CC2N(C(=O)OCCc3ccccc3)O[C@H](CCC(N)=O)C(=O)N2[C@@H](Cc2ccccc2)C1=O] | phenethyl (3R,6S)-3-(3- amino-3-oxopropyl)-6- benzyl-8-((S)-1- (isopentylamino)-1-oxo-3- phenylpropan-2-yl)-4,7- dioxohexahydropyrazino[2,1- c][1,2,4]oxadiazine-1(6H)- carboxylate | 697.8 | 697.3 | 698.4 | [M + H]⁺ | A |
| I-91 | 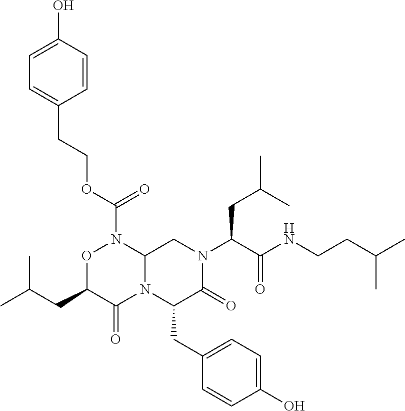 [SMILES: CC(C)CCNC(=O)[C@H](CC(C)C)N1CC2N(C(=O)OCCc3ccc(O)cc3)O[C@H](CC(C)C)C(=O)N2[C@@H](Cc2ccc(O)cc2)C1=O] | 4-hydroxyphenethyl (3R,6S)- 6-(4-hydroxybenzyl)-3- isobutyl-8-((S)-1- (isopentylamino)-4-methyl- 1-oxopentan-2-yl)-4,7- dioxohexahydropyrazino[2,1- c][1,2,4]oxadiazine-1(6H)- carboxylate | 680.8 | 680.4 | 681.3 | [M + H]⁺ | A |
| I-92 | 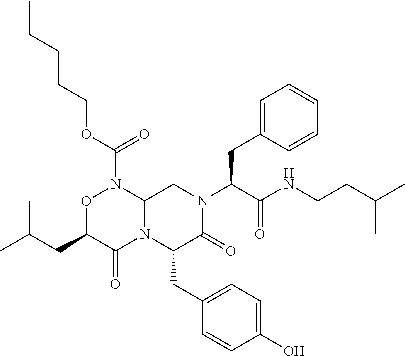 [SMILES: CCCCCOC(=O)N1O[C@H](CC(C)C)C(=O)N2C1CN([C@@H](Cc1ccccc1)C(=O)NCCC(C)C)C(=O)[C@@H]2Cc1ccc(O)cc1] | pentyl (3R,6S)-6-(4- hydroxybenzyl)-3-isobutyl-8- ((S)-1-(isopentylamino)-1- oxo-3-phenylpropan-2-yl)- 4,7- dioxohexahydropyrazino[2,1- c][1,2,4]oxadiazine-1(6H)- carboxylate | 664.8 | 664.4 | 665.4 | [M + H]⁺ | A |
| I-93 | 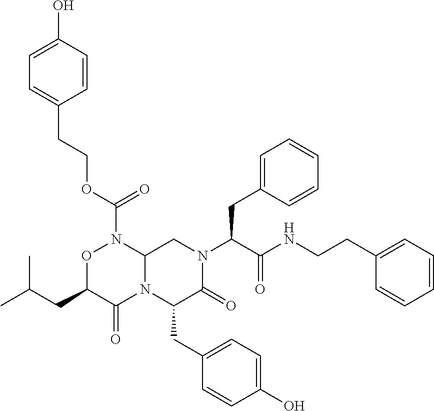 [SMILES: CC(C)C[C@H]1ON(C(=O)OCCc2ccc(O)cc2)C2CN([C@@H](Cc3ccccc3)C(=O)NCCc3ccccc3)C(=O)[C@H](Cc3ccc(O)cc3)N2C1=O] | 4-hydroxyphenethyl (3R,6S)- 6-(4-hydroxybenzyl)-3- isobutyl-4,7-dioxo-8-((S)-1- oxo-1-(phenethylamino)-3- phenylpropan-2- yl)hexahydropyrazino[2,1- c][1,2,4]oxadiazine-1(6H)- carboxylate | 748.9 | 748.3 | 749.3 | [M + H]⁺ | A |

TABLE 12-15

| | | | | | | | |
|---|---|---|---|---|---|---|---|
| I-94 | 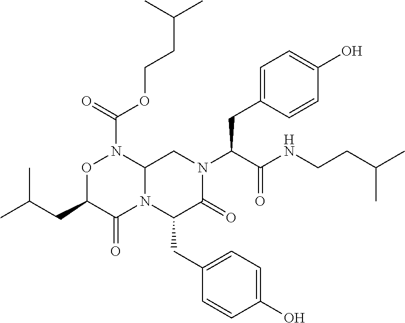 [SMILES: CC(C)CCNC(=O)[C@H](Cc1ccc(O)cc1)N1CC2N(C(=O)OCCC(C)C)O[C@H](CC(C)C)C(=O)N2[C@@H](Cc2ccc(O)cc2)C1=O] | isopentyl (3R,6S)-6-(4- hydroxybenzyl)-8-((S)-3-(4- hydroxyphenyl)-1- (isopentylamino)-1- oxopropan- 2-yl)-3-isobutyl-4,7- dioxohexahydropyrazino[2,1- c][1,2,4]oxadiazine-1(6H)- carboxylate | 680.8 | 680.4 | 681.3 | [M + H]⁺ | A |
| I-95 | 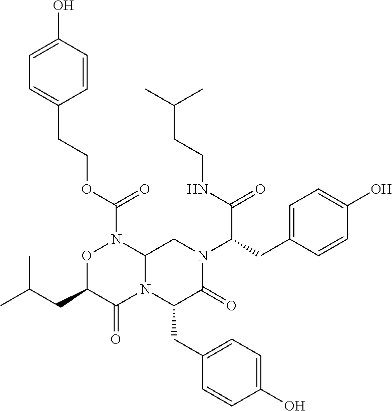 [SMILES: CC(C)CCNC(=O)[C@H](Cc1ccc(O)cc1)N1CC2N(C(=O)OCCc3ccc(O)cc3)O[C@H](CC(C)C)C(=O)N2[C@@H](Cc2ccc(O)cc2)C1=O] | 4-hydroxyphenethyl (3R,6S)-6-(4- hydroxybenzyl)-8-((S)-3-(4- hydroxyphenyl)-1- (isopentylamino)-1- oxopropan- 2-yl)-3-isobutyl-4,7- dioxohexahydropyrazino[2,1- c][1,2,4]oxadiazine-1(6H)- carboxylate | 730.9 | 730.4 | 731.2 | [M + H]⁺ | A |
| I-96 | 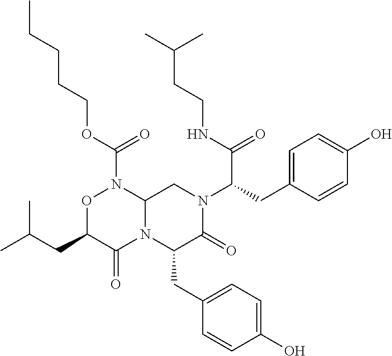 [SMILES: CCCCCOC(=O)N1O[C@H](CC(C)C)C(=O)N2C1CN([C@@H](Cc1ccc(O)cc1)C(=O)NCCC(C)C)C(=O)[C@@H]2Cc1ccc(O)cc1] | pentyl (3R,6S)-6-(4- hydroxybenzyl)-8-((S)-3-(4- hydroxyphenyl)-1- (isopentylamino)- 1-oxopropan- 2-yl)-3-isobutyl-4,7- dioxohexahydropyrazino[2,1- c][1,2,4]oxadiazine-1(6H)- carboxylate | 680.8 | 680.4 | 681.3 | [M + H]⁺ | A |
| I-97 | 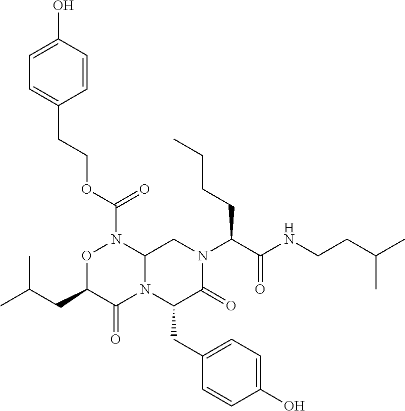 [SMILES: CCCC[C@@H](C(=O)NCCC(C)C)N1CC2N(C(=O)OCCc3ccc(O)cc3)O[C@H](CC(C)C)C(=O)N2[C@@H](Cc2ccc(O)cc2)C1=O] | 4-hydroxyphenethyl (3R,6S)-6-(4- hydroxybenzyl)-3-isobutyl- 8-((S)-1-(isopentylamino)- 1-oxohexan-2-yl)-4,7- dioxohexahydropyrazino[2,1- c][1,2,4]oxadiazine-1(6H)- carboxylate | 680.8 | 680.4 | 681.3 | [M + H]⁺ | A |
| I-98 | 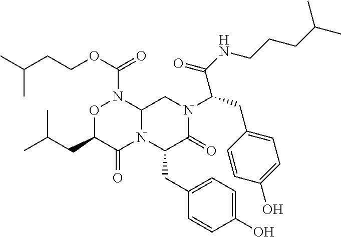 [SMILES: CC(C)CCCNC(=O)[C@H](Cc1ccc(O)cc1)N1CC2N(C(=O)OCCC(C)C)O[C@H](CC(C)C)C(=O)N2[C@@H](Cc2ccc(O)cc2)C1=O] | isopentyl (3R,6S)-6-(4- hydroxybenzyl)-8-((S)-3-(4- hydroxyphenyl)-1-((4- methylpentyl)amino)-1- oxopropan-2-yl)-3-isobutyl- 4,7-dioxohexahydropyrazino [2,1-c][1,2,4]oxadiazine- 1(6H)-carboxylate | 694.9 | 694.4 | 695.3 | [M + H]⁺ | A |
| I-99 | 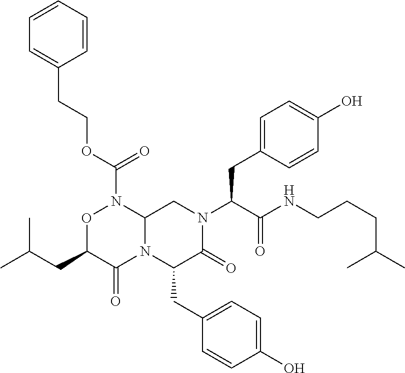 [SMILES: CC(C)CCCNC(=O)[C@H](Cc1ccc(O)cc1)N1CC2N(C(=O)OCCc3ccccc3)O[C@H](CC(C)C)C(=O)N2[C@@H](Cc2ccc(O)cc2)C1=O] | phenethyl (3R,6S)-6-(4- hydroxybenzyl)-8-((S)-3-(4- hydroxyphenyl)-1-((4- methylpentyl)amino)-1- oxopropan-2-yl)-3-isobutyl- 4,7-dioxohexahydropyrazino [2,1-c][1,2,4]oxadiazine- 1(6H)-carboxylate | 728.9 | 728.4 | 729.3 | [M + H]⁺ | A |
| I-100 | 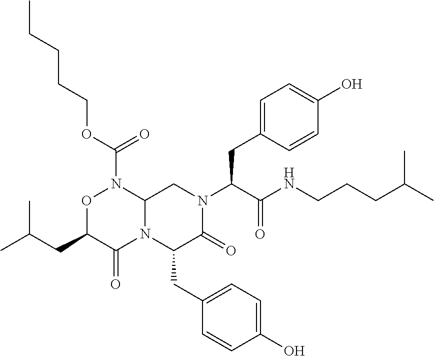 [SMILES: CCCCCOC(=O)N1O[C@H](CC(C)C)C(=O)N2C1CN([C@@H](Cc1ccc(O)cc1)C(=O)NCCCC(C)C)C(=O)[C@@H]2Cc1ccc(O)cc1] | pentyl (3R,6S)-6-(4- hydroxybenzyl)-8-((S)-3-(4- hydroxyphenyl)-1-((4- methylpentyl)amino)-1- oxopropan-2-yl)-3-isobutyl- 4,7-dioxohexahydropyrazino [2,1-c][1,2,4]oxadiazine- 1(6H)-carboxylate | 694.9 | 694.4 | 695.3 | [M + H]⁺ | A |

TABLE 12-16

| | | | | | | | |
|---|---|---|---|---|---|---|---|
| I-101 | 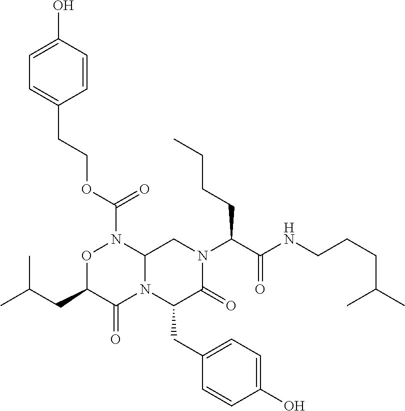 [SMILES: CCCC[C@@H](C(=O)NCCCC(C)C)N1CC2N(C(=O)OCCc3ccc(O)cc3)O[C@H](CC(C)C)C(=O)N2[C@@H](Cc2ccc(O)cc2)C1=O] | 4-hydroxyphenethyl (3R,6S)- 6-(4-hydroxybenzyl)-3- isobutyl-8-((S)-1-((4- methylpentyl)amino)-1- oxohexan-2-yl)-4,7- dioxohexahydropyrazino[2,1- c][1,2,4]oxadiazine-1(6H)- carboxylate | 694.9 | 694.4 | 695.3 | [M + H]⁺ | A |
| I-102 | 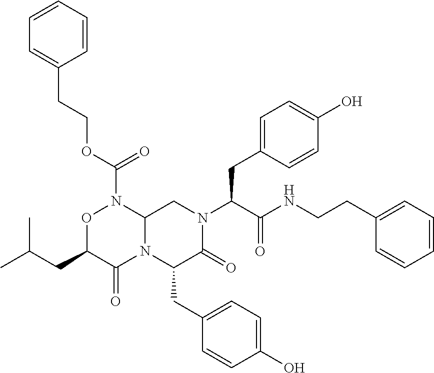 [SMILES: CC(C)C[C@H]1ON(C(=O)OCCc2ccccc2)C2CN([C@@H](Cc3ccc(O)cc3)C(=O)NCCc3ccccc3)C(=O)[C@H](Cc3ccc(O)cc3)N2C1=O] | phenethyl (3R,6S)-6-(4- hydroxybenzyl)-8-((S)-3-(4- hydroxyphenyl)-1-oxo-1- (phenethylamino)propan-2- yl)-3-isobutyl-4,7- dioxohexahydropyrazino[2,1- c][1,2,4]oxadiazine-1(6H)- carboxylate | 748.9 | 748.3 | 749.2 | [M + H]⁺ | A |
| I-103 | 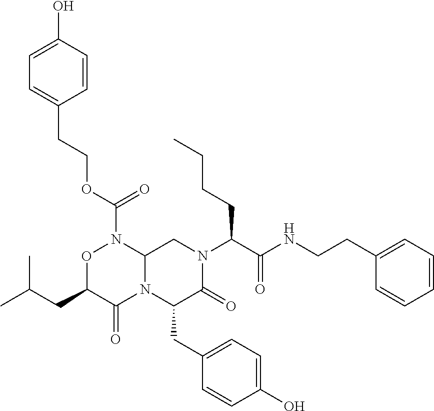 [SMILES: CCCC[C@@H](C(=O)NCCc1ccccc1)N1CC2N(C(=O)OCCc3ccc(O)cc3)O[C@H](CC(C)C)C(=O)N2[C@@H](Cc2ccc(O)cc2)C1=O] | 4-hydroxyphenethyl (3R,6S)- 6-(4-hydroxybenzyl)-3- isobutyl-4,7-dioxo-8-((S)-1- oxo-1- (phenethylamino)hexan-2- yl)hexahydropyrazino[2,1- c][1,2,4]oxadiazine-1(6H)- carboxylate | 714.9 | 714.4 | 715.2 | [M + H]⁺ | A |
| I-104 | 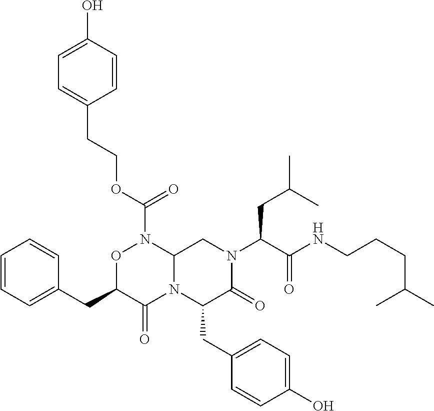 [SMILES: CC(C)CCCNC(=O)[C@H](CC(C)C)N1CC2N(C(=O)OCCc3ccc(O)cc3)O[C@H](Cc3ccccc3)C(=O)N2[C@@H](Cc2ccc(O)cc2)C1=O] | 4-hydroxyphenethyl (3R,6S)- 3-benzyl-6-(4- hydroxybenzyl)-8-((S)-4- methyl-1-((4- methylpentyl)amino)-1- oxopentan-2-yl)-4,7- dioxohexahydropyrazino[2,1- c][1,2,4]oxadiazine-1(6H)- carboxylate | 728.9 | 728.4 | 729.3 | [M + H]⁺ | A |
| I-105 | 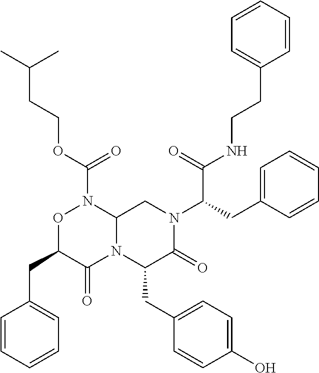 [SMILES: CC(C)CCOC(=O)N1O[C@H](Cc2ccccc2)C(=O)N2C1CN([C@@H](Cc1ccccc1)C(=O)NCCc1ccccc1)C(=O)[C@@H]2Cc1ccc(O)cc1] | isopentyl (3R,6S)-3-benzyl-6- (4-hydroxybenzyl)-4,7-dioxo- 8-((S)-1-oxo-1- (phenethylamino)-3- phenylpropan-2- yl)hexahydropyrazino[2,1- c][1,2,4]oxadiazine-1(6H)- carboxylate | 732.9 | 732.4 | 733.3 | [M + H]⁺ | A |
| I-106 | 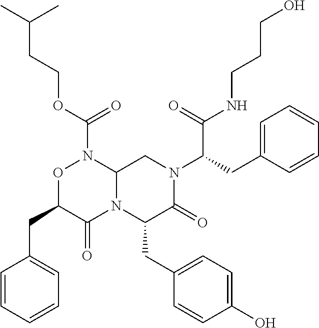 [SMILES: CC(C)CCOC(=O)N1O[C@H](Cc2ccccc2)C(=O)N2C1CN([C@@H](Cc1ccccc1)C(=O)NCCCO)C(=O)[C@@H]2Cc1ccc(O)cc1] | isopentyl (3R,6S)-3-benzyl-6- (4-hydroxybenzyl)-8-((S)-1- ((3-hydroxypropyl)amino)-1- oxo-3-phenylpropan-2-yl)- 4,7- dioxohexahydropyrazino[2,1- c][1,2,4]oxadiazine-1(6H)- carboxylate | 686.8 | 686.3 | 687.3 | [M + H]⁺ | A |

TABLE 12-17

| | | | | | | | |
|---|---|---|---|---|---|---|---|
| I-107 | 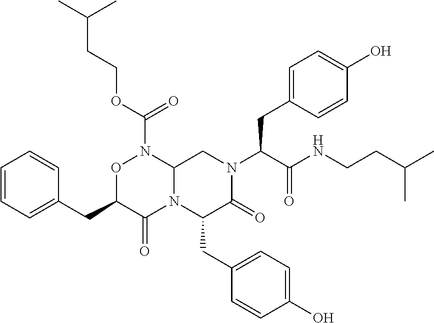 [SMILES: CC(C)CCNC(=O)[C@H](Cc1ccc(O)cc1)N1CC2N(C(=O)OCCC(C)C)O[C@H](Cc3ccccc3)C(=O)N2[C@@H](Cc2ccc(O)cc2)C1=O] | isopentyl (3R,6S)-3-benzyl-6- (4-hydroxybenzyl)-8-((S)-3- (4-hydroxyphenyl)-1- (isopentylamino)-1- oxopropan-2-yl)-4,7- dioxohexahydropyrazino[2,1- c][1,2,4]oxadiazine-1(6H)- carboxylate | 714.9 | 714.4 | 715.3 | [M + H]⁺ | A |
| I-108 | 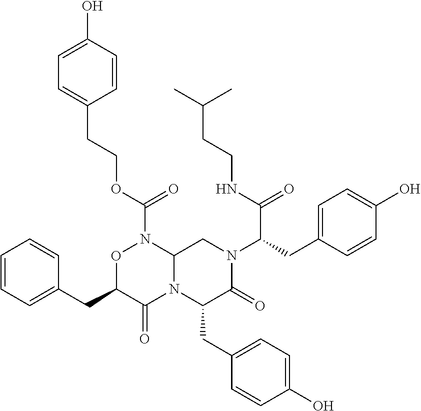 [SMILES: CC(C)CCNC(=O)[C@H](Cc1ccc(O)cc1)N1CC2N(C(=O)OCCc3ccc(O)cc3)O[C@H](Cc3ccccc3)C(=O)N2[C@@H](Cc2ccc(O)cc2)C1=O] | 4-hydroxyphenethyl (3R,6S)- 3-benzyl-6-(4- hydroxybenzyl)-8-((S)-3-(4- hydroxyphenyl)-1- (isopentylamino)-1- oxopropan-2-yl)-4,7- dioxohexahydropyrazino[2,1- c][1,2,4]oxadiazine-1(6H)- carboxylate | 764.9 | 764.3 | 765.2 | [M + H]⁺ | A |
| I-109 | 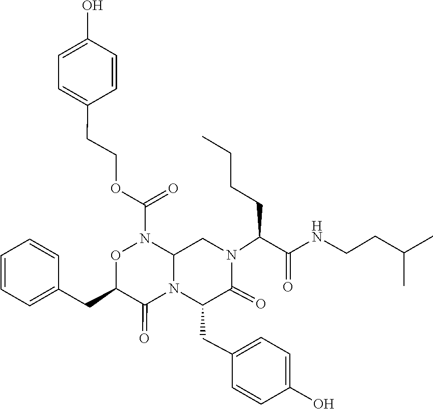 [SMILES: CCCC[C@@H](C(=O)NCCC(C)C)N1CC2N(C(=O)OCCc3ccc(O)cc3)O[C@H](Cc3ccccc3)C(=O)N2[C@@H](Cc2ccc(O)cc2)C1=O] | 4-hydroxyphenethyl (3R,6S)- 3-benzyl-6-(4- hydroxybenzyl)-8-((S)-1- (isopentylamino)-1- oxohexan-2-yl)-4,7- dioxohexahydropyrazino[2,1- c][1,2,4]oxadiazine-1(6H)- carboxylate | 714.9 | 714.4 | 715.3 | [M + H]⁺ | A |
| I-110 | 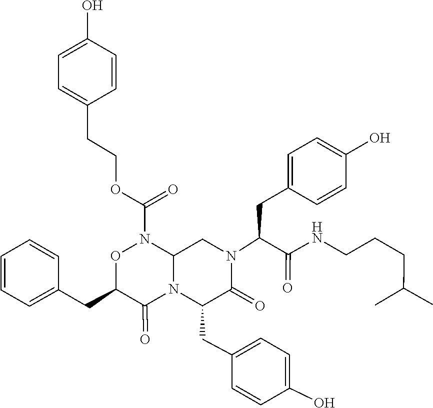 [SMILES: CC(C)CCCNC(=O)[C@H](Cc1ccc(O)cc1)N1CC2N(C(=O)OCCc3ccc(O)cc3)O[C@H](Cc3ccccc3)C(=O)N2[C@@H](Cc2ccc(O)cc2)C1=O] | 4-hydroxyphenethyl (3R,6S)- 3-benzyl-6-(4- hydroxybenzyl)-8-((S)-3-(4- hydroxyphenyl)-1-((4- methylpentyl)amino)-1- oxopropan-2-yl)-4,7- dioxohexahydropyrazino[2,1- c][1,2,4]oxadiazine-1(6H)- carboxylate | 778.9 | 778.4 | 779.2 | [M + H]⁺ | A |
| I-111 | 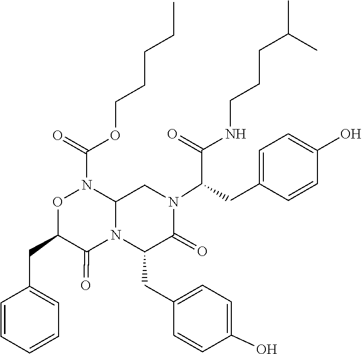 [SMILES: CCCCCOC(=O)N1O[C@H](Cc2ccccc2)C(=O)N2C1CN([C@@H](Cc1ccc(O)cc1)C(=O)NCCCC(C)C)C(=O)[C@@H]2Cc1ccc(O)cc1] | pentyl (3R,6S)-3-benzyl-6-(4- hydroxybenzyl)-8-((S)-3-(4- hydroxyphenyl)-1-((4- methylpentyl)amino)-1- oxopropan-2-yl)-4,7- dioxohexahydropyrazino[2,1- c][1,2,4]oxadiazine-1(6H)- carboxylate | 728.9 | 728.4 | 729.3 | [M + H]⁺ | A |
| I-112 | 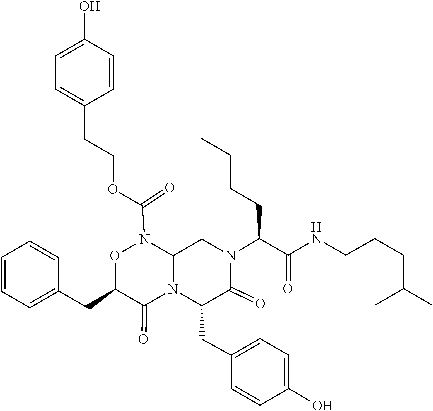 [SMILES: CCCC[C@@H](C(=O)NCCCC(C)C)N1CC2N(C(=O)OCCc3ccc(O)cc3)O[C@H](Cc3ccccc3)C(=O)N2[C@@H](Cc2ccc(O)cc2)C1=O] | 4-hydroxyphenethyl (3R,6S)- 3-benzyl-6-(4- hydroxybenzyl)-8-((S)-1-((4- methylpentyl)amino)-1- oxohexan-2-yl)-4,7- dioxohexahydropyrazino[2,1- c][1,2,4]oxadiazine-1(6H)- carboxylate | 728.9 | 728.4 | 729.3 | [M + H]⁺ | A |

TABLE 12-18

| | | | | | | | |
|---|---|---|---|---|---|---|---|
| I-113 | 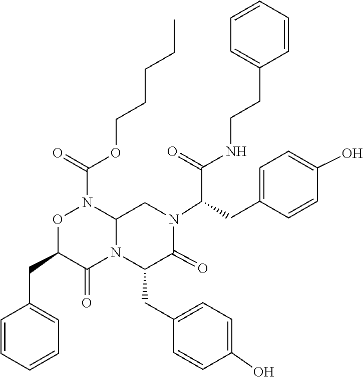 [SMILES: CCCCCOC(=O)N1O[C@H](Cc2ccccc2)C(=O)N2C1CN([C@@H](Cc1ccc(O)cc1)C(=O)NCCc1ccccc1)C(=O)[C@@H]2Cc1ccc(O)cc1] | pentyl (3R,6S)-3-benzyl-6-(4- hydroxybenzyl)-8-((S)-3-(4- hydroxyphenyl)-1-oxo-1- (phenethylamino)propan-2- yl)-4,7- dioxohexahydropyrazino[2,1- c][1,2,4]oxadiazine-1(6H)- carboxylate | 748.9 | 748.3 | 749.3 | [M + H]⁺ | A |
| I-114 | 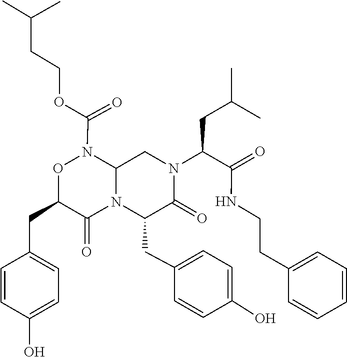 [SMILES: CC(C)CCOC(=O)N1O[C@H](Cc2ccc(O)cc2)C(=O)N2C1CN([C@@H](CC(C)C)C(=O)NCCc1ccccc1)C(=O)[C@@H]2Cc1ccc(O)cc1] | isopentyl (3R,6S)-3,6-bis(4- hydroxybenzyl)-8-((S)-4- methyl-1-oxo-1- (phenethylamino)pentan-2- yl)-4,7- dioxohexahydropyrazino[2,1- c][1,2,4]oxadiazine-1(6H)- carboxylate | 714.9 | 714.4 | 715.3 | [M + H]⁺ | A |
| I-115 | 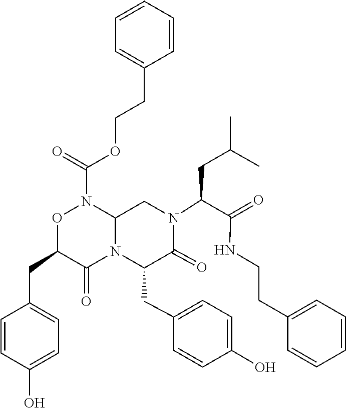 [SMILES: CC(C)C[C@@H](C(=O)NCCc1ccccc1)N1CC2N(C(=O)OCCc3ccccc3)O[C@H](Cc3ccc(O)cc3)C(=O)N2[C@@H](Cc2ccc(O)cc2)C1=O] | phenethyl (3R,6S)-3,6-bis(4- hydroxybenzyl)-8-((S)-4- methyl-1-oxo-1- (phenethylamino)pentan-2- yl)-4,7- dioxohexahydropyrazino[2,1- c][1,2,4]oxadiazine-1(6H)- carboxylate | 748.9 | 748.3 | 749.3 | [M + H]⁺ | A |
| I-116 | 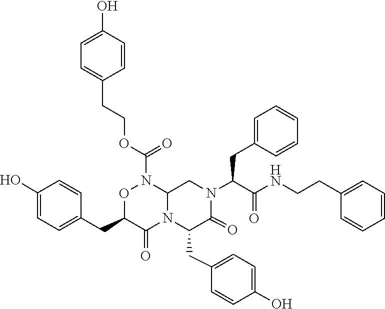 [SMILES: O=C(NCCc1ccccc1)[C@H](Cc1ccccc1)N1CC2N(C(=O)OCCc3ccc(O)cc3)O[C@H](Cc3ccc(O)cc3)C(=O)N2[C@@H](Cc2ccc(O)cc2)C1=O] | 4-hydroxyphenethyl (3R,6S)- 3,6-bis(4-hydroxybenzyl)- 4,7-dioxo-8-((S)-1-oxo-1- (phenethylamino)-3- phenylpropan-2- yl)hexahydropyrazino[2,1- c][1,2,4]oxadiazine-1(6H)- carboxylate | 798.9 | 798.3 | 799.2 | [M + H]⁺ | A |
| I-117 | 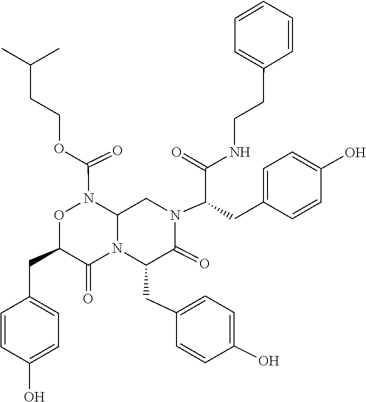 [SMILES: CC(C)CCOC(=O)N1O[C@H](Cc2ccc(O)cc2)C(=O)N2C1CN([C@@H](Cc1ccc(O)cc1)C(=O)NCCc1ccccc1)C(=O)[C@@H]2Cc1ccc(O)cc1] | isopentyl (3R,6S)-3,6-bis(4- hydroxybenzyl)-8-((S)-3-(4- hydroxyphenyl)-1-oxo-1- (phenethylamino)propan-2- yl)-4,7- dioxohexahydropyrazino[2,1- c][1,2,4]oxadiazine-1(6H)- carboxylate | 764.9 | 764.3 | 765.2 | [M + H]⁺ | A |
| I-118 | 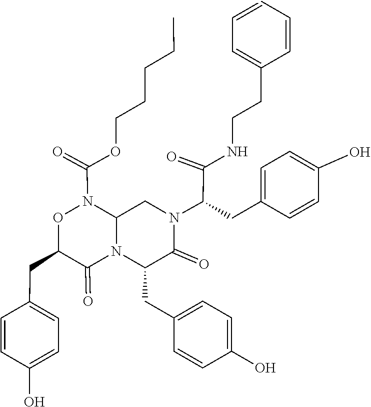 | pentyl (3R,6S)-3,6-bis(4- hydroxybenzyl)-8-((S)-3-(4- hydroxyphenyl)-1-oxo-1- (phenethylamino)propan-2- yl)-4,7- dioxohexahydropyrazino[2,1- c][1,2,4]oxadiazine-1(6H)- carboxylate | 764.9 | 764.3 | 765.3 | [M + H]⁺ | A |

TABLE 12-19

| | | | | | | | |
|---|---|---|---|---|---|---|---|
| I-119 | 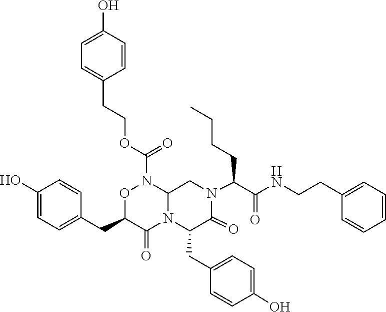 [SMILES: CCCC[C@@H](C(=O)NCCc1ccccc1)N1CC2N(C(=O)OCCc3ccc(O)cc3)O[C@H](Cc3ccc(O)cc3)C(=O)N2[C@@H](Cc2ccc(O)cc2)C1=O] | 4-hydroxyphenethyl (3R,6S)- 3,6-bis(4-hydroxybenzyl)- 4,7-dioxo-8-((S)-1-oxo-1- (phenethylamino)hexan-2- yl)hexahydropyrazino[2,1- c][1,2,4]oxadiazine-1(6H)- carboxylate | 764.9 | 764.3 | 765.2 | [M + H]⁺ | A |
| I-120 | 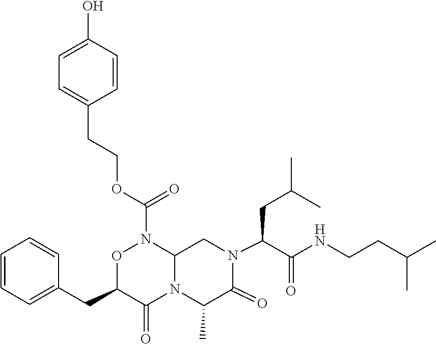 [SMILES: CC(C)CCNC(=O)[C@H](CC(C)C)N1CC2N(C(=O)OCCc3ccc(O)cc3)O[C@H](Cc3ccccc3)C(=O)N2[C@@H](C)C1=O] | 4-hydroxyphenethyl (3R,6S)-3-benzyl-8-((S)-1- (isopentylamino)-4-methyl- 1-oxopentan-2-yl)-6-methyl- 4,7- dioxohexahydropyrazino [2,1-c][1,2,4]oxadiazine- 1(6H)-carboxylate | 622.8 | 622.3 | 623.3 | [M + H]⁺ | A |
| I-121 | 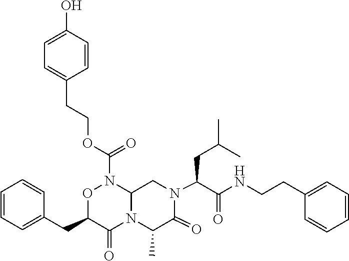 [SMILES: CC(C)C[C@@H](C(=O)NCCc1ccccc1)N1CC2N(C(=O)OCCc3ccc(O)cc3)O[C@H](Cc3ccccc3)C(=O)N2[C@@H](C)C1=O] | 4-hydroxyphenethyl (3R,6S)-3-benzyl-6- methyl-8-((S)-4- methyl-1-oxo-1- (phenethylamino)pentan-2- yl)-4,7- dioxohexahydropyrazino [2,1-c][1,2,4]oxadiazine- 1(6H)-carboxylate | 656.8 | 656.3 | 657.3 | [M + H]⁺ | A |
| I-122 | 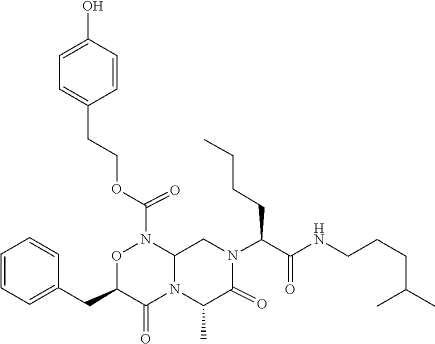 [SMILES: CCCC[C@@H](C(=O)NCCCC(C)C)N1CC2N(C(=O)OCCc3ccc(O)cc3)O[C@H](Cc3ccccc3)C(=O)N2[C@@H](C)C1=O] | 4-hydroxyphenethyl (3R,6S)-3-benzyl-6- methyl-8-((S)-1- ((4-methylpentyl)amino)-1- oxohexan-2-yl)-4,7- dioxohexahydropyrazino [2,1-c][1,2,4]oxadiazine- 1(6H)-carboxylate | 636.8 | 636.4 | 637.3 | [M + H]⁺ | A |
| I-123 | 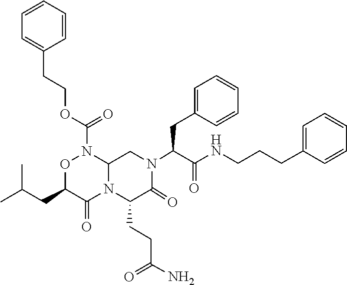 [SMILES: CC(C)C[C@H]1ON(C(=O)OCCc2ccccc2)C2CN([C@@H](Cc3ccccc3)C(=O)NCCCc3ccccc3)C(=O)[C@H](CCC(N)=O)N2C1=O] | phenethyl (3R,6S)-6-(3- amino-3-oxopropyl)-3- isobutyl-4,7-dioxo-8-((S)-1- oxo-3-phenyl-1-((3- phenylpropyl)amino)propan- 2-yl)hexahydropyrazino[2,1- c][1,2,4]oxadiazine-1(6H)- carboxylate | 711.9 | 711.4 | 712.3 | [M + H]⁺ | A |
| I-124 | 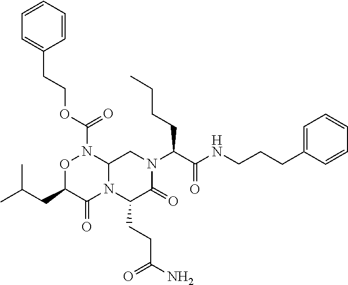 [SMILES: CCCC[C@@H](C(=O)NCCCc1ccccc1)N1CC2N(C(=O)OCCc3ccccc3)O[C@H](CC(C)C)C(=O)N2[C@@H](CCC(N)=O)C1=O] | phenethyl (3R,6S)-6-(3- amino-3-oxopropyl)-3- isobutyl-4,7-dioxo-8-((S)-1- oxo-1-((3- phenylpropyl)amino)hexan- 2-yl)hexahydropyrazino[2,1- c][1,2,4]oxadiazine-1(6H)- carboxylate | 677.8 | 677.4 | 678.4 | [M + H]⁺ | A |

TABLE 12-20

| | | | | | | | |
|---|---|---|---|---|---|---|---|
| I-125 | 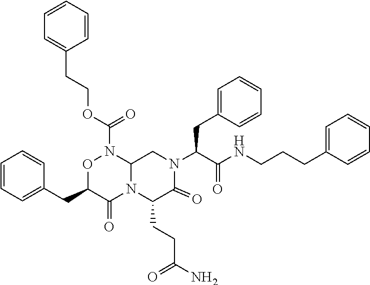 [SMILES: NC(=O)CC[C@H]1C(=O)N([C@@H](Cc2ccccc2)C(=O)NCCCc2ccccc2)CC2N(C(=O)OCCc3ccccc3)O[C@H](Cc3ccccc3)C(=O)N21] | phenethyl (3R,6S)-6-(3- amino-3-oxopropyl)-3- benzyl-4,7-dioxo-8-((S)-1- oxo-3-phenyl-1-((3- phenylpropyl) amino)propan- 2-yl)hexahydropyrazino [2,1-c][1,2,4]oxadiazine- 1(6H)-carboxylate | 745.9 | 745.3 | 746.3 | [M + H]⁺ | A |
| I-126 | 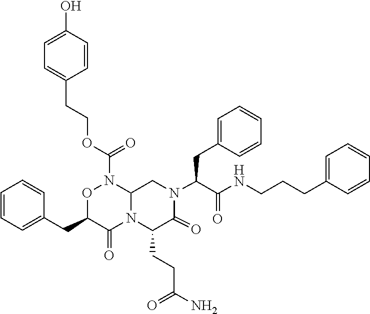 [SMILES: NC(=O)CC[C@H]1C(=O)N([C@@H](Cc2ccccc2)C(=O)NCCCc2ccccc2)CC2N(C(=O)OCCc3ccc(O)cc3)O[C@H](Cc3ccccc3)C(=O)N21] | 4-hydroxyphenethyl (3R,6S)-6-(3-amino- 3-oxopropyl)-3- benzyl-4,7-dioxo-8-((S)-1- oxo-3-phenyl-1-((3- phenylpropyl) amino)propan- 2-yl)hexahydropyrazino [2,1-c][1,2,4]oxadiazine- 1(6H)-carboxylate | 761.9 | 761.3 | 762.3 | [M + H]⁺ | A |
| I-127 | 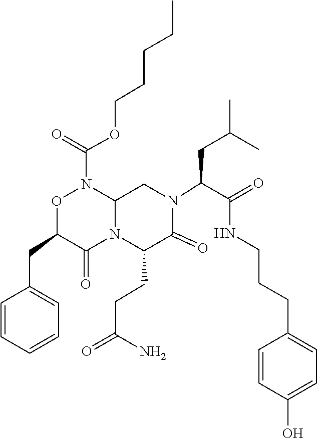 [SMILES: CCCCCOC(=O)N1O[C@H](Cc2ccccc2)C(=O)N2C1CN([C@@H](CC(C)C)C(=O)NCCCc1ccc(O)cc1)C(=O)[C@@H]2CCC(N)=O] | pentyl (3R,6S)-6-(3-amino- 3-oxopropyl)-3-benzyl-8- ((S)-1-((3-(4- hydroxyphenyl) propyl)amino)- 4-methyl-1-oxopentan-2- yl)-4,7- dioxohexahydropyrazino [2,1-c][1,2,4]oxadiazine- 1(6H)-carboxylate | 693.8 | 693.4 | 694.4 | [M + H]⁺ | A |
| I-128 | 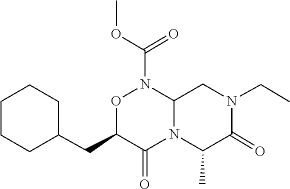 [SMILES: CCN1CC2N(C(=O)OC)O[C@H](CC3CCCCC3)C(=O)N2[C@@H](C)C1=O] | (3R,6S)-methyl 6-methyl-3- cyclohexylmethyl-8-ethyl- 4,7- dioxohexahydropyrazino [2,1-c][1,2,4]oxadiazine- 1(6H)-carboxylate | 367.4 | 367.2 | 368.2 | [M + H]⁺ | A |
| I-129 | 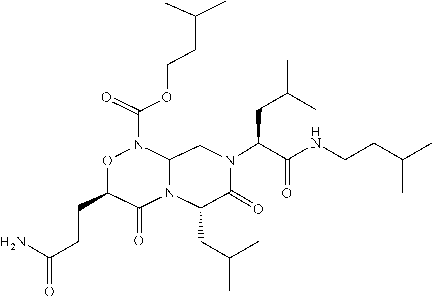 [SMILES: CC(C)CCNC(=O)[C@H](CC(C)C)N1CC2N(C(=O)OCCC(C)C)O[C@H](CCC(N)=O)C(=O)N2[C@@H](CC(C)C)C1=O] | isopentyl (3R,6S)-3-(3- amino-3-oxopropyl)-6- isobutyl-8-((S)-1- (isopentylamino)-4-methyl- 1-oxopentan-2-yl)-4,7- dioxohexahydropyrazino [2,1-c][1,2,4]oxadiazine- 1(6H)-carboxylate | 595.8 | 595.4 | 596.4 | [M + H]⁺ | A |
| I-130 | 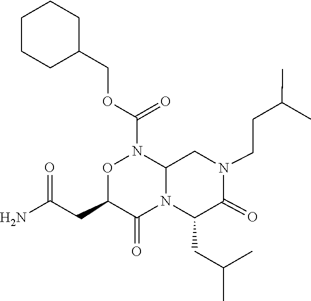 [SMILES: CC(C)CCN1CC2N(C(=O)OCC3CCCCC3)O[C@H](CC(N)=O)C(=O)N2[C@@H](CC(C)C)C1=O] | cyclohexylmethyl (3R,6S)- 3-(2-amino-2-oxoethyl)-6- isobutyl-8-isopentyl-4,7- dioxohexahydropyrazino [2,1-c][1,2,4]oxadiazine- 1(6H)-carboxylate | 494.6 | 494.3 | 495.3 | [M + H]⁺ | A |

TABLE 12-21

| | | | | | | | |
|---|---|---|---|---|---|---|---|
| I-131 | 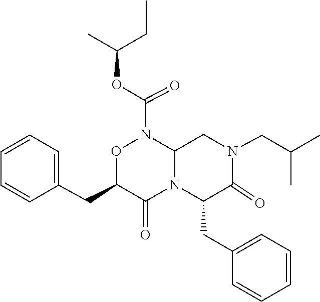 [SMILES: CC[C@H](C)OC(=O)N1O[C@H](Cc2ccccc2)C(=O)N2C1CN(CC(C)C)C(=O)[C@@H]2Cc1ccccc1] | (S)-sec-butyl (3R,6S)-3,6- dibenzyl-8-isobutyl-4,7- dioxohexahydropyrazino[2,1- c][1,2,4]oxadiazine-1(6H)- carboxylate | 507.6 | 507.3 | 508.3 | [M + H]⁺ | A |
| I-132 | 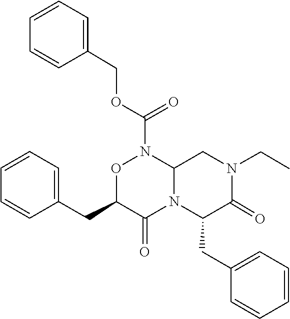 [SMILES: CCN1CC2N(C(=O)OCc3ccccc3)O[C@H](Cc3ccccc3)C(=O)N2[C@@H](Cc2ccccc2)C1=O] | benzyl (3R,6S)-3,6-dibenzyl- 8-ethyl-4,7- dioxohexahydropyrazino[2,1- c][1,2,4]oxadiazine-1(6H)- carboxylate | 513.6 | 513.2 | 514.3 | [M + H]⁺ | A |
| I-133 | 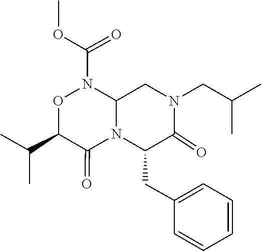 [SMILES: COC(=O)N1O[C@H](C(C)C)C(=O)N2C1CN(CC(C)C)C(=O)[C@@H]2Cc1ccccc1] | methyl (3R,6S)-6-benzyl-8- isobutyl-3-isopropyl-4,7- dioxohexahydropyrazino[2,1- c][1,2,4]oxadiazine-1(6H)- carboxylate | 417.5 | 417.2 | 418.3 | [M + H]⁺ | A |
| I-134 | 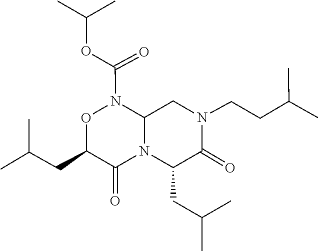 [SMILES: CC(C)CCN1CC2N(C(=O)OC(C)C)O[C@H](CC(C)C)C(=O)N2[C@@H](CC(C)C)C1=O] | isopropyl (3R,6S)-3,6- diisobutyl-8-isopentyl-4,7- dioxohexahydropyrazino[2,1- c][1,2,4]oxadiazine-1(6H)- carboxylate | 439.6 | 439.3 | 440.3 | [M + H]⁺ | A |
| I-135 | 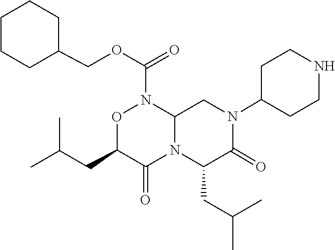 [SMILES: CC(C)C[C@H]1ON(C(=O)OCC2CCCCC2)C2CN(C3CCNCC3)C(=O)[C@H](CC(C)C)N2C1=O] | cyclohexylmethyl (3R,6S)- 3,6-diisobutyl-4,7-dioxo-8- (piperidin-4- yl)hexahydropyrazino[2,1- c][1,2,4]oxadiazine-1(6H)- carboxylate | 506.7 | 506.3 | 507.4 | [M + H]⁺ | B |
| I-136 | 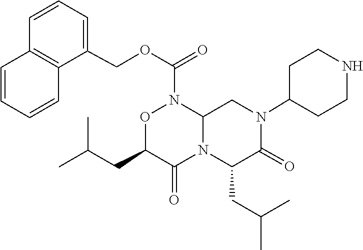 [SMILES: CC(C)C[C@H]1ON(C(=O)OCc2cccc3ccccc23)C2CN(C3CCNCC3)C(=O)[C@H](CC(C)C)N2C1=O] | naphthalen-1-ylmethyl (3R,6S)-3,6-diisobutyl-4,7- dioxo-8-(piperidin-4- yl)hexahydropyrazino[2,1- c][1,2,4]oxadiazine-1(6H)- carboxylate | 550.7 | 550.3 | 551.4 | [M + H]⁺ | B |
| I-137 | 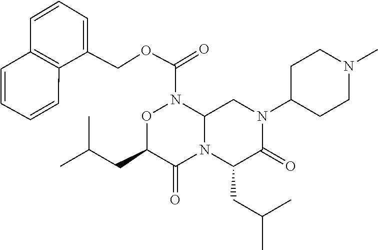 [SMILES: CC(C)C[C@H]1ON(C(=O)OCc2cccc3ccccc23)C2CN(C3CCN(C)CC3)C(=O)[C@H](CC(C)C)N2C1=O] | naphthalen-1-ylmethyl (3R,6S)-3,6-diisobutyl-8-(1- methylpiperidin-4-yl)-4,7- dioxohexahydropyrazino[2,1- c][1,2,4]oxadiazine-1(6H)- carboxylate | 564.7 | 564.3 | 565.4 | [M + H]⁺ | B |
| I-138 | 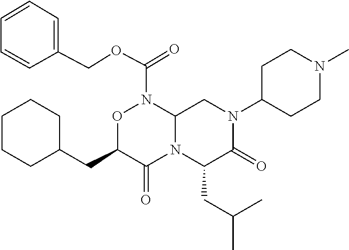 [SMILES: CC(C)C[C@H]1C(=O)N(C2CCN(C)CC2)CC2N(C(=O)OCc3ccccc3)O[C@H](CC3CCCCC3)C(=O)N21] | benzyl (3R,6S)-3- (cyclohexylmethyl)-6- isobutyl-8-(1- methylpiperidin-4-yl)-4,7- dioxohexahydropyrazino[2,1- c][1,2,4]oxadiazine-1(6H)- carboxylate | 554.7 | 554.3 | 555.5 | [M + H]⁺ | B |

TABLE 12-22

| | | | | | | | |
|---|---|---|---|---|---|---|---|
| I-139 | 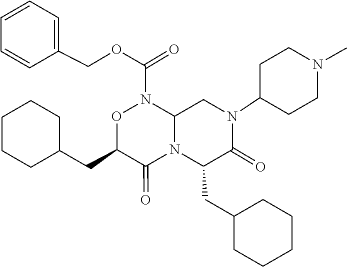 [SMILES: CN1CCC(N2CC3N(C(=O)OCc4ccccc4)O[C@H](CC4CCCCC4)C(=O)N3[C@@H](CC3CCCCC3)C2=O)CC1] | benzyl (3R,6S)-3,6-bis (cyclohexylmethyl)-8-(1- methylpiperidin-4-yl)-4,7- dioxohexahydropyrazino [2,1-c][1,2,4]oxadiazine- 1(6H)-carboxylate | 594.8 | 594.4 | 595.5 | [M + H]⁺ | B |
| I-140 | 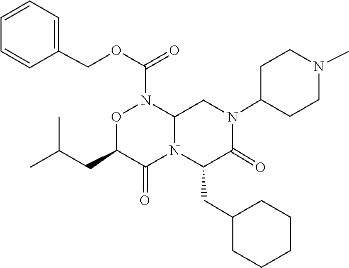 [SMILES: CC(C)C[C@H]1ON(C(=O)OCc2ccccc2)C2CN(C3CCN(C)CC3)C(=O)[C@H](CC3CCCCC3)N2C1=O] | benzyl (3R,6S)-6- (cyclohexylmethyl)-3- isobutyl-8-(1- methylpiperidin-4-yl)-4,7- dioxohexahydropyrazino [2,1-c][1,2,4]oxadiazine- 1(6H)-carboxylate | 554.7 | 554.3 | 555.5 | [M + H]⁺ | B |
| I-141 | 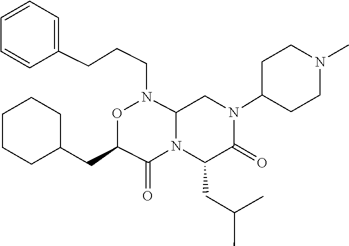 [SMILES: CC(C)C[C@H]1C(=O)N(C2CCN(C)CC2)CC2N(CCCc3ccccc3)O[C@H](CC3CCCCC3)C(=O)N21] | (3R,6S)-3- (cyclohexylmethyl)-6- isobutyl-8-(1- methylpiperidin-4-yl)- 1-(3-phenylpropyl) tetrahydropyrazino [2,1-c][1,2,4]oxadiazine- 4,7(3H,6H)-dione | 538.8 | 538.4 | 539.6 | [M + H]⁺ | B |
| I-142 | 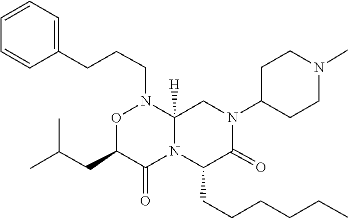 [SMILES: CCCCCC[C@H]1C(=O)N(C2CCN(C)CC2)C[C@@H]2N(CCCc3ccccc3)O[C@H](CC(C)C)C(=O)N21] | (3R,6S,9aS)-6-hexyl-3- isobutyl-8-(1- methylpiperidin-4-yl)- 1-(3-phenylpropyl) tetrahydropyrazino [2,1-c][1,2,4]oxadiazine- 4,7(3H,6H)-dione | 526.8 | 526.4 | 527.5 | [M + H]⁺ | B |
| I-143 | 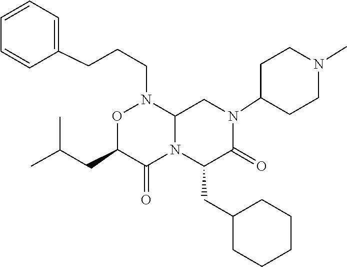 [SMILES: CC(C)C[C@H]1ON(CCCc2ccccc2)C2CN(C3CCN(C)CC3)C(=O)[C@H](CC3CCCCC3)N2C1=O] | (3R,6S)-6- (cyclohexylmethyl)-3- isobutyl-8-(1- methylpiperidin-4-yl)- 1-(3-phenylpropyl) tetrahydropyrazino [2,1-c][1,2,4]oxadiazine- 4,7(3H,6H)-dione | 538.8 | 538.4 | 539.5 | [M + H]⁺ | B |
| I-144 | 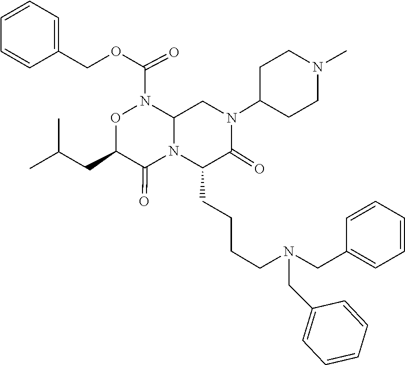 [SMILES: CC(C)C[C@H]1ON(C(=O)OCc2ccccc2)C2CN(C3CCN(C)CC3)C(=O)[C@H](CCCCN(Cc3ccccc3)Cc3ccccc3)N2C1=O] | benzyl (3R,6S)-6-(4- (dibenzylamino)butyl)-3- isobutyl-8-(1- methylpiperidin-4-yl)-4,7- dioxohexahydropyrazino [2,1-c][1,2,4]oxadiazine- 1(6H)-carboxylate | 709.9 | 709.4 | 710.5 | [M + H]⁺ | B |
| I-145 | 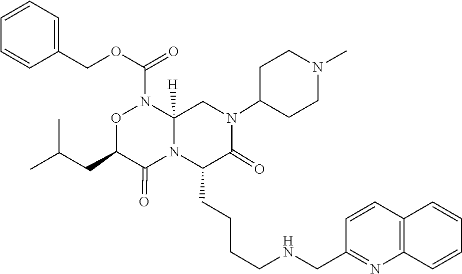 [SMILES: CC(C)C[C@H]1ON(C(=O)OCc2ccccc2)[C@H]2CN(C3CCN(C)CC3)C(=O)[C@H](CCCCNCc3ccc4ccccc4n3)N2C1=O] | benzyl (3R,6S,9aS)-3- isobutyl-8-(1- methylpiperidin-4-yl)-4,7- dioxo-6-(4-((quinolin-2- ylmethyl)amino)butyl) hexahydropyrazino[2,1- c][1,2,4]oxadiazine-1(6H)- carboxylate | 670.9 | 670.4 | 671.4 | [M + H]⁺ | B |

TABLE 12-23

| | | | | | | | |
|---|---|---|---|---|---|---|---|
| I-146 | 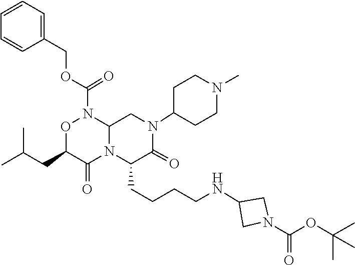 [SMILES: CC(C)C[C@H]1ON(C(=O)OCc2ccccc2)C2CN(C3CCN(C)CC3)C(=O)[C@H](CCCCNC3CN(C(=O)OC(C)(C)C)C3)N2C1=O] | benzyl (3R,6S)-6-(4-((1-(tert- butoxycarbonyl)azetidin-3- yl)amino)butyl)-3-isobutyl-8- (1-methylpiperidin-4-yl)-4,7- dioxohexahydropyrazino[2,1- c][1,2,4]oxadiazine-1(6H)- carboxylate | 684.9 | 684.4 | 685.5 | [M + H]⁺ | B |
| I-147 | 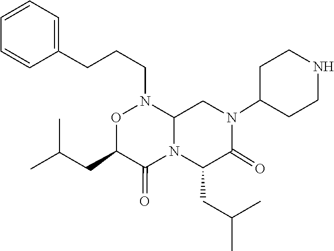 [SMILES: CC(C)C[C@H]1ON(CCCc2ccccc2)C2CN(C3CCNCC3)C(=O)[C@H](CC(C)C)N2C1=O] | (3R,6S)-3,6-diisobutyl-1-(3- phenylpropyl)-8-(piperidin-4- yl)tetrahydropyrazino[2,1- c][1,2,4]oxadiazine- 4,7(3H,6H)-dione | 484.7 | 484.3 | 485.1 | [M + H]⁺ | D |
| I-148 | 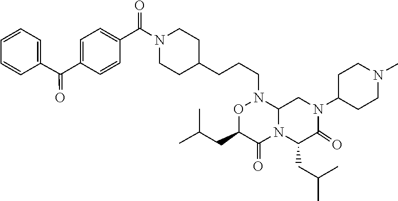 [SMILES: CC(C)C[C@H]1ON(CCCC2CCN(C(=O)c3ccc(C(=O)c4ccccc4)cc3)CC2)C2CN(C3CCN(C)CC3)C(=O)[C@H](CC(C)C)N2C1=O] | (3R,6S)-1-(3-(1-(4- benzoylbenzoyl)piperidin-4- yl)propyl)-3,6-diisobutyl-8- (1-methylpiperidin-4- yl)tetrahydropyrazino[2,1- c][1,2,4]oxadiazine- 4,7(3H,6H)-dione | 714.0 | 713.5 | 714.6 | [M + H]⁺ | B |
| I-149 | 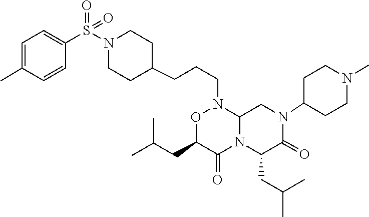 | (3R,6S)-3,6-diisobutyl-8-(1- methylpiperidin-4-yl)-1-(3- (1-tosylpiperidin-4- yl)propyl)tetrahydropyrazino [2,1-c][1,2,4]oxadiazine- 4,7(3H,6H)-dione | 659.9 | 659.4 | 660.5 | [M + H]⁺ | B |
| I-150 | 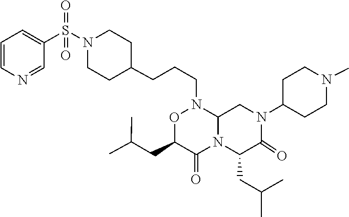 [SMILES: CC(C)C[C@H]1ON(CCCC2CCN(S(=O)(=O)c3cccnc3)CC2)C2CN(C3CCN(C)CC3)C(=O)[C@H](CC(C)C)N2C1=O] | (3R,6S)-3,6-diisobutyl-8-(1- methylpiperidin-4-yl)-1-(3- (1-(pyridin-3- ylsulfonyl)piperidin-4- yl)propyl)tetrahydropyrazino [2,1-c][1,2,4]oxadiazine- 4,7(3H,6H)-dione | 646.9 | 646.4 | 647.5 | [M + H]⁺ | B |
| I-151 | 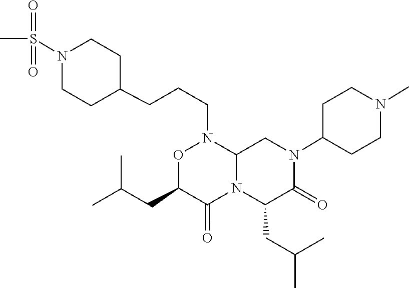 | (3R,6S)-3,6-diisobutyl-8-(1- methylpiperidin-4-yl)-1-(3- (1-(methylsulfonyl)piperidin- 4-yl)propyl) tetrahydropyrazino [2,1-c][1,2,4]oxadiazine- 4,7(3H,6H)-dione | 583.8 | 583.4 | 584.5 | [M + H]⁺ | B |
| I-152 | 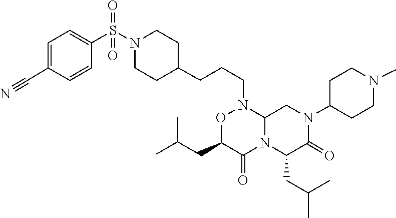 [SMILES: CC(C)C[C@H]1ON(CCCC2CCN(S(=O)(=O)c3ccc(C#N)cc3)CC2)C2CN(C3CCN(C)CC3)C(=O)[C@H](CC(C)C)N2C1=O] | 4-((4-(3-((3R,6S)-3,6- diisobutyl-8-(1- methylpiperidin-4-yl)-4,7- dioxohexahydro- pyrazino[2,1- c][1,2,4]oxadiazin-1(6H)- yl)propyl)piperidin-1- yl)sulfonyl)benzonitrile | 670.9 | 670.4 | 671.5 | [M + H]⁺ | B |
| I-153 | 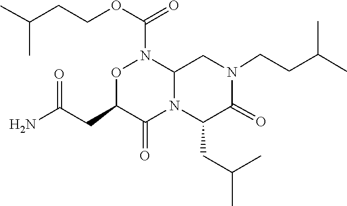 [SMILES: CC(C)CCOC(=O)N1O[C@H](CC(N)=O)C(=O)N2C1CN(CCC(C)C)C(=O)[C@@H]2CC(C)C] | isopentyl (3R,6S)-3-(2- amino-2-oxoethyl)-6- isobutyl-8-isopentyl-4,7- dioxohexahydro- pyrazino[2,1- c][1,2,4]oxadiazine-1(6H)- carboxylate | 468.6 | 468.3 | 469.4 | [M + H]⁺ | D |

TABLE 12-24

| | | | | | | | |
|---|---|---|---|---|---|---|---|
| I-154 | 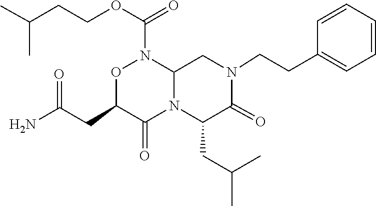 [SMILES: CC(C)CCOC(=O)N1O[C@H](CC(N)=O)C(=O)N2C1CN(CCc1ccccc1)C(=O)[C@@H]2CC(C)C] | isopentyl (3R,6S)-3-(2- amino-2-oxoethyl)-6- isobutyl-4,7-dioxo-8- phenethylhexahydropyrazino [2,1-c][1,2,4]oxadiazine- 1(6H)-carboxylate | 502.6 | 502.3 | 503.4 | [M + H]⁺ | D |
| I-155 | 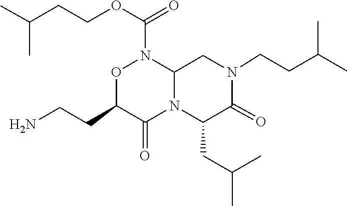 [SMILES: CC(C)CCOC(=O)N1O[C@H](CCN)C(=O)N2C1CN(CCC(C)C)C(=O)[C@@H]2CC(C)C] | isopentyl (3R,6S)-3-(2- aminoethyl)-6-isobutyl-8- isopentyl-4,7- dioxohexahydropyrazino[2,1- c][1,2,4]oxadiazine-1(6H)- carboxylate | 454.6 | 454.3 | 455.4 | [M + H]⁺ | D |
| I-156 | 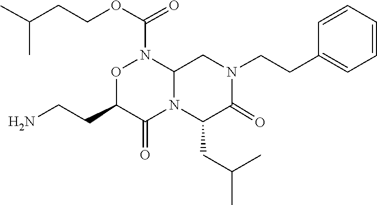 [SMILES: CC(C)CCOC(=O)N1O[C@H](CCN)C(=O)N2C1CN(CCc1ccccc1)C(=O)[C@@H]2CC(C)C] | isopentyl (3R,6S)-3-(2- aminoethyl)-6-isobutyl-4,7- dioxo-8- phenethylhexahydropyrazino [2,1-c][1,2,4]oxadiazine- 1(6H)-carboxylate | 488.6 | 488.3 | 489.4 | [M + H]⁺ | D |
| I-157 | 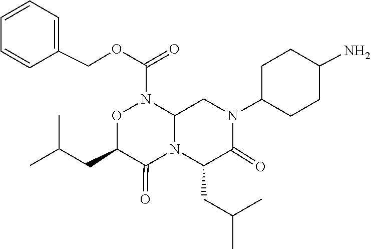 [SMILES: CC(C)C[C@H]1ON(C(=O)OCc2ccccc2)C2CN(C3CCC(N)CC3)C(=O)[C@H](CC(C)C)N2C1=O] | benzyl (3R,6S)-8-(4- aminocyclohexyl)-3,6- diisobutyl-4,7- dioxohexahydropyrazino[2,1- c][1,2,4]oxadiazine-1(6H)- carboxylate | 514.7 | 514.3 | 515.4 | [M + H]⁺ | B |
| I-158 | 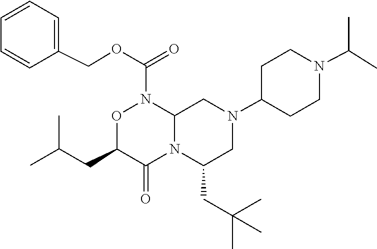 [SMILES: CC(C)C[C@H]1ON(C(=O)OCc2ccccc2)C2CN(C3CCN(C(C)C)CC3)C[C@H](CC(C)(C)C)N2C1=O] | benzyl (3R,6S)-3-isobutyl-8- (1-isopropylpiperidin-4-yl)-6- neopentyl-4- oxohexahydropyrazino[2,1- c][1,2,4]oxadiazine-1(6H)- carboxylate | 542.8 | 542.4 | 543.4 | [M + H]⁺ | B |
| I-159 | 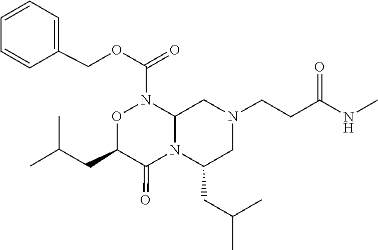 [SMILES: CNC(=O)CCN1CC2N(C(=O)OCc3ccccc3)O[C@H](CC(C)C)C(=O)N2[C@@H](CC(C)C)C1] | benzyl (3R,6S)-3,6- diisobutyl-8-(3- (methylamino)-3-oxopropyl)- 4-oxohexahydropyrazino[2,1- c][1,2,4]oxadiazine-1(6H)- carboxylate | 488.6 | 488.3 | 489.1 | [M + H]⁺ | D |
| I-160 | 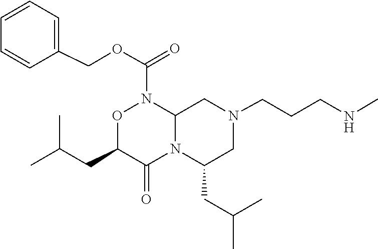 [SMILES: CNCCCN1CC2N(C(=O)OCc3ccccc3)O[C@H](CC(C)C)C(=O)N2[C@@H](CC(C)C)C1] | benzyl (3R,6S)-3,6- diisobutyl-8-(3- (methylamino)propyl)-4- oxohexahydropyrazino[2,1- c][1,2,4]oxadiazine-1(6H)- carboxylate | 474.6 | 474.3 | 475.2 | [M + H]⁺ | D |

TABLE 12-25

| | | | | | | | |
|---|---|---|---|---|---|---|---|
| I-161 | 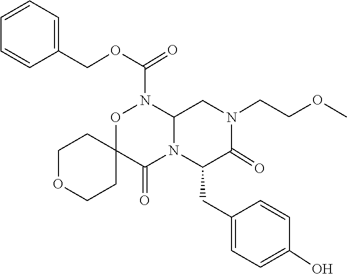 [SMILES: COCCN1CC2N(C(=O)OCc3ccccc3)OC3(CCOCC3)C(=O)N2[C@@H](Cc2ccc(O)cc2)C1=O] | benzyl (6′S)-6′-(4- hydroxybenzyl)-8′-(2- methoxyethyl)-4′,7′- dioxooctahydro-4′H- spiro[pyran-4,3′- pyrazino[2,1- [c][1,2,4]oxadiazine]-1′(6′H)- carboxylate | 539.6 | 539.2 | 540.2 | [M + H]⁺ | D |
| I-162 | 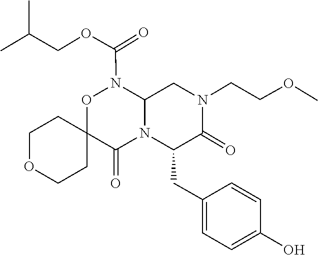 [SMILES: COCCN1CC2N(C(=O)OCC(C)C)OC3(CCOCC3)C(=O)N2[C@@H](Cc2ccc(O)cc2)C1=O] | isobutyl (6′S)-6′-(4- hydroxybenzyl)-8′-(2- methoxyethyl)-4′,7′- dioxooctahydro-4′H- spiro[pyran-4,3′- pyrazino[2,1- c][1,2,4]oxadiazine]-1′(6′H)- carboxylate | 505.6 | 505.2 | 506.3 | [M + H]⁺ | D |
| I-163 | 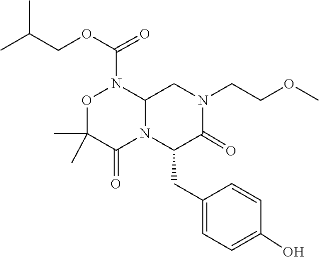 [SMILES: COCCN1CC2N(C(=O)OCC(C)C)OC(C)(C)C(=O)N2[C@@H](Cc2ccc(O)cc2)C1=O] | isobutyl (6S)-6-(4- hydroxybenzyl)-8-(2- methoxyethyl)-3,3-dimethyl- 4,7- dioxohexahydropyrazino[2,1- c][1,2,4]oxadiazine-1(6H)- carboxylate | 463.5 | 463.2 | 464.2 | [M + H]⁺ | D |
| I-164 | 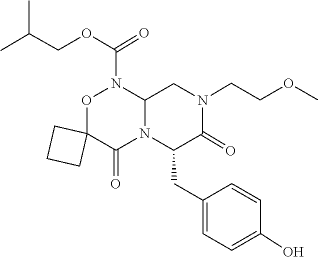 [SMILES: COCCN1CC2N(C(=O)OCC(C)C)OC3(CCC3)C(=O)N2[C@@H](Cc2ccc(O)cc2)C1=O] | isobutyl (6′S)-6′-(4- hydroxybenzyl)-8′-(2- methoxyethyl)-4′,7′- dioxotetrahydro-4′H- spiro[cyclobutane-1,3′- pyrazino[2,1- c][1,2,4]oxadiazine]-1′(6′H)- carboxylate | 475.5 | 475.2 | 476.2 | [M + H]⁺ | D |
| I-165 | 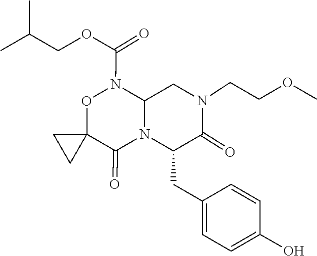 [SMILES: COCCN1CC2N(C(=O)OCC(C)C)OC3(CC3)C(=O)N2[C@@H](Cc2ccc(O)cc2)C1=O] | isobutyl (6′S)-6′-(4- hydroxybenzyl)-8′-(2- methoxyethyl)-4′,7′- dioxotetrahydro-4′H- spiro[cyclopropane-1,3′- pyrazino[2,1- c][1,2,4]oxadiazine]-1′(6′H)- carboxylate | 461.5 | 461.2 | 462.3 460.2 | [M + H]⁺ [M − H]⁻ | D |
| I-166 | 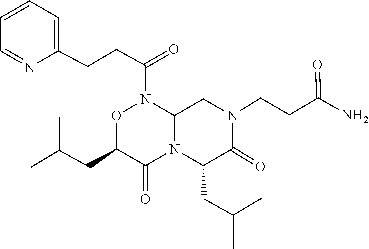 [SMILES: CC(C)C[C@H]1ON(C(=O)CCc2ccccn2)C2CN(CCC(N)=O)C(=O)[C@H](CC(C)C)N2C1=O] | 3-((3R,6S)-3,6-diisobutyl-4,7- dioxo-1-(3-(pyridin-2- yl)propanoyl)hexahydropyrazino [2,1-c][1,2,4]oxadiazin- 8(1H)-yl)propanamide | 487.6 | 487.3 | 488.2 | [M + H]⁺ | D |

TABLE 12-26

| | | | | | | | |
|---|---|---|---|---|---|---|---|
| I-167 | 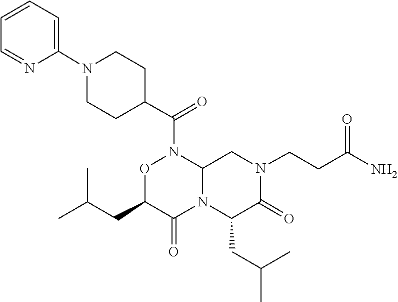 [SMILES: CC(C)C[C@H]1ON(C(=O)C2CCN(c3ccccn3)CC2)C2CN(CCC(N)=O)C(=O)[C@H](CC(C)C)N2C1=O] | 3-((3R,6S)-3,6-diisobutyl- 4,7-dioxo-1-(1-(pyridin-2- yl)piperidine-4- carbonyl)hexahydropyrazino [2,1-c][1,2,4]oxadiazin- 8(1H)-yl)propanamide | 542.7 | 542.3 | 543.2 | [M + H]⁺ | D |
| I-168 | 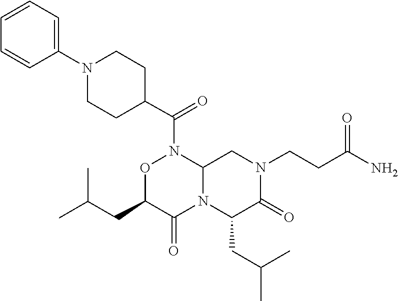 [SMILES: CC(C)C[C@H]1ON(C(=O)C2CCN(c3ccccc3)CC2)C2CN(CCC(N)=O)C(=O)[C@H](CC(C)C)N2C1=O] | 3-((3R,6S)-3,6-diisobutyl- 4,7-dioxo-1-(1- phenylpiperidine-4- carbonyl)hexahydropyrazino [2,1-c][1,2,4]oxadiazin- 8(1H)-yl)propanamide | 541.7 | 541.3 | 542.2 | [M + H]⁺ | D |
| I-169 | 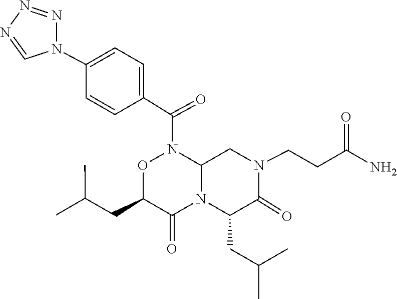 [SMILES: CC(C)C[C@H]1ON(C(=O)c2ccc(-n3cnnn3)cc2)C2CN(CCC(N)=O)C(=O)[C@H](CC(C)C)N2C1=O] | 3-((3R,6S)-1-(4-(1H-tetrazol- 1-yl)benzoyl)-3,6-diisobutyl- 4,7- dioxohexahydropyrazino[2,1- c][1,2,4]oxadiazin-8(1H)- yl)propanamide | 526.6 | 526.3 | 527.2 | [M + H]⁺ | D |
| I-170 | 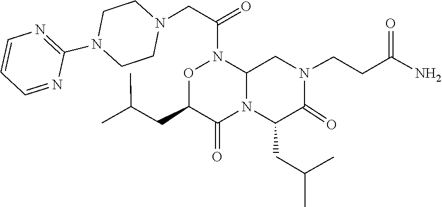 [SMILES: CC(C)C[C@H]1ON(C(=O)CN2CCN(c3ncccn3)CC2)C2CN(CCC(N)=O)C(=O)[C@H](CC(C)C)N2C1=O] | 3-((3R,6S)-3,6-diisobutyl-4,7- dioxo-1-(2-(4-(pyrimidin-2- yl)piperazin-1- yl)acetyl)hexahydropyrazino [2,1-c][1,2,4]oxadiazin- 8(1H)-yl)propanamide | 558.7 | 558.3 | 559.1 | [M + H]⁺ | D |
| I-171 | 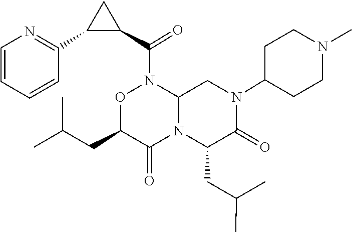 [SMILES: CC(C)C[C@H]1ON(C(=O)[C@@H]2C[C@H]2c2ccccn2)C2CN(C3CCN(C)CC3)C(=O)[C@H](CC(C)C)N2C1=O] | (3R,6S)-3,6-diisobutyl-8-(1- methylpiperidin-4-yl)-1- ((1R,2R)-2-(pyridin-2- yl)cyclopropane-1- carbonyl)tetrahydropyrazino [2,1-c][1,2,4]oxadiazine- 4,7(3H,6H)-dione | 525.7 | 525.3 | 526.5 | [M + H]⁺ | B |
| I-172 | 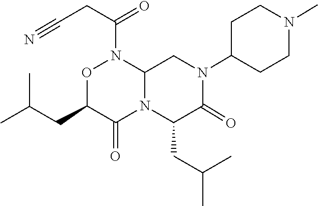 [SMILES: CC(C)C[C@H]1ON(C(=O)CC#N)C2CN(C3CCN(C)CC3)C(=O)[C@H](CC(C)C)N2C1=O] | 3-((3R,6S)-3,6-diisobutyl-8- (1-methylpiperidin-4-yl)-4,7- dioxohexahydropyrazino[2,1- c][1,2,4]oxadiazin-1(6H)-yl)- 3-oxopropanenitrile | 447.6 | 447.3 | 448.4 | [M + H]⁺ | B |
| I-173 | 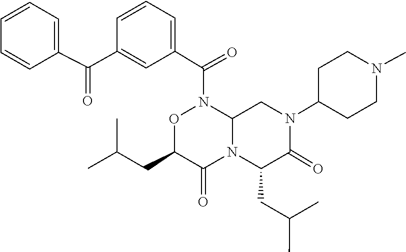 [SMILES: CC(C)C[C@H]1ON(C(=O)c2cccc(C(=O)c3ccccc3)c2)C2CN(C3CCN(C)CC3)C(=O)[C@H](CC(C)C)N2C1=O] | (3R,6S)-1-(3- benzoylbenzoyl)-3,6- diisobutyl-8-(1- methylpiperidin-4- yl)tetrahydropyrazino[2,1- c][1,2,4]oxadiazine- 4,7(3H,6H)-dione | 588.7 | 588.3 | 589.4 | [M + H]⁺ | B |

TABLE 12-27

| | | | | | | | |
|---|---|---|---|---|---|---|---|
| I-174 | 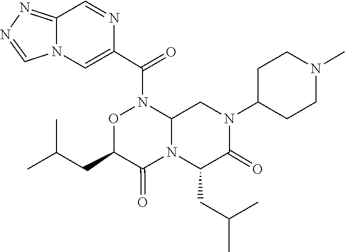 [SMILES: CC(C)C[C@H]1ON(C(=O)c2cn3cnnc3cn2)C2CN(C3CCN(C)CC3)C(=O)[C@H](CC(C)C)N2C1=O] | (3R,6S)-1- ([1,2,4]triazolo[4,3- a]pyrazine-6-carbonyl)-3,6 diisobutyl-8-(1- methylpiperidin-4- yl)tetrahydropyrazino[2,1- c][1,2,4]oxadiazine- 4,7(3H,6H)-dione | 526.6 | 526.3 | 527.4 | [M + H]⁺ | B |
| I-175 | 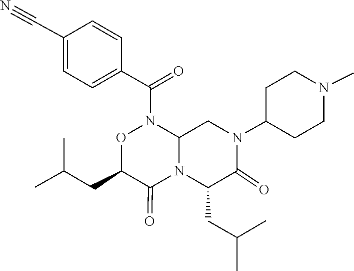 [SMILES: CC(C)C[C@H]1ON(C(=O)c2ccc(C#N)cc2)C2CN(C3CCN(C)CC3)C(=O)[C@H](CC(C)C)N2C1=O] | 4-((3R,6S)-3,6-diisobutyl-8- (1-methylpiperidin-4-yl)-4,7- dioxooctahydropyrazino[2,1- c][1,2,4]oxadiazine-1- carbonyl)benzonitrile | 509.7 | 509.3 | 510.4 | [M + H]⁺ | B |
| I-176 | 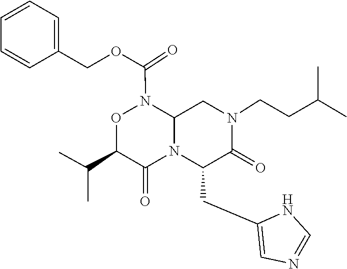 [SMILES: CC(C)CCN1CC2N(C(=O)OCc3ccccc3)O[C@H](C(C)C)C(=O)N2[C@@H](Cc2cnc[nH]2)C1=O] | benzyl (3R,6S)-6-((1H- imidazol-5-yl)methyl)-8- isopentyl-3-isopropyl-4,7- dioxohexahydropyrazino[2,1- c][1,2,4]oxadiazine-1(6H)- carboxylate | 497.6 | 497.3 | 498.2 | [M + H]⁺ | B |
| I-177 | 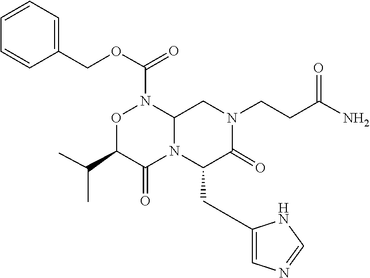 [SMILES: CC(C)[C@H]1ON(C(=O)OCc2ccccc2)C2CN(CCC(N)=O)C(=O)[C@H](Cc3cnc[nH]3)N2C1=O] | benzyl (3R,6S)-6-(1H- imidazol-5-yl)methyl)-8-(3- amino-3-oxopropyl)-3- isopropyl-4,7- dioxohexahydropyrazino[2,1- c][1,2,4]oxadiazine-1(6H)- carboxylate | 498.5 | 498.2 | 499.2 | [M + H]⁺ | B |
| I-178 | 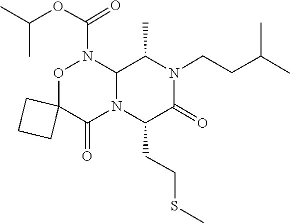 [SMILES: CSCC[C@H]1C(=O)N(CCC(C)C)[C@@H](C)C2N(C(=O)OC(C)C)OC3(CCC3)C(=O)N21] | isopropyl (6′S,9′S)-8′- isopentyl-9′-methyl-6′-(2- (methylthio)ethyl)-4′,7′- dioxotetrahydro-4′H- spiro[cyclobutane-1,3′- pyrazino[2,1- c][1,2,4]oxadiazine]-1′(6′H)- carboxylate | 455.6 | 455.2 | 456.0 478.1 | [M + H]⁺ [M + Na]⁺ | D |
| I-179 | 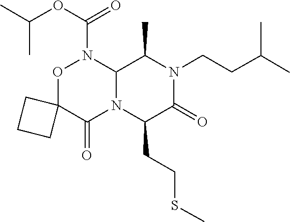 [SMILES: CSCC[C@@H]1C(=O)N(CCC(C)C)[C@H](C)C2N(C(=O)OC(C)C)OC3(CCC3)C(=O)N21] | isopropyl (6′R,9′R)-8′- isopentyl-9′-methyl-6′-(2- (methylthio)ethyl)-4′,7′- dioxotetrahydro-4′H- spiro[cyclobutane-1,3′- pyrazino[2,1- c][1,2,4]oxadiazine]-1′(6′H)- carboxylate | 455.6 | 455.2 | 456.0 478.2 | [M + H]⁺ [M + Na]⁺ | D |
| I-180 | 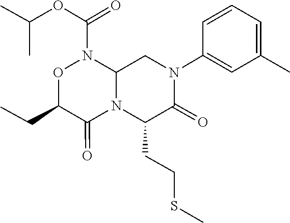 [SMILES: CC[C@H]1ON(C(=O)OC(C)C)C2CN(c3cccc(C)c3)C(=O)[C@H](CCSC)N2C1=O] | isopropyl (3R,6S)-3-ethyl-6- (2-(methylthio)ethyl)-4,7- dioxo-8-(m- tolyl)hexahydropyrazino[2,1- c][1,2,4]oxadiazine-1(6H)- carboxylate | 449.6 | 449.2 | 450.1 | [M + H]⁺ | D |

TABLE 12-28

| | | | | | | | |
|---|---|---|---|---|---|---|---|
| I-181 | 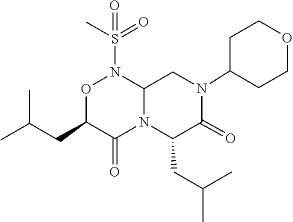 [SMILES: CC(C)C[C@H]1ON(S(C)(=O)=O)C2CN(C3CCOCC3)C(=O)[C@H](CC(C)C)N2C1=O] | (3R,6S)-3,6-diisobutyl-1- (methylsulfonyl)-8- (tetrahydro-2H-pyran-4- yl)tetrahydropyrazino[2,1- c][1,2,4]oxadiazine- 4,7(3H,6H)-dione | 445.6 | 445.2 | 446.2 | [M + H]⁺ | B |
| I-182 | 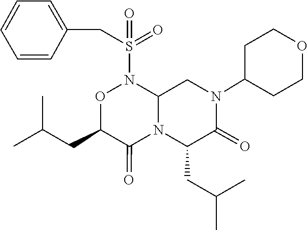 [SMILES: CC(C)C[C@H]1ON(S(=O)(=O)Cc2ccccc2)C2CN(C3CCOCC3)C(=O)[C@H](CC(C)C)N2C1=O] | (3R,6S)-1-(benzylsulfonyl)- 3,6-diisobutyl-8-(tetrahydro- 2H-pyran-4- yl)tetrahydropyrazino[2,1- c][1,2,4]oxadiazine- 4,7(3H,6H)-dione | 521.7 | 521.3 | 522.2 | [M + H]⁺ | B |
| I-183 | 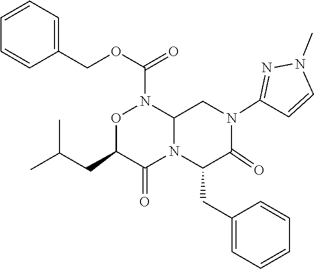 [SMILES: CC(C)C[C@H]1ON(C(=O)OCc2ccccc2)C2CN(c3ccn(C)n3)C(=O)[C@H](Cc3ccccc3)N2C1=O] | benzyl (3R,6S)-6-benzyl-3- isobutyl-8-(1-methyl-1H- pyrazol-3-yl)-4,7- dioxohexahydropyrazino[2,1- c][1,2,4]oxadiazine-1(6H)- carboxylate | 531.6 | 531.2 | 532.2 | [M + H]⁺ | B |

### Experimental Example: Notch Assay

We used the CellSensor T-REx™ NICD CSL-bla HeLa cell line, which was engineered by lentiviral transduction of HeLa cells with a Notch response element driving beta-lactamase reporter gene expression (CSL-bla) along with DOX (doxycycline)-inducible NICD (Notch intracellular domain) constructs. Addition of DOX to these cells allows for regulated NICD transcription factor expression and subsequent beta-lactamase expression.

Specifically, the following protocol was used.

### NOTCH ASSAY PROTOCOL (Method 2)

1. Add 40 nL/well of working concentration of 1000× compound to 384-well assay plate with Echo.
2. Plate Cells: 35 μl/well of HeLa/T-REx™ NICD CSL-bla cells at 10³ cells/well complete medium into 384-well plate.
3. Add 5 μl 8× Doxycycline in Assay Medium to the treated wells, and 5 μl Assay Medium to the Untreated and cell free control wells.
4. Incubate the assay plate in a humidified 37° C./5% CO2 incubator for 16-20 hours.
5. Preparation of 6× LiveBLAzer™-FRET B/G Substrate (CCF4-AM) Mixture according to the manual and cell loading should be done in the absence of direct strong lighting.
6. Remove assay plate from the humidified 37° C./5% CO2 incubator. Add 8 μl of 6× Substrate Mixture from Step 5 to each well. Cover the plate to protect it from light and evaporation. Incubate at room temperature for 4 hours.
7. Reading an Assay Plate using the following filter selections:

| Envision setting | Scan 1 | Scan 2 |
|---|---|---|
| Purpose | Measure fluorescence | Measure fluorescence |
| | in the blue channel | in the green channel |
| Excitation filter | 409/20 nm | 409/20 nm |
| Emission filter | 460/40 nm | 530/30 nm |

8. Data analysis: Use the assay plate layout to identify the location of the Cell-free wells. These control wells are used for background subtraction. Determine the average emission from the Cell-free wells at both 460 nm (Average Blue Background) and 530 nm(Average Green Background). Subtract the Average Blue Background (data collected at 460 nm) from all of the blue emission data. Subtract the Average Green background (data collected at 530 nm) from all of the green emission data. Calculate the Blue/Green Emission Ratio for each well, by dividing the background-subtracted blue emission values by the background-subtracted green emission values.

Instead of Step 1 to Step 3, the following Step a to Step d can also be performed. The subsequent steps are the same as Method 2, after Step 4 (Method 1).
a. Plate Cells: 32 μL/well of HeLa/T-REx™ NICD CSL-bla cells at 10³ cells/well complete medium into 384-well plate.
b. Prepare 10× compounds in assay medium.
c. Add 4 μL compounds or 1% DMSO in assay medium to the cells.
d. Add 4 μL 10×Doxycycline in Assay Medium to the treated wells, and 4 μL Assay Medium to the Untreated and cell free control wells.

The results are shown in Table 13 (Table 13-1 to Table 13-2).

TABLE 13-1

| Compound | inhibiton (%) | | Compound | inhibiton (%) | |
|---|---|---|---|---|---|
| No. | at 10 μM | Method | No. | at 10 μM | Method |
| I-1 | 55.9 | 1 | I-85 | 66.5 | 2 |
| I-2 | 53.5 | 1 | I-86 | 70.9 | 2 |
| I-3 | 53.6 | 1 | I-87 | 58.3 | 2 |
| I-4 | 57.1 | 1 | I-88 | 63.9 | 2 |
| I-5 | 66.1 | 1 | I-89 | 58.3 | 2 |
| I-6 | 62.1 | 1 | I-90 | 62.4 | 2 |
| I-7 | 55.5 | 1 | I-91 | 51.0 | 2 |
| I-8 | 60.2 | 1 | I-92 | 50.1 | 2 |
| I-9 | 70.0 | 1 | I-93 | 76.3 | 2 |
| I-10 | 57.2 | 1 | I-94 | 57.8 | 2 |
| I-11 | 51.7 | 1 | I-95 | 60.1 | 2 |
| I-12 | 71.0 | 1 | I-96 | 56.3 | 2 |
| I-13 | 55.5 | 1 | I-97 | 57.3 | 2 |
| I-14 | 64.8 | 1 | I-98 | 60.4 | 2 |
| I-15 | 52.6 | 1 | I-99 | 58.0 | 2 |
| I-16 | 79.8 | 1 | I-100 | 63.8 | 2 |
| I-17 | 88.0 | 1 | I-101 | 54.4 | 2 |
| I-18 | 54.0 | 1 | I-102 | 67.3 | 2 |
| I-19 | 67.3 | 1 | I-103 | 59.9 | 2 |
| I-20 | 66.1 | 1 | I-104 | 70.2 | 2 |
| I-21 | 60.0 | 1 | I-105 | 60.9 | 2 |
| I-22 | 72.8 | 1 | I-106 | 52.0 | 2 |
| I-23 | 50.4 | 1 | I-107 | 61.1 | 2 |
| I-24 | 68.1 | 1 | I-108 | 72.4 | 2 |
| I-25 | 70.3 | 1 | I-109 | 74.0 | 2 |
| I-26 | 68.4 | 1 | I-110 | 75.2 | 2 |
| I-27 | 62.9 | 1 | I-111 | 53.9 | 2 |
| I-28 | 99.7 | 2 | I-112 | 54.2 | 2 |
| I-29 | 101.2 | 2 | I-113 | 62.9 | 2 |
| I-30 | 117.2 | 2 | I-114 | 56.3 | 2 |

TABLE 13-2

| | | | | | |
|---|---|---|---|---|---|
| I-31 | 67.1 | 2 | I-115 | 51.1 | 2 |
| I-32 | 59.8 | 2 | I-116 | 62.5 | 2 |
| I-33 | 59.6 | 2 | I-117 | 59.7 | 2 |
| I-34 | 119.6 | 2 | I-118 | 63.3 | 2 |
| I-35 | 54.0 | 2 | I-119 | 55.7 | 2 |
| I-36 | 52.2 | 2 | I-120 | 67.1 | 2 |
| I-37 | 87.3 | 2 | I-121 | 63.3 | 2 |
| I-38 | 97.3 | 2 | I-122 | 79.9 | 2 |
| I-39 | 52.1 | 2 | I-123 | 52.3 | 2 |
| I-40 | 123.5 | 2 | I-124 | 57.1 | 2 |
| I-41 | 59.3 | 2 | I-125 | 59.6 | 2 |
| I-42 | 95.0 | 2 | I-126 | 67.2 | 2 |
| I-43 | 52.9 | 2 | I-127 | 62.1 | 2 |
| I-44 | 63.1 | 2 | I-135 | 110.1 | 2 |
| I-45 | 54.9 | 2 | I-136 | 79.7 | 2 |
| I-46 | 75.6 | 2 | I-137 | 81.8 | 2 |
| I-47 | 71.4 | 2 | I-138 | 98.2 | 2 |
| I-48 | 57.3 | 2 | I-139 | 116.3 | 2 |
| I-49 | 92.6 | 2 | I-140 | 60.9 | 2 |
| I-50 | 64.6 | 2 | I-141 | 83.1 | 2 |
| I-51 | 55.3 | 2 | I-142 | 113.4 | 2 |
| I-52 | 51.4 | 2 | I-143 | 105.0 | 2 |
| I-53 | 53.6 | 2 | I-144 | 78.1 | 2 |
| I-54 | 60.4 | 2 | I-145 | 89.0 | 2 |
| I-55 | 58.7 | 2 | I-146 | 105.4 | 2 |
| I-56 | 53.0 | 2 | I-147 | 50.2 | 2 |
| I-57 | 63.4 | 2 | I-148 | 76.6 | 2 |
| I-80 | 63.0 | 2 | I-149 | 113.8 | 2 |
| I-81 | 53.0 | 2 | I-150 | 69.1 | 2 |
| I-82 | 51.2 | 2 | I-151 | 88.0 | 2 |
| I-83 | 76.2 | 2 | I-152 | 93.5 | 2 |
| I-84 | 78.4 | 2 | | | |

### INDUSTRIAL APPLICABILITY

The compound of the present invention inhibits Notch signal transduction, and thus can be used for treating diseases involving Notch signal transduction.

Although only some exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention.

This application is based on U.S. provisional patent application No. 63/090,185 (filing date: Oct. 10, 2020) filed in US, the contents of which are incorporated in full herein.

1. A compound represented by the following formula (I): 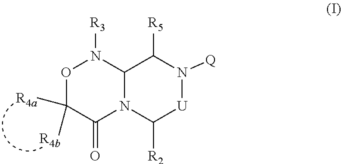
wherein
Q is represented by any of the following formulas (I-1) to (I-2): 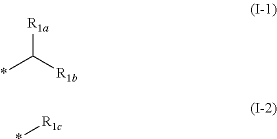
* is a binding site with N (nitrogen atom);
R₁ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl;
R_{1b} is hydrogen, optionally substituted alkyl or —W₁₁-W₁₂—R₁₃
wherein
W₁₁ is —(CO)— or —(SO₂)—,
W₁₂ is a bond, —O— or —N(R₁₄)—,
R₁₃ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl,
R₁₄ is hydrogen or optionally substituted alkyl,
R₁₃ and R₁₄ may combine to form a saturated or unsaturated 4 to 7-membered ring, which may contain carbon atom, nitrogen atom, or oxygen atom, and an aryl ring or a heteroaryl ring may be fused to the ring,
a substituent —X₁₅—R₁₅ may be substituted on the formed saturated or unsaturated 4-7 membered ring or on the fused aryl ring or heteroaryl ring,
X₁₅ is —O—, —NH— or single bond,
R₁₅ is hydrogen, optionally substituted alkyl, optionally substituted aryl or optionally substituted heteroaryl;
R_{1c} is optionally substituted cycloalkyl, optionally substituted heterocycloalkyl, optionally substituted aryl or optionally substituted heteroaryl;
U is —(CO)— or —CH₂—,
R₂ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ is —W₃₁-W₃₂—R₃₃
wherein
W₃₁ is —(CO)—, —(SO₂)—, or —CH₂—
W₃₂ is —O—, —NH—, or single bond, and
R₃₃ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4b} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐ and R_{4b} optionally form a spiro ring together with a carbon bonded thereto, which optionally has oxygen atom or nitrogen atom in the ring; and
R₅ is hydrogen or optionally substituted alkyl,
or a pharmaceutically acceptable salt thereof;
provided that (3R,6S)isopropyl-3-ethyl-8-isopentyl-6-(2-(methylthio)ethyl)-4,7-dioxohexahydropyrazino[2,1-c][1,2,4]oxadiazine-1(6H)-carboxylate is excluded.

2. The compound according to claim 1, wherein
R₁ₐ is hydrogen, optionally substituted alkyl, optionally substituted aryl, optionally substituted heteroaryl, optionally substituted cycloalkyl, or optionally substituted arylalkyl;
R_{1b} is hydrogen, optionally substituted alkyl or —W_{11′}-W_{12′}—R_{13′}
wherein
W_{11′} is —(CO)—,
W_{12′} is —NH—, and
R_{13′} is optionally substituted alkyl, or optionally substituted arylalkyl;
R₂ is optionally substituted alkyl, optionally substituted arylalkyl, optionally substituted heteroarylalkyl, or optionally substituted cycloalkylalkyl;
R₃ is —W_{31′}-W_{32′}—R_{33′}
wherein
W_{31′} is —(CO)—, —(SO₂)—, or —CH₂—,
W_{32′} is —O—, —NH—, or single bond, and
R_{33′} is optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐ is optionally substituted alkyl, optionally substituted arylalkyl, or optionally substituted cycloalkylalkyl;
R_{4b} is hydrogen or optionally substituted alkyl;
R₄ₐ and R_{4b} optionally form a spiro ring together with a carbon bonded thereto, which optionally has oxygen atom or nitrogen atom in the ring;
R₅ is hydrogen or optionally substituted alkyl;
or a pharmaceutically acceptable salt thereof.

3. The compound according to claim 1, which is represented by the following formula (Ia): 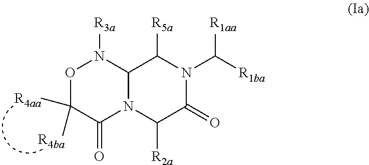
wherein
R₁ₐₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl, or optionally substituted heterocycloalkylalkyl;
R_{1ba} is hydrogen, optionally substituted alkyl or —W₁₁ₐ-W₁₂ₐ—R₁₃ₐ
wherein
W₁₁ₐ is —(CO)— or —(SO₂)—,
W₁₂ₐ is a bond, —O— or —NH—, and
R₁₃ₐ is hydrogen, optionally substituted alkyl, optionally substituted alkenyl, optionally substituted alkynyl, optionally substituted aryl, optionally substituted heteroaryl, optionally substituted cycloalkyl, optionally substituted arylalkyl, optionally substituted heteroarylalkyl, optionally substituted cycloalkylalkyl or optionally substituted heterocycloalkylalkyl;
R₂ₐ is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₃ₐ is —W₃₁ₐ-W₃₂ₐ—R₃₃ₐ
wherein
W₃₁ₐ is —(CO)— or —(SO₂)—,
W₃₂ₐ is —O— or —NH—, and
R₃₃ₐ is hydrogen, optionally substituted alkyl, optionally substituted aryl, optionally substituted heteroaryl, optionally substituted cycloalkyl, optionally substituted heterocycloalkyl, optionally substituted arylalkyl, optionally substituted heteroarylalkyl, optionally substituted cycloalkylalkyl or optionally substituted heterocycloalkylalkyl;
R₄ₐₐ is optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R_{4ba} is hydrogen, optionally substituted alkyl, optionally substituted alkenyl, optionally substituted alkynyl, optionally substituted arylalkyl, optionally substituted heteroarylalkyl, optionally substituted cycloalkylalkyl or optionally substituted heterocycloalkylalkyl;
R₄ₐₐ and R_{4ba} optionally form a spiro ring together with a carbon bonded thereto; and
R₅ₐ is hydrogen or optionally substituted alkyl,
or a pharmaceutically acceptable salt thereof;
provided that (3R,6S)isopropyl-3-ethyl-8-isopentyl-6-(2-(methylthio)ethyl)-4,7-dioxohexahydropyrazino[2,1-c][1,2,4]oxadiazine-1(6H)-carboxylate is excluded.

4. The compound according to claim 3, wherein
R₁ₐₐ is hydrogen, optionally substituted alkyl, optionally substituted aryl, optionally substituted heteroaryl, optionally substituted cycloalkyl, or optionally substituted arylalkyl;
R_{1ba} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl, or optionally substituted arylalkyl;
R₂ₐ is optionally substituted alkyl, optionally substituted arylalkyl, or optionally substituted cycloalkylalkyl;
R₃ₐ is —W_{31a′}-W_{32a′}—R_{33a′}
wherein
W_{31a′} is —(CO)—,
W_{32a′} is —O— or —NH—, and
R_{33a′} is optionally substituted alkyl, optionally substituted arylalkyl, optionally substituted heteroarylalkyl, or optionally substituted cycloalkylalkyl;
R₄ₐₐ is optionally substituted alkyl, optionally substituted arylalkyl, or optionally substituted cycloalkylalkyl;
R_{4ba} is hydrogen;
R₅ₐ is hydrogen,
or a pharmaceutically acceptable salt thereof.

5. The compound according to claim 1, wherein
R₁ₐ is optionally substituted alkyl, optionally substituted aryl, optionally substituted cycloalkyl, or optionally substituted arylalkyl;
R_{1b} is hydrogen, optionally substituted alkyl or —W_{11′}-W_{12′}—R_{13′}
wherein
W_{11′} is —(CO)—,
W_{12′} is —NH—, and
R_{13′} is optionally substituted alkyl, or optionally substituted arylalkyl,
or a pharmaceutically acceptable salt thereof.

6. The compound according to claim 3, wherein
R₁ₐₐ is optionally substituted alkyl, optionally substituted aryl, optionally substituted cycloalkyl, or optionally substituted arylalkyl;
R_{1ba} is hydrogen, optionally substituted alkyl or —W_{11a′}-W_{12a′}—R_{13a′}
wherein
W_{11a′} is —(CO)—,
W_{12a′} is —NH—, and
R_{13a′} is optionally substituted alkyl, or optionally substituted arylalkyl,
or a pharmaceutically acceptable salt thereof.

7. The compound according to claim 1, wherein
R_{1c} is optionally substituted heterocycloalkyl,
or a pharmaceutically acceptable salt thereof.

8. The compound according to claim 1, wherein
U is —(CO)—,
or a pharmaceutically acceptable salt thereof.

9. The compound according to claim 1, wherein
R₂ is optionally substituted alkyl, optionally substituted arylalkyl, or optionally substituted cycloalkylalkyl,
or a pharmaceutically acceptable salt thereof.

10. The compound according to claim 3, wherein
R₂ₐ is optionally substituted alkyl, optionally substituted arylalkyl, or optionally substituted cycloalkylalkyl,
or a pharmaceutically acceptable salt thereof.

11. The compound according to claim 1, wherein
R₃ is —W_{31′}-W_{32′}—R_{33′}
wherein
W_{31′} is —(CO)— or —CH₂—,
W_{32′} is —O—, —NH—, or single bond, and
R_{33′} is optionally substituted alkyl, optionally substituted arylalkyl, optionally substituted cycloalkylalkyl or optionally substituted heterocycloalkylalkyl,
or a pharmaceutically acceptable salt thereof.

12. The compound according to claim 1, wherein
R₄ₐ is optionally substituted alkyl, optionally substituted arylalkyl, or optionally substituted cycloalkylalkyl,
R_{4b} is hydrogen, and
R₅ is hydrogen,
or a pharmaceutically acceptable salt thereof.

13. The compound according to claim 3, wherein
R₄ₐₐ is optionally substituted alkyl, optionally substituted arylalkyl, or optionally substituted cycloalkylalkyl,
R_{4ba} is hydrogen, and
R₅ₐ is hydrogen,
or a pharmaceutically acceptable salt thereof.

14. (canceled)

15. (canceled)

16. A method of treating or preventing a disease involving Notch signal transduction comprising administering to a subject in need thereof a compound according to claim 1 or a pharmaceutically acceptable salt thereof, in an amount effective to treat or prevent the disease.

17. (canceled)

18. (canceled)
